SEQUENCE LISTING (228 sequences)

SEQ ID NO: 1 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 115 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLS

SEQ ID NO: 3 - protein, 23 aa, Homo sapiens
VIGFRILLLKVAGFNLLMTLRLW

SEQ ID NO: 4 - skipped

SEQ ID NO: 5 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 6 - protein, 149 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYE

SEQ ID NO: 7 - protein, 21 aa, Homo sapiens
ILLGKATLYAVLVSALVLMAM

SEQ ID NO: 8 - protein, 6 aa, Homo sapiens
VKRKDF

SEQ ID NO: 9 - protein, 178 aa, Homo sapiens
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 10 - protein, 144 aa, Homo sapiens
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSA

SEQ ID NO: 11 - protein, 23 aa, Homo sapiens
TILYEILLGKATLYAVLVSALVL

SEQ ID NO: 12 - protein, 11 aa, Homo sapiens
MAMVKRKDSRG

SEQ ID NO: 13 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 14 - protein, 138 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSA

SEQ ID NO: 15 - protein, 23 aa, Homo sapiens
YYMYLLLLLKSVVYFAIITCCLL

SEQ ID NO: 16 - protein, 12 aa, Homo sapiens
RRTAFCCNGEKS

SEQ ID NO: 17 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 18 - protein, 154 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSA

SEQ ID NO: 19 - protein, 23 aa, Homo sapiens
YYTYLLLLLKSVVYFAIITCCLL

SEQ ID NO: 20 - protein, 12 aa, Homo sapiens
RRTAFCCNGEKS

SEQ ID NO: 21 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 22 - protein, 129 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 23 - protein, 23 aa, Homo sapiens
LGLRMLFAKTVAVNFLLTAKLFF

SEQ ID NO: 24 - skipped

SEQ ID NO: 25 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 26 - protein, 22 aa, Homo sapiens
MQSGTHWRVLGLCLLSVGVWGQ

SEQ ID NO: 27 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 28 - protein, 26 aa, Homo sapiens
VMSVATIVIVDICITGGLLLLVYYWS

SEQ ID NO: 29 - protein, 55 aa, Homo sapiens
KNRKAKAKPVTRGAGAGGRQRGQNKERPPPVPNPDYEPIRKGQRDLYSGLNQRRI

SEQ ID NO: 30 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 31 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 32 - protein, 21 aa, Homo sapiens
MEHSTFLSGLVLATLLSQVSP

SEQ ID NO: 33 - protein, 84 aa, Homo sapiens [sequence omitted]

SEQ ID NO: 34 - protein, 21 aa, Homo sapiens
GIIVTDVIATLLLALGVFCFA

SEQ ID NO: 35 - protein, 45 aa, Homo sapiens
GHETGRLSGAADTQALLRNDQVYQPLRDRDDAQYSHLGGNWARNK

SEQ ID NO: 36 - protein, 150 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 37 - protein, 171 aa, Homo sapiens; identical to SEQ ID NO: 31

SEQ ID NO: 38 - protein, 21 aa, Homo sapiens
MEHSTFLSGLVLATLLSQVSP

SEQ ID NO: 39 - protein, 84 aa, Homo sapiens [sequence omitted]

SEQ ID NO: 40 - protein, 21 aa, Homo sapiens
GIIVTDVIATLLLALGVFCFA

SEQ ID NO: 41 - protein, 45 aa, Homo sapiens; identical to SEQ ID NO: 35

SEQ ID NO: 42 - protein, 150 aa, Homo sapiens; identical to SEQ ID NO: 36

SEQ ID NO: 43 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 21 aa, Homo sapiens
MKWKALFTAAILQAQLPITEA

SEQ ID NO: 45 - protein, 9 aa, Homo sapiens
QSFGLLDPK

SEQ ID NO: 46 - protein, 21 aa, Homo sapiens
LCYLLDGILFIYGVILTALFL

SEQ ID NO: 47 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 48 - protein, 143 aa, Homo sapiens
QSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 49 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 142 aa, Homo sapiens
QSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 140 aa, Homo sapiens
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 53 - protein, 176 aa, Homo sapiens
DLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 54 - protein, 178 aa, Homo sapiens
DLKNVFPPKVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 55 - protein, 119 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSS

SEQ ID NO: 56 - protein, 5 aa, synthetic construct
RYWMN

SEQ ID NO: 57 - protein, 17 aa, synthetic construct
EITPDSSTINYTPSLKG

SEQ ID NO: 58 - protein, 10 aa, synthetic construct
PYDYGAWFAS

SEQ ID NO: 59 - protein, 30 aa, synthetic construct
EVQLVESGGGLVQPGGSLRLSCAASGFDFS

SEQ ID NO: 60 - protein, 14 aa, synthetic construct
WVRQAPGKGLEWVG

SEQ ID NO: 61 - protein, 32 aa, synthetic construct
RFTISRDNAKNSLYLQMNSLRAEDTAVYYCVR

SEQ ID NO: 62 - protein, 11 aa, synthetic construct
WGQGTLVTVSS

SEQ ID NO: 63 - protein, 119 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSS

SEQ ID NO: 64 - protein, 17 aa, synthetic construct
EITPDSSTINYAPSLKG

SEQ ID NO: 65 - protein, 119 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSS

SEQ ID NO: 66 - protein, 30 aa, synthetic construct
EVQLVESGGGLVQPGGSLRLSCAASGFTFS

SEQ ID NO: 67 - protein, 32 aa, synthetic construct
RFTISRDNAKNSLYLQMNSLRAEDTAVYYCAR

SEQ ID NO: 68 - protein, 109 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 69 - protein, 14 aa, synthetic construct
RSSTGAVTTSNYAN

SEQ ID NO: 70 - protein, 7 aa, synthetic construct
GTNKRAP

SEQ ID NO: 71 - protein, 9 aa, synthetic construct
ALWYSNHWV

SEQ ID NO: 72 - protein, 22 aa, synthetic construct
QAVVTQEPSLTVSPGGTVTLTC

SEQ ID NO: 73 - protein, 15 aa, synthetic construct
WVQEKPDHLFTGLIG

SEQ ID NO: 74 - protein, 32 aa, synthetic construct
GTPARFSGSLLGGKAALTLSGAQPEDEAEYYC

SEQ ID NO: 75 - protein, 10 aa, synthetic construct
FGGGTKLTVL

SEQ ID NO: 76 - protein, 121 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSS

SEQ ID NO: 77 - protein, 5 aa, synthetic construct
SYGMS

SEQ ID NO: 78 - protein, 6 aa, synthetic construct
SSGGSY

SEQ ID NO: 79 - protein, 12 aa, synthetic construct
LGMITTGYAMDY

SEQ ID NO: 80 - protein, 30 aa, synthetic construct
EVQLVESGGDLVKPGGSLKLSCAASGFTFS

SEQ ID NO: 81 - protein, 16 aa, synthetic construct
WVRQTPDKRLEWVATI

SEQ ID NO: 82 - protein, 41 aa, synthetic construct
IYYPDSVKGRFTISRDNAKNTLYLQMSSLKSEDTAMYYCAR

SEQ ID NO: 83 - protein, 11 aa, synthetic construct
WGQGTSVTVSS

SEQ ID NO: 84 - protein, 112 aa, synthetic construct
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIK

SEQ ID NO: 85 - protein, 16 aa, synthetic construct
RSSQTIVHSTGHTYLE

SEQ ID NO: 86 - protein, 7 aa, synthetic construct
KVSNRFS

SEQ ID NO: 87 - protein, 9 aa, synthetic construct
FQGSHVPYT

SEQ ID NO: 88 - protein, 23 aa, synthetic construct
DVLMTQTPLSLPVSLGDQASISC

SEQ ID NO: 89 - protein, 15 aa, synthetic construct
WFLQKPGQSPKLLIY

SEQ ID NO: 90 - protein, 32 aa, synthetic construct
GVPDRFSGSGSGTDFTLKISRVEAEDLGVYYC

SEQ ID NO: 91 - protein, 10 aa, synthetic construct
FGGGTKLEIK

SEQ ID NO: 92 - protein, 5 aa, synthetic construct
GGGGS

SEQ ID NO: 93 - protein, 15 aa, synthetic construct
GGGGSGGGGSGGGGS

SEQ ID NO: 94 - protein, 243 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 95 - protein, 243 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 96 - protein, 243 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 97 - protein, 248 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSGGGGSGGGGSGGGGSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIK

SEQ ID NO: 98 - protein, 19 aa, synthetic construct
MGWSCIILFLVATATGVHS

SEQ ID NO: 99 - protein, 48 aa, Homo sapiens
AKPTTTPAPRPPTPAPTIASQPLSLRPEACRPAAGGAVHTRGLDFACD

SEQ ID NO: 100 - protein, 21 aa, Homo sapiens
IYIWAPLAGTCGVLLLSLVIT

SEQ ID NO: 101 - protein, 42 aa, Homo sapiens
KRGRKKLLYIFKQPFMRPVQTTQEEDGCSCRFPEEEEGGCEL

SEQ ID NO: 102 - protein, 113 aa, Homo sapiens; identical to SEQ ID NO: 47

SEQ ID NO: 103 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 104 - protein, 21 aa, synthetic construct
GSGEGRGSLLTCGDVEENPGP

SEQ ID NO: 105 - protein, 750 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 106 - protein, 731 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 107 - protein, 491 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 108 - protein, 472 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 109 - protein, 23 aa, synthetic construct
GSGQCTNYALLKLAGDVESNPGP

SEQ ID NO: 110 - protein, 721 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 111 - protein, 702 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 112 - protein, 462 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 113 - protein, 443 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 114 - protein, 721 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 115 - protein, 702 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 116 - protein, 462 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 117 - protein, 443 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 118 - protein, 721 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 119 - protein, 702 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 120 - protein, 462 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 121 - protein, 443 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 122 - protein, 726 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSGGGGSGGGGSGGGGSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 123 - protein, 707 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSGGGGSGGGGSGGGGSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 124 - protein, 467 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSGGGGSGGGGSGGGGSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 125 - protein, 448 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSGGGGSGGGGSGGGGSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 126 - protein, 537 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 127 - protein, 518 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 128 - protein, 278 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 129 - protein, 259 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 130 - protein, 537 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 131 - protein, 518 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 132 - protein, 278 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 133 - protein, 259 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 134 - protein, 537 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 135 - protein, 518 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 136 - protein, 278 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 137 - protein, 259 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 138 - protein, 527 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 139 - protein, 508 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 140 - protein, 268 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 141 - protein, 249 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 142 - protein, 539 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 143 - protein, 520 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 144 - protein, 280 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 145 - protein, 261 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 146 - protein, 530 aa, synthetic construct
MGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 147 - protein, 511 aa, synthetic construct
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 148 - protein, 271 aa, synthetic construct
MGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 149 - protein, 252 aa, synthetic construct
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 150 - protein, 573 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 151 - protein, 554 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 152 - protein, 314 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 153 - protein, 295 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 154 - protein, 573 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 155 - protein, 554 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 156 - protein, 314 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 157 - protein, 295 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 158 - protein, 573 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 159 - protein, 554 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 160 - protein, 314 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 161 - protein, 295 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 162 - protein, 563 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 163 - protein, 544 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 164 - protein, 304 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 165 - protein, 285 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 166 - protein, 575 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 167 - protein, 556 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 168 - protein, 316 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 169 - protein, 297 aa, synthetic construct
EVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 170 - protein, 566 aa, synthetic construct
MGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 171 - protein, 547 aa, synthetic construct
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 172 - protein, 307 aa, synthetic construct
MGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 173 - protein, 288 aa, synthetic construct
DVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 174 - protein, 862 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 175 - protein, 603 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 176 - protein, 862 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 177 - protein, 603 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 178 - protein, 862 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 179 - protein, 603 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 180 - protein, 867 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 181 - protein, 608 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 182 - protein, 862 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 183 - protein, 603 aa, synthetic construct
MGWSCIILFLVATATGVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 184 - protein, 862 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 185 - protein, 603 aa, synthetic construct
MGWSCIILFLVATATGVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 186 - protein, 862 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 187 - protein, 603 aa, synthetic construct
MGWSCIILFLVATATGVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 188 - protein, 867 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 189 - protein, 608 aa, synthetic construct
MGWSCIILFLVATATGVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 190 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 191 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 192 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 193 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 194 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 195 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 196 - protein, 899 aa, synthetic construct
MGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 197 - protein, 640 aa, synthetic construct
MGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGWSCIILFLVATATGVHSDVLMTQTPLSLPVSLGDQASISCRSSQTIVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 198 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 199 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 200 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 201 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 202 - protein, 894 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 203 - protein, 635 aa, synthetic construct
MGWSCIILFLVATATGVHSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 204 - protein, 899 aa, synthetic construct
MGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 205 - protein, 640 aa, synthetic construct
MGWSCIILFLVATATGVHSDVLMTQTPLSLPVSLGDQASISCRSSQTIVHSTGHTYLEWFLQKPGQSPKLLIYKVSNRFSGVPDRFSGSGSGTDFTLKISRVEAEDLGVYYCFQGSHVPYTFGGGTKLEIKDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGEGRGSLLTCGDVEENPGPMGWSCIILFLVATATGVHSDVLMTQTPLSLPVSLGDQASISCRSSQTIVHSEVQLVESGGDLVKPGGSLKLSCAASGFTFSSYGMSWVRQTPDKRLEWVATISSGGSYIYYPDSVKGRFTISRDNAKNTLYLQMSSLKSEDTAMYYCARLGMITTGYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 206 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 209 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 210 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 211 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 217 aa, Homo sapiens
APELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 217 aa, Homo sapiens
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 217 aa, Homo sapiens
APELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 217 aa, Homo sapiens
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 100 aa, synthetic construct
GGUGGCCACAAUUGUCAUAGGUUUUAGAGCUAGAAAUAGCAAGUUAAAAUAAGGCUAGUCCGUUAUCAACUUGAAAAAGUGGCACCGAGUCGGUGCUUUU

SEQ ID NO: 218 - protein, 100 aa, synthetic construct
GGAGAAUGACGAGUGGACCCGUUUUAGAGCUAGAAAUAGCAAGUUAAAAUAAGGCUAGUCCGUUAUCAACUUGAAAAAGUGGCACCGAGUCGGUGCUUUU

SEQ ID NO: 219 - protein, 100 aa, synthetic construct
AGAGUCUCUCAGCUGGUACAGUUUUAGAGCUAGAAAUAGCAAGUUAAAAUAAGGCUAGUCCGUUAUCAACUUGAAAAAGUGGCACCGAGUCGGUGCUUUU

SEQ ID NO: 220 - protein, 22 aa, synthetic construct
GSGATNFSLLKQAGDVEENPGP

SEQ ID NO: 221 - protein, 25 aa, synthetic construct
GSGVKQTLNFDLLKLAGDVESNPGP

SEQ ID NO: 222 - skipped

SEQ ID NO: 223 - protein, 18 aa, synthetic construct
  VARIANT 2..3: note = wherein X is any amino acid
  VARIANT 6..13: note = wherein X is 6-8 instances of any amino acid
  VARIANT 15..16: note = wherein X is any amino acid
YXXLIXXXXXXXXYXXLI

SEQ ID NO: 224 - DNA, 1329 bp, synthetic construct
  source 1..1329: mol_type = other DNA
gaagtgcagctggttgagtctggcggaggactggttcaacctggcggaagcctgagactgtcttgtgccgccagcggcttcaccttcagcagatactggatgaactgggtgagacaggcccctggcaaaggactggagtgggtgggagagatcacccctgacagcagcaccatcaattacgcccctagcctgaagggcagattcaccatctccagagacaacgccaagaacagcctgtacctgcagatgaactccctgagagccgaggacaccgccgtgtactactgtgccagaccttacgattacggcgcctggtttgcttcttggggccagggaacactggtgacagttagctctggaggaggaggatcaggcggaggcggaagtggtggtggaggatctcaggctgtggtgacacaagagccaagcctgacagtgtctccaggcggcacagtgaccctgacctgtagatcttctaccggagccgtgaccaccagcaactacgccaattgggtgcaagagaagcccgaccacctgtttacaggcctgatcggcggcaccaataagagagcacctggcacaccagccagattctctggatctctgctcggaggaaaggccgctctgacactgtctggagcccagccagaggatgaggccgagtattattgtgccctgtggtacagcaaccactgggtgtttggcggcggaacaaagctgacagttcttggaggtggtggaagcggtggaggtggatctggaggtggcggatccgatggaaatgaggagatgggcggcatcacccagacaccttacaaggtgagcatcagcggcaccaccgtgatcctgacatgtcctcagtaccctggcagcgagatcctgtggcagcacaacgataagaacatcggcggcgacgaggacgacaagaatatcggctctgacgaggatcacctgtctctgaaggagttcagcgagctggagcagagcggctactacgtgtgttaccctagaggcagcaagcccgaggacgccaacttctacctgtatctgagagccagagtgtgcgagaactgcatggagatggatgtgatgagtgtcgctaccatcgtgatcgtcgacatctgtatcacaggcggcctgctgctgctggtgtactattggagcaagaaccggaaggccaaagccaagccagtgacaagaggagcaggagccggaggtagacagagaggccagaacaaagaaaggcctccaccggtgcctaatcctgactacgagcccatcagaaagggccagagagatctgtacagcggcctgaaccagagaagaatc

SEQ ID NO: 225 - DNA, 777 bp, synthetic construct
  source 1..777: mol_type = other DNA
gaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcatccagaaccccgaccccgcagtctatcaattgcgtgatagtaagagtagcgacaagtctgtgtgcctgttcaccgatttcgacagtcagacaaatgtttctcagagtaaggacagtgatgtgtacatcaccgacaaatgcgtcctggacatgcgctccatggacttcaagagtaatagcgccgtagcctggagcaacaaaagcgacttcgcatgtgccaacgcgttcaataactccattatccctgaagatacttttttcccctcccccgaaagtagctgcgatgttaaactcgttgagaagtccttcgaaactgacaccaacctgaacttccagaacctgagtgtgattggcttccgcatcctgctcctgaaagtggccggctttaacctgctcatgacactgcgtctgtggtcaagc

SEQ ID NO: 226 - DNA, 855 bp, synthetic construct
  source 1..855: mol_type = other DNA
caggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtcctagacctgaataaagtgtttcccccagaggtcgccgtgtttgaaccgagcgaggctgagatctcccacacccagaaggctacactggtctgtcttgccaccggtttctttcctgaccacgtggaactgtcttggtgggttaacggtaaggaggtgcactccggtgtgtgcactgatccccagccgttgaaagaacagccggctttgaacgactcccggtactgccttagctcccgcctgcgcgtgtctgctacattctggcagaacccccgcaaccactttcggtgccaggtgcagttttacggcctgtccgagaacgatgaatggacacaagaccgggctaagcccgtgactcagatcgtgagtgctgaggcttgggggcgcgccgattgcggcttcacctcagtcagctaccaacagggcgttctgagtgccacaatcctgtatgagatcctcctgggaaaggcaactctgtacgctgttctcgtctccgcgcttgttctgatggctatggtgaagagaaaggatttc

SEQ ID NO: 227 - DNA, 747 bp, synthetic construct
  source 1..747: mol_type = other DNA
caggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtcctaatccagaaccccgaccccgcagtctatcaattgcgtgatagtaagagtagcgacaagtctgtgtgcctgttcaccgatttcgacagtcagacaaatgtttctcagagtaaggacagtgatgtgtacatcaccgacaaatgcgtcctggacatgcgctccatggacttcaagagtaatagcgccgtagcctggagcaacaaaagcgacttcgcatgtgccaacgcgttcaataactccattatccctgaagatacttttttcccctcccccgaaagtagctgcgatgttaaactcgttgagaagtccttcgaaactgacaccaacctgaacttccagaacctgagtgtgattggcttccgcatcctgctcctgaaagtggccggctttaacctgctcatgacactgcgtctgtggtcaagc

SEQ ID NO: 228 - DNA, 885 bp, synthetic construct
  source 1..885: mol_type = other DNA
gaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcgacctgaataaagtgtttcccccagaggtcgccgtgtttgaaccgagcgaggctgagatctcccacacccagaaggctacactggtctgtcttgccaccggtttctttcctgaccacgtggaactgtcttggtgggttaacggtaaggaggtgcactccggtgtgtgcactgatccccagccgttgaaagaacagccggctttgaacgactcccggtactgccttagctcccgcctgcgcgtgtctgctacattctggcagaacccccgcaaccactttcggtgccaggtgcagttttacggcctgtccgagaacgatgaatggacacaagaccgggctaagcccgtgactcagatcgtgagtgctgaggcttgggggcgcgccgattgcggcttcacctcagtcagctaccaacagggcgttctgagtgccacaatcctgtatgagatcctcctgggaaaggcaactctgtacgctgttctcgtctccgcgcttgttctgatggctatggtgaagagaaaggatttc

(19) United States
(12) Patent Application Publication
(10) Pub. No.: US 2025/0302879 A1
(43) Pub. Date: Oct. 2, 2025

(54) RECOMBINANT T CELL RECEPTORS

(71) Applicant: Hoffmann-La Roche Inc., Little Falls, NJ (US)

(72) Inventors: Stephan GASSER, Fislisbach (CH); Christian KLEIN, Bonstetten (CH); Simone Ricarda LANG, Zuerich (CH); Dario VENETZ, Bonstetten (CH)

(73) Assignee: Hoffmann-La Roche Inc., Little Falls, NJ (US)

(21) Appl. No.: 19/074,066

(22) Filed: Mar. 7, 2025

Related U.S. Application Data
(63) Continuation of application No. PCT/EP2023/074410, filed on Sep. 6, 2023.

(30) Foreign Application Priority Data
Sep. 8, 2022 (EP) 22194514.0
Jan. 5, 2023 (EP) 23150463.0

(51) Int. Cl. A61K 35/17; A61K 40/11; A61K 40/31; A61K 40/42; A61P 35/00; C07K 14/725; C07K 16/28

(52) U.S. Cl. CPC A61K 35/17; A61K 40/11; A61K 40/31; A61K 40/421; A61P 35/00; C07K 14/7051; C07K 16/283; A61K 2239/13; C07K 2317/524; C07K 2317/526; C07K 2317/56; C07K 2317/622; C07K 2319/00

(57) ABSTRACT

The present disclosure relates to the fields of molecular biology, more specifically antigen-binding molecule technology. The present disclosure also relates to methods of medical treatment and prophylaxis, particularly cellular immunotherapy.

### SEQUENCE LISTING

This application contains a Sequence Listing which has been submitted electronically in XML format and is hereby incorporated by reference in its entirety. Said XML copy, created on May 2, 2025, is named P37595-US_sequence_listing_corrected.xml and is 266,260 bytes in size.

### TECHNICAL FIELD

The present disclosure relates to the fields of molecular biology, more specifically antigen-binding molecule technology. The present disclosure also relates to methods of medical treatment and prophylaxis, particularly cellular immunotherapy.

### BACKGROUND

Chimeric antigen receptor (CAR) expressing T cells have shown clinical efficacy in different hematologic malignancies. For solid tumor indications CAR-T cell therapies have yet to provide meaningful clinical benefits over conventional chemo- and immunotherapies. A variety of strategies have been pursued to overcome the limitations of CAR-T cell therapy which can be summarized as systemic toxicities and insufficient CAR-T cell-mediated anti-tumor immune responses. The most widespread CAR format to date are second generation CARs, featuring costimulatory signaling domain, typically from CD28 or 4-1BB costimulatory receptors, and the CD3 zeta T cell receptor (TCR) signaling domain. While this format is widely used, it exhibits inherent limitations which are well documented. Expression levels of second generation CARs are higher than for natural T cells receptors and some of the commonly used antibody-derived single chain fragment variable (scFv) domains may exhibit a tendency to misspair and aggregate on the cell surface under these conditions. This may lead to constitutive CAR signaling in absence of the tumor antigen resulting in toxicity and T cell exhaustion. Hence, research efforts have been devoted to generating CAR formats which exhibit more physiological and strictly antigen-dependent CAR signaling.

One early described strategy aims to add or substitute the antigen specificity of the natural TCR complex. Different approaches to achieve this have been described. By adding antibody variable domains to the CD3 epsilon domains of the TCR, TCR complexes with a second antigen-specificity can be generated (Nolan et al., Clin. Cancer Res. (1999) 5:3928-3941; Baeuerle et al., Nat. Comms. (2019) 10:2087). This additional specificity can mediate peptide-human leukocyte antigen (pHLA)-independent T cell activation via the TCR complex. Another approach aims to substitute the variable alpha and beta domains of the TCR with antibody-derived variable light and variable heavy chain domains (Kuwana et al., Biochem. Biophys. Res. Commun. (1987) 149:960-968; Liu et al., Sci. Transl. Med. (2021) 13:1-16; Mansilla-Soto et. al., Nat. Med. (2022) 28:345-352). This combined with enzyme-mediated gene knock-outs of the endogenous TCR alpha and beta chain encoding genes, results in the loss of the natural T cell specificity and the gain of a new antigen specificity of interest. With both of the above mentioned formats as well as with previously described second generation CAR formats, the final recombinant T cell product will feature a single new antigen specificity unless several different CAR genes are introduced.

We and others have previously described modular CARs which can bind defined adaptor molecules with specificity for different tumor antigens (Darowski et al. MAbs (2019) 11 (4): 621-631). T cells expressing a chimeric antigen receptor (CAR) construct comprising an antigen-binding moiety specific for a variant Fc domain are disclosed in WO 2018/177966 A1. The CAR construct is a second generation CAR, comprising a costimulatory sequence in conjunction with the intracellular domain of CD3Z.

### SUMMARY

In a first aspect, the present disclosure provides a recombinant CD3-TCR complex polypeptide, comprising:
(i) an antigen-binding moiety, or a component thereof, wherein the antigen-binding moiety binds to a variant Fc domain having an amino acid sequence comprising at least one amino acid difference relative to a reference Fc domain, to which the antigen-binding moiety does not bind; and
(ii) a CD3-TCR complex association domain having an amino acid sequence derived from a CD3-TCR complex polypeptide.

In some embodiments, the recombinant CD3-TCR complex polypeptide is capable of associating through its CD3-TCR complex association domain with one or more CD3-TCR complex polypeptides to form a CD3-TCR complex.

In some embodiments, the amino acid sequence derived from a CD3-TCR complex polypeptide is derived from CD3ε, TCRα or TCRβ.

In some embodiments, the CD3-TCR complex association domain comprises, or consists of, an amino acid sequence having at least 70% amino acid sequence identity to one of SEQ ID NOs: 30, 52, 1, 53, 5, 54 or 9.

In some embodiments, the amino acid sequence derived from a CD3-TCR complex polypeptide is derived from CD3ε. In some embodiments, the CD3-TCR complex association domain comprises, or consists of, an amino acid sequence having at least 70% amino acid sequence identity to SEQ ID NO:30.

In some embodiments, the amino acid sequence derived from a CD3-TCR complex polypeptide is derived from TCRα or TCRβ. In some embodiments, the CD3-TCR complex association domain comprises, or consists of, an amino acid sequence having at least 70% amino acid sequence identity to one of SEQ ID NOs: 52, 1, 53, 5, 54 or 9.

In some embodiments, the antigen-binding moiety that binds to a variant Fc domain comprises the heavy chain variable (VH) region and light chain variable (VL) region of an antibody that binds to the variant Fc domain.

In some embodiments, the antigen-binding moiety is or comprises an Fv, scFV, Fab, Fab′, Fab′-SH, F(ab′)₂, crossFab, scFab or dAb moiety. In some embodiments, the antigen-binding moiety is or comprises an scFv.

In some embodiments, the component of the antigen-binding moiety is or comprises the heavy chain variable (VH) region or the light chain variable (VL) region of an antibody that binds to the variant Fc domain.

In some embodiments, the antigen-binding moiety or component thereof is connected at its C-terminus to the N-terminus of the CD3-TCR complex association domain, optionally through a linker sequence.

In some embodiments, the variant Fc domain binds to an Fc receptor with lower affinity than the affinity with which the reference Fc domain binds to the Fc receptor, optionally wherein the Fc receptor is an Fcγ receptor or neonatal Fc receptor (FcRn).

In some embodiments, the variant Fc domain comprises a CH2-CH3 region comprising an amino acid difference at one or more of the following positions, relative to the amino acid sequence of a CH2-CH3 region of the reference Fc domain according to EU numbering: L234, L235, I253, N297, S298, H310, P329, E333, K334 or H435.

In some embodiments, the variant Fc domain comprises a CH2-CH3 region comprising G329 according to EU numbering.

In some embodiments, the antigen-binding moiety comprises a VL region incorporating the following CDRs:
LC-CDR1 having the amino acid sequence of SEQ ID NO:69;
LC-CDR2 having the amino acid sequence of SEQ ID NO:70; and
LC-CDR3 having the amino acid sequence of SEQ ID NO:71.

In some embodiments, the antigen-binding moiety comprises a VH region incorporating:
(i) the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:58;
or
(ii) the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58.

In some embodiments, the antigen-binding moiety comprises:
(a) (i) a VH region incorporating the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:68;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71;
or
(b) (i) a VH region incorporating the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71.

In some embodiments, the antigen-binding moiety comprises a VL having an amino acid sequence having at least 70% amino acid sequence identity to SEQ ID NO:68.

In some embodiments, the antigen-binding moiety comprises a VH having an amino acid sequence having at least 70% amino acid sequence identity to SEQ ID NO:65, 63 or 55.

In some embodiments, the antigen-binding moiety comprises:
(a) (i) a VH having an amino acid sequence having at least 70% amino acid sequence identity to SEQ ID NO:65; and
   (ii) a VL having an amino acid sequence having at least 70% amino acid sequence identity to SEQ ID NO:68;
or
(b) (i) a VH having an amino acid sequence having at least 70% amino acid sequence identity to SEQ ID NO:63; and
   (ii) a VL having an amino acid sequence having at least 70% amino acid sequence identity to SEQ ID NO:68;
or
(c) (i) a VH having an amino acid sequence having at least 70% amino acid sequence identity to SEQ ID NO:55; and
   (ii) a VL having an amino acid sequence having at least 70% amino acid sequence identity to SEQ ID NO:68.

In some embodiments, the variant Fc domain comprises a CH2-CH3 region comprising A298, A333 and A334 according to EU numbering.

In some embodiments, the antigen-binding moiety comprises a VH region incorporating the following CDRs:
HC-CDR1 having the amino acid sequence of SEQ ID NO:77;
HC-CDR2 having the amino acid sequence of SEQ ID NO:78; and
HC-CDR3 having the amino acid sequence of SEQ ID NO:79.

In some embodiments, the antigen-binding moiety comprises a VL region incorporating the following CDRs:
LC-CDR1 having the amino acid sequence of SEQ ID NO:85;
LC-CDR2 having the amino acid sequence of SEQ ID NO:86; and
LC-CDR3 having the amino acid sequence of SEQ ID NO:87.

In some embodiments, the antigen-binding moiety comprises:
(i) a VH region incorporating the following CDRs:
   HC-CDR1 having the amino acid sequence of SEQ ID NO:77;
   HC-CDR2 having the amino acid sequence of SEQ ID NO:78; and
   HC-CDR3 having the amino acid sequence of SEQ ID NO:79;
and
(ii) a VL region incorporating the following CDRs:
   LC-CDR1 having the amino acid sequence of SEQ ID NO:85;
   LC-CDR2 having the amino acid sequence of SEQ ID NO:86; and
   LC-CDR3 having the amino acid sequence of SEQ ID NO:87.

In some embodiments, the antigen-binding moiety comprises a VH having an amino acid sequence having at least 70% amino acid sequence identity to SEQ ID NO:76.

In some embodiments, the antigen-binding moiety comprises a VL having an amino acid sequence having at least 70% amino acid sequence identity to SEQ ID NO:84.

In some embodiments, the antigen-binding moiety comprises:
(i) a VH having an amino acid sequence having at least 70% amino acid sequence identity to SEQ ID NO:76; and
(ii) a VL having an amino acid sequence having at least 70% amino acid sequence identity to SEQ ID NO: 84.

The present disclosure also provides a recombinant CD3-TCR complex polypeptide, comprising:
(i) an antigen-binding moiety, which is an scFV, or a component thereof, wherein the antigen-binding moiety binds to a variant Fc domain having an amino acid sequence comprising at least one amino acid difference relative to a reference Fc domain, to which the antigen-binding moiety does not bind, wherein the variant Fc domain comprises a CH2-CH3 region comprising G329 according to EU numbering; and
(ii) a CD3-TCR complex association domain having an amino acid sequence derived from a CD3-TCR complex polypeptide, wherein the amino acid sequence derived from a CD3-TCR complex polypeptide is derived from CD38.

The present disclosure also provides a polypeptide complex, comprising:
(a) a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
   (ii) a CD3-TCR complex association domain having an amino acid sequence derived from a CD3-TCR complex polypeptide; and
(b) a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form the antigen-binding moiety.

In some embodiments, the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.

In some embodiments:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

In some embodiments, the CD3-TCR complex association domain derived from TCRα comprises, or consists of, an amino acid sequence having at least 70% amino acid sequence identity to SEQ ID NO:52 or 1.

In some embodiments, the CD3-TCR complex association domain derived from TCRβ comprises, or consists of, an amino acid sequence having at least 70% amino acid sequence identity to one of SEQ ID NOs: 53, 5, 54 or 9.

The present disclosure also provides a polypeptide complex, comprising:
(a) a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind, wherein the variant Fc domain comprises a CH2-CH3 region comprising G329 according to EU numbering; and
   (ii) a CD3-TCR complex association domain having an amino acid sequence derived from a CD3-TCR complex polypeptide; and
(b) a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form the antigen-binding moiety;
wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain;
and wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

The present disclosure also provides a CD3-TCR polypeptide complex, wherein the CD3-TCR polypeptide complex comprises a recombinant CD3-TCR complex polypeptide or polypeptide complex according to the present disclosure.

The present disclosure also provides a composite polypeptide comprising:
(a) an amino acid sequence encoding a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
   (ii) a CD3-TCR complex association domain having an amino acid sequence derived from a CD3-TCR complex polypeptide; and
(b) an amino acid sequence encoding a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form a CD3-TCR complex comprising the antigen-binding moiety;
and wherein the composite polypeptide further comprises a cleavage site between the amino acid sequences of (a) and (b).

In some embodiments, the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.

In some embodiments:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

In some embodiments, the CD3-TCR complex association domain derived from TCRα comprises, or consists of, an amino acid sequence having at least 70% amino acid sequence identity to SEQ ID NO:52 or 1.

In some embodiments, the CD3-TCR complex association domain derived from TCRβ comprises, or consists of, an amino acid sequence having at least 70% amino acid sequence identity to one of SEQ ID NOs: 53, 5, 54 or 9.

The present disclosure also provides a nucleic acid, or a plurality of nucleic acids, encoding a recombinant CD3-TCR complex polypeptide, a polypeptide complex, or composite polypeptide according to the present disclosure.

The present disclosure also provides a nucleic acid, or a plurality of nucleic acids, encoding:
(a) a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
   (ii) a CD3-TCR complex association domain having an amino acid sequence derived from a CD3-TCR complex polypeptide; and
(b) a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form a CD3-TCR complex comprising the antigen-binding moiety.

The present disclosure also provides an expression vector, or a plurality of expression vectors, comprising a nucleic acid or plurality of nucleic acids according to the present disclosure.

The present disclosure also provides a cell comprising a recombinant CD3-TCR complex polypeptide, polypeptide complex, CD3-TCR polypeptide complex, composite polypeptide, nucleic acid or plurality of nucleic acids, or an expression vector or plurality of expression vectors according to the present disclosure.

The present disclosure also provides a pharmaceutical composition comprising a cell according to the present disclosure.

The present disclosure also provides a cell or pharmaceutical composition according to the present disclosure, for use in a method of medical treatment or prophylaxis.

The present disclosure also provides a cell or pharmaceutical composition according to the present disclosure,
for use in a method of treating or preventing a disease in which cells comprising or expressing a target antigen are pathologically-implicated, wherein the method comprises administering the cell or pharmaceutical composition to a subject to which an antigen-binding molecule has been or is to be administered;
wherein the antigen-binding molecule comprises: (a) an antigen-binding domain that binds to the target antigen, and (b) a variant Fc domain having an amino acid sequence comprising at least one amino acid difference relative to a reference Fc domain;
and wherein the antigen-binding moiety of the recombinant CD3-TCR complex polypeptide, polypeptide complex, CD3-TCR polypeptide complex or composite polypeptide comprised in the cell according to the present disclosure, or the cell comprised in the pharmaceutical composition according to the present disclosure, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell according to the present disclosure, or the cell comprised in the pharmaceutical composition according to the present disclosure, binds to the variant Fc domain.

The present disclosure also provides a method for depleting or killing cells comprising or expressing a target antigen, comprising contacting cells comprising/expressing a target antigen with:
(i) a cell or pharmaceutical composition according to the present disclosure; and
(ii) an antigen-binding molecule comprising: (a) an antigen-binding domain that binds to the target antigen, and (b) a variant Fc domain having an amino acid sequence comprising at least one amino acid difference relative to a reference Fc domain;
wherein the antigen-binding moiety of the recombinant CD3-TCR complex polypeptide, polypeptide complex, CD3-TCR polypeptide complex or composite polypeptide comprised in the cell according to the present disclosure, or the cell comprised in the pharmaceutical composition according to the present disclosure, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell according to the present disclosure, or the cell comprised in the pharmaceutical composition according to the present disclosure, binds to the variant Fc domain.

The present disclosure also provides a kit, comprising:
(i) a cell or pharmaceutical composition according to the present disclosure; and
(ii) an antigen-binding molecule comprising: (a) an antigen-binding domain that binds to a target antigen, and (b) a variant Fc domain having an amino acid sequence comprising at least one amino acid difference relative to a reference Fc domain;
wherein the antigen-binding moiety of the recombinant CD3-TCR complex polypeptide, polypeptide complex, CD3-TCR polypeptide complex or composite polypeptide comprised in the cell of according to the present disclosure, or the cell comprised in the pharmaceutical composition according to the present disclosure, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell according to the present disclosure, or the cell comprised in the pharmaceutical composition according to the present disclosure, binds to the variant Fc domain.

The present disclosure also provides a kit, comprising:
(i) a nucleic acid or plurality of nucleic acids according to the present disclosure, or an expression vector or plurality of expression vectors according to the present disclosure; and
(ii) an antigen-binding molecule comprising: (a) an antigen-binding domain that binds to a target antigen, and (b) a variant Fc domain having an amino acid sequence comprising at least one amino acid difference relative to a reference Fc domain;
wherein the antigen-binding moiety of the CD3-TCR complex polypeptide, polypeptide complex, CD3-TCR polypeptide complex or composite polypeptide encoded by the nucleic acid or plurality thereof according to the present disclosure, or encoded by the nucleic acid or plurality thereof comprised in the expression vector or plurality thereof according to the present disclosure, binds to the variant Fc domain.

### DESCRIPTION

The present disclosure provides recombinant CD3-TCR complex polypeptides, polypeptide complexes (including CD3-TCR polypeptide complexes) comprising such recombinant CD3-TCR complex polypeptides, nucleic acids and vectors encoding such polypeptides and polypeptide complexes, cells comprising such polypeptides, polypeptide complexes, nucleic acids and vectors, compositions comprising such cells, and uses thereof.

More specifically, the present disclosure is directed to novel recombinant CD3-TCR complex polypeptides and polypeptide complexes comprising the same, having an antigen-binding moiety specific for a variant Fc domain, and comprising a CD3-TCR complex association domain, such that when they are expressed by a cell (particularly a T cell), the recombinant CD3-TCR complex polypeptides are able to associate with CD3-TCR complex polypeptides to form a CD3-TCR complex that recognises the variant Fc domain. Upon binding of the antigen-binding moiety to its cognate variant Fc domain, CD3-TCR complex-mediated signalling is triggered, activating the CD3-TCR polypeptide complex-expressing cells.

Unexpectedly, the present disclosure demonstrates that novel recombinant CD3-TCR complex polypeptides that do not comprise a domain capable of intrinsically triggering CD3-TCR complex-mediated signalling (e.g. a CD3ζ intracellular domain) are able to associate with endogenous CD3-TCR complex polypeptides expressed by the cell, to form functional CD3-TCR complexes that nevertheless cause T cell activation in response to the variant Fc domain to which the antigen-binding moiety binds. The present disclosure moreover demonstrates that this technical effect is attained across different embodiments, particularly (i) an embodiment wherein the recombinant CD3-TCR complex polypeptide comprises an scFv that binds to the variant Fc domain and a CD3-TCR complex association domain derived from CD3ε, and (ii) an embodiment wherein the antigen-binding moiety specific for the variant Fc domain is formed by VH region and VL region moieties provided on separate recombinant CD3-TCR complex polypeptides, respectively comprising CD3-TCR complex association domains derived from TRAC and TRBC1.

More unexpectedly still, in experiments providing for direct comparison of the level of activation of T cells in response to cells presenting the variant Fc domain, T cells expressing the novel CD3-TCR complexes according to (i) and (ii) in the preceding paragraph are shown to be activated to a similar or greater extent than T cells expressing a CAR construct having the same antigen-binding moiety (in scFv format) which comprises costimulatory and CD3ζ intracellular signalling domains (disclosed in WO 2018/177966 A1)-see FIGS. 6, 7, 10 and 11.

### Recombinant CD3-TCR Complex Polypeptides

Aspects and embodiments of the present disclosure pertain to recombinant CD3-TCR complex polypeptides.

As referred to herein, a ‘recombinant’ polypeptide refers to a non-naturally-occurring polypeptide. A recombinant polypeptide may also be referred to as a ‘synthetic’ polypeptide. A recombinant polypeptide may comprise or consist of an amino acid sequence that is not encoded by the genome of a naturally-occurring organism (e.g. a wildtype organism). That is, a recombinant polypeptide may comprise or consist of an amino acid sequence which is not comprised in the amino acid sequence of a polypeptide produced by a naturally-occurring organism. A recombinant polypeptide may be encoded by nucleic acid produced using recombinant nucleic acid techniques. A recombinant polypeptide may be produced by expression (e.g. by transcription, translation and any subsequent post-translational processing) from recombinant nucleic acid encoding the polypeptide. Recombinant nucleic acid techniques include techniques for constructing and manipulating the nucleotide sequences of nucleic acids, and include molecular cloning.

CD3-TCR complexes (also sometimes referred to as TCR-CD3 complexes, see e.g. Dong et al., Nature (2019) 573 (7775): 546-552) are polypeptide complexes expressed at the cell surface of T cells that are involved in antigen-specific T cell activation. The structure and function of CD3-TCR complexes is reviewed e.g. in Mariuzza et al., J Biol Chem. 2020 Jan. 24; 295 (4): 914-925, which is hereby incorporated by reference in its entirety.

In mammals, CD3-TCR complexes comprise diverse TCR polypeptides that together form the heterodimeric TCR for antigen recognition (either TCRα and TCRβ, or TCRγ and TCRδ), provided in non-covalent association with invariant CD3ε, CD3δ, CD3γ and CD3ζ polypeptides. The classical, octameric CD3-TCR complex comprise a TCRα and TCRβ heterodimer (i.e. TCRαβ) or a TCRγ and TCRδ heterodimer (i.e. TCRγδ), a heterodimer comprising CD3ε and CD30 (i.e. CD308), heterodimer comprising CD3ε and CD3γ (i.e. CD3γε)), and a CD3ζ homodimer (i.e. CD3ζζ). Such TCR-CD3 complexes may be represented respectively as CD3γε/CD3δε/CD3ζζ/TCRαβ and CD3γε/CD3δεl /CD3ζζ (see e.g. Zheng et al., Nature (2019) 573 (7775): 546-552).

In some embodiments, the CD3-TCR complex is a CD3-TCRα/β complex. In some embodiments, the CD3-TCR complex is a CD3-TCRγ/δ complex. A CD3-TCRα/β complex may comprise TCRα and TCRβ polypeptides, and also CD3γ, CD3ε, CD3δ and/or CD3ζ polypeptides. A CD3-TCRα/β complex may comprise or consist of CD3γε/CD3δε/CD3ζζ/TCRαβ. A CD3-TCRΓ/δ complex may comprise TCRγ and TCRδ polypeptides, and also CD3γ, CD3ε, CD3δ and/or CD3ζ polypeptides. A CD3-TCRγ/δ complex may comprise or consist of CD3γε/CD3δε/CD3ζζ/TCRγ/δ.

Herein, a ‘CD3-TCR complex polypeptide’ refers to a constituent polypeptide of a CD3-TCR complex. In some embodiments, a CD3-TCR complex polypeptide is selected from TCRα, TCRβ, TCRγ, TCRδ, TRAC, TRBC1, TRBC2, TRGC1, TRGC2, TRDC, CD3ε, CD3δ, CD3γ, CD3ζ and CD3η. In some embodiments, a CD3-TCR complex polypeptide is a recombinant CD3-TCR complex polypeptide as described herein.

In this specification, ‘TCRα’, ‘TCRβ’, TCRγ′, ‘TCRδ ’, ‘TRAC’, ‘TRBC1’, ‘TRBC2’, ‘TRGC1’, ‘TRGC2’, ‘TRDC’, ‘CD3ε’, ‘CD30’, ‘CD3γ’, ‘CD3Z’ and ‘CD3n’ refer respectively to TCRα, TCRβ, TCRγ, TCRδ, TRAC, TRBC1, TRBC2, TRGC1, TRGC2, TRDC, CD3ε, CD3δ, CD3γ, CD3ζ and CD3η from any species, and include isoforms, fragments, variants or homologues from any species. In some embodiments, TCRα, TCRβ, TCRγ, TCRδ, TRAC, TRBC1, TRBC2, TRGC1, TRGC2, TRDC, CD3ε, CD30, CD3γ, CD3Z or CD3η is from a mammal (e.g. a therian, placental, epitherian, preptotheria, archontan, primate (rhesus, cynomolgous, non-human primate or human)). In some embodiments, the TCRα, TCRβ, TCRγ, TCRδ, TRAC, TRBC1, TRBC2, TRGC1, TRGC2, TRDC, CD3ε, CD3δ, CD3γ, CD3ζ or CD3η is human.

As used herein, isoforms, fragments, variants or homologues of a given reference protein (e.g. TCRα, TCRβ, TCRγ, TCRδ, TRAC, TRBC1, TRBC2, TRGC1, TRGC2, TRDC, CD3ε, CD3δ, CD3γ, CD3ζ or CD3η) may be characterised as having at least 70% sequence identity, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of the reference protein.

A ‘fragment’ generally refers to a fraction of the reference protein. A ‘variant’ generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An ‘isoform’ generally refers to a variant of the reference protein expressed by the same species as the species of the reference protein. A ‘homologue’ generally refers to a variant of the reference protein produced by a different species as compared to the species of the reference protein. Homologues include orthologues.

Isoforms, fragments, variants or homologues of a given reference protein may optionally be characterised as having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of an immature or mature (i.e. after processing to remove signal peptide) form of a specified isoform of the relevant protein from a given species, e.g. human.

In some embodiments, TCRα comprises an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 1. In some embodiments, TRAC comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:1.

In some embodiments, TCRβ comprises an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:5 or 9. In some embodiments, TRBC1 comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:5. In some embodiments, TRBC2 comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:9.

In some embodiments, TCRγ comprises an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 13 or 17. In some embodiments, TRGC1 comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 13. In some embodiments, TRGC2 comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 17.

In some embodiments, TCRδ comprises an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:21. In some embodiments, TRDC comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:21.

In some embodiments, CD3ε comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:25 or 30.

In some embodiments, CD30 comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:31 or 36.

In some embodiments, CD3γ comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:37 or 42.

In some embodiments, CD3Z comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:43 or 48.

In some embodiments, CD3η comprises, or consists of, an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:49 or 51.

In some embodiments, a recombinant CD3-TCR complex polypeptide according to the present disclosure comprises:
(i) an antigen-binding moiety, or a component thereof, as described herein; and
(ii) a CD3-TCR complex association domain as described herein.

In some embodiments, the amino acid sequence of the antigen-binding moiety/component thereof is N-terminal to the amino acid sequence of the CD3-TCR complex association domain, in the amino acid sequence of the recombinant CD3-TCR complex polypeptide. That is, in some embodiments, the recombinant CD3-TCR complex polypeptide comprises the structure: N term-[ . . . ]-[antigen-binding moiety/component thereof]-[CD3-TCR complex association domain]-[ . . . ]-C term.

In some embodiments, a recombinant CD3-TCR complex polypeptide according to the present disclosure does not comprise a domain or amino acid sequence comprising an immunoreceptor tyrosine-based activation motif (ITAM). ITAMs comprise an amino acid sequence according to YXXL/I (SEQ ID NO:222), wherein ‘X’ denotes any amino acid. In ITAM-containing proteins, sequences according to YXXL/I are often separated by 6 to 8 amino acids (i.e. they conform to the formula: YXXL/I(X)₆₋₈YXXL/I; SEQ ID NO: 223). When phosphate groups are added to the tyrosine residue of an ITAM by tyrosine kinases, a signalling cascade is initiated within the cell. ITAM-containing sequences include the intracellular domains of CD3ζ and FcγRI. In some embodiments, a recombinant CD3-TCR complex polypeptide according to the present disclosure does not comprise the amino acid sequence shown in SEQ ID NO:47. In some embodiments, a recombinant CD3-TCR complex polypeptide does not comprise an amino acid sequence according to SEQ ID NO:223. In some embodiments, a recombinant CD3-TCR complex polypeptide does not comprise an amino acid sequence according to SEQ ID NO:222.

In some embodiments, a recombinant CD3-TCR complex polypeptide according to the present disclosure does not comprise a costimulatory sequence. As referred to herein, a ‘costimulatory sequence’ refers to an amino acid sequence which provides for costimulation of an immune cell expressing the recombinant CD3-TCR complex polypeptide. Costimulation promotes proliferation and survival, and may also promote cytokine production, differentiation, cytotoxic function and memory formation. Molecular mechanisms of T cell costimulation are reviewed e.g. in Chen and Flies, (2013) Nat Rev Immunol 13 (4): 227-242. A costimulatory sequence may be, or may be derived from, the intracellular domain of a costimulatory protein. Costimulatory proteins include CD28, 4-1BB, ICOS, CD27, OX40, HVEM, CD2, SLAM, TIM-1, CD30, GITR, DR3, CD226 and LIGHT. In some embodiments, a recombinant CD3-TCR complex polypeptide according to the present disclosure does not comprise the amino acid sequence shown in SEQ ID NO: 101 (the intracellular domain of human 4-1BB).

### CD3-TCR Complex Association Domain

The recombinant CD3-TCR complex polypeptides of the present disclosure comprise a CD3-TCR complex association domain. The essential function of the CD3-TCR complex association domain is to provide for the formation of polypeptide complexes comprising the recombinant CD3-TCR complex polypeptide according to the present disclosure, and one or more CD3-TCR complex polypeptides.

A ‘CD3-TCR complex association domain’ refers to a domain through which a polypeptide comprising the domain is able to associate with a CD3-TCR complex polypeptide (e.g. a CD3-TCR complex polypeptide as described hereinabove). Thus, a CD3-TCR complex association domain according to the present disclosure comprises or consists of an amino acid sequence conferring to a polypeptide comprising the domain the ability to associate with a CD3-TCR complex polypeptide, to form a polypeptide complex comprising the CD3-TCR complex polypeptide and the polypeptide bearing the CD3-TCR complex association domain.

Association between the CD3-TCR complex association domain/polypeptide comprising the CD3-TCR complex association domain and the CD3-TCR complex polypeptide may be characterised by non-covalent, protein: protein interaction. In some embodiments, the association comprises electrostatic interaction (e.g. ionic bonding, hydrogen bonding) and/or Van der Waals forces.

In some embodiments, the CD3-TCR complex association domain is, or is derived from, the amino acid sequence of a CD3-TCR complex polypeptide. It will be appreciated that the CD3-TCR complex association domain may be, or may be derived from, the region of a CD3-TCR complex polypeptide through which the CD3-TCR complex polypeptide interacts with other CD3-TCR complex polypeptides to form polypeptide complexes. In some embodiments, the CD3-TCR complex association domain is, or is derived from, the amino acid sequence of the region of a CD3-TCR complex polypeptide required for association between the CD3-TCR complex polypeptide other CD3-TCR complex polypeptides, to form a polypeptide complex comprising such polypeptides.

The region of a CD3-TCR complex polypeptide required for such interaction can be determined by site-directed mutagenesis and/or truncation studies, in which the amino acid sequence of the CD3-TCR complex polypeptide is altered or truncated, and the effect of such alteration/truncation on its ability to associate with other CD3-TCR complex polypeptides is evaluated. Suitable techniques for investigating such protein: protein interaction include e.g. resonance energy transfer techniques such as fluorescence resonance energy transfer (FRET) and Bioluminescence Resonance Energy Transfer (BRET), using appropriate labelled interaction partners, e.g. as described in Ciruela, Curr. Opin. Biotechnol. (2008) 19 (4): 338-43.

As used herein, polypeptides, domains and amino acid sequences which are ‘derived from’ a reference polypeptide/domain/amino acid sequence have at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of the reference polypeptide/domain/amino acid sequence. Polypeptides, domains and amino acid sequences which are ‘derived from’ a reference polypeptide/domain/amino acid sequence preferably retain the functional and/or structural properties of the reference polypeptide/domain/amino acid sequence.

In some embodiments, the CD3-TCR complex association domain comprises modification to promote association with a CD3-TCR complex polypeptide. In some embodiments, the CD3-TCR complex association domain comprises modification to promote heteromerisation, i.e. association with a non-identical CD3-TCR complex polypeptide.

As used herein, a ‘modification’ refers to a difference relative to a reference amino acid sequence. A reference amino acid sequence may be the amino acid sequence encoded by the most common nucleotide sequence of the gene encoding the relevant protein. In embodiments herein (and also in the art more generally), a ‘modification’ may also be referred to as a ‘substitution’ or a ‘mutation’. A modification typically comprises substitution of an amino acid residue. Substitution of an amino acid residue comprises substitution of an amino acid residue a non-identical ‘replacement’ amino acid residue. A replacement amino acid residue of a modification according to the present disclosure may be a naturally-occurring amino acid residue (i.e. encoded by the genetic code) which is non-identical to the amino acid residue at the relevant position of the amino acid sequence prior to modification, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid residue of a modification may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

By way of illustration, in embodiments herein, a CD3-TCR complex association domain derived from TRAC comprises a modification to replace the threonine residue at position 47 (numbered relative to SEQ ID NO: 1) with a cysteine residue (SEQ ID NO:52), and a CD3-TCR complex association domain derived from TCRβ comprises a modification to replace the serine residue at position 56 (numbered relative to SEQ ID NO:5) with a cysteine residue (SEQ ID NO:53). The introduction of these cysteine residues promotes heteromerisation between the modified domains via formation of an interchain disulfide bridge.

Embodiments in which the CD3-TCR complex association domain further comprises modification to promote association with a CD3-TCR complex polypeptide are contemplated in particular in connection with aspects and embodiments of the present disclosure wherein the recombinant CD3-TCR complex polypeptide comprising such a CD3-TCR complex association domain is provided to be employed with another recombinant CD3-TCR complex polypeptide. For example, such CD3-TCR complex association domains are contemplated, in particular, to be employed in a first recombinant CD3-TCR complex polypeptide and/or a second recombinant CD3-TCR complex polypeptide of a polypeptide complex of the present disclosure comprising such recombinant CD3-TCR complex polypeptides.

In some embodiments, the CD3-TCR complex association domain is, or is derived from, the CD3-TCR complex association domain of CD3ε. In some embodiments, the CD3-TCR complex association domain is, or is derived from, the region of CD3ε required for association with CD3γ and/or CD30 (i.e. to form a CD3&: CD3γ polypeptide complex, or a CD3&: CD30 polypeptide complex). In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:30.

In some embodiments, the CD3-TCR complex association domain is, or is derived from, the CD3-TCR complex association domain of TRAC. In some embodiments, the CD3-TCR complex association domain is, or is derived from, the region of TRAC required for association with TCRβ, TRBC1 and/or TRBC2 (i.e. to form a TRAC: TCRβ polypeptide complex, or a TRAC: TRBC1 polypeptide complex, or a TRAC: TRBC2 polypeptide complex). In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:1. In some embodiments, the CD3-TCR complex association domain is derived from TRAC, and further comprises modification to promote association with another CD3-TCR complex polypeptide. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98% or ≥99% amino acid sequence identity to SEQ ID NO: 1, and comprises a cysteine residue at the position corresponding to position 47 numbered according to SEQ ID NO:1. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:52.

In some embodiments, the CD3-TCR complex association domain is, or is derived from, the CD3-TCR complex association domain of TRBC1. In some embodiments, the CD3-TCR complex association domain is, or is derived from, the region of TRBC1 required for association with TCRα and/or TRAC (i.e. to form a TRBC1: TCRα polypeptide complex, or a TRBC1: TRAC polypeptide complex). In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:5. In some embodiments, the CD3-TCR complex association domain is derived from TRBC1, and further comprises modification to promote association with another CD3-TCR complex polypeptide. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98% or ≥99% amino acid sequence identity to SEQ ID NO:5, and comprises a cysteine residue at the position corresponding to position 56 numbered according to SEQ ID NO:5. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:53.

In some embodiments, the CD3-TCR complex association domain is, or is derived from, the CD3-TCR complex association domain of TRBC2. In some embodiments, the CD3-TCR complex association domain is, or is derived from, the region of TRBC2 required for association with TCRα and/or TRAC (i.e. to form a TRBC2: TCRα polypeptide complex, or a TRBC2: TRAC polypeptide complex). In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:9. In some embodiments, the CD3-TCR complex association domain is derived from TRBC2, and further comprises modification to promote association with another CD3-TCR complex polypeptide. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98% or ≥99% amino acid sequence identity to SEQ ID NO:9, and comprises a cysteine residue at the position corresponding to position 56 numbered according to SEQ ID NO:9. In some embodiments, the CD3-TCR complex association domain comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:54.

### Antigen-Binding Moieties

The recombinant CD3-TCR complex polypeptides of the present disclosure comprise an antigen-binding moiety, or a component thereof. The essential function of the antigen-binding moiety is to provide for binding to a variant Fc domain, as described herein below.

As used herein, an ‘antigen-binding moiety’ refers to a moiety that binds to a given target antigen.

Antigen-binding moieties include antibodies (i.e. immunoglobulins (Igs)), and antigen-binding fragments and derivatives thereof. In some embodiments, an antigen-binding moiety according to the present disclosure comprises, or consists of, a monoclonal antibody, a monospecific antibody, a multispecific (e.g., bispecific, trispecific, etc.) antibody, a variable fragment (Fv) moiety, a single-chain Fv (scFv) moiety, a fragment antigen-binding (Fab) moiety, a single-chain Fab moiety (scFab), a crossFab moiety, a Fab′ moiety, a Fab′-SH moiety, a F(ab′)₂ moiety, a diabody moiety, a triabody moiety, an scFv-Fc moiety, a minibody moiety, a heavy chain only antibody (HCAb) moiety, or a single domain antibody (dAb, VHH) moiety.

Antigen-binding moieties according to the present disclosure also include further target antigen-binding peptides/polypeptides such as peptide aptamers, thioredoxins, anticalins, Kunitz domains, avimers, knottins, fynomers, atrimers, DARPins, affibodys, affilins, armadillo repeat proteins (ArmRPs), OBodys and adnectins (reviewed e.g. in Reverdatto et al., Curr Top Med Chem. 2015; 15 (12): 1082-1101, which is hereby incorporated by reference in its entirety (see also e.g. Boersma et al., J Biol Chem (2011) 286:41273-85 and Emanuel et al., Mabs (2011) 3:38-48)). Antigen-binding moieties according to the present disclosure also include target antigen-binding nucleic acids, e.g. nucleic acid aptamers (reviewed, for example, in Zhou and Rossi Nat Rev Drug Discov. 2017 16 (3): 181-202). Antigen-binding moieties according to the present disclosure also include target antigen-binding small molecules (e.g. low molecular weight (<1000 daltons, typically between ˜300-700 daltons) organic compounds).

The antigen-binding moiety of the recombinant CD3-TCR complex polypeptides of the present disclosure are capable of binding to a variant Fc domain according to the present disclosure. Antigen-binding moieties that are capable of binding to a variant Fc domain according to the present disclosure may also be described as antigen-binding moieties that bind to a variant Fc domain according to the present disclosure.

The antigen-binding moieties described herein preferably display specific binding to a variant Fc domain according to the present disclosure. As used herein, ‘specific binding’ refers to binding which is selective for the target antigen, and which can be discriminated from non-specific binding to non-target antigen. An antigen-binding moiety that specifically binds to a given target antigen preferably binds the target antigen with greater affinity, and/or with greater duration than it binds to other, non-target antigens.

The ability of a given moiety to bind specifically to a given target antigen can be determined by analysis according to methods known in the art, such as by ELISA, Surface Plasmon Resonance (SPR; see e.g. Hearty et al., Methods Mol Biol (2012) 907:411-442), Bio-Layer Interferometry (BLI; see e.g. Lad et al., (2015) J Biomol Screen 20 (4): 498-507), flow cytometry, or by a radiolabeled antigen-binding assay (RIA) enzyme-linked immunosorbent assay. Through such analysis binding to a given target antigen can be measured and quantified. In some embodiments, the level of binding may be the response detected in a given assay.

In some embodiments, the antigen-binding moiety described herein binds to a variant Fc domain according to the present disclosure with an affinity (e.g. determined by SPR or BLI) in the micromolar range, i.e. K_{D}=9.9×10⁻⁴ to 1×10⁻⁶ M. In some embodiments, the antigen-binding moiety described herein binds to a variant Fc domain according to the present disclosure with sub-micromolar affinity, i.e. K_{D}<1×10⁻⁶ M. In some embodiments, the antigen-binding moiety described herein binds to a variant Fc domain according to the present disclosure with an affinity in the nanomolar range, i.e. K_{D}=9.9×10⁻⁷ to 1×10⁻⁹ M. In some embodiments, the antigen-binding moiety described herein binds to a variant Fc domain according to the present disclosure with sub-nanomolar affinity, i.e. K_{D}<1×10⁻⁹ M. In some embodiments, the antigen-binding moiety described herein binds to a variant Fc domain according to the present disclosure with an affinity in the picomolar range, i.e. K_{D}=9.9×10⁻¹⁰ to 1×10⁻¹² M. In some embodiments, the antigen-binding moiety described herein binds to a variant Fc domain according to the present disclosure with sub-picomolar affinity, i.e. K_{D}<1×10⁻¹² M.

The antigen-binding moieties of the recombinant CD3-TCR complex polypeptides according to the present disclosure preferably do not display specific binding to a reference Fc domain according to the present disclosure. In some embodiments, the antigen-binding moiety does not bind, or displays substantially no binding, to a reference Fc domain according to the present disclosure.

An antigen-binding moiety that ‘does not bind’ or that ‘displays substantially no binding’ to a given antigen displays a level of binding to the given antigen which is similar to the level of binding to an antigen that the antigen-binding moiety is known not to bind, or known to not to bind specifically, e.g. a non-target antigen. In some embodiments, the level of binding of an antigen-binding moiety that does not bind, or that displays substantially no binding, to a given antigen is ≥0.5 times and ≤2 times, e.g. one of ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of binding displayed by the antigen-binding moiety to an antigen that the antigen-binding moiety is known not to bind, or known to not to bind specifically, e.g. a non-target antigen.

In some embodiments, the level of binding of the antigen-binding moiety to a reference Fc domain according to the present disclosure is ≤10% of the binding of the antigen-binding moiety to a variant Fc domain according to the present disclosure as determined e.g. by ELISA, SPR, BLI or RIA. In some embodiments, the antigen-binding moiety binds to a reference Fc domain according to the present disclosure with an equilibrium dissociation constant (K_{D}; e.g. determined by SPR or BLI) that is at least 0.1 order of magnitude greater than the K_{D} of the antigen-binding moiety for a variant Fc domain according to the present disclosure.

An antigen-binding moiety according to the present disclosure may be, or may comprise, an antigen-binding peptide/polypeptide, or an antigen-binding peptide/polypeptide complex. An antigen-binding moiety may comprise more than one peptide/polypeptide that together form an antigen-binding domain.

The peptides/polypeptides may associate covalently or non-covalently. In some embodiments, the peptides/polypeptides form part of a larger polypeptide comprising the peptides/polypeptides (e.g. in the case of an scFv moiety comprising a VH region and a VL region, or in the case of a scFab moiety comprising VH-CH1 and VL-CL).

In some embodiments, the antigen-binding moiety of the present disclosure comprises an antibody heavy chain variable (VH) region and an antibody light chain variable (VL) region of an antibody capable of binding to a given target antigen. In some embodiments, the antigen-binding moiety comprises, or consists of, an Fv moiety formed by the VH region and a VL region of an antibody capable of binding to a given target antigen. In some embodiments, the VH region and a VL region may be provided in the same polypeptide, and joined by a linker sequence. In some embodiments, the antigen-binding moiety comprises, or consists of, an scFv moiety that binds to a given target antigen.

Antigen-binding moieties of the present disclosure generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antigen-binding moiety, which is the part of the moiety that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as (i) the Kabat system, described in Kabat et al., Sequences of Proteins of Immunological Interest, 5th Ed. Public Health Service, National Institutes of Health, Bethesda, MD (1991); (ii) the Chothia system, described in Chothia et al., J. Mol. Biol. 196:901-917 (1987); and (iii) the international IMGT (ImMunoGeneTics) information system (LeFranc et al., Nucleic Acids Res. (2015) 43 (Database issue): D413-22), which uses the IMGT V-DOMAIN numbering rules as described in Lefranc et al., Dev. Comp. Immunol. (2003) 27:55-77.

The CDRs and FRs of the VH regions and VL regions of the antigen-binding moieties described herein are defined according to the Kabat system.

In some embodiments, the antigen-binding moiety comprises the CDRs of an antigen-binding moiety that binds to a variant Fc domain according to the present disclosure. In some embodiments, the antigen-binding moiety comprises the FRs of an antigen-binding moiety that binds to a variant Fc domain according to the present disclosure. In some embodiments, the antigen-binding moiety comprises the CDRs and the FRs of an antigen-binding moiety that binds to a variant Fc domain according to the present disclosure. That is, in some embodiments, the antigen-binding moiety comprises the VH region and the VL region of an antigen-binding moiety that binds to a variant Fc domain according to the present disclosure.

Wessels et al. Bioanal. (2017) 9 (11): 849-59 describes the identification of an antibody that binds to antibodies comprising an Fc domain derived from human IgG1 comprising P329G, but that does not bind to antibodies comprising the equivalent Fc domain lacking the P329G substitution. The antibody also binds to antibodies having a hIgG1-derived Fc region comprising P329G and further comprising L234A and L235A. Darowski et al., Protein Eng. Des. Sel. (2019) 32 (5): 207-218 and Stock et al., Journal for ImmunoTherapy of Cancer (2022) 10: e005054 provide the structure of the anti-P329G Fab with Fc comprising P329G, L234A and L235A. The anti-P329G Fab interacts with Fc comprising P329G, L234A and L235A with 1:1 stoichiometry. The epitope is disclosed to include positions N325 to P331 (including G329), and also S267 to E272.

In some embodiments, the antigen-binding moiety comprises the CDRs, FRs and/or the VH and/or VL regions of an antigen-binding molecule described herein that binds to a variant Fc domain according to the present disclosure, or comprises CDRs, FRs and/or VH and/or VL regions which are derived from those of an antigen-binding molecule described herein that binds to a variant Fc domain according to the present disclosure. In some embodiments, an antigen-binding molecule that binds to a variant Fc domain according to the present disclosure is selected from: αP329G_VH1/VL1, αP329G_VH2/VL1, αP329G_VH3/VL1 and αAAA.

In some embodiments, the antigen-binding moiety comprises a VH region according to (1) or (2) below:

(1) a VH region incorporating the following CDRs:
HC-CDR1 having the amino acid sequence of SEQ ID NO:56
HC-CDR2 having the amino acid sequence of SEQ ID NO:57
HC-CDR3 having the amino acid sequence of SEQ ID NO:58,

or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.

(2) a VH region incorporating the following CDRs:
HC-CDR1 having the amino acid sequence of SEQ ID NO:56
HC-CDR2 having the amino acid sequence of SEQ ID NO:64
HC-CDR3 having the amino acid sequence of SEQ ID NO:58,

or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.

In some embodiments, the antigen-binding moiety comprises a VH region according to (3) or (4) below:

(3) a VH region incorporating the following FRs:
HC-FR1 having the amino acid sequence of SEQ ID NO:59
HC-FR2 having the amino acid sequence of SEQ ID NO:60
HC-FR3 having the amino acid sequence of SEQ ID NO:61
HC-FR4 having the amino acid sequence of SEQ ID NO:62,
or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.

(4) a VH region incorporating the following FRs:
HC-FR1 having the amino acid sequence of SEQ ID NO:66
HC-FR2 having the amino acid sequence of SEQ ID NO:60
HC-FR3 having the amino acid sequence of SEQ ID NO:67
HC-FR4 having the amino acid sequence of SEQ ID NO:62,
or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.

In some embodiments, the antigen-binding moiety comprises a VH region comprising the CDRs according to (1) or (2) above, and the FRs according to (3) or (4) above.

In some embodiments, the antigen-binding moiety comprises a VH region according to (5) or (6) below:

(5) a VH region comprising the CDRs according to (1) and the FRs according to (3).

(6) a VH region comprising the CDRs according to (2) and the FRs according to (4).

(7) a VH region comprising the CDRs according to (2) and the FRs according to (3).

In some embodiments, the antigen-binding moiety comprises a VH region according to one of (8) to (10) below:

(8) a VH region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO: 55.

(9) a VH region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO: 63.

(10) a VH region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO: 65.

In some embodiments, the antigen-binding moiety comprises a VL region according to (11) below:

(11) a VL region incorporating the following CDRs:
LC-CDR1 having the amino acid sequence of SEQ ID NO:69
LC-CDR2 having the amino acid sequence of SEQ ID NO:70
LC-CDR3 having the amino acid sequence of SEQ ID NO:71,

or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.

In some embodiments, the antigen-binding moiety comprises a VL region according to (12) below:

(12) a VL region incorporating the following FRs:
LC-FR1 having the amino acid sequence of SEQ ID NO:72
LC-FR2 having the amino acid sequence of SEQ ID NO:73
LC-FR3 having the amino acid sequence of SEQ ID NO:74
LC-FR4 having the amino acid sequence of SEQ ID NO:75,
or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.

In some embodiments, the antigen-binding moiety comprises a VL region according to (13) below:

(13) a VL region comprising the CDRs according to (11) and the FRs according to (12).

In some embodiments, the antigen-binding moiety comprises a VL region according to (14) below:

(14) a VL region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO: 68.

In some embodiments, the antigen-binding moiety comprises a VH region according to any one of (1) to (10) above, and a VL region according to any one of (11) to (14) above.

In some embodiments, the antigen-binding moiety comprises a VH region according to (15) below:

(15) a VH region incorporating the following CDRs:
HC-CDR1 having the amino acid sequence of SEQ ID NO:77
HC-CDR2 having the amino acid sequence of SEQ ID NO:78
HC-CDR3 having the amino acid sequence of SEQ ID NO:79,

or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.

In some embodiments, the antigen-binding moiety comprises a VH region according to (16) below:

(16) a VH region incorporating the following FRs:
HC-FR1 having the amino acid sequence of SEQ ID NO:80
HC-FR2 having the amino acid sequence of SEQ ID NO:81
HC-FR3 having the amino acid sequence of SEQ ID NO:82
HC-FR4 having the amino acid sequence of SEQ ID NO: 83,
or a variant thereof in which 1 or 2 or 3 amino acids in HC-FR1, and/or in which 1 or 2 or 3 amino acids in HC-FR2, and/or in which 1 or 2 or 3 amino acids in HC-FR3, and/or in which 1 or 2 or 3 amino acids in HC-FR4 are substituted with another amino acid.

In some embodiments, the antigen-binding moiety comprises a VH region according to (17) below:

(17) a VH region comprising the CDRs according to (15) and the FRs according to (16).

In some embodiments, the antigen-binding moiety comprises a VH region according to (18) below:

(18) a VH region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO: 76.

In some embodiments, the antigen-binding moiety comprises a VL region according to (19) below:

(19) a VL region incorporating the following CDRs:
LC-CDR1 having the amino acid sequence of SEQ ID NO:85
LC-CDR2 having the amino acid sequence of SEQ ID NO:86
LC-CDR3 having the amino acid sequence of SEQ ID NO:87,

or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.

In some embodiments, the antigen-binding moiety comprises a VL region according to (20) below:

(20) a VL region incorporating the following FRs:
LC-FR1 having the amino acid sequence of SEQ ID NO:88
LC-FR2 having the amino acid sequence of SEQ ID NO:89
LC-FR3 having the amino acid sequence of SEQ ID NO:90
LC-FR4 having the amino acid sequence of SEQ ID NO:91,
or a variant thereof in which 1 or 2 or 3 amino acids in LC-FR1, and/or in which 1 or 2 or 3 amino acids in LC-FR2, and/or in which 1 or 2 or 3 amino acids in LC-FR3, and/or in which 1 or 2 or 3 amino acids in LC-FR4 are substituted with another amino acid.

In some embodiments, the antigen-binding moiety comprises a VL region according to (21) below:

(21) a VL region comprising the CDRs according to (19) and the FRs according to (20).

In some embodiments, the antigen-binding moiety comprises a VL region according to (22) below:

(22) a VL region comprising an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100%, sequence identity, to the amino acid sequence of SEQ ID NO: 84.

In some embodiments, the antigen-binding moiety comprises a VH region according to any one of (15) to (18) above, and a VL region according to any one of (19) to (22) above.

Substitutions of amino acids in accordance with the present disclosure may be biochemically conservative. In some embodiments, where an amino acid to be substituted is provided in one of rows 1 to 5 of the table below, the replacement amino acid of the substitution is another, non-identical amino acid provided in the same row.

| Row | Shared property | Amino acids |
|---|---|---|
| 1 | Hydrophobic | Met, Ala, Val, Leu, Ile, Trp, Tyr, |
| | | Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, Ile, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | | Side-chain charge |
|---|---|---|
| Amino Acid | Side-chain polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target antigen binding) of the antigen-binding moiety comprising the substitution, as compared to the equivalent unsubstituted molecule.

In some embodiments, an antigen-binding moiety of the present disclosure comprises a VH as described herein. In some embodiments, an antigen-binding moiety comprises a VL as described herein. In some embodiments, an antigen-binding moiety comprises one or more antibody heavy chain constant regions (CH). In some embodiments, an antigen-binding moiety comprises one or more antibody light chain constant regions (CL). In some embodiments, an antigen-binding moiety comprises a CH1, CH2 region and/or a CH3 region of an immunoglobulin (Ig). In some embodiments, an antigen-binding moiety comprises a linker sequence as described herein.

In some embodiments, the antigen-binding moiety of the present disclosure comprises a polypeptide or polypeptides comprising: (i) a VH region comprising HC-CDR1, HC-CDR2 and HC-CDR3 as indicated in column A of Table A, and (ii) a VL region comprising LC-CDR1, LC-CDR2 and LC-CDR3 as indicated in column B of Table A, wherein the sequences of columns A and B are selected from the same row of Table A.

In some embodiments, the antigen-binding moiety of the present disclosure comprises a polypeptide or polypeptides comprising: (i) a VH region comprising HC-CDR1 according to SEQ ID NO:56, HC-CDR2 according to SEQ ID NO:64, and HC-CDR3 according to SEQ ID NO:58, and (ii) a VL region comprising LC-CDR1 according to SEQ ID NO:69, LC-CDR2 according to SEQ ID NO:70, and LC-CDR3 according to SEQ ID NO:71.

In some embodiments, the antigen-binding moiety of the present disclosure comprises a polypeptide or polypeptides comprising: (i) a VH region comprising HC-FR1, HC-FR2, HC-FR3 and HC-FR4 as indicated in column A of Table B, and (ii) a VL region comprising LC-FR1, LC-FR2, LC-FR3, and LC-FR4 as indicated in column B of Table B, wherein the sequences of columns A and B are selected from the same row of Table B.

In some embodiments, the antigen-binding moiety of the present disclosure comprises a polypeptide or polypeptides comprising: (i) a VH region comprising HC-FR1 according to SEQ ID NO:66, HC-FR2 according to SEQ ID NO:60, HC-FR3 according to SEQ ID NO:67, and HC-FR4 according to SEQ ID NO: 62, and (ii) a VL region comprising LC-FR1 according to SEQ ID NO:72, LC-FR2 according to SEQ ID NO: 73, LC-FR3 according to SEQ ID NO:74, and LC-FR4 according to SEQ ID NO:75.

In some embodiments, the antigen-binding moiety of the present disclosure comprises a polypeptide or polypeptides comprising: (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column A of Table C, and (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column B of Table C, wherein the sequences of columns A and B are selected from the same row of Table C.

In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:55, 63 or 65. In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:68.

In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:65. In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:68.

In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:76. In some embodiments, an antigen-binding moiety of the present disclosure comprises an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:84.

In some embodiments, an antigen-binding moiety of the present disclosure comprises, or consists of, an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:94. In some embodiments, an antigen-binding moiety comprises, or consists of, an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:95. In some embodiments, an antigen-binding moiety comprises, or consists of, an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:96. In some embodiments, an antigen-binding moiety comprises, or consists of, an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:97.

In aspects and embodiments of the present disclosure, a recombinant CD3-TCR complex polypeptide comprises a component of an antigen-binding moiety. This is particularly the case when it is contemplated to employ the recombinant CD3-TCR complex polypeptide with another, non-identical and complementary recombinant CD3-TCR complex polypeptide. In such aspects and embodiments, the two non-identical and complementary polypeptides preferably associate with one another to form a polypeptide complex comprising the antigen-binding moiety. That is, association between the recombinant CD3-TCR complex polypeptides reconstitutes the functional antigen-binding moiety.

By way of illustration, in embodiments described herein, a recombinant CD3-TCR complex polypeptide comprises the VH region of an antigen-binding moiety specific for a variant Fc domain and the ECD, TMD and ICD of TRAC (T47C), and it is contemplated to employ this recombinant CD3-TCR complex polypeptide in conjunction with a recombinant CD3-TCR complex polypeptide comprising the VL region of the antigen-binding moiety specific for a variant Fc domain, and the ECD, TMD and ICD of TRBC1 (S56C). When expressed in a cell, the two recombinant CD3-TCR complex polypeptides associate to form a polypeptide complex comprising an Fv specific for a variant Fc domain, formed by the VH region from the first polypeptide, and the VL region from the second polypeptide.

In some aspects and embodiments of the present disclosure, first and second components of an antigen-binding moiety are provided. In accordance with such aspects and embodiments, it will be appreciated that the first and second components of an antigen-binding moiety are complementary, and capable of associating to form the (complete, functional) antigen-binding moiety.

In some embodiments according to the present disclosure, a component of an antigen-binding moiety may be or comprise the VH region of an antigen-binding moiety specific for a variant Fc domain (e.g. as described herein). In some embodiments, a component of an antigen-binding moiety may be or comprise the VL region of an antigen-binding moiety specific for a variant Fc domain (e.g. as described herein). In preferred embodiments, the VH region and VL region may be from the same antigen-binding moiety.

In some embodiments, a component of an antigen-binding moiety comprises, or consists of, a VH as described herein. In some embodiments, a component of an antigen-binding moiety comprises, or consists of, a VL as described herein. In some embodiments, a component of an antigen-binding moiety comprises one or more antibody heavy chain constant regions (CH). In some embodiments, a component of an antigen-binding moiety comprises one or more antibody light chain constant regions (CL). In some embodiments, a component of an antigen-binding moiety comprises a CH1, CH2 region and/or a CH3 region of an immunoglobulin (Ig).

In some embodiments, a component of an antigen-binding moiety comprises or consists of a polypeptide or polypeptides comprising a VH region comprising HC-CDR1, HC-CDR2 and HC-CDR3 as indicated in column A of Table A. In some embodiments, a component of an antigen-binding moiety comprises or consists of a polypeptide or polypeptides comprising a VL region comprising LC-CDR1, LC-CDR2 and LC-CDR3 as indicated in column B of Table A.

In some embodiments, a component of an antigen-binding moiety comprises or consists of a polypeptide or polypeptides comprising a VH region comprising HC-FR1, HC-FR2, HC-FR3 and HC-FR4 as indicated in column A of Table B. In some embodiments, a component of an antigen-binding moiety comprises or consists of a polypeptide or polypeptides comprising a VL region comprising LC-FR1, LC-FR2, LC-FR3, and LC-FR4 as indicated in column B of Table B.

In some embodiments, a component of an antigen-binding moiety comprises or consists of a polypeptide or polypeptides comprising an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column A of Table C. In some embodiments, a component of an antigen-binding moiety comprises or consists of a polypeptide or polypeptides comprising an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column B of Table C.

In some embodiments, a component of an antigen-binding moiety comprises or consists of an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:55, 63 or 65. In some embodiments, a component of an antigen-binding moiety comprises or consists of an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:68.

In some embodiments, a component of an antigen-binding moiety comprises or consists of an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:76. In some embodiments, a component of an antigen-binding moiety comprises or consists of an amino acid having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:84.

It will be appreciated that where components of an antigen-binding moiety are provided in aspects and embodiments of the present disclosure, it is intended that the components provided are complementary, and capable of associating to form the (complete, functional) antigen-binding moiety.

### Variant Fc Domains

The antigen-binding moieties of the recombinant CD3-TCR complex polypeptides of the present disclosure provide for binding to a variant Fc domain.

As used herein, an ‘Fc domain’ refers to a polypeptide complex formed by interaction between two polypeptides, each polypeptide comprising the CH2-CH3 region of an immunoglobulin (Ig) heavy chain constant sequence.

Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (A).

Herein, a ‘CH2 domain’ refers to an amino acid sequence corresponding to the CH2 domain of an immunoglobulin (Ig). The CH2 domain is the region of an Ig formed by positions 231 to 340 of the immunoglobulin constant domain, according to the EU numbering system described in Edelman et al., Proc Natl Acad Sci USA (1969) 63 (1): 78-85. A ‘CH3 domain’ refers to an amino acid sequence corresponding to the CH3 domain of an immunoglobulin (Ig). The CH3 domain is the region of an Ig formed by positions 341 to 447 of the immunoglobulin constant domain, according to the EU numbering system described in Edelman et al., Proc Natl Acad Sci USA (1969) 63 (1): 78-85. A ‘CH2-CH3 region’ refers to an amino acid sequence corresponding to the CH2 and CH3 domains of an immunoglobulin (Ig). The CH2-CH3 region is the region of an Ig formed by positions 231 to 447 of the immunoglobulin constant domain, according to the EU numbering system described in Edelman et al., Proc Natl Acad Sci USA (1969) 63 (1): 78-85.

In some embodiments, a CH2 domain, CH3 domain and/or a CH2-CH3 region according to the present disclosure corresponds to the CH2 domain/CH3 domain/CH2-CH3 region of an IgG (e.g. IgG1, IgG2, IgG3, IgG4), IgA (e.g. IgA1, IgA2), IgD, IgE or IgM. In some embodiments, the CH2 domain, CH3 domain and/or a CH2-CH3 region corresponds to the CH2 domain/CH3 domain/CH2-CH3 region of a human IgG (e.g. hIgG1, hIgG2, hIgG3, hIgG4), hIgA (e.g. hIgA1, hIgA2), hIgD, hIgE or hIgM. In some embodiments, the CH2 domain, CH3 domain and/or a CH2-CH3 region corresponds to the CH2 domain/CH3 domain/CH2-CH3 region of a human IgG1 allotype (e.g. G1 ml, G1m2, G1m3 or G1m17).

It will be appreciated that an Fc domain according to the present disclosure may form part of a larger molecule comprising the Fc domain. For example, a variant Fc domain according to the present disclosure may be comprised in an antigen-binding molecule (e.g. an antibody) comprising an antigen-binding moiety specific for a target antigen, and a variant Fc domain according to the present disclosure.

Fc domains provide for interaction with Fc receptors and other molecules of the immune system to bring about functional effects. Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

The CH2-CH3 region sequence of the human IgG1 G1 ml allotype is shown in SEQ ID NO:206. The CH2-CH3 region sequence of the human IgG1 G1m3 allotype is shown in SEQ ID NO:207. The CH2-CH3 region sequence of human IgG2 is shown in SEQ ID NO:208. The CH2-CH3 region sequence of human IgG3 is shown in SEQ ID NO:209. The CH2-CH3 region sequence of human IgG4 is shown in SEQ ID NO:210.

Variant Fc domains according to the present disclosure comprise an amino acid sequence comprising at least one amino acid difference relative to a reference Fc domain.

In some embodiments, a reference Fc domain according to the present disclosure comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:206. In some embodiments, a reference Fc domain comprises two polypeptides, wherein each polypeptide comprises a CH2-CH3 region comprising or consisting of SEQ ID NO:206.

In some embodiments, a reference Fc domain comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:207. In some embodiments, a reference Fc domain comprises two polypeptides, wherein each polypeptide comprises a CH2-CH3 region comprising or consisting of SEQ ID NO: 207.

In some embodiments, a reference Fc domain comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:208. In some embodiments, a reference Fc domain comprises two polypeptides, wherein each polypeptide comprises a CH2-CH3 region comprising or consisting of SEQ ID NO: 208.

In some embodiments, a reference Fc domain comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:209. In some embodiments, a reference Fc domain comprises two polypeptides, wherein each polypeptide comprises a CH2-CH3 region comprising or consisting of SEQ ID NO: 209.

In some embodiments, a reference Fc domain comprises two polypeptides, wherein each polypeptide comprises a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:210. In some embodiments, a reference Fc domain comprises two polypeptides, wherein each polypeptide comprises a CH2-CH3 region comprising or consisting of SEQ ID NO: 210.

A variant Fc domain according to the present disclosure may comprise an amino acid difference relative to the amino acid sequence of one or both of the polypeptides of a reference Fc domain according to the present disclosure.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:206. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:206. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:206, wherein one or both of the CH2-CH3 regions comprises an amino acid sequence which is non-identical to SEQ ID NO: 206. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:206, wherein one or both of the CH2-CH3 regions comprise an amino acid sequence having one or more (e.g. 1, 2, 3, 4, 5 or more) amino acid differences relative to SEQ ID NO:206.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:207. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:207. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:207, wherein one or both of the CH2-CH3 regions comprises an amino acid sequence which is non-identical to SEQ ID NO: 207. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:207, wherein one or both of the CH2-CH3 regions comprise an amino acid sequence having one or more (e.g. 1, 2, 3, 4, 5 or more) amino acid differences relative to SEQ ID NO:207.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:208. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:208. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:208, wherein one or both of the CH2-CH3 regions comprises an amino acid sequence which is non-identical to SEQ ID NO: 208. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:208, wherein one or both of the CH2-CH3 regions comprise an amino acid sequence having one or more (e.g. 1, 2, 3, 4, 5 or more) amino acid differences relative to SEQ ID NO:208.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:209. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:209. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:209, wherein one or both of the CH2-CH3 regions comprises an amino acid sequence which is non-identical to SEQ ID NO: 209. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:209, wherein one or both of the CH2-CH3 regions comprise an amino acid sequence having one or more (e.g. 1, 2, 3, 4, 5 or more) amino acid differences relative to SEQ ID NO:209.

In some embodiments, a variant Fc domain according to the present disclosure comprises two polypeptides, each polypeptide comprising a CH2-CH3 region, and wherein one or both of the CH2-CH3 regions comprise an amino acid sequence which is non-identical to SEQ ID NO:210. In some embodiments, a variant Fc domain according to the present disclosure comprises two polypeptides, each polypeptide comprising a CH2-CH3 region, and wherein one or both of the CH2-CH3 regions comprise an amino acid sequence having one or more (e.g. 1, 2, 3, 4, 5 or more) amino acid differences relative to SEQ ID NO:210. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:210, wherein one or both of the CH2-CH3 regions comprises an amino acid sequence which is non-identical to SEQ ID NO: 210. In some embodiments, each CH2-CH3 region of the variant Fc domain comprises or consists of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% sequence identity, to the amino acid sequence of SEQ ID NO:210, wherein one or both of the CH2-CH3 regions comprise an amino acid sequence having one or more (e.g. 1, 2, 3, 4, 5 or more) amino acid differences relative to SEQ ID NO:210.

In some embodiments, each CH2-CH3 region of a variant Fc domain according to the present disclosure comprises an amino acid difference relative to a reference Fc domain according to the present disclosure. In some embodiments, the amino acid sequences of the CH2-CH3 regions of the constituent polypeptides of a variant Fc domain according to the present disclosure are identical (i.e. they have the same amino acid sequence).

The amino acid difference of a variant Fc domain according to the present disclosure (relative to a reference Fc domain) may influence an Fc-mediated function.

Modifications to Fc domains that influence Fc-mediated function are known in the art, such as those described e.g. in Wang et al., Protein Cell (2018) 9 (1): 63-73 and Saunders et al., Front Immunol. (2019) 10:1296, both of which are hereby incorporated by reference in their entirety. Exemplary Fc domain modifications known to influence Fc-mediated function are summarised in Table 1 of Wang et al., Protein Cell (2018) 9 (1): 63-73, and in Tables 1, 2 and 3 of Saunders et al., Front Immunol. (2019) 10:1296. In some embodiments, the variant Fc domain of the present disclosure comprises an Fc domain comprising an amino acid difference relative to a reference Fc domain (e.g. a reference Fc domain according to the present disclosure) that increases or reduces an Fc-mediated function.

In some embodiments, the variant Fc domain comprises an amino acid difference relative to a reference Fc domain that increases an Fc-mediated function. In some embodiments, the variant Fc domain comprises an amino acid difference relative to a reference Fc domain that increases ADCC, ADCP and/or CDC. Accordingly, in some embodiments the variant Fc domain displays an increased level of an Fc-mediated function as compared to the reference Fc domain. In some embodiments, the variant Fc domain displays increased ADCC, ADCP and/or CDC as compared to the reference Fc domain.

In some embodiments, the variant Fc domain comprises an amino acid difference relative to a reference Fc domain that increases binding to an Fc receptor (e.g. a Fcγ receptor, e.g. FcγRI, FcγRIIa, FcγRIIb, FcγRIIc, FcγRIIIa and/or FcγRIIIb). In some embodiments, the variant Fc domain comprises an amino acid difference relative to a reference Fc domain that increases binding to FcRn. In some embodiments, the variant Fc domain comprises an amino acid difference relative to a reference Fc domain that increases binding to a complement protein (e.g. C1q). In some embodiments, the variant Fc domain comprises an amino acid difference relative to a reference Fc domain to increase hexamerisation of an antigen-binding molecule comprising the variant Fc domain. In some embodiments, the Fc domain comprises an amino acid difference relative to a reference Fc domain that increases the half-life of an antigen-binding molecule comprising the variant Fc domain. Accordingly, in some embodiments the variant Fc domain displays increased binding to an Fc receptor (e.g. a Fcγ receptor, e.g. FcγRI, FcγRIIa, FcγRIIb, FcγRIIc, FcγRIIIa and/or FcγRIIIb) as compared to the reference Fc domain. In some embodiments the variant Fc domain displays increased binding to FcRn as compared to the reference Fc domain. In some embodiments the variant Fc domain displays increased binding to a complement protein (e.g. C1q) as compared to the reference Fc domain. In some embodiments an antigen-binding molecule comprising the variant Fc domain displays increased hexamerisation as compared to an antigen-binding molecule comprising the reference Fc domain. In some embodiments an antigen-binding molecule comprising the variant Fc domain displays an increased half-life as compared to an antigen-binding molecule comprising the reference Fc domain.

In some embodiments, the variant Fc domain comprises an amino acid difference relative to a reference Fc domain that reduces an Fc-mediated function. In some embodiments, the variant Fc domain comprises an amino acid difference relative to a reference Fc domain that reduces ADCC, ADCP and/or CDC. Accordingly, in some embodiments the variant Fc domain displays a reduced level of an Fc-mediated function as compared to the reference Fc domain. In some embodiments, the variant Fc domain displays reduced ADCC, ADCP and/or CDC as compared to the reference Fc domain.

In some embodiments, the variant Fc domain comprises an amino acid difference relative to a reference Fc domain that reduces binding to an Fc receptor (e.g. a Fcγ receptor, e.g. FcγRI, FcγRIIa, FcγRIIb, FcγRIIc, FcγRIIIa and/or FcγRIIIb). In some embodiments, the variant Fc domain comprises an amino acid difference relative to a reference Fc domain that reduces binding to FcRn. In some embodiments, the variant Fc domain comprises an amino acid difference relative to a reference Fc domain that reduces binding to a complement protein (e.g. C1q). In some embodiments, the variant Fc domain comprises an amino acid difference relative to a reference Fc domain to increase hexamerisation of an antigen-binding molecule comprising the variant Fc domain. In some embodiments, the Fc domain comprises an amino acid difference relative to a reference Fc domain that reduces the half-life of an antigen-binding molecule comprising the variant Fc domain. Accordingly, in some embodiments the variant Fc domain displays reduced binding to an Fc receptor (e.g. a Fcγ receptor, e.g. FcγRI, FcγRIIa, FcγRIIb, FcγRIIc, FcγRIIIa and/or FcγRIIIb) as compared to the reference Fc domain. In some embodiments the variant Fc domain displays reduced binding to FcRn as compared to the reference Fc domain. In some embodiments the variant Fc domain displays reduced binding to a complement protein (e.g. C1q) as compared to the reference Fc domain. In some embodiments an antigen-binding molecule comprising the variant Fc domain displays reduced hexamerisation as compared to an antigen-binding molecule comprising the reference Fc domain. In some embodiments an antigen-binding molecule comprising the variant Fc domain displays a reduced half-life as compared to an antigen-binding molecule comprising the reference Fc domain.

In some embodiments, the variant Fc domain comprises a CH2-CH3 region comprising an amino acid difference at one or more of the following positions, relative to the amino acid sequence of a CH2-CH3 region of the reference Fc domain: 233, 234, 235, 253, 297, 298, 310, 329, 331, 333, 334 or 435 (according to the EU numbering system). In some embodiments, the variant Fc domain comprises a CH2-CH3 region comprising an amino acid difference at one or more of the following positions, relative to the amino acid sequence of a CH2-CH3 region of the reference Fc domain: 234, 235, 253, 297, 298, 310, 329, 333, 334 or 435. In some embodiments, the variant Fc domain comprises a CH2-CH3 region comprising an amino acid difference at position 329, relative to the amino acid sequence of a CH2-CH3 region of the reference Fc domain. In some embodiments, the variant Fc domain comprises a CH2-CH3 region comprising an amino acid difference at positions 234, 235 and 329, relative to the amino acid sequence of a CH2-CH3 region of the reference Fc domain. In some embodiments, the variant Fc domain comprises a CH2-CH3 region comprising an amino acid difference at positions 298, 333 and 334, relative to the amino acid sequence of a CH2-CH3 region of the reference Fc domain.

In some embodiments, the variant Fc domain comprises a CH2-CH3 region comprising an amino acid difference at one or more of the following positions, relative to the amino acid sequence of a CH2-CH3 region of the reference Fc domain: E233, L234, L235, I253, N297, S298, H310, P329, P331, E333, K334 or H435 (according to the EU numbering system). In some embodiments, the variant Fc domain comprises a CH2-CH3 region comprising an amino acid difference at one or more of the following positions, relative to the amino acid sequence of a CH2-CH3 region of the reference Fc domain: L234, L235, 1253, N297, S298, H310, P329, E333, K334 or H435. In some embodiments, the variant Fc domain comprises a CH2-CH3 region comprising an amino acid difference at P329, relative to the amino acid sequence of a CH2-CH3 region of the reference Fc domain. In some embodiments, the variant Fc domain comprises a CH2-CH3 region comprising an amino acid difference at positions L234, L235 and P329, relative to the amino acid sequence of a CH2-CH3 region of the reference Fc domain. In some embodiments, the variant Fc domain comprises a CH2-CH3 region comprising an amino acid difference at positions S298, E333 and K334, relative to the amino acid sequence of a CH2-CH3 region of the reference Fc domain.

In some embodiments, the variant Fc domain comprises a CH2-CH3 region comprising one or more of the following specified amino acid residues: P233, A234, A235, A253, A297, A298, A310, G329, S331, A333, A334 or A435 (according to the EU numbering system). In some embodiments, the variant Fc domain comprises a CH2-CH3 region comprising one or more of the following specified amino acid residues: A234, A235, A253, A297, A298, A310, G329, A333, A334 or A435. In some embodiments, the variant Fc domain comprises a CH2-CH3 region comprising G329. In some embodiments, the variant Fc domain comprises a CH2-CH3 region comprising A234, A235 and G329. In some embodiments, the variant Fc domain comprises a CH2-CH3 region comprising A298, A333 and A334.

In some embodiments, the variant Fc domain comprises a CH2-CH3 region comprising one or more of the following amino acid substitutions, relative to the amino acid sequence of a CH2-CH3 region of the reference Fc domain: E233P, L234A, L235A, 1253A, N297A, S298A, H310A, P329G, P331S, E333A, K334A or H435A (according to the EU numbering system). In some embodiments, the variant Fc domain comprises a CH2-CH3 region comprising one or more of the following amino acid substitutions, relative to the amino acid sequence of a CH2-CH3 region of the reference Fc domain: L234A, L235A, 1253A, N297A, S298A, H310A, P329G, E333A, K334A or H435A. In some embodiments, the variant Fc domain comprises a CH2-CH3 region comprising the amino acid substitution P329G, relative to the amino acid sequence of a CH2-CH3 region of the reference Fc domain. In some embodiments, the variant Fc domain comprises a CH2-CH3 region comprising the amino acid substitutions L234A, L235A and P329G relative to the amino acid sequence of a CH2-CH3 region of the reference Fc domain. In some embodiments, the variant Fc domain comprises a CH2-CH3 region comprising the amino acid substitutions S298A, E333A and K334A relative to the amino acid sequence of a CH2-CH3 region of the reference Fc domain.

In some embodiments, a variant Fc domain according to the present disclosure comprises a polypeptide comprising a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:211 or 214, wherein the CH2-CH3 region comprises G329. In some embodiments, a variant Fc domain according to the present disclosure comprises two polypeptides, each polypeptide comprising a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:211 or 214, wherein the CH2-CH3 region comprises G329. In some embodiments, a variant Fc domain according to the present disclosure comprises one or more (e.g. two) polypeptides comprising the amino acid sequence of SEQ ID NO:211 or 214.

In some embodiments, a variant Fc domain according to the present disclosure comprises a polypeptide comprising a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:213 or 216, wherein the CH2-CH3 region comprises A234, A235 and G329. In some embodiments, a variant Fc domain according to the present disclosure comprises two polypeptides, each polypeptide comprising a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:213 or 216, wherein the CH2-CH3 region comprises A234, A235 and G329. In some embodiments, a variant Fc domain according to the present disclosure comprises one or more (e.g. two) polypeptides comprising the amino acid sequence of SEQ ID NO:213 or 216.

In some embodiments, a variant Fc domain according to the present disclosure comprises a polypeptide comprising a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:212 or 215, wherein the CH2-CH3 region comprises A298, A333 and A334. In some embodiments, a variant Fc domain according to the present disclosure comprises two polypeptides, each polypeptide comprising a CH2-CH3 region comprising or consisting of an amino acid sequence having at least 70% sequence identity, more preferably one of at least ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity, to the amino acid sequence of SEQ ID NO:212 or 215, wherein the CH2-CH3 region comprises A298, A333 and A334. In some embodiments, a variant Fc domain according to the present disclosure comprises one or more (e.g. two) polypeptides comprising the amino acid sequence of SEQ ID NO:212 or 215.

### Composite Polypeptides

Aspects and embodiments of the present disclosure pertain to composite polypeptides.

The composite polypeptides of the present disclosure comprise an amino acid sequence encoding at least two recombinant CD3-TCR complex polypeptides according to the present disclosure. The amino acid sequences of the recombinant CD3-TCR complex polypeptides of the composite polypeptide are provided in tandem. A cleavage site may be provided in the composite polypeptide, between the amino acid sequences of the recombinant CD3-TCR complex polypeptides, as described hereinbelow.

In some embodiments, the individual recombinant CD3-TCR complex polypeptides comprised in a composite polypeptide of the present disclosure are non-identical. In some embodiments, recombinant CD3-TCR complex polypeptides comprised in a composite polypeptide of the present disclosure are complementary. That is, in some embodiments recombinant CD3-TCR complex polypeptides comprised in a composite polypeptide of the present disclosure are capable of associating with one another (e.g. via non-covalent, protein: protein interaction) to form a polypeptide complex (e.g. a polypeptide complex as described herein).

In further preferred embodiments, the individual recombinant CD3-TCR complex polypeptides comprised in a composite polypeptide of the present disclosure are capable of associating with one another to form an antigen-binding moiety according to the present disclosure. In some embodiments, the recombinant CD3-TCR complex polypeptides comprised in a composite polypeptide of the present disclosure comprise complementary components of an antigen-binding moiety according to the present disclosure. Complementary components of an antigen-binding moiety are capable of association (e.g. via non-covalent, protein: protein interaction) to form an antigen-binding moiety. In some embodiments, association between recombinant CD3-TCR complex polypeptides comprised in a composite polypeptide of the present disclosure results in the formation of an antigen-binding moiety according to the present disclosure. In some embodiments, a recombinant CD3-TCR complex polypeptide of a composite polypeptide of the present disclosure comprises the VH region of an antigen-binding moiety that binds to a variant Fc domain (e.g. as described herein), and another CD3-TCR complex polypeptide of the composite polypeptide comprises the VL region of the antigen-binding moiety.

By way of illustration, in embodiments described herein, a composite polypeptide comprises (i) a recombinant CD3-TCR complex polypeptide comprising the VH of an antigen-binding moiety specific for a variant Fc domain and the ECD, TMD and ICD of TRAC (T47C), and (ii) a recombinant CD3-TCR complex polypeptide comprising the VL region of the antigen-binding moiety specific for a variant Fc domain, and the ECD, TMD and ICD of TRBC1 (S56C), and (iii) further comprising a T2A cleavage site between polypeptides (i) and (ii). Following cleavage of the composite polypeptide, polypeptides (i) and (ii) associate to form a polypeptide complex comprising an Fv specific for a variant Fc domain, formed by the VH from (i) and the VL from (ii).

### Linkers, Labels and Conjugates

The polypeptides of the present disclosure (e.g. recombinant CD3-TCR complex polypeptides and composite polypeptides) may additionally comprise further amino acids or sequences of amino acids.

The polypeptides may comprise one or more linker sequences between sequences of amino acids. By way of example, a linker sequence may be provided between different domains of a recombinant CD3-TCR complex polypeptide (e.g. between the antigen-binding moiety and CD3-TCR complex association domain). By way of further example, a linker sequence may be provided between subsequences of the domains of a recombinant CD3-TCR complex polypeptide (e.g. between VH region and VL region of an antigen-binding moiety).

Linker sequences are known to the skilled person, and are described, for example in Chen et al., Adv Drug Deliv Rev (2013) 65 (10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65 (10): 1357-1369. Flexible linker sequences often comprise high proportions of glycine and/or serine residues.

In some embodiments, a linker sequence comprises at least one glycine residue and/or at least one serine residue. In some embodiments, the linker sequence comprises or consists of glycine and serine residues. In some embodiments, the linker sequence has the structure: (GxS)n or (GxS)nGm; wherein G=glycine, S=serine, x=3 or 4, n=2, 3, 4, 5 or 6, and m=0, 1, 2 or 3. In some embodiments, the linker sequence comprises one or more (e.g. 1, 2, 3, 4, 5 or 6) copies (e.g. in tandem) of the sequence motif G₄S. In some embodiments, a linker sequence comprises or consists of (G₄S)₃ or (G₄S)₄. In some embodiments, the linker sequence has a length of 1-2, 1-3, 1-4, 1-5, 1-10, 1-15, 1-20, 1-25, or 1-30 amino acids.

In some embodiments, the linker sequence comprises one or more copies of an amino acid sequence according to SEQ ID NO:92. In some embodiments, the linker sequence comprises at least 1, 2, 3 or 4 copies of an amino acid sequence according to SEQ ID NO:92.

In some embodiments, the linker sequence comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:93.

In some embodiments, the linker sequence comprises a cleavage site, e.g. a cleavage site as described hereinbelow.

The polypeptides of the present disclosure may comprise amino acid sequence(s) to facilitate expression, folding, trafficking, processing, purification or detection of the antigen-binding molecule/polypeptide. For example, recombinant CD3-TCR complex polypeptides of the present disclosure may additionally comprise a sequence of amino acids forming a detectable moiety, e.g. as described hereinbelow.

The polypeptides may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides. Signal peptides are known for many proteins, and are recorded in databases such as GenBank, UniProt and Ensembl, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

The signal peptide may be present at the N-terminus of the recombinant CD3-TCR complex polypeptide, and may be present in the newly-synthesised polypeptide. The signal peptide provides for efficient trafficking of the recombinant CD3-TCR complex polypeptide. Signal peptides are often removed by cleavage, and thus are not comprised in the mature recombinant CD3-TCR complex polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

In some embodiments, the signal peptide comprises, or consists of, an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:98.

In some embodiments, polypeptides of the present disclosure (e.g. recombinant CD3-TCR complex polypeptides and composite polypeptides) comprise a detectable moiety. In some embodiments, a detectable moiety is provided at the N-terminus and/or C-terminus of the polypeptide.

In some embodiments, a detectable moiety is a fluorescent label, phosphorescent label, luminescent label, immuno-detectable label (e.g. an epitope tag), radiolabel, chemical, nucleic acid or enzymatic label. The recombinant CD3-TCR complex polypeptide may be covalently or non-covalently labelled with the detectable moiety.

Fluorescent labels include e.g. fluorescein, rhodamine, allophycocyanin, eosine and NDB, green fluorescent protein (GFP), enhanced GFP (eGFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Hydrogen³, Sulfur³⁵, Carbon¹⁴, Phosphorus³², Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium 103, Ruthenium 105, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, ThuliumI¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers.

In some embodiments, the recombinant CD3-TCR complex polypeptide comprises an epitope tag, e.g. a His, (e.g. 6XHis), FLAG, c-Myc, StrepTag, haemagglutinin, E, calmodulin-binding protein (CBP), glutathione-s-transferase (GST), maltose-binding protein (MBP), thioredoxin, S-peptide, T7 peptide, SH2 domain, avidin, streptavidin, and haptens (e.g. biotin, digoxigenin, dinitrophenol), optionally at the N- or C-terminus of the recombinant CD3-TCR complex polypeptide.

In some embodiments, the recombinant CD3-TCR complex polypeptide comprises a moiety having a detectable activity, e.g. an enzymatic moiety. Enzymatic moieties include e.g. luciferases, glucose oxidases, galactosidases (e.g. beta-galactosidase), glucorinidases, phosphatases (e.g. alkaline phosphatase), peroxidases (e.g. horseradish peroxidase) and cholinesterases.

In some embodiments, a polypeptide of the present disclosure comprises a fluorescent label. In some embodiments, the polypeptide comprises an eGFP moiety. In some embodiments, the polypeptide comprises an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 103.

Polypeptides of the present disclosure may also comprise one or more cleavage sites. A cleavage site refers to a sequence of amino acids that acts as a substrate for an enzyme capable of cleaving peptide bonds.

Many such cleavage sites are known to, and can be employed by, the person skilled in the art of molecular biology. In some embodiments, the cleavage sequence comprises an autocleavage site. Autocleavage sites include the 2A cleavage sequence from Picornavirus ‘NPGP’, which is cleaved at ‘G/P’. Further autocleavage sites are described e.g. in Kim et al., PLOS ONE (2011) 6: e18556 (hereby incorporated by reference in its entirety), and include e.g. T2A, P2A, E2A and F2A cleavage sites. The amino acid sequences of T2A, P2A, E2A and F2A cleavage sites are shown in SEQ ID NOs: 104, 220, 109 and 221, respectively.

A cleavage site may be included in a polypeptide according to the present disclosure to provide for removal of a moiety or domain. It might be desirable to remove a given moiety or domain so that it is not comprised in the polypeptide complex formed by the polypeptide. For example, in embodiments of recombinant CD3-TCR complex polypeptides of the present disclosure, a cleavage site (specifically, a T2A cleavage site) is provided upstream of an eGFP moiety, to provide for its removal such that the eGFP moiety is not included in CD3-TCR complexes comprising the CD3-TCR complex polypeptide. Accordingly, in some embodiments, a polypeptide according to the present disclosure comprises a cleavage site adjacent to (i.e. in the amino acid sequence of the polypeptide, e.g. immediately upstream or downstream of) a detectable moiety according to the present disclosure.

A cleavage site may be included in a composite polypeptide according to the present disclosure, to provide for separation of the recombinant CD3-TCR complex polypeptides comprised in the composite polypeptide. For example, in embodiments of composite polypeptides of the present disclosure, a cleavage site (specifically, a T2A cleavage site) is provided between amino acid sequences encoding complementary recombinant CD3-TCR complex polypeptides, and provides for physical separation of the recombinant CD3-TCR complex polypeptides. Accordingly, in some embodiments, a polypeptide according to the present disclosure comprises a cleavage site adjacent to (i.e. in the amino acid sequence of the polypeptide, e.g. immediately upstream or downstream of) an amino acid sequence encoding a recombinant CD3-TCR complex polypeptide according to the present disclosure.

In some embodiments, a cleavage site according to the present disclosure is a 2A cleavage site, e.g. selected from a T2A, P2A, E2A and F2A cleavage site. In some embodiments, the cleavage site is a T2A cleavage site.

In some embodiments, a polypeptide according to the present disclosure comprises an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 104, 220, 109 or 221. In some embodiments, a polypeptide according to the present disclosure comprises an amino acid sequence having at least 60%, preferably one of ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 104.

### Polypeptide Complexes

The present disclosure also provides polypeptide complexes. A ‘polypeptide complex’ refers to a molecule formed by non-covalent, protein: protein interaction between two or more polypeptides. In some embodiments, the association comprises electrostatic interaction (e.g. ionic bonding, hydrogen bonding) and/or Van der Waals forces.

Aspects and embodiments of the present disclosure relate to polypeptide complexes formed by association between non-identical, complementary recombinant CD3-TCR complex polypeptides according to the present disclosure.

In some embodiments, the constituent polypeptides of such polypeptide complexes comprise complementary CD3-TCR complex association domains. In some embodiments, association between the constituent polypeptides of such polypeptide complexes comprises interaction between the CD3-TCR complex association domains of the recombinant CD3-TCR complex polypeptides. For example, one of the recombinant CD3-TCR complex polypeptides may comprise a CD3-TCR complex association domain derived from TRAC, and the other may comprise a CD3-TCR complex association domain derived from TRBC1 or TRBC2. In some embodiments, the CD3-TCR complex association domains of the recombinant CD3-TCR complex polypeptides of such complexes comprise complementary modifications to promote their association. In some embodiments, the CD3-TCR complex association domains of the recombinant CD3-TCR complex polypeptides comprise modifications introducing cysteine residues for the formation of an interchain disulfide bond between the CD3-TCR complex association domains.

For example, a polypeptide complex according to the present disclosure may comprise (i) a recombinant CD3-TCR complex polypeptide comprising a CD3-TCR complex association domain derived from TRAC, further comprising the modification T47C, and (ii) a recombinant CD3-TCR complex polypeptide comprising a CD3-TCR complex association domain derived from TRBC1 or TRBC2, further comprising the modification S56C.

In some embodiments, the constituent polypeptides of such polypeptide complexes comprise complementary components of an antigen-binding moiety specific for a variant Fc domain (e.g. as described herein). In some embodiments, association between the constituent polypeptides comprises interaction between the individual components of an antigen-binding moiety provided by the different recombinant CD3-TCR complex polypeptides. For example, one of the recombinant CD3-TCR complex polypeptides may comprise the VH region of an antigen-binding moiety specific for a variant Fc domain, and the other may comprise the VL region of the antigen-binding moiety.

Aspects and embodiments of the present disclosure also relate to CD3-TCR polypeptide complexes.

A CD3-TCR polypeptide complex according to the present disclosure comprises a recombinant CD3-TCR complex polypeptide according to the present disclosure. In some embodiments, a CD3-TCR polypeptide complex may comprise one or more (e.g. two, three, four or more) recombinant CD3-TCR complex polypeptides according to the present disclosure.

In some aspects and embodiments wherein a CD3-TCR polypeptide complex comprises plural recombinant CD3-TCR complex polypeptides according to the present disclosure, the recombinant CD3-TCR complex polypeptides may be identical.

In some aspects and embodiments, a CD3-TCR polypeptide complex comprises non-identical recombinant CD3-TCR complex polypeptides according to the present disclosure. In some aspects and embodiments, a CD3-TCR polypeptide complex comprises a polypeptide complex as described hereinabove (i.e. a polypeptide complex formed by association of complementary recombinant CD3-TCR complex polypeptides according to the present disclosure). That is, the polypeptide complex may be a CD3-TCR polypeptide complex, or may form part of a CD3-TCR polypeptide complex.

It will be appreciated that in addition to (a) recombinant CD3-TCR complex polypeptide(s), a CD3-TCR polypeptide complex according to the present disclosure may comprise (b) one or more further CD3-TCR complex polypeptides, e.g. selected from TCRα, TCRβ, TCRγ, TCRδ, TRAC, TRBC1, TRBC2, TRGC1, TRGC2, TRDC, CD3ε, CD3δ, CD3γ, CD3ζ and CD3η.

A CD3-TCR polypeptide complex according to the present disclosure may optionally be characterised by the ability to elicit one or more of the following by an immune cell (e.g. a T cell) expressing the CD3-TCR polypeptide complex, in response to the antigen for which CD3-TCR polypeptide complex comprises an antigen-binding moiety (or in response to a cell comprising or expressing the antigen): CD3-TCR complex-mediated signalling, proliferation/population expansion, growth factor (e.g. IL-2) expression, IFNγ expression, CD107a expression, TNFα expression, GM-CSF expression, perforin expression, granzyme expression, granulysin expression, and/or FAS ligand (FASL) expression.

For example, an immune cell (e.g. a T cell) expressing a CD3-TCR polypeptide complex according to the present disclosure may be capable of eliciting one or more of the functional properties recited in the preceding paragraph in response to a variant Fc domain according to the present disclosure (i.e. a variant Fc domain that is bound by antigen-binding moiety of a recombinant CD3-TCR complex polypeptide/complex thereof comprised in the CD3-TCR polypeptide complex), or in response to a cell comprising/expressing such a variant Fc domain.

### Particular Exemplary CD3-TCR Complex Polypeptides, Composite Polypeptides and Polypeptide Complexes

In some embodiments, a CD3-TCR complex polypeptide according to the present disclosure comprises or consists of one of the following structures:
N term-[signal peptide]-[antigen binding moiety, or a component thereof]-[CD3-TCR complex association domain]-C term
N term-[antigen binding moiety, or a component thereof]-[CD3-TCR complex association domain]-C term
N term-[signal peptide]-[antigen binding moiety, or a component thereof]-[CD3-TCR complex association domain]-[cleavage site]-[detectable moiety]-C term
N term-[antigen binding moiety, or a component thereof]-[CD3-TCR complex association domain]-[cleavage site]-[detectable moiety]-C term

In some embodiments, a composite polypeptide according to the present disclosure comprises or consists of one of the following structures:
N term-[signal peptide]-[antigen-binding moiety component]-[CD3-TCR complex association domain]-[cleavage site]-[signal peptide]-[antigen-binding moiety component]-[CD3-TCR complex association domain]-C term
N term-[signal peptide]-[antigen-binding moiety component]-[CD3-TCR complex association domain]-[cleavage site]-[signal peptide]-[antigen-binding moiety component]-[CD3-TCR complex association domain]-[cleavage site]-[detectable moiety]-C term

In some embodiments, a CD3-TCR complex polypeptide according to the present disclosure comprises or consists of (e.g. from N-terminus to C-terminus):

(1) (i) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:98;
(ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 1; and
(iii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 1;
wherein the sequence selected from Column A of Table 1 and the sequence selected from Column B of Table 1 are selected from the same row of Table 1.

(2) (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 1; and
(ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 1;
wherein the sequence selected from Column A of Table 1 and the sequence selected from Column B of Table 1 are selected from the same row of Table 1.

(3) (i) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:98;
(ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 1;
(iii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 1;
(iv) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 104; and
(v) an amino acid sequence encoding a detectable moiety, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 103;
wherein the sequence selected from Column A of Table 1 and the sequence selected from Column B of Table 1 are selected from the same row of Table 1.

(4) (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 1;
(ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 1;
(iii) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 104; and
(iv) an amino acid sequence encoding a detectable moiety, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:103;
wherein the sequence selected from Column A of Table 1 and the sequence selected from Column B of Table 1 are selected from the same row of Table 1.

TABLE 1

| Row | Column A | Column B |
|---|---|---|
| 1 | SEQ ID NO: 94 | SEQ ID NO: 30 |
| 2 | SEQ ID NO: 95 | SEQ ID NO: 30 |
| 3 | SEQ ID NO: 96 | SEQ ID NO: 30 |
| 4 | SEQ ID NO: 97 | SEQ ID NO: 30 |
| 5 | SEQ ID NO: 55 | SEQ ID NO: 1 |
| 6 | SEQ ID NO: 55 | SEQ ID NO: 52 |
| 7 | SEQ ID NO: 55 | SEQ ID NO: 5 |
| 8 | SEQ ID NO: 55 | SEQ ID NO: 53 |
| 9 | SEQ ID NO: 55 | SEQ ID NO: 9 |
| 10 | SEQ ID NO: 55 | SEQ ID NO: 54 |
| 11 | SEQ ID NO: 63 | SEQ ID NO: 1 |
| 12 | SEQ ID NO: 63 | SEQ ID NO: 52 |
| 13 | SEQ ID NO: 63 | SEQ ID NO: 5 |
| 14 | SEQ ID NO: 63 | SEQ ID NO: 53 |
| 15 | SEQ ID NO: 63 | SEQ ID NO: 9 |
| 16 | SEQ ID NO: 63 | SEQ ID NO: 54 |
| 17 | SEQ ID NO: 65 | SEQ ID NO: 1 |
| 18 | SEQ ID NO: 65 | SEQ ID NO: 52 |
| 19 | SEQ ID NO: 65 | SEQ ID NO: 5 |
| 20 | SEQ ID NO: 65 | SEQ ID NO: 53 |
| 21 | SEQ ID NO: 65 | SEQ ID NO: 9 |
| 22 | SEQ ID NO: 65 | SEQ ID NO: 54 |
| 23 | SEQ ID NO: 68 | SEQ ID NO: 1 |
| 24 | SEQ ID NO: 68 | SEQ ID NO: 52 |
| 25 | SEQ ID NO: 68 | SEQ ID NO: 5 |
| 26 | SEQ ID NO: 68 | SEQ ID NO: 53 |
| 27 | SEQ ID NO: 68 | SEQ ID NO: 9 |
| 28 | SEQ ID NO: 68 | SEQ ID NO: 54 |
| 29 | SEQ ID NO: 76 | SEQ ID NO: 1 |
| 30 | SEQ ID NO: 76 | SEQ ID NO: 52 |
| 31 | SEQ ID NO: 76 | SEQ ID NO: 5 |
| 32 | SEQ ID NO: 76 | SEQ ID NO: 53 |
| 33 | SEQ ID NO: 76 | SEQ ID NO: 9 |
| 34 | SEQ ID NO: 76 | SEQ ID NO: 54 |
| 35 | SEQ ID NO: 84 | SEQ ID NO: 1 |
| 36 | SEQ ID NO: 84 | SEQ ID NO: 52 |
| 37 | SEQ ID NO: 84 | SEQ ID NO: 5 |
| 38 | SEQ ID NO: 84 | SEQ ID NO: 53 |
| 39 | SEQ ID NO: 84 | SEQ ID NO: 9 |
| 40 | SEQ ID NO: 84 | SEQ ID NO: 54 |

In some embodiments, a CD3-TCR complex polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 110 to 173. In some embodiments, a CD3-TCR complex polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 121.

In some embodiments, a composite polypeptide according to the present disclosure comprises or consists of (e.g. from N-terminus to C-terminus):

(1) (i) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:98;
(ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 2; and
(iii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 2;
(iv) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 104;
(v) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:98;
(vi) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column C of Table 2; and
(vii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column D of Table 2;
wherein the sequence selected from Column A of Table 2 and the sequence selected from Column B of Table 2 and the sequence selected from Column C of Table 2 and the sequence selected from Column D of Table 2 are selected from the same row of Table 2.

(2) (i) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:98;

(ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 2; and
(iii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column B of Table 2;
(iv) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 104;
(v) an amino acid sequence encoding a signal peptide, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:98;
(vi) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column C of Table 2;
(vii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from of Column D of Table 2;
(viii) an amino acid sequence encoding a cleavage site, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 104; and
(ix) an amino acid sequence encoding a detectable moiety, e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:103;
wherein the sequence selected from Column A of Table 2 and the sequence selected from Column B of Table 2 and the sequence selected from Column C of Table 2 and the sequence selected from Column D of Table 2 are selected from the same row of Table 2.

TABLE 2

| Row | Column A | Column B | Column C | Column D |
|---|---|---|---|---|
| 1 | SEQ ID NO: 55 | SEQ ID NO: 1 | SEQ ID NO: 68 | SEQ ID NO: 5 |
| 2 | SEQ ID NO: 63 | SEQ ID NO: 1 | SEQ ID NO: 68 | SEQ ID NO: 5 |
| 3 | SEQ ID NO: 65 | SEQ ID NO: 1 | SEQ ID NO: 68 | SEQ ID NO: 5 |
| 4 | SEQ ID NO: 68 | SEQ ID NO: 1 | SEQ ID NO: 55 | SEQ ID NO: 5 |
| 5 | SEQ ID NO: 68 | SEQ ID NO: 1 | SEQ ID NO: 63 | SEQ ID NO: 5 |
| 6 | SEQ ID NO: 68 | SEQ ID NO: 1 | SEQ ID NO: 65 | SEQ ID NO: 5 |
| 7 | SEQ ID NO: 76 | SEQ ID NO: 1 | SEQ ID NO: 84 | SEQ ID NO: 5 |
| 8 | SEQ ID NO: 84 | SEQ ID NO: 1 | SEQ ID NO: 76 | SEQ ID NO: 5 |
| 9 | SEQ ID NO: 55 | SEQ ID NO: 1 | SEQ ID NO: 68 | SEQ ID NO: 9 |
| 10 | SEQ ID NO: 63 | SEQ ID NO: 1 | SEQ ID NO: 68 | SEQ ID NO: 9 |
| 11 | SEQ ID NO: 65 | SEQ ID NO: 1 | SEQ ID NO: 68 | SEQ ID NO: 9 |
| 12 | SEQ ID NO: 68 | SEQ ID NO: 1 | SEQ ID NO: 55 | SEQ ID NO: 9 |
| 13 | SEQ ID NO: 68 | SEQ ID NO: 1 | SEQ ID NO: 63 | SEQ ID NO: 9 |
| 14 | SEQ ID NO: 68 | SEQ ID NO: 1 | SEQ ID NO: 65 | SEQ ID NO: 9 |
| 15 | SEQ ID NO: 76 | SEQ ID NO: 1 | SEQ ID NO: 84 | SEQ ID NO: 9 |
| 16 | SEQ ID NO: 84 | SEQ ID NO: 1 | SEQ ID NO: 76 | SEQ ID NO: 9 |
| 17 | SEQ ID NO: 55 | SEQ ID NO: 52 | SEQ ID NO: 68 | SEQ ID NO: 53 |
| 18 | SEQ ID NO: 63 | SEQ ID NO: 52 | SEQ ID NO: 68 | SEQ ID NO: 53 |
| 19 | SEQ ID NO: 65 | SEQ ID NO: 52 | SEQ ID NO: 68 | SEQ ID NO: 53 |
| 20 | SEQ ID NO: 68 | SEQ ID NO: 52 | SEQ ID NO: 55 | SEQ ID NO: 53 |
| 21 | SEQ ID NO: 68 | SEQ ID NO: 52 | SEQ ID NO: 63 | SEQ ID NO: 53 |
| 22 | SEQ ID NO: 68 | SEQ ID NO: 52 | SEQ ID NO: 65 | SEQ ID NO: 53 |
| 23 | SEQ ID NO: 76 | SEQ ID NO: 52 | SEQ ID NO: 84 | SEQ ID NO: 53 |
| 24 | SEQ ID NO: 84 | SEQ ID NO: 52 | SEQ ID NO: 76 | SEQ ID NO: 53 |
| 25 | SEQ ID NO: 55 | SEQ ID NO: 52 | SEQ ID NO: 68 | SEQ ID NO: 54 |
| 26 | SEQ ID NO: 63 | SEQ ID NO: 52 | SEQ ID NO: 68 | SEQ ID NO: 54 |
| 27 | SEQ ID NO: 65 | SEQ ID NO: 52 | SEQ ID NO: 68 | SEQ ID NO: 54 |
| 28 | SEQ ID NO: 68 | SEQ ID NO: 52 | SEQ ID NO: 55 | SEQ ID NO: 54 |
| 29 | SEQ ID NO: 68 | SEQ ID NO: 52 | SEQ ID NO: 63 | SEQ ID NO: 54 |
| 30 | SEQ ID NO: 68 | SEQ ID NO: 52 | SEQ ID NO: 65 | SEQ ID NO: 54 |
| 31 | SEQ ID NO: 76 | SEQ ID NO: 52 | SEQ ID NO: 84 | SEQ ID NO: 54 |
| 32 | SEQ ID NO: 84 | SEQ ID NO: 52 | SEQ ID NO: 76 | SEQ ID NO: 54 |
| 33 | SEQ ID NO: 55 | SEQ ID NO: 5 | SEQ ID NO: 68 | SEQ ID NO: 1 |
| 34 | SEQ ID NO: 63 | SEQ ID NO: 5 | SEQ ID NO: 68 | SEQ ID NO: 1 |
| 35 | SEQ ID NO: 65 | SEQ ID NO: 5 | SEQ ID NO: 68 | SEQ ID NO: 1 |
| 36 | SEQ ID NO: 68 | SEQ ID NO: 5 | SEQ ID NO: 55 | SEQ ID NO: 1 |
| 37 | SEQ ID NO: 68 | SEQ ID NO: 5 | SEQ ID NO: 63 | SEQ ID NO: 1 |
| 38 | SEQ ID NO: 68 | SEQ ID NO: 5 | SEQ ID NO: 65 | SEQ ID NO: 1 |
| 39 | SEQ ID NO: 76 | SEQ ID NO: 5 | SEQ ID NO: 84 | SEQ ID NO: 1 |
| 40 | SEQ ID NO: 84 | SEQ ID NO: 5 | SEQ ID NO: 76 | SEQ ID NO: 1 |
| 41 | SEQ ID NO: 55 | SEQ ID NO: 9 | SEQ ID NO: 68 | SEQ ID NO: 1 |
| 42 | SEQ ID NO: 63 | SEQ ID NO: 9 | SEQ ID NO: 68 | SEQ ID NO: 1 |
| 43 | SEQ ID NO: 65 | SEQ ID NO: 9 | SEQ ID NO: 68 | SEQ ID NO: 1 |
| 44 | SEQ ID NO: 68 | SEQ ID NO: 9 | SEQ ID NO: 55 | SEQ ID NO: 1 |
| 45 | SEQ ID NO: 68 | SEQ ID NO: 9 | SEQ ID NO: 63 | SEQ ID NO: 1 |
| 46 | SEQ ID NO: 68 | SEQ ID NO: 9 | SEQ ID NO: 65 | SEQ ID NO: 1 |
| 47 | SEQ ID NO: 76 | SEQ ID NO: 9 | SEQ ID NO: 84 | SEQ ID NO: 1 |
| 48 | SEQ ID NO: 84 | SEQ ID NO: 9 | SEQ ID NO: 76 | SEQ ID NO: 1 |
| 49 | SEQ ID NO: 55 | SEQ ID NO: 53 | SEQ ID NO: 68 | SEQ ID NO: 52 |
| 50 | SEQ ID NO: 63 | SEQ ID NO: 53 | SEQ ID NO: 68 | SEQ ID NO: 52 |
| 51 | SEQ ID NO: 65 | SEQ ID NO: 53 | SEQ ID NO: 68 | SEQ ID NO: 52 |
| 52 | SEQ ID NO: 68 | SEQ ID NO: 53 | SEQ ID NO: 55 | SEQ ID NO: 52 |
| 53 | SEQ ID NO: 68 | SEQ ID NO: 53 | SEQ ID NO: 63 | SEQ ID NO: 52 |
| 54 | SEQ ID NO: 68 | SEQ ID NO: 53 | SEQ ID NO: 65 | SEQ ID NO: 52 |
| 55 | SEQ ID NO: 76 | SEQ ID NO: 53 | SEQ ID NO: 84 | SEQ ID NO: 52 |
| 56 | SEQ ID NO: 84 | SEQ ID NO: 53 | SEQ ID NO: 76 | SEQ ID NO: 52 |
| 57 | SEQ ID NO: 55 | SEQ ID NO: 54 | SEQ ID NO: 68 | SEQ ID NO: 52 |
| 58 | SEQ ID NO: 63 | SEQ ID NO: 54 | SEQ ID NO: 68 | SEQ ID NO: 52 |
| 59 | SEQ ID NO: 65 | SEQ ID NO: 54 | SEQ ID NO: 68 | SEQ ID NO: 52 |
| 60 | SEQ ID NO: 68 | SEQ ID NO: 54 | SEQ ID NO: 55 | SEQ ID NO: 52 |
| 61 | SEQ ID NO: 68 | SEQ ID NO: 54 | SEQ ID NO: 63 | SEQ ID NO: 52 |
| 62 | SEQ ID NO: 68 | SEQ ID NO: 54 | SEQ ID NO: 65 | SEQ ID NO: 52 |
| 63 | SEQ ID NO: 76 | SEQ ID NO: 54 | SEQ ID NO: 84 | SEQ ID NO: 52 |
| 64 | SEQ ID NO: 84 | SEQ ID NO: 54 | SEQ ID NO: 76 | SEQ ID NO: 52 |

In some embodiments, a composite polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 174 to 205. In some embodiments, a composite polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 178. In some embodiments, a composite polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 186.

In some embodiments, a polypeptide complex according to the present disclosure comprises a CD3-TCR complex polypeptide according to an embodiment described herein.
In some embodiments, a polypeptide complex according to the present disclosure comprises: (a) a polypeptide comprising (e.g. from N-terminus to C-terminus):
   (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column A of Table 2; and
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column B of Table 2;
      and
(b) a polypeptide comprising (e.g. from N-terminus to C-terminus):
   (i) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column C of Table 2; and
   (ii) an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to a sequence selected from Column D of Table 2;
      wherein the sequence selected from Column A of Table 2 and the sequence selected from Column B of Table 2 and the sequence selected from Column C of Table 2 and the sequence selected from Column D of Table 2 are selected from the same row of Table 2.

In some embodiments, a polypeptide complex according to the present disclosure comprises:

(1) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 129; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 165;
or

(2) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 133; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 165;

or

(3) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 137; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 165;

or

(4) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 145; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 173;

or

(5) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 141; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 153;

or

(6) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 141; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 157;

or

(7) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 141; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 161;

or

(8) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 149; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 169.

In preferred embodiments, a polypeptide complex according to the present disclosure comprises:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 137; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 165.

In preferred embodiments, a polypeptide complex according to the present disclosure comprises:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 141; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 161.

### Nucleic Acids and Vectors

The present disclosure provides a nucleic acid, or a plurality of nucleic acids, encoding a recombinant CD3-TCR complex polypeptide, composite polypeptide or polypeptide complex according to the present disclosure. In some embodiments, the nucleic acid(s) comprise or consist of DNA and/or RNA.

A recombinant CD3-TCR complex polypeptide, composite polypeptide or polypeptide complex according to the present disclosure may be produced within a cell by translation of RNA encoding the recombinant CD3-TCR complex polypeptide, composite polypeptide or polypeptide complex. A recombinant CD3-TCR complex polypeptide, composite polypeptide or polypeptide complex according to the present disclosure may be produced within a cell by transcription from nucleic acid encoding the recombinant CD3-TCR complex polypeptide, composite polypeptide or polypeptide complex, and subsequent translation of the transcribed RNA.

In some embodiments, the nucleic acid(s) may be, or may be comprised/contained in, a vector, or a plurality of vectors. A ‘vector’ as used herein is a nucleic acid molecule used as a vehicle to transfer exogenous nucleic acid into a cell.

Accordingly, the present disclosure also provides a vector, or plurality of vectors, comprising the nucleic acid or plurality of nucleic acids according to the present disclosure. The vector may facilitate delivery of the nucleic acid(s) encoding a recombinant CD3-TCR complex polypeptide, composite polypeptide or polypeptide complex according to the present disclosure to a cell. The vector may be an expression vector comprising elements required for expressing a recombinant CD3-TCR complex polypeptide, composite polypeptide or polypeptide complex according to the present disclosure. The vector may comprise elements facilitating integration of the nucleic acid(s) into the genomic DNA of cell into which the vector is introduced.

Nucleic acids and vectors according to the present disclosure may be provided in purified or isolated form, i.e. from other nucleic acid, or naturally-occurring biological material.

A vector may be a vector for expression of the nucleic acid in the cell (i.e. an expression vector). Such vectors may include a promoter sequence operably linked to a nucleotide sequence encoding a recombinant CD3-TCR complex polypeptide, composite polypeptide or polypeptide complex according to the present disclosure. A vector may also include a termination codon (i.e. 3′ in the nucleotide sequence of the vector to the nucleotide sequence encoding the recombinant CD3-TCR complex polypeptide/composite polypeptide/polypeptide complex) and expression enhancers. Any suitable vectors, promoters, enhancers and termination codons known in the art may be used to express a peptide or polypeptide from a vector according to the present disclosure.

The term ‘operably linked’ may include the situation where nucleic acid encoding a recombinant CD3-TCR complex polypeptide, composite polypeptide or polypeptide complex according to the present disclosure and regulatory nucleic acid sequence(s) (e.g. a promoter and/or enhancers) are covalently linked in such a way as to place the expression of the nucleic acid encoding a recombinant CD3-TCR complex polypeptide, composite polypeptide or polypeptide complex under the influence or control of the regulatory nucleic acid sequence(s) (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into the desired polypeptide(s).

Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

In some embodiments, a nucleic acid/plurality or vector/plurality according to the present disclosure comprises an EF1α promoter.

In some embodiments, a nucleic acid/plurality or vector/plurality according to the present disclosure encodes a CD3-TCR complex polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 110 to 173. In some embodiments, a nucleic acid/plurality or vector/plurality according to the present disclosure encodes a CD3-TCR complex polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 121.

In preferred embodiments, a nucleic acid/plurality or vector/plurality according to the present disclosure comprises the nucleotide sequence of SEQ ID NO:224, or a codon degenerate nucleotide sequence thereof encoding the amino acid sequence encoded by SEQ ID NO:224.

As used herein, a ‘codon degenerate nucleotide sequence’ of a reference nucleotide sequence refers to a nucleotide sequence having a non-identical nucleotide sequence to the nucleotide of the reference nucleotide sequence, but encoding the same amino acid sequence as the amino acid sequence encoded by the reference nucleotide sequence, as a consequence of degeneracy of the genetic code.

Constituent polypeptides of a polypeptide complex according to the present disclosure may be encoded by different nucleic acids of a plurality of nucleic acids according to the present disclosure, or by different vectors of a plurality of nucleic acids according to the present disclosure.

In aspects and embodiments of the present disclosure, a nucleic acid, or a plurality of nucleic acids, according to the present disclosure encodes two or more (e.g. 2, 3, 4 or more) recombinant CD3-TCR complex polypeptides according to the present disclosure. In aspects and embodiments of the present disclosure, a vector, or a plurality of vectors, according to the present disclosure encodes two or more (e.g. 2, 3, 4 or more) recombinant CD3-TCR complex polypeptides according to the present disclosure.

In some embodiments in which a nucleic acid/plurality or vector/plurality encodes two or more (e.g. 2, 3, 4 or more) recombinant CD3-TCR complex polypeptides, the recombinant CD3-TCR complex polypeptides are non-identical. In some embodiments, the nucleic acid/plurality or vector/plurality encodes recombinant CD3-TCR complex polypeptides that are complementary. That is, in some embodiments, the nucleic acid/plurality or vector/plurality encodes CD3-TCR complex polypeptides that are capable of associating with one another (e.g. via non-covalent, protein: protein interaction) to form a polypeptide complex (e.g. a polypeptide complex as described herein).

In some embodiments, the nucleic acid/plurality or vector/plurality encodes CD3-TCR complex polypeptides that are capable of associating with one another to form an antigen-binding moiety according to the present disclosure. In some embodiments, the nucleic acid/plurality or vector/plurality encodes CD3-TCR complex polypeptides comprising complementary components of an antigen-binding moiety according to the present disclosure (i.e. components of an antigen-binding moiety that are capable of association (e.g. via non-covalent, protein: protein interaction) to form the antigen-binding moiety).

By way of illustration, in one embodiment a nucleic acid/plurality or vector/plurality encodes (i) a recombinant CD3-TCR complex polypeptide comprising the VH of an antigen-binding moiety specific for a variant Fc domain and the ECD, TMD and ICD of TRAC (T47C), and (ii) a recombinant CD3-TCR complex polypeptide comprising the VL region of the antigen-binding moiety specific for a variant Fc domain, and the ECD, TMD and ICD of TRBC1 (S56C). Following expression from the nucleic acid/plurality or vector/plurality, polypeptides (i) and (ii) associate to form a polypeptide complex comprising an Fv specific for a variant Fc domain, formed by the VH from (i) and the VL from (ii).

In some embodiments, a nucleic acid/plurality or vector/plurality according to the present disclosure encodes:

(1) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 129; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 165;

or

(2) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 133; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 165;

or

(3) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 137; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 165;

or

(4) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 145; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 173;

or

(5) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 153;

or

(6) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 141; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 157;

or

(7) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 141; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 161;

or

(8) (i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 149; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 169.

In preferred embodiments, a nucleic acid/plurality or vector/plurality according to the present disclosure encodes:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 137; and
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 165.

In preferred embodiments, a nucleic acid/plurality or vector/plurality according to the present disclosure encodes:
(i) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or
(ii) a polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 161.

In preferred embodiments, a nucleic acid/plurality or vector/plurality according to the present disclosure comprises:
(i) the nucleotide sequence of SEQ ID NO:225, or a codon degenerate nucleotide sequence thereof encoding the amino acid sequence encoded by SEQ ID NO:225; and
(i) the nucleotide sequence of SEQ ID NO:226, or a codon degenerate nucleotide sequence thereof encoding the amino acid sequence encoded by SEQ ID NO:226.

In preferred embodiments, a nucleic acid/plurality or vector/plurality according to the present disclosure comprises:
(i) the nucleotide sequence of SEQ ID NO:227, or a codon degenerate nucleotide sequence thereof encoding the amino acid sequence encoded by SEQ ID NO:227; and
(i) the nucleotide sequence of SEQ ID NO:228, or a codon degenerate nucleotide sequence thereof encoding the amino acid sequence encoded by SEQ ID NO:228.

In some embodiments, a nucleic acid/plurality or vector/plurality according to the present disclosure encodes a composite polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 174 to 205. In some embodiments, a nucleic acid/plurality or vector/plurality according to the present disclosure encodes a composite polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 178. In some embodiments, a nucleic acid/plurality or vector/plurality according to the present disclosure encodes a composite polypeptide comprising or consisting of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO: 186.

In some embodiments, wherein a nucleic acid/plurality or vector/plurality encodes two or more (e.g. 2, 3, 4 or more) recombinant CD3-TCR complex polypeptides according to the present disclosure, transcription of nucleic acid encoding the two or more recombinant CD3-TCR complex polypeptides is under the control of the same promoter.

In some embodiments, the nucleic acid/plurality or vector/plurality comprises nucleic acid encoding an internal ribosome entry site (IRES). In some embodiments, the IRES is provided in between nucleotide sequences encoding recombinant CD3-TCR complex polypeptides. In some embodiments, the nucleic acid/plurality or vector/plurality comprises nucleic acid permitting the two or more recombinant CD3-TCR complex polypeptides to be translated separately from the same RNA transcript.

In some embodiments, the two or more recombinant CD3-TCR complex polypeptides are encoded by nucleotide sequences provided in the same reading frame. In some embodiments, the nucleic acid/plurality or vector/plurality encodes a fusion protein comprising the two or more recombinant CD3-TCR complex polypeptides. In some embodiments, the fusion protein encoded by the nucleic acid/plurality or vector/plurality comprises a cleavage site (e.g. a cleavage site as described herein) between the amino acid sequences of the recombinant CD3-TCR complex polypeptides. In some embodiments, the nucleic acid/plurality or vector/plurality encodes a composite polypeptide according to the present disclosure.

In some embodiments, transcription of nucleic acid encoding the two or more recombinant CD3-TCR complex polypeptides is under the control of different promoters.

In some embodiments, the nucleic acid/plurality or vector/plurality is multicistronic (e.g. bicistronic, tricistronic, etc.). That is, in some embodiments the nucleic acid/plurality or vector/plurality vector comprises multiple polypeptide-encoding nucleotide sequences. In some embodiments, nucleic acid encoding two or more recombinant CD3-TCR complex polypeptides is provided in different cistrons.

### Cells Comprising/Expressing the Polypeptides, Polypeptide Complexes and Nucleic Acids/Vectors of the Disclosure

The present disclosure also provides a cell comprising a recombinant CD3-TCR complex polypeptide, composite polypeptide or polypeptide complex according to the present disclosure, or a nucleic acid/plurality or vector/plurality according to the present disclosure.

It will be appreciated that where cells are referred to herein in the singular (i.e. ‘a/the cell’), pluralities/populations of such cells are also contemplated.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In preferred embodiments, the cell is a human cell.

In some embodiments, the cell is an immune cell. An immune cell may be a cell of hematopoietic origin, e.g. a neutrophil, eosinophil, basophil, dendritic cell, lymphocyte, or monocyte. A lymphocyte may be e.g. a T cell, B cell, NK cell, NKT cell or innate lymphoid cell (ILC), or a precursor thereof. The immune cell may express one or more CD3-TCR complex polypeptides, e.g. TCRα, TCRβ, TCRγ, TCRδ, TRAC, TRBC1, TRBC2, TRGC1, TRGC2, TRDC, CD3ε, CD3δ, CD3γ, CD3ζ and/or CD3η. The immune cell may express CD27, CD28, CD4 and/or CD8. In some embodiments, the immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell). In some embodiments, the T cell is a cytotoxic T cell (e.g. a cytotoxic T lymphocyte (CTL)).

Aspects and embodiments of the present disclosure relate particularly to T cells comprising/expressing CD3-TCR complexes comprising recombinant CD3-TCR complex polypeptides according to the present disclosure.

In some aspects and embodiments, a cell according to the present disclosure expresses/presents a recombinant CD3-TCR complex polypeptide or a polypeptide complex according to the present disclosure at the cell surface. That is, the recombinant CD3-TCR complex polypeptide or a polypeptide complex may be present in or at the cell membrane. Cells can be evaluated for surface expression of CD3-TCR complex polypeptides and polypeptide complexes (e.g. following introduction in the cell of nucleic acid encoding the same), e.g. using antibody-based methods such as flow cytometry (e.g. as described in Example 1 of the present disclosure).

In aspects and embodiments of the present disclosure, a cell according to the present disclosure comprises or expresses a recombinant CD3-TCR complex polypeptide, composite polypeptide or polypeptide complex according to the present disclosure. In some aspects and embodiments, a cell according to the present disclosure comprises nucleic acid encoding a recombinant CD3-TCR complex polypeptide, composite polypeptide or polypeptide complex according to the present disclosure. In some aspects and embodiments, a cell according to the present disclosure comprises a nucleic acid/plurality or vector/plurality according to the present disclosure.

In aspects and embodiments of the present disclosure, a cell according to the present disclosure comprises or expresses a polypeptide complex according to the present disclosure that binds to a variant Fc domain as described herein. It will be appreciated that binding to the variant Fc domain is achieved though the binding moiety of recombinant CD3-TCR complex polypeptide/polypeptide complex. The cell may express/comprise a polypeptide complex according to the present disclosure as a consequence of expression of nucleic acid encoding such a polypeptide complex. The cell may have been engineered to comprise nucleic acid encoding such a polypeptide complex.

In some embodiments, a cell according to the present disclosure may comprise modification to reduce expression of a CD3-TCR complex polypeptide (i.e. as compared to the level of expression of the CD3-TCR complex polypeptide by an equivalent unmodified cell). In some embodiments, the cell comprises modification to reduce expression of an endogenous CD3-TCR complex polypeptide, i.e. a CD3-TCR complex polypeptide encoded by the genome of an equivalent unmodified cell.

In some embodiments, the cell comprises modification to reduce expression of the CD3-TCR complex polypeptide from which the CD3-TCR complex association domain of the recombinant polypeptide CD3-TCR complex polypeptide is derived. By way of illustration, in embodiments wherein the cell comprises or expresses a recombinant CD3-TCR complex polypeptide comprising a CD3-TCR complex association domain derived from CD3ε (or a composite polypeptide or polypeptide complex comprising such a recombinant CD3-TCR complex polypeptide), the cell may comprise modification to reduce expression by the cell of CD3ε (i.e. endogenous CD3ε). By way of further illustration, in embodiments wherein the cell comprises or expresses recombinant CD3-TCR complex polypeptide(s) comprising a CD3-TCR complex association domain(s) derived from TRAC, TRBC1 and/or TRBC2 (or a composite polypeptide or polypeptide complex comprising such recombinant CD3-TCR complex polypeptide(s)), the cell may comprise modification to reduce expression by the cell of TRAC/TRBC1/TRBC2 (i.e. endogenous TRAC/TRBC1/TRBC2).

In some embodiments, the cell comprises modification to nucleic acid (e.g. endogenous nucleic acid) encoding the CD3-TCR complex polypeptide. In some embodiments, one or more alleles of the gene encoding the CD3-TCR complex polypeptide in the cell are modified. In some embodiments, the modification comprises an insertion, substitution or deletion in the nucleotide sequence of nucleic acid encoding the CD3-TCR complex polypeptide. In some embodiments, the modification reduces or prevents the endogenous expression of the CD3-TCR complex polypeptide from the modified nucleotide sequence. In some embodiments, the modified cell lacks endogenous nucleic acid encoding the CD3-TCR complex polypeptide. In some embodiments, the modification introduces a premature stop codon in the nucleotide sequence of RNA transcribed from endogenous nucleic acid encoding the CD3-TCR complex polypeptide. In some embodiments, the nucleotide sequence of the modified nucleic acid encodes a truncated and/or non-functional version of the CD3-TCR complex polypeptide. In some embodiments, the nucleotide sequence of the modified nucleic acid encodes a version of the CD3-TCR complex polypeptide that is misfolded and/or degraded. In some embodiments, the nucleotide sequence of the modified nucleic acid encodes a version of the CD3-TCR complex polypeptide that is incapable of participating in a functional CD3-TCR polypeptide complex.

In some embodiments, the cell comprises modification to nucleic acid (e.g. endogenous nucleic acid) encoding CD3ε (e.g. the polypeptide having the sequence of SEQ ID NO:25). In some embodiments, one or more alleles of CD3E are modified. In some embodiments, the modification comprises an insertion, substitution or deletion in the nucleotide sequence of CD3E. In some embodiments, the modification reduces or prevents the endogenous expression of CD3ε by the cell. In some embodiments, the modified cell lacks endogenous nucleic acid encoding CD3ε. In some embodiments, the modification introduces a premature stop codon in the nucleotide sequence of RNA transcribed from endogenous nucleic acid encoding CD3ε. In some embodiments, the nucleotide sequence of the modified nucleic acid encodes a truncated and/or non-functional version of CD3ε. In some embodiments, the nucleotide sequence of the modified nucleic acid encodes a version of CD3ε that is misfolded and/or degraded. In some embodiments, the nucleotide sequence of the modified nucleic acid encodes a version of CD3ε that is incapable of participating in a functional CD3-TCR polypeptide complex.

In some embodiments, the cell comprises modification to nucleic acid (e.g. endogenous nucleic acid) encoding TRAC (e.g. the polypeptide having the sequence of SEQ ID NO: 1), TRBC1 (e.g. the polypeptide having the sequence of SEQ ID NO:5) and/or TRBC2 (e.g. the polypeptide having the sequence of SEQ ID NO:9). In some embodiments, the cell comprises modification to nucleic acid (e.g. endogenous nucleic acid) encoding TRAC and TRBC1. In some embodiments, one or more alleles of TRAC, TRBC1 and/or TRBC2 (e.g. TRAC and TRBC1) are modified. In some embodiments, the modification comprises an insertion, substitution or deletion in the nucleotide sequence of TRAC, TRBC1 and/or TRBC2 (e.g. TRAC and TRBC1). In some embodiments, the modification reduces or prevents the endogenous expression of TRAC, TRBC1 and/or TRBC2 (e.g. TRAC and TRBC1) by the cell. In some embodiments, the modified cell lacks endogenous nucleic acid encoding TRAC, TRBC1 and/or TRBC2 (e.g. TRAC and TRBC1). In some embodiments, the modification introduces a premature stop codon in the nucleotide sequence of RNA transcribed from endogenous nucleic acid encoding TRAC, TRBC1 and/or TRBC2 (e.g. TRAC and TRBC1). In some embodiments, the nucleotide sequence of the modified nucleic acid encodes a truncated and/or non-functional version of TRAC, TRBC1 and/or TRBC2 (e.g. TRAC and TRBC1). In some embodiments, the nucleotide sequence of the modified nucleic acid encodes a version of TRAC, TRBC1 and/or TRBC2 (e.g. TRAC and TRBC1) that is misfolded and/or degraded. In some embodiments, the nucleotide sequence of the modified nucleic acid encodes a version of TRAC, TRBC1 and/or TRBC2 (e.g. TRAC and TRBC1) that is incapable of participating in a functional CD3-TCR polypeptide complex.

An immune cell (e.g. a T cell) according to the present disclosure may be characterised by certain functional properties in response to the antigen for which CD3-TCR polypeptide complex comprises an antigen-binding moiety (or in response to a cell comprising or expressing the antigen): CD3-TCR complex-mediated signalling; cell proliferation/population expansion, growth factor (e.g. IL-2) expression, IFNγ expression, CD107a expression, TNFα expression, GM-CSF expression, perforin expression, granzyme expression, granulysin expression, and/or FAS ligand (FASL) expression.

For example, an immune cell (e.g. a T cell) according to the present disclosure may display one or more of the functional properties recited in the preceding paragraph in response to a variant Fc domain according to the present disclosure (i.e. a variant Fc domain that is bound by antigen-binding moiety comprised in a CD3-TCR polypeptide complex expressed by the cell), or in response to a cell comprising/expressing such a variant Fc domain.

CD3-TCR complex-mediated signalling can be investigated by analysing one or more correlates of CD3-TCR complex-mediated signalling. For example, CD3-TCR complex-mediated signalling can be investigated by evaluating phosphorylation of one or more signal transduction molecules of CD3-TCR complex signalling pathway. The level of CD3-TCR complex-mediated signalling can be analysed by detection and quantification of the level of phosphorylation of CD37, ZAP-70, Lck, LAT and/or SLP-76. The level of CD3-TCR complex-mediated signalling can also be analysed using reporter-based methods, e.g. methods quantifying the activity of a transcription factor whose expression/activity is upregulated in response to signalling through the CD3-TCR complex, e.g. NFAT, NF-κB and/or AP-1, or through methods quantifying expression of a gene whose expression is upregulated by signalling through the CD3-TCR complex, e.g. IL2. For example, CD3-TCR complex-mediated signalling can be investigated using a reporter cell line stably expressing a luciferase reporter driven by CD3-TCR complex-mediated signalling (e.g. GloResponse Jurkat NFAT-RE-luc2P (Promega #CS176501) or T Cell Activation Bioassay TCRαβ-KO CD4+ (Promega #GA1172), as in Example 1 of the present disclosure.

Cell proliferation/population expansion can be investigated by analysing cell division or the number of cells over a period of time. Cell division can be analysed, for example, by in vitro analysis of incorporation of ³H-thymidine or by CFSE dilution assay, e.g. as described in Fulcher and Wong, Immunol Cell Biol (1999) 77 (6): 559-564, hereby incorporated by reference in entirety. Proliferating cells can also be identified by analysis of incorporation of 5-ethynyl-2′-deoxyuridine (EdU), as described e.g. in Buck et al., Biotechniques. 2008 June; 44 (7): 927-9, and Sali and Mitchison, PNAS USA 2008 Feb. 19; 105 (7): 2415-2420, both hereby incorporated by reference in their entirety.

As used herein, ‘expression’ may be gene or protein expression. Gene expression encompasses transcription of DNA to RNA, and can be measured by various means known to those skilled in the art, for example by measuring levels of mRNA by quantitative real-time PCR (qRT-PCR), or using reporter-based methods. Similarly, protein expression can be measured by various methods well known in the art, e.g. antibody-based methods, for example by western blot, immunohistochemistry, immunocytochemistry, flow cytometry, ELISA, ELISPOT, or reporter-based methods.

An immune cell (e.g. a T cell) according to the present disclosure may display cytotoxicity to cells comprising/expressing a variant Fc domain according to the present disclosure. That is, an immune cell (e.g. a T cell) according to the present disclosure may posses the ability to kill cells comprising/expressing a variant Fc domain according to the present disclosure.

A cell comprising a variant Fc domain according to the present disclosure may do so as a consequence of binding of an antigen-binding molecule comprising the variant Fc domain to an antigen expressed by the cell (e.g. at the surface of the cell, i.e. in or at the cell membrane). In some embodiments, a cell comprising a variant Fc domain according to the present disclosure comprises (e.g. at the surface of the cell) a polypeptide complex comprising (i) an antigen-binding molecule comprising the variant Fc domain, and (ii) the target antigen for the antigen-binding molecule.

Cytotoxicity and cell killing can be investigated, for example, using any of the methods reviewed in Zaritskaya et al., Expert Rev Vaccines (2011), 9 (6): 601-616, hereby incorporated by reference in its entirety. Examples of in vitro assays of cytotoxicity/cell killing assays include release assays such as the 51 Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing by a given test cell type (e.g. an immune cell (e.g. a T cell) according to the present disclosure) can be analysed e.g. by co-culturing the test cells with the given target cell type (e.g. a cell comprising a variant Fc domain according to the present disclosure), and measuring the number/proportion of viable (i.e. non-lysed)/dead (e.g. lysed) target cells after a suitable period of time. Other suitable assays include the xCELLigence real-time cytolytic in vitro potency assay described in Cerignoli et al., PLOS One. (2018) 13 (3): e0193498 (hereby incorporated by reference in its entirety), and the Incucyte immune cell killing assay, which is employed in the experimental examples of the present disclosure.

An immune cell (e.g. a T cell) according to the present disclosure may possess one or more novel, similar or improved functional properties as compared to a CAR comprising the same antigen-binding moiety (i.e. the antigen-binding moiety of the recombinant CD3-TCR complex polypeptide/polypeptide complex expressed by the cell). In some embodiments, an immune cell according to the present disclosure may possess one or more novel, similar or improved functional properties as compared to a CAR-expressing cell described in WO 2018/177966 A1, which is hereby incorporated by reference in its entirety.

In some embodiments, an immune cell (e.g. a T cell) according to the present disclosure may display a level of CD3-TCR complex-mediated signalling in response to the antigen for which CD3-TCR polypeptide complex comprises an antigen-binding moiety, or in response to a cell comprising or expressing the antigen (e.g. in response to a variant Fc domain according to the present disclosure (i.e. a variant Fc domain that is bound by antigen-binding moiety comprised in a CD3-TCR polypeptide complex expressed by the cell), or in response to a cell comprising/expressing such a variant Fc domain) that is similar to, or greater than, the level of CD3-TCR complex-mediated signalling displayed by a CAR comprising the same antigen-binding moiety.

A level of CD3-TCR complex-mediated signalling which is ‘similar to’ a reference level of CD3-TCR complex-mediated signalling may be ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the reference level of CD3-TCR complex-mediated signalling. In some embodiments, a level of CD3-TCR complex-mediated signalling which is ‘greater than’ a reference level of CD3-TCR complex-mediated signalling may be greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times or ≥5 times the reference level of CD3-TCR complex-mediated signalling.

In some embodiments, a T cell expressing a recombinant CD3-TCR complex polypeptide/polypeptide complex according to the present disclosure comprising an antigen-binding moiety comprising a VH region according to SEQ ID NO:65 and a VL region according to SEQ ID NO:68 may be evaluated in an assay comprising:
(i) contacting T cells expressing a recombinant CD3-TCR complex polypeptide/polypeptide complex according to the present disclosure comprising an antigen-binding moiety comprising a VH region according to SEQ ID NO:65 and a VL region according to SEQ ID NO:68 with antigen-presenting cells, which are cells expressing a given target antigen that have been contacting with an antigen-binding molecule that binds to the given target antigen, and which comprises an Fc domain having a CH2-CH3 region according to SEQ ID NO:213, and subsequently analysing the level of CD3-TCR complex-mediated signalling by the T cells;
(ii) contacting T cells (e.g. equivalent T cells, i.e. derived from the same source as the T cells of (i)) expressing a CAR according to SEQ ID NO: 108, with antigen-presenting cells as defined in (i), and subsequently analysing the level of CD3-TCR complex-mediated signalling by the T cells; and
(iii) comparing the level of CD3-TCR complex-mediated signalling by the T cells of (i) with the T cells of (ii).

In some embodiments, in an assay performed as described in the preceding paragraph, T cells according to (i) display a level of CD3-TCR complex-mediated signalling which is ≥0.5 times and ≤2 times, e.g. one of ≥0.55 times and ≤1.9 times, ≥0.6 times and ≤1.8 times, ≥0.65 times and ≤1.7 times, ≥0.7 times and ≤1.6 times, ≥0.75 times and ≤1.5 times, ≥0.8 times and ≤1.4 times, ≥0.85 times and ≤1.3 times, ≥0.9 times and ≤1.2 times, ≥0.95 times and ≤1.1 times the level of CD3-TCR complex-mediated signalling displayed by T cells according to (ii). In some embodiments, in an assay performed as described in the preceding paragraph, T cells according to (i) display a level of CD3-TCR complex-mediated signalling which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times or ≥5 times the level of CD3-TCR complex-mediated signalling displayed by T cells according to (ii).

The present disclosure also provides methods for producing a cell according to the present disclosure, and the cells obtained or obtainable by such methods.

Methods for producing cells comprising/expressing a polypeptide/polypeptide complex of interest are well known to the skilled person, and generally comprise introducing nucleic acid(s)/vector(s) encoding the polypeptide(s) of interest into the cells.

Such methods may comprise nucleic acid transfer for permanent (i.e. stable) or transient expression of the transferred nucleic acid. In some embodiments, following introduction into a cell nucleic acid(s) encoding the polypeptide(s) of interest may be integrated into or form part of the genomic DNA of the cell. In some embodiments, following introduction into a cell nucleic acid(s) encoding the polypeptide(s) of interest may be maintained extrachromosomally.

Any suitable genetic engineering platform may be used, and include gammaretroviral vectors, lentiviral vectors, adenovirus vectors, DNA transfection, transposon-based gene delivery and RNA transfection, for example as described in Maus et al., Annu Rev Immunol (2014) 32:189-225, hereby incorporated by reference in its entirety. Methods also include those described e.g. in Wang and Rivière Mol Ther Oncolytics. (2016) 3:16015, which is hereby incorporated by reference in its entirety. Suitable methods for introducing nucleic acid(s)/vector(s) into cells include transduction, transfection and electroporation.

Methods for generating/expanding populations of cells comprising/expressing polypeptide(s) of interest in vitro/ex vivo are well known to the skilled person. Suitable culture conditions (i.e. cell culture media, additives, stimulations, temperature, gaseous atmosphere), cell numbers, culture periods and methods for introducing nucleic acid(s)/vector(s) encoding polypeptide(s) of interest into cells, etc. can be determined by reference e.g. to WO 2018/177966 A1. In some embodiments, a cell/population of cells according to the present disclosure is prepared under GMP (good manufacturing practice; e.g. as described in the guidelines for good manufacturing practice published by the European Commission (Volume 4 of ‘The rules governing medicinal products in the European Union’ contains guidance for the interpretation of the principles and guidelines of good manufacturing practices for medicinal products for human and veterinary use laid down in Commission Directives 91/356/EEC, as amended by Directive 2003/94/EC, and 91/412/EEC respectively) conditions.

Conveniently, cultures of cells according to the present disclosure may be maintained at 37° C. in a humidified atmosphere containing 5% CO₂. The cells of cell cultures can be established and/or maintained at any suitable density, as can readily be determined by the skilled person. Cultures can be performed in any vessel suitable for the volume of the culture, e.g. in wells of a cell culture plate, cell culture flasks, a bioreactor, etc. In some embodiments cells are cultured in a bioreactor, e.g. a bioreactor described in Somerville and Dudley, Oncoimmunology (2012) 1 (8): 1435-1437, which is hereby incorporated by reference in its entirety. Immune cells (e.g. T cells) may be activated prior to introduction of nucleic acid(s) encoding the polypeptide(s) of interest. For example, T cells within a population of PBMCs may be non-specifically activated by stimulation in vitro with agonist anti-CD3 and agonist anti-CD28 antibodies, in the presence of IL-2.

Introducing nucleic acid(s) into a cell may comprise transduction, e.g. lentiviral transduction. Transduction of immune cells with viral vectors is described e.g. in Simmons and Alberola-IIa, Methods Mol Biol. (2016) 1323:99-108, which is hereby incorporated by reference in its entirety.

Agents may be employed to enhance the efficiency of transduction. Hexadimethrine bromide (polybrene) is a cationic polymer which is commonly used to improve transduction, through neutralising charge repulsion between virions and sialic acid residues expressed on the cell surface. Other agents commonly used to enhance transduction include e.g. the poloxamer-based agents such as LentiBOOST (Sirion Biotech), Retronectin (Takara), Vectofusin (Miltenyi Biotech) and also SureENTRY (Qiagen) and ViraDuctin (Cell Biolabs). In some embodiments the methods comprise centrifuging the cells into which it is desired to introduce nucleic acid encoding the polypeptide(s) of interest in the presence of cell culture medium comprising viral vector comprising the nucleic acid (referred to in the art as ‘spinfection’).

The methods generally comprise introducing a nucleic acid encoding polypeptide(s) of interest into a cell, and culturing the cell under conditions suitable for expression of the polypeptide(s) of interest by the cell. In some embodiments, the methods comprise culturing immune cells into which nucleic acid encoding polypeptide(s) of interest has been introduced in order to expand their number.

In some embodiments, the methods comprise analysing the cells to confirm successful introduction of the nucleic acid into the cells. In some embodiments, the methods comprise analysing the cells to confirm expression of the polypeptide(s) of interest by the cells (e.g. via evaluation of a detectable entity).

In some embodiments the methods further comprise cells expressing the polypeptide(s) of interest, e.g. from other cells (e.g. cells which do not express the polypeptide(s) of interest). Methods for purifying/isolating immune cells from heterogeneous populations of cells are well known in the art, and may employ e.g. FACS- or MACS-based methods for sorting populations of cells based on the expression of markers of the immune cells. In some embodiments the methods purifying/isolating cells of a particular type, e.g. CD8+ T cells or CTLs expressing the polypeptide(s) of interest.

Methods for producing cells according to the present disclosure may comprise modifying the cells to reduce the expression of a CD3-TCR complex polypeptide. In some embodiments, the methods comprise modifying nucleic acid (e.g. endogenous nucleic acid) encoding the CD3-TCR complex polypeptide.

Modification of a given target nucleic acid can be achieved in a variety of ways known to the skilled person, including modification of the target nucleic acid by homologous recombination, and target nucleic acid editing using site-specific nucleases (SSNs).

Suitable methods may employ targeting by homologous recombination, which is reviewed, for example, in Mortensen Curr Protoc Neurosci. (2007) Chapter 4: Unit 4.29 and Vasquez et al., PNAS 2001, 98 (15): 8403-8410 both of which are hereby incorporated by reference in their entirety. Targeting by homologous recombination involves the exchange of nucleic acid sequence through crossover events guided by homologous sequences. Other suitable techniques include nucleic acid editing using SSNs. Gene editing using SSNs is reviewed e.g. in Eid and Mahfouz, Exp Mol Med. 2016 October; 48 (10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively, DSBs may be repaired by homology-directed repair (HDR), a high-fidelity mechanism in which a DNA template with ends homologous to the break site is supplied and introduced at the site of the DSB.

SSNs capable of being engineered to generate target nucleic acid sequence-specific DSBs include zinc-finger nucleases (ZFNs), transcription activator-like effector nucleases (TALENs) and clustered regularly interspaced palindromic repeats/CRISPR-associated-9 (CRISPR/Cas9) systems. ZFN systems are reviewed e.g. in Umov et al., Nat Rev Genet. (2010) 11 (9): 636-46, which is hereby incorporated by reference in its entirety. ZFNs comprise a programmable Zinc Finger DNA-binding domain and a DNA-cleaving domain (e.g. a FokI endonuclease domain). The DNA-binding domain may be identified by screening a Zinc Finger array capable of binding to the target nucleic acid sequence. TALEN systems are reviewed e.g. in Mahfouz et al., Plant Biotechnol J. (2014) 12 (8): 1006-14, which is hereby incorporated by reference in its entirety. TALENs comprise a programmable DNA-binding TALE domain and a DNA-cleaving domain (e.g. a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: ‘HD’ binds to C, ‘NI’ binds to A, ‘NG’ binds to T and ‘NN’ or ‘NK’ binds to G (Moscou and Bogdanove, Science (2009) 326 (5959): 1501.). CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8 (3): 265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some embodiments, modifying nucleic acid (e.g. endogenous nucleic acid) encoding the CD3-TCR complex polypeptide in accordance with the present disclosure employs a site-specific nuclease (SSN) system targeting nucleic acid encoding the CD3-TCR complex polypeptide. The SSN system may be a ZFN system, a TALEN system, CRISPR/Cas9 system, a CRISPR/Cpf1 system, a CRISPR/C2c1 system, a CRISPR/C2c2 system or a CRISPR/C2c3 system.

In some embodiments, a method for producing a cell according to the present disclosure comprises introducing nucleic acid(s) encoding a CRISPR/Cas9 system targeting CD3E into a cell. In some embodiments, the nucleic acid(s) encode a CRISPR RNA (crRNA) targeting CD3E (e.g. an exon of CD3E, e.g. exon 7 of CD3E) and a trans-activating crRNA (tracrRNA) for processing the crRNA to its mature form.

In some embodiments, a method for producing a cell according to the present disclosure comprises introducing nucleic acid(s) encoding CRISPR/Cas9 system(s) targeting TRAC, TRBC1 and/or TRBC2 (e.g. TRAC and TRBC1) into a cell. In some embodiments, the nucleic acid(s) encode a CRISPR RNA (crRNA) targeting TRAC, TRBC1 and/or TRBC2 (e.g. TRAC and TRBC1; e.g. an exon of TRAC, TRBC1 and/or TRBC2 (e.g. TRAC and TRBC1)) and a trans-activating crRNA (tracrRNA) for processing the crRNA to its mature form.

### Compositions

The present disclosure also provides compositions comprising the polypeptides, polypeptide complexes, nucleic acids, expression vectors and cells described herein.

The polypeptides, polypeptide complexes, nucleic acids, expression vectors and cells described herein (and particularly the nucleic acids, expression vectors and cells described herein) may be formulated as pharmaceutical compositions or medicaments for clinical use and may comprise a pharmaceutically-acceptable carrier, diluent, excipient or adjuvant. In preferred aspects and embodiments, the present disclosure provides a pharmaceutical composition or medicament comprising a cell according to the present disclosure. Thus, the present disclosure also provides a pharmaceutical composition/medicament comprising a polypeptide, polypeptide complex, nucleic acid/plurality, expression vector/plurality or cell described herein. In preferred embodiments, a pharmaceutical composition/medicament according to the present disclosure comprises a nucleic acid/plurality, expression vector/plurality or cell described herein.

The pharmaceutical compositions/medicaments of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Pharmaceutical compositions and medicaments of the present disclosure may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the cell provided in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the pharmaceutical compositions/medicament is formulated for injection or infusion, e.g. into a blood vessel, tissue/organ of interest, or a tumor.

The present disclosure also provides methods for the production of pharmaceutically useful compositions, such methods of production may comprise one or more steps selected from:
producing a cell described herein;
isolating/purifying a cell described herein; and/or
mixing a cell described herein with a pharmaceutically-acceptable carrier, adjuvant, excipient or diluent.

For example, a further aspect the present disclosure relates to a method of formulating or producing a medicament or pharmaceutical composition for use in the treatment of a disease/condition (e.g. a disease/condition described herein), the method comprising formulating a pharmaceutical composition or medicament by mixing a cell described herein with a pharmaceutically-acceptable carrier, adjuvant, excipient or diluent.

### Therapeutic and Prophylactic Applications

The articles of the present disclosure find use in therapeutic and prophylactic methods. In particular, a cell according to the present disclosure, e.g. a cell comprising/expressing a CD3-TCR polypeptide complex comprising a recombinant CD3-TCR complex polypeptide according to the present disclosure, finds use in therapeutic and prophylactic methods. Similarly, a composition according to the present disclosure, e.g. a pharmaceutical composition comprising a cell according to the present disclosure, e.g. a cell comprising/expressing a CD3-TCR polypeptide complex comprising a recombinant CD3-TCR complex polypeptide according to the present disclosure finds use in such methods.

Accordingly, the present disclosure provides a cell or composition described herein for use in a method of medical treatment or prophylaxis. Also provided is a cell or composition described herein for use in a method of treating or preventing a disease or condition described herein. Also provided is the use of a cell or composition described herein in the manufacture of a medicament for treating or preventing a disease or condition described herein. Also provided is a method of treating or preventing a disease or condition described herein, comprising administering to a subject a therapeutically- or prophylactically-effective amount of a cell or composition described herein.

The intervention described in the preceding paragraph may be effective to reduce the development or progression of a disease/condition, alleviate the symptoms of a disease/condition or reduce the pathology of a disease/condition. The intervention may be effective to prevent progression of the disease/condition, e.g. to prevent worsening of, or to slow the rate of development of, the disease/condition. In some embodiments, the intervention may lead to an improvement in the disease/condition, e.g. a reduction in the symptoms of the disease/condition or reduction in some other correlate of the severity/activity of the disease/condition. In some embodiments, the intervention may prevent progression/development of the disease/condition a later stage (e.g. a chronic stage or metastasis).

Therapeutic or prophylactic intervention in accordance with the present disclosure generally comprises administering a cell or pharmaceutical composition according to the present disclosure to a subject to which an antigen-binding molecule comprising: (a) an antigen-binding domain that binds to the target antigen, and (b) a variant Fc domain according to the present disclosure, has been or is to be administered.

It will be appreciated that in accordance with such intervention, the cell (or the cell of the composition) comprises/expresses a CD3-TCR complex comprising an antigen-binding moiety that binds to the variant Fc domain of the antigen-binding molecule. By way of illustration, the intervention may comprise administering a T cell expressing a CD3-TCR polypeptide complex comprising a recombinant CD3-TCR complex polypeptide according to SEQ ID NO: 121 to a subject that has been, or is to be, administered an antibody comprising an Fc domain comprising a CH2-CH3 region according to SEQ ID NO:213. By way of illustration, the intervention may comprise administering a T cell expressing a CD3-TCR polypeptide complex comprising (i) a recombinant CD3-TCR complex polypeptide according to SEQ ID NO: 137 and (ii) a recombinant CD3-TCR complex polypeptide according to SEQ ID NO: 165 to a subject that has been, or is to be, administered an antibody comprising an Fc domain comprising a CH2-CH3 region according to SEQ ID NO:213. By way of further illustration, the intervention may comprise administering a T cell expressing a CD3-TCR polypeptide complex comprising a recombinant CD3-TCR complex polypeptide according to SEQ ID NO: 125 to a subject that has been, or is to be, administered an antibody comprising an Fc domain comprising a CH2-CH3 region according to SEQ ID NO:212.

In the therapeutic/prophylactic intervention of the present disclosure, the variant Fc domain-bearing antigen-binding molecule serves as an adaptor molecule, and directs the activity of a cell according to the present disclosure against the antigen to which the antigen-binding molecule binds. That is, in embodiments wherein the cell is an immune cell (e.g. a T cell), the variant Fc domain-bearing antigen-binding molecule directs a cell-mediated immune response (e.g. a T cell-mediated immune response) against cells expressing the antigen to which the antigen-binding molecule binds.

By way of illustration, in the Examples of the present disclosure, a T cell expressing a CD3-TCR polypeptide complex comprising a recombinant CD3-TCR complex polypeptide according to SEQ ID NO: 121 is employed with an anti-FolR1 antibody comprising an Fc domain comprising P329G, such that the T cells are directed against FolR1-expressing cells. By way of further illustration, in the Examples of the present disclosure, a T cell expressing a CD3-TCR polypeptide complex comprising a recombinant CD3-TCR complex polypeptide according to SEQ ID NO: 121 is employed with an anti-CD19 antibody comprising an Fc domain comprising P329G, such that the T cells are directed against CD19-expressing cells. By way of further illustration, in the Examples of the present disclosure, a T cell expressing a CD3-TCR polypeptide complex comprising (i) a recombinant CD3-TCR complex polypeptide according to SEQ ID NO: 137 and (ii) a recombinant CD3-TCR complex polypeptide according to SEQ ID NO: 165 is employed with an anti-FolR1 antibody comprising an Fc domain comprising P329G, such that the T cells are directed against FolR1-expressing cells. By way of further illustration, in the Examples of the present disclosure, a T cell expressing a CD3-TCR polypeptide complex comprising (i) a recombinant CD3-TCR complex polypeptide according to SEQ ID NO: 137 and (ii) a recombinant CD3-TCR complex polypeptide according to SEQ ID NO: 165 is employed with an anti-CD19 antibody comprising an Fc domain comprising P329G, such that the T cells are directed against CD19-expressing cells.

The variant Fc domain-bearing antigen-binding molecule employed with a cell or composition according to the present disclosure may bind to any given target antigen.

The target antigen may be any target antigen expressed by a cell that it is desired to kill/deplete in order to attain a therapeutic/prophylactic effect. In some embodiments, the target antigen is an antigen whose expression/activity, or whose upregulated expression/activity, is positively associated with a disease/condition (e.g. a cancer, an infectious disease or an autoimmune disease). The target antigen is preferably expressed at the cell surface of a cell expressing the target antigen.

In some embodiments, the target antigen may be a cancer cell antigen. A cancer cell antigen is an antigen which is expressed or over-expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

Cancer cell antigens are reviewed by Zarour H M, DeLeo A, Finn O J, et al. Categories of Tumor Antigens. In: Kufe D W, Pollock R E, Weichselbaum R R, et al., editors. Holland-Frei Cancer Medicine. 6th edition. Hamilton (ON): BC Decker; 2003. Cancer cell antigens include oncofetal antigens: CEA, Immature laminin receptor, TAG-72; oncoviral antigens such as HPV E6 and E7; overexpressed proteins: BING-4, calcium-activated chloride channel 2, cyclin-B1, 9D7, Ep-CAM, EphA3, HER2/neu, telomerase, mesothelin, SAP-1, survivin; cancer-testis antigens: BAGE, CAGE, GAGE, MAGE, SAGE, XAGE, CT9, CT10, NY-ESO-1, PRAME, SSX-2; lineage restricted antigens: MART1, Gp100, tyrosinase, TRP-1/2, MC1R, prostate specific antigen; mutated antigens: β-catenin, BRCA1/2, CDK4, CML66, Fibronectin, MART-2, p53, Ras, TGF-βRII; post-translationally altered antigens: MUC1, idiotypic antigens: Ig, TCR. Other cancer cell antigens include heat-shock protein 70 (HSP70), heat-shock protein 90 (HSP90), glucose-regulated protein 78 (GRP78), vimentin, nucleolin, feto-acinar pancreatic protein (FAPP), alkaline phosphatase placental-like 2 (ALPPL-2), siglec-5, stress-induced phosphoprotein 1 (STIP1), protein tyrosine kinase 7 (PTK7), and cyclophilin B. In some embodiments the cancer cell antigen is a cancer cell antigen described in Zhao and Cao, Front Immunol. (2019) 10:2250, which is hereby incorporated by reference in its entirety.

In some embodiments, the target antigen is selected from: FAP (fibroblast activation protein), CEA (carcinoembryonic antigen), p95 (p95HER2), BCMA (B-cell maturation antigen), EpCAM (epithelial cell adhesion molecule), MSLN (mesothelin), MCSP (melanoma chondroitin sulfate proteoglycan), HER-1 (human epidermal growth factor 1), HER-2 (human epidermal growth factor 2), HER-3 (human epidermal growth factor 3), CD19, CD20, CD22, CD33, CD38, CD52FIt3, folate receptor 1 (FOLR1), human trophoblast cell-surface antigen 2 (Trop-2) cancer antigen 12-5 (CA-12-5), human leukocyte antigen-antigen D related (HLA-DR), MUC-1 (Mucin-1), A33-antigen, PSMA (prostate-specific membrane antigen), FMS-like tyrosine kinase 3 (FLT-3), PSMA (prostate specific membrane antigen), PSCA (prostate stem cell antigen), transferrin-receptor, TNC (tenascin), carbon anhydrase IX (CA-IX), and/or a peptide bound to a molecule of the human major histocompatibility complex (MHC). In some embodiments, the target antigen is CD19. In some embodiments, the target antigen is FOLR1.

It will be appreciated that the cells and composition of the present disclosure may be used for the treatment/prevention of any disease/condition that would derive therapeutic or prophylactic benefit from a reduction in the level/activity of a given target antigen, or a reduction in the number/proportion/activity of cells comprising/expressing a given target antigen.

For example, the disease/condition may be a disease/condition in which the target antigen, or cells comprising/expressing target antigen are pathologically-implicated, e.g. a disease/condition in which an increased level/activity of the target antigen, or an increase in the number/proportion/activity of cells comprising/expressing target antigen is positively associated with the onset, development or progression of the disease/condition, and/or severity of one or more symptoms of the disease/condition. In some embodiments, an increased level/activity of the target antigen, or an increase in the number/proportion/activity of cells comprising/expressing target antigen may be a risk factor for the onset, development or progression of the disease/condition.

In some embodiments, the disease/condition to be treated/prevented in accordance with the present disclosure is a disease/condition characterised by an increase in the level of expression or activity of the target antigen, e.g. as compared to the level of expression/activity in the absence of the disease/condition. In some embodiments, the disease/condition to be treated/prevented is a disease/condition characterised by an increase in the number/proportion/activity of cells expressing target antigen, e.g. as compared to the level/number/proportion/activity in the absence of the disease/condition (e.g. in a healthy subject, or in equivalent non-diseased tissue). Where the disease/condition is a cancer, the level of expression or activity of the target antigen may be greater than the level of expression or activity of the target antigen in equivalent non-cancerous cells/non-tumor tissue. A cancer/cell thereof may comprise one or more mutations (e.g. relative to equivalent non-cancerous cells/non-tumor tissue) causing upregulation of expression or activity of the target antigen.

Therapeutic/prophylactic intervention in accordance with the present disclosure may achieve one or more of the following in a subject (compared to an equivalent untreated subject, or subject treated with an appropriate control): a reduction in the level of the target antigen; a reduction in the activity of the target antigen; and/or a reduction in the number/proportion/activity of cells comprising/expressing the target antigen.

In particular, use of the cells and compositions according to the present disclosure in methods to treat/prevent diseases/conditions by adoptive cell transfer (ACT) is contemplated.

Adoptive cell transfer generally refers to a process by which cells (e.g. immune cells) are obtained from a subject, typically by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject. Adoptive transfer may be performed with the aim of introducing a cell or population of cells into a subject, and/or increasing the frequency of a cell or population of cells in a subject.

Adoptive transfer of immune cells is described, for example, in Kalos and June (2013), Immunity 39 (1): 49-60, and Davis et al. (2015), Cancer J. 21 (6): 486-491, both of which are hereby incorporated by reference in their entirety. The skilled person is able to determine appropriate reagents and procedures for adoptive transfer of cells according to the present disclosure, for example by reference to Dai et al., 2016 J Nat Cancer Inst 108 (7): djv439, which is incorporated by reference in its entirety.

The cells and compositions according to the present disclosure may be employed in the treatment/prevention of diseases/conditions by allotransplantation or autotransplantation.

As used herein, ‘allotransplantation’ refers to the transplantation to a recipient subject of cells, tissues or organs which are genetically non-identical to the recipient subject. The cells, tissues or organs may be from, or may be derived from, cells, tissues or organs of a donor subject that is genetically non-identical to the recipient subject. Allotransplantation is distinct from autotransplantation, which refers to the transplantation of cells, tissues or organs which are from/derived from a donor subject genetically identical to the recipient subject (i.e. autologous material). It will be appreciated that adoptive transfer of allogeneic immune cells is a form of allotransplantation, and that adoptive transfer of autologous immune cells is a form of autotransplantation.

The present disclosure provides methods comprising administering cells and compositions according to the present disclosure to a subject.

In some embodiments, the methods comprise modifying an immune cell to comprise/express polypeptide(s) (e.g. recombinant CD3-TCR complex polypeptide(s), a composite polypeptide) according to the present disclosure.

In some embodiments, the methods comprise:
modifying an immune cell to express or comprise a CD3-TCR complex comprising one or more recombinant CD3-TCR complex polypeptides according to the present disclosure (e.g. as described herein), and
administering the modified immune cell to a subject.

In some embodiments, the methods further comprise:
administering an antigen-binding molecule comprising a variant Fc domain according to the present disclosure to the subject, wherein the CD3-TCR complex of the modified immune comprises an antigen-binding moiety that binds to the variant Fc domain of the antigen-binding molecule.

In some embodiments, the methods further comprise:
modifying an immune cell to reduce/prevent expression of a CD3-TCR complex polypeptide (e.g. as described herein), wherein the CD3-TCR complex polypeptide is the CD3-TCR complex polypeptide from which the CD3-TCR complex association domain of a recombinant CD3-TCR complex polypeptide of the CD3-TCR complex is derived.

It will be appreciated that the method steps recited in the preceding three paragraphs may be performed in any suitable order.

In some embodiments, the methods comprise:
administering to a subject an immune cell modified to express or comprise a CD3-TCR complex comprising one or more recombinant CD3-TCR complex polypeptides according to the present disclosure.

In some embodiments, the methods comprise:
administering to a subject an immune cell modified (i) to express or comprise a CD3-TCR complex comprising one or more recombinant CD3-TCR complex polypeptides according to the present disclosure, and (ii) to reduce/prevent expression of a CD3-TCR complex polypeptide;
wherein a recombinant CD3-TCR complex polypeptide of the CD3-TCR complex of (i) comprises a CD3-TCR complex association domain derived from the CD3-TCR complex polypeptide of (ii).

In some embodiments, in accordance with the preceding two paragraphs, the subject is a subject to which an antigen-binding molecule comprising a variant Fc domain according to the present disclosure has been administered, or is to be administered, wherein the CD3-TCR complex comprises an antigen-binding moiety that binds to the variant Fc domain of the antigen-binding molecule.

In some embodiments, the methods comprise:
(a) modifying an immune cell to express or comprise a CD3-TCR complex comprising one or more recombinant CD3-TCR complex polypeptides according to the present disclosure (e.g. as described herein); and
(b) administering an antigen-binding molecule comprising a variant Fc domain according to the present disclosure to a subject; and
(c) administering the modified immune cell to the subject;
wherein the CD3-TCR complex of (a) comprises an antigen-binding moiety that binds to the variant Fc domain of the antigen-binding molecule of (b).

In some embodiments in accordance with the method of the preceding paragraph, step (c) may be performed before step (b).

In some embodiments, the methods comprise:
(a) modifying an immune cell to reduce/prevent expression of a CD3-TCR complex polypeptide;
(b) modifying an immune cell to express or comprise a CD3-TCR complex comprising one or more recombinant CD3-TCR complex polypeptides according to the present disclosure (e.g. as described herein); and
(c) administering an antigen-binding molecule comprising a variant Fc domain according to the present disclosure to a subject; and
(d) administering the modified immune cell to the subject;
wherein a recombinant CD3-TCR complex polypeptide of the CD3-TCR complex of (b) comprises a CD3-TCR complex association domain derived from the CD3-TCR complex polypeptide of (a); and
wherein the CD3-TCR complex of (b) comprises an antigen-binding moiety that binds to the variant Fc domain of the antigen-binding molecule of (c).

In some embodiments in accordance with the method of the preceding paragraph, step (d) may be performed before step (c).

In some embodiments, the subject from which the immune cells are isolated/obtained is the same subject to which cells are administered (i.e., adoptive transfer may be of autologous/autogeneic cells). In some embodiments, the subject from which the immune cells are isolated/obtained is a different subject to the subject to which cells are administered (i.e., adoptive transfer may be of allogeneic cells).

In some embodiments, the methods may further comprise one or more of:
obtaining a blood sample from a subject;
isolating immune cells (e.g. PBMCs) from a blood sample which has been obtained from a subject;
generating/expanding a population of immune cells;
culturing the immune cells in in vitro or ex vivo cell culture;
culturing immune cells expressing/comprising a CD3-TCR complex comprising one or more recombinant CD3-TCR complex polypeptides according to the present disclosure in in vitro or ex vivo cell culture;
collecting/isolating immune cells expressing/comprising a CD3-TCR complex comprising one or more recombinant CD3-TCR complex polypeptides according to the present disclosure according to the present disclosure;
formulating immune cells expressing/comprising a CD3-TCR complex comprising one or more recombinant CD3-TCR complex polypeptides according to the present disclosure to a pharmaceutical composition, e.g. by mixing the cells with a pharmaceutically-acceptable adjuvant, diluent, or carrier.

Administration of the articles of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

Administration of the articles of the present disclosure may be parenteral, systemic, intravenous, intra-arterial, intramuscular, intracavitary, intrathecal, intraocular, intravitreal, intraconjunctival, subretinal, suprachoroidal, subcutaneous, intradermal, intrathecal, oral, nasal, topical or transdermal. Administration may be by injection or infusion. Administration of the articles of the present disclosure may be intratumoral. In some cases, the articles of the present disclosure may be formulated for targeted delivery to specific cells, a tissue, an organ and/or a tumor.

Multiple doses of an article of the present disclosure may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months.

Administration of a cell or composition according to the present disclosure with an antigen-binding molecule described herein to a subject in accordance with the therapeutic and prophylactic intervention described herein may be simultaneous or sequential.

Simultaneous administration refers to administration of (i) a cell or composition according to the present disclosure, and (ii) an antigen-binding molecule described herein together, for example as a pharmaceutical composition containing both agents (i.e. a combined preparation), or immediately after one another, and optionally via the same route of administration, e.g. to the same artery, vein or other blood vessel.

Sequential administration refers to administration of one of (i) a cell or composition according to the present disclosure, and (ii) an antigen-binding molecule described herein, followed after a given time interval by separate administration of the other agent. It is not required that the two agents are administered by the same route, although this is the case in some embodiments. The time interval may be any time interval.

The present disclosure also provides methods for depleting or killing cells comprising or expressing a target antigen, comprising contacting cells comprising/expressing a target antigen with:
(i) an antigen-binding molecule comprising: (a) an antigen-binding domain that binds to the target antigen, and (b) a variant Fc domain according to the present disclosure; and
(ii) an immune cell comprising/expressing a CD3-TCR complex comprising one or more recombinant CD3-TCR complex polypeptides according to the present disclosure;
wherein the CD3-TCR complex of (ii) comprises an antigen-binding moiety that binds to the variant Fc domain of the antigen-binding molecule of (i).

### Subjects

A subject in accordance with the various aspects of the present disclosure may be any animal or human. Therapeutic and prophylactic applications may be in human or animals (veterinary use).

The subject to be administered with an article of the present disclosure (e.g. in accordance with therapeutic or prophylactic intervention) may be a subject in need of such intervention. The subject is preferably mammalian, more preferably human. The subject may be a non-human mammal, but is more preferably human. The subject may be male or female. The subject may be a patient.

A subject may have (e.g. may have been diagnosed with) a disease or condition described herein, may be suspected of having such a disease/condition, or may be at risk of developing/contracting such a disease/condition. In embodiments according to the present disclosure, a subject may be selected for treatment according to the methods based on characterisation for one or more markers of such a disease/condition.

In some embodiments, a subject may be selected for therapeutic or prophylactic intervention as described herein based on the detection of cells/tissue expressing a target antigen (i.e. the target antigen of an antigen-binding molecule to be employed in conjunction with a cell or composition according to the present disclosure), or of cells/tissue overexpressing the target antigen, e.g. in a sample obtained from the subject.

A subject may be an allogeneic or non-autologous subject with respect to an intervention in accordance with the present disclosure. As used herein, where a subject is referred to herein as being ‘allogeneic’ or ‘non-autologous’ with respect to an intervention, the subject is a subject other than the subject from which the cell of the intervention (i.e. the cell to be administered, or the cell of the pharmaceutical composition/medicament to be administered) is derived. A subject to be treated/prevented in accordance with the present disclosure may be genetically non-identical to the subject from which the cell (e.g. the cell of the pharmaceutical composition/medicament) to be administered to the subject is derived. A subject to be treated/prevented in accordance with the present disclosure may comprise MHC/HLA genes encoding MHC/HLA molecules (e.g. MHC class I α and/or MHC class II molecules) that are non-identical to the MHC/HLA molecules (e.g. MHC class I α and/or MHC class II molecules) encoded by the cell (e.g. the cell of the pharmaceutical composition/medicament) to be administered to the subject. A subject to be treated/prevented in accordance with the present disclosure may be HLA-mismatched with respect to the subject from which the cell (e.g. the cell of the pharmaceutical composition/medicament) to be administered to the subject is derived.

The subject to which cells are administered in accordance with the present disclosure may be allogeneic/non-autologous with respect to the source from which the cell (e.g. the cell of the pharmaceutical composition/medicament) to be administered to the subject is derived. The subject to which cells are administered may be a different subject to the subject from which cells are/were obtained for the production of the cell (e.g. the cell of the pharmaceutical composition/medicament) to be administered to the subject. The subject to which the cell is administered may be genetically non-identical to the subject from which the cell (e.g. the cell of the pharmaceutical composition/medicament) to be administered to the subject cells are/were obtained for the production of the cells.

A subject may be an autogeneic/autologous subject with respect to an intervention in accordance with the present disclosure. As used herein, where a subject is referred to herein as being ‘autogeneic’ or ‘autologous’ with respect to an intervention, the subject is the same subject from which the cell of the intervention (i.e. the cell to be administered, or the cell of the pharmaceutical composition/medicament to be administered) is derived. A subject to be treated/prevented in accordance with the present disclosure may be genetically identical to the subject from which the cell (e.g. the cell of the pharmaceutical composition/medicament) to be administered to the subject is derived. A subject to be treated/prevented in accordance with the present disclosure may comprise MHC/HLA genes encoding MHC/HLA molecules (e.g. MHC class I a and/or MHC class II molecules) that are identical to the MHC/HLA molecules (e.g. MHC class I a and/or MHC class II molecules) encoded by the cell (e.g. the cell of the pharmaceutical composition/medicament) to be administered to the subject. A subject to be treated/prevented in accordance with the present disclosure may be HLA-matched with respect to the subject from which the cell (e.g. the cell of the pharmaceutical composition/medicament) to be administered to the subject is derived.

The subject to which cells are administered in accordance with the present disclosure may be autogeneic/autologous with respect to the source from which the cell (e.g. the cell of the pharmaceutical composition/medicament) to be administered to the subject is derived. The subject to which cells are administered may be the same subject as the subject from which cells are/were obtained for the production of the cell (e.g. the cell of the pharmaceutical composition/medicament) to be administered to the subject. The subject to which the cell is administered may be genetically identical to the subject from which the cell (e.g. the cell of the pharmaceutical composition/medicament) to be administered to the subject cells are/were obtained for the production of the cells.

### Kits

The present disclosure also provides kits of parts.

In some aspects and embodiments, a kit of parts according to the present disclosure comprises (i) a cell according to the present disclosure, and (ii) an antigen-binding molecule comprising: (a) an antigen-binding domain that binds to the target antigen, and (b) a variant Fc domain according to the present disclosure. It will be appreciated that in accordance with such aspects and embodiments, the cell of (i) comprises/expresses a CD3-TCR complex comprising an antigen-binding moiety that binds to the variant Fc domain of the antigen-binding molecule of (ii).

In some aspects and embodiments, a kit of parts according to the present disclosure comprises (i) a composition according to the present disclosure, and (ii) an antigen-binding molecule comprising: (a) an antigen-binding domain that binds to the target antigen, and (b) a variant Fc domain according to the present disclosure. It will be appreciated that in accordance with such aspects and embodiments, the composition of (i) comprises a cell comprising/expressing a CD3-TCR complex comprising an antigen-binding moiety that binds to the variant Fc domain of the antigen-binding molecule of (ii).

In some aspects and embodiments, a kit of parts according to the present disclosure comprises (i) a nucleic acid/plurality or an expression vector/plurality according to the present disclosure, and (ii) an antigen-binding molecule comprising: (a) an antigen-binding domain that binds to the target antigen, and (b) a variant Fc domain according to the present disclosure. It will be appreciated that in accordance with such aspects and embodiments, the nucleic acid/plurality or expression vector/plurality of (i) encode polypeptide(s) for engineering a cell to comprise/express a CD3-TCR complex comprising an antigen-binding moiety that binds to the variant Fc domain of the antigen-binding molecule of (ii).

Kits of parts according to the present disclosure may comprise a predetermined quantity of articles according to (i) and/or (ii), as described in the preceding three paragraphs. In some embodiments, articles according to (i) and/or (ii) are provided in containers (e.g. in vials or bottles). The kit may provide articles according to (i) and/or (ii) together with instructions (e.g. a protocol) as to how to employ them in accordance with a therapeutic or prophylactic intervention as described herein.

In some embodiments, a kit of parts comprises materials for producing a polypeptide according to the present disclosure, e.g. a recombinant CD3-TCR complex polypeptide according to the present disclosure. In some embodiments, a kit of parts comprises materials for producing a polypeptide complex according to the present disclosure, e.g. a CD3-TCR polypeptide complex comprising a recombinant CD3-TCR complex polypeptide according to the present disclosure. In some embodiments, a kit of parts comprises materials for producing a cell according to the present disclosure, e.g. a cell comprising/expressing a CD3-TCR polypeptide complex comprising a recombinant CD3-TCR complex polypeptide according to the present disclosure. In some embodiments, a kit of parts comprises materials for producing a composition according to the present disclosure, e.g. a pharmaceutical composition comprising a cell according to the present disclosure, e.g. a cell comprising/expressing a CD3-TCR polypeptide complex comprising a recombinant CD3-TCR complex polypeptide according to the present disclosure.

In some embodiments, the kit of parts may comprise a nucleic acid/plurality or an expression vector/plurality according to the present disclosure, and optionally materials for introducing the nucleic acid/plurality or an expression vector/plurality into a cell. In some embodiments, the kit of parts may comprise a system for producing a cell according to the present disclosure in accordance with GMP conditions. In some embodiments, the kit of parts may comprise a (closed) bag cell incubation system in which a nucleic acid/plurality or an expression vector/plurality according to the present disclosure can be introduced into a cell and subsequently cultured under GMP conditions.

In some embodiments, the kit of parts may comprise materials for formulating a cell according to the present disclosure to a pharmaceutical composition, e.g. a pharmaceutically-acceptable carrier, diluent, excipient or adjuvant.

The manufacture of kits of parts according to the present disclosure preferably follows standard procedures which are known to the person skilled in the art.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6 (298)) and MAFFT (Katoh and Standley 2013, Molecular Biology and Evolution, 30 (4) 772-780) software. When using such software, the default parameters, e.g. for gap penalty and extension penalty, are preferably used.

### Numbered Paragraphs

The following numbered paragraphs (paras) provide further statements of features and combinations of features which are contemplated in connection with the present invention:

1. A recombinant CD3-TCR complex polypeptide, comprising:
(i) an antigen-binding moiety, or a component thereof, wherein the antigen-binding moiety binds to a variant Fc domain having an amino acid sequence comprising at least one amino acid difference relative to a reference Fc domain, to which the antigen-binding moiety does not bind; and
(ii) a CD3-TCR complex association domain having an amino acid sequence derived from a CD3-TCR complex polypeptide.

2. The recombinant CD3-TCR complex polypeptide according to para 1, wherein the recombinant CD3-TCR complex polypeptide is capable of associating through its CD3-TCR complex association domain with one or more CD3-TCR complex polypeptides to form a CD3-TCR complex.

3. The recombinant CD3-TCR complex polypeptide according to para 1 or para 2, wherein the amino acid sequence derived from a CD3-TCR complex polypeptide is derived from CD3ε, TCRα or TCRβ.

4. The recombinant CD3-TCR complex polypeptide according to any one of paras 1 to 3, wherein the CD3-TCR complex association domain comprises, or consists of, an amino acid sequence having at least 70% amino acid sequence identity to one of SEQ ID NOs: 30, 52, 1, 53, 5, 54 or 9.

5. The recombinant CD3-TCR complex polypeptide according to any one of paras 1 to 4, wherein the amino acid sequence derived from a CD3-TCR complex polypeptide is derived from CD3ε.

6. The recombinant CD3-TCR complex polypeptide according to any one of paras 1 to 5, wherein the CD3-TCR complex association domain comprises, or consists of, an amino acid sequence having at least 70% amino acid sequence identity to SEQ ID NO:30.

7. The recombinant CD3-TCR complex polypeptide according to any one of paras 1 to 4, wherein the amino acid sequence derived from a CD3-TCR complex polypeptide is derived from TCRα or TCRβ.

8. The recombinant CD3-TCR complex polypeptide according to any one of paras 1 to 4 or para 7, wherein the CD3-TCR complex association domain comprises, or consists of, an amino acid sequence having at least 70% amino acid sequence identity to one of SEQ ID NOs: 52, 1, 53, 5, 54 or 9.

9. The recombinant CD3-TCR complex polypeptide according to para 7 or para 8, wherein the CD3-TCR complex association domain comprises, or consists of, an amino acid sequence having at least 70% amino acid sequence identity to SEQ ID NO:52 or 1, and comprises a cysteine residue at the position corresponding to position 47 numbered according to SEQ ID NO: 1.

10. The recombinant CD3-TCR complex polypeptide according to any one of paras 7 to 9, wherein the CD3-TCR complex association domain comprises, or consists of, an amino acid sequence having at least 70% amino acid sequence identity to any one of SEQ ID NOs: 53, 5, 54 or 9, and comprises a cysteine residue at the position corresponding to position 56 numbered according to SEQ ID NO:5.

11. The recombinant CD3-TCR complex polypeptide according to any one of paras 1 to 10, wherein the antigen-binding moiety that binds to a variant Fc domain comprises the heavy chain variable (VH) region and light chain variable (VL) region of an antibody that binds to the variant Fc domain.

12. The recombinant CD3-TCR complex polypeptide according to any one of paras 1 to 11, wherein the antigen-binding moiety is or comprises an FV, scFv, Fab, Fab′, Fab′-SH, F(ab′)₂, crossFab, scFab or dAb moiety.

13. The recombinant CD3-TCR complex polypeptide according to any one of paras 1 to 12, wherein the antigen-binding moiety is or comprises an scFV.

14. The recombinant CD3-TCR complex polypeptide according to any one of paras 1 to 13, wherein the component of the antigen-binding moiety is or comprises the VH region or the VL region an antibody that binds to the variant Fc domain.

15. The recombinant CD3-TCR complex polypeptide according to any one of paras 1 to 14, wherein the antigen-binding moiety or component thereof is connected at its C-terminus to the N-terminus of the CD3-TCR complex association domain, optionally through a linker sequence.

16. The recombinant CD3-TCR complex polypeptide according to any one of paras 1 to 15, wherein the variant Fc domain binds to an Fc receptor with lower affinity than the affinity with which the reference Fc domain binds to the Fc receptor, optionally wherein the Fc receptor is an Fcγ receptor or neonatal Fc receptor (FcRn).

17. The recombinant CD3-TCR complex polypeptide according to any one of paras 1 to 16, wherein the variant Fc domain comprises a CH2-CH3 region comprising an amino acid difference at one or more of the following positions, relative to the amino acid sequence of a CH2-CH3 region of the reference Fc domain according to EU numbering: L234, L235, I253, N297, S298, H310, P329, E333, K334 or H435.

18. The recombinant CD3-TCR complex polypeptide according to any one of paras 1 to 17, wherein the variant Fc domain comprises a CH2-CH3 region comprising G329 according to EU numbering.

19. The recombinant CD3-TCR complex polypeptide according to any one of paras 1 to 18, wherein the antigen-binding moiety comprises a VL region incorporating the following CDRs:
LC-CDR1 having the amino acid sequence of SEQ ID NO:69;
LC-CDR2 having the amino acid sequence of SEQ ID NO:70; and
LC-CDR3 having the amino acid sequence of SEQ ID NO:71.

20. The recombinant CD3-TCR complex polypeptide according to any one of paras 1 to 19, wherein the antigen-binding moiety comprises a VH incorporating:
(i) the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:58;
or
(ii) the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58.

21. The recombinant CD3-TCR complex polypeptide according to any one of paras 1 to 20, wherein the antigen-binding moiety comprises:
(a) (i) a VH region incorporating the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:68;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71;
or
(b) (i) a VH region incorporating the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71.

22. The recombinant CD3-TCR complex polypeptide according to any one of paras 1 to 21, wherein the antigen-binding moiety comprises a VL having an amino acid sequence having at least 70% amino acid sequence identity to SEQ ID NO:68.

23. The recombinant CD3-TCR complex polypeptide according to any one of paras 1 to 22, wherein the antigen-binding moiety comprises a VH having an amino acid sequence having at least 70% amino acid sequence identity to SEQ ID NO:65, 63 or 55.

24. The recombinant CD3-TCR complex polypeptide according to any one of paras 1 to 23, wherein the antigen-binding moiety comprises:
(a) (i) a VH having an amino acid sequence having at least 70% amino acid sequence identity to SEQ ID NO:65; and
   (ii) a VL having an amino acid sequence having at least 70% amino acid sequence identity to SEQ ID NO:68;
or
(b) (i) a VH having an amino acid sequence having at least 70% amino acid sequence identity to SEQ ID NO:63; and
   (ii) a VL having an amino acid sequence having at least 70% amino acid sequence identity to SEQ ID NO:68;
or
(c) (i) a VH having an amino acid sequence having at least 70% amino acid sequence identity to SEQ ID NO:55; and
   (ii) a VL having an amino acid sequence having at least 70% amino acid sequence identity to SEQ ID NO:68.

25. The recombinant CD3-TCR complex polypeptide according to any one of paras 1 to 17, wherein the variant Fc domain comprises a CH2-CH3 region comprising A298, A333 and A334 according to EU numbering.

26. The recombinant CD3-TCR complex polypeptide according to any one of paras 1 to 17 or para 25, wherein the antigen-binding moiety comprises a VH region incorporating the following CDRs:
HC-CDR1 having the amino acid sequence of SEQ ID NO:77;
HC-CDR2 having the amino acid sequence of SEQ ID NO:78; and
HC-CDR3 having the amino acid sequence of SEQ ID NO:79.

27. The recombinant CD3-TCR complex polypeptide according to any one of paras 1 to 17, para 25 or para 26, wherein the antigen-binding moiety comprises a VL region incorporating the following CDRs:
LC-CDR1 having the amino acid sequence of SEQ ID NO:85;
LC-CDR2 having the amino acid sequence of SEQ ID NO:86; and
LC-CDR3 having the amino acid sequence of SEQ ID NO:87.

28. The recombinant CD3-TCR complex polypeptide according to any one of paras 1 to 17 or 25 to 27, wherein the antigen-binding moiety comprises:
(i) a VH region incorporating the following CDRs:
   HC-CDR1 having the amino acid sequence of SEQ ID NO:77;
   HC-CDR2 having the amino acid sequence of SEQ ID NO:78; and
   HC-CDR3 having the amino acid sequence of SEQ ID NO:79;
and
(ii) a VL region incorporating the following CDRs:
   LC-CDR1 having the amino acid sequence of SEQ ID NO:85;
   LC-CDR2 having the amino acid sequence of SEQ ID NO:86; and
   LC-CDR3 having the amino acid sequence of SEQ ID NO:87.

29. The recombinant CD3-TCR complex polypeptide according to any one of paras 1 to 17 or 25 to 28, wherein the antigen-binding moiety comprises a VH having an amino acid sequence having at least 70% amino acid sequence identity to SEQ ID NO:76.

30. The recombinant CD3-TCR complex polypeptide according to any one of paras 1 to 17 or 25 to 29, wherein the antigen-binding moiety comprises a VL having an amino acid sequence having at least 70% amino acid sequence identity to SEQ ID NO:84.

31. The recombinant CD3-TCR complex polypeptide according to any one of paras 1 to 17 or 25 to 30, wherein the antigen-binding moiety comprises:
(i) a VH having an amino acid sequence having at least 70% amino acid sequence identity to SEQ ID NO:76; and
(ii) a VL having an amino acid sequence having at least 70% amino acid sequence identity to SEQ ID NO:84.

32. A recombinant CD3-TCR complex polypeptide, comprising:
(i) an antigen-binding moiety, which is an scFv, wherein the antigen-binding moiety binds to a variant Fc domain having an amino acid sequence comprising at least one amino acid difference relative to a reference Fc domain, to which the antigen-binding moiety does not bind, wherein the variant Fc domain comprises a CH2-CH3 region comprising G329 according to EU numbering; and
(ii) a CD3-TCR complex association domain having an amino acid sequence derived from a CD3-TCR complex polypeptide, wherein the amino acid sequence derived from a CD3-TCR complex polypeptide is derived from CD38.

33. The recombinant CD3-TCR complex polypeptide according to para 32, wherein the recombinant CD3-TCR complex polypeptide is capable of associating through its CD3-TCR complex association domain with one or more CD3-TCR complex polypeptides to form a CD3-TCR complex.

34. The recombinant CD3-TCR complex polypeptide according to para 32 or para 33, wherein the CD3-TCR complex association domain comprises, or consists of, an amino acid sequence having at least 70% amino acid sequence identity to SEQ ID NO:30.

35. The recombinant CD3-TCR complex polypeptide according to any one of paras 32 to 34, wherein the antigen-binding moiety is connected at its C-terminus to the N-terminus of the CD3-TCR complex association domain, optionally through a linker sequence.

36. The recombinant CD3-TCR complex polypeptide according to any one of paras 32 to 35, wherein the antigen-binding moiety comprises a VL region incorporating the following CDRs:
LC-CDR1 having the amino acid sequence of SEQ ID NO:69;
LC-CDR2 having the amino acid sequence of SEQ ID NO:70; and
LC-CDR3 having the amino acid sequence of SEQ ID NO:71.

37. The recombinant CD3-TCR complex polypeptide according to any one of paras 32 to 36, wherein the antigen-binding moiety comprises a VH incorporating:
(i) the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:58;
or
(ii) the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58.

38. The recombinant CD3-TCR complex polypeptide according to any one of paras 32 to 37, wherein the antigen-binding moiety comprises:
(a) (i) a VH region incorporating the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:68;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71;
or
(b) (i) a VH region incorporating the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71.

39. The recombinant CD3-TCR complex polypeptide according to any one of paras 32 to 38, wherein the antigen-binding moiety comprises a VL having an amino acid sequence having at least 70% amino acid sequence identity to SEQ ID NO:68.

40. The recombinant CD3-TCR complex polypeptide according to any one of paras 32 to 39, wherein the antigen-binding moiety comprises a VH having an amino acid sequence having at least 70% amino acid sequence identity to SEQ ID NO:65, 63 or 55.

41. The recombinant CD3-TCR complex polypeptide according to any one of paras 32 to 40, wherein the antigen-binding moiety comprises:
(a) (i) a VH having an amino acid sequence having at least 70% amino acid sequence identity to SEQ ID NO:65; and
   (ii) a VL having an amino acid sequence having at least 70% amino acid sequence identity to SEQ ID NO:68;
or
(b) (i) a VH having an amino acid sequence having at least 70% amino acid sequence identity to SEQ ID NO:63; and
   (ii) a VL having an amino acid sequence having at least 70% amino acid sequence identity to SEQ ID NO:68;
or
(c) (i) a VH having an amino acid sequence having at least 70% amino acid sequence identity to SEQ ID NO:55; and
   (ii) a VL having an amino acid sequence having at least 70% amino acid sequence identity to SEQ ID NO:68.

42. A polypeptide complex comprising an antigen-binding moiety, wherein the antigen-binding moiety binds to a variant Fc domain having an amino acid sequence comprising at least one amino acid difference relative to a reference Fc domain to which the antigen-binding moiety does not bind, and wherein the polypeptide complex comprises a first recombinant CD3-TCR complex polypeptide and a second recombinant CD3-TCR complex polypeptide, wherein the first and second recombinant CD3-TCR complex polypeptides each comprise: (i) a CD3-TCR complex association domain having an amino acid sequence derived from a CD3-TCR complex polypeptide, and (ii) a component of the antigen-binding moiety.

43. A polypeptide complex, comprising:
(a) a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
   (ii) a CD3-TCR complex association domain having an amino acid sequence derived from a CD3-TCR complex polypeptide; and
(b) a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form the antigen-binding moiety.

44. The polypeptide complex according to para 42 or para 43, wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.

45. The polypeptide complex according to any one of paras 42 to 44, wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

46. The polypeptide complex according to para 45, wherein the CD3-TCR complex association domain derived from TCRα comprises, or consists of, an amino acid sequence having at least 70% amino acid sequence identity to SEQ ID NO:52 or 1.

47. The polypeptide complex according to para 46, wherein the CD3-TCR complex association domain derived from TCRα comprises a cysteine residue at the position corresponding to position 47 numbered according to SEQ ID NO: 1.

48. The polypeptide complex according to any one of paras 45 to 47, wherein the CD3-TCR complex association domain derived from TCRβ comprises, or consists of, an amino acid sequence having at least 70% amino acid sequence identity to one of SEQ ID NOs: 53, 5, 54 or 9.

49. The polypeptide complex according to para 48, wherein the CD3-TCR complex association domain derived from TCRβ comprises a cysteine residue at the position corresponding to position 56 numbered according to SEQ ID NO:5.

50. The polypeptide complex according to any one of paras 42 to 49, wherein:
(i) in the first recombinant CD3-TCR complex polypeptide, the first component of an antigen-binding moiety is connected at its C-terminus to the N-terminus of the CD3-TCR complex association domain of the first recombinant CD3-TCR complex polypeptide, optionally through a linker sequence; and/or
(ii) in the second recombinant CD3-TCR complex polypeptide, the second component of an antigen-binding moiety is connected at its C-terminus to the N-terminus of the CD3-TCR complex association domain of the second recombinant CD3-TCR complex polypeptide, optionally through a linker sequence.

51. The polypeptide complex according to any one of paras 42 to 50, wherein the variant Fc domain binds to an Fc receptor with lower affinity than the affinity with which the reference Fc domain binds to the Fc receptor, optionally wherein the Fc receptor is an Fcγ receptor or neonatal Fc receptor (FcRn).

52. The polypeptide complex according to any one of paras 42 to 51, wherein the variant Fc domain comprises a CH2-CH3 region comprising an amino acid difference at one or more of the following positions, relative to the amino acid sequence of a CH2-CH3 region of the reference Fc domain according to EU numbering: L234, L235, I253, N297, S298, H310, P329, E333, K334 or H435.

53. The polypeptide complex according to any one of paras 42 to 52, wherein the variant Fc domain comprises a CH2-CH3 region comprising G329 according to EU numbering.

54. The polypeptide complex according to any one of paras 42 to 53, wherein the antigen-binding moiety comprises a VL region incorporating the following CDRs:
LC-CDR1 having the amino acid sequence of SEQ ID NO:69;
LC-CDR2 having the amino acid sequence of SEQ ID NO:70; and
LC-CDR3 having the amino acid sequence of SEQ ID NO:71.

55. The polypeptide complex according to any one of paras 42 to 54, wherein the antigen-binding moiety comprises a VH incorporating:
(i) the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:58;
or
(ii) the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58.

56. The polypeptide complex according to any one of paras 42 to 55, wherein the antigen-binding moiety comprises:
(a) (i) a VH region incorporating the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:68;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71;
or
(b) (i) a VH region incorporating the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71.

57. The polypeptide complex according to any one of paras 42 to 56, wherein the antigen-binding moiety comprises a VL having an amino acid sequence having at least 70% amino acid sequence identity to SEQ ID NO:68.

58. The polypeptide complex according to any one of paras 42 to 57, wherein the antigen-binding moiety comprises a VH having an amino acid sequence having at least 70% amino acid sequence identity to SEQ ID NO:65, 63 or 55.

59. The polypeptide complex according to any one of paras 42 to 58, wherein the antigen-binding moiety comprises:
(a) (i) a VH having an amino acid sequence having at least 70% amino acid sequence identity to SEQ ID NO:65; and
   (ii) a VL having an amino acid sequence having at least 70% amino acid sequence identity to SEQ ID NO:68;
or
(b) (i) a VH having an amino acid sequence having at least 70% amino acid sequence identity to SEQ ID NO:63; and
   (ii) a VL having an amino acid sequence having at least 70% amino acid sequence identity to SEQ ID NO:68;
or
(c) (i) a VH having an amino acid sequence having at least 70% amino acid sequence identity to SEQ ID NO:55; and
   (ii) a VL having an amino acid sequence having at least 70% amino acid sequence identity to SEQ ID NO:68.

60. The polypeptide complex according to any one of paras 42 to 52, wherein the variant Fc domain comprises a CH2-CH3 region comprising A298, A333 and A334 according to EU numbering.

61. The polypeptide complex according to any one of paras 42 to 52 or para 60, wherein the antigen-binding moiety comprises a VH region incorporating the following CDRs:
HC-CDR1 having the amino acid sequence of SEQ ID NO:77;
HC-CDR2 having the amino acid sequence of SEQ ID NO:78; and
HC-CDR3 having the amino acid sequence of SEQ ID NO:79.

62. The polypeptide complex according to any one of 42 to 52, para 60 or para 61, wherein the antigen-binding moiety comprises a VL region incorporating the following CDRs:
LC-CDR1 having the amino acid sequence of SEQ ID NO:85;
LC-CDR2 having the amino acid sequence of SEQ ID NO:86; and
LC-CDR3 having the amino acid sequence of SEQ ID NO:87.

63. The polypeptide complex according to any one of paras 42 to 52 or 60 to 62, wherein the antigen-binding moiety comprises:
(i) a VH region incorporating the following CDRs:
   HC-CDR1 having the amino acid sequence of SEQ ID NO:77;
   HC-CDR2 having the amino acid sequence of SEQ ID NO:78; and
   HC-CDR3 having the amino acid sequence of SEQ ID NO:79;
and
(ii) a VL region incorporating the following CDRs:
   LC-CDR1 having the amino acid sequence of SEQ ID NO:85;
   LC-CDR2 having the amino acid sequence of SEQ ID NO:86; and
   LC-CDR3 having the amino acid sequence of SEQ ID NO:87.

64. The polypeptide complex according to any one of paras 42 to 52 or 60 to 63, wherein the antigen-binding moiety comprises a VH having an amino acid sequence having at least 70% amino acid sequence identity to SEQ ID NO:76.

65. The polypeptide complex according to any one of paras 42 to 52 or 60 to 64, wherein the antigen-binding moiety comprises a VL having an amino acid sequence having at least 70% amino acid sequence identity to SEQ ID NO:84.

66. The polypeptide complex according to any one of paras 42 to 52 or 60 to 65, wherein the antigen-binding moiety comprises:
(i) a VH having an amino acid sequence having at least 70% amino acid sequence identity to SEQ ID NO: 76; and
(ii) a VL having an amino acid sequence having at least 70% amino acid sequence identity to SEQ ID NO: 84.

67. A polypeptide complex comprising an antigen-binding moiety, wherein the antigen-binding moiety binds to a variant Fc domain having an amino acid sequence comprising at least one amino acid difference relative to a reference Fc domain to which the antigen-binding moiety does not bind, wherein the variant Fc domain comprises a CH2-CH3 region comprising G329 according to EU numbering, and wherein the polypeptide complex comprises a first recombinant CD3-TCR complex polypeptide and a second recombinant CD3-TCR complex polypeptide, wherein:
(1) (a) the first recombinant CD3-TCR complex polypeptide comprises: (i) a first component of the antigen-binding moiety, wherein the first component of the antigen-binding moiety comprises or consists of the heavy chain variable (VH) region of an antibody that binds to the variant Fc domain, and (ii) a CD3-TCR complex association domain having an amino acid sequence derived from TCRα, and
   (b) the second recombinant CD3-TCR complex polypeptide comprises: (i) a second component of the antigen-binding moiety, wherein the second component of the antigen-binding moiety comprises or consists of the light chain variable (VL) region of the antibody that binds to the variant Fc domain, and (ii) a CD3-TCR complex association domain having an amino acid sequence derived from TCRβ;
or
(2) (a) the first recombinant CD3-TCR complex polypeptide comprises: (i) a first component of the antigen-binding moiety, wherein the first component of the antigen-binding moiety comprises or consists of the heavy chain variable (VH) region of an antibody that binds to the variant Fc domain, and (ii) a CD3-TCR complex association domain having an amino acid sequence derived from TCRβ, and
   (b) the second recombinant CD3-TCR complex polypeptide comprises: (i) a second component of the antigen-binding moiety, wherein the second component of the antigen-binding moiety comprises or consists of the light chain variable (VL) region of the antibody that binds to the variant Fc domain, and (ii) a CD3-TCR complex association domain having an amino acid sequence derived from TCRα.

68. A polypeptide complex, comprising:
(a) a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind, wherein the variant Fc domain comprises a CH2-CH3 region comprising G329 according to EU numbering; and
   (ii) a CD3-TCR complex association domain having an amino acid sequence derived from a CD3-TCR complex polypeptide; and
(b) a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form the antigen-binding moiety;
wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain;
and wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

69. The polypeptide complex according to para 67 or para 68, wherein the CD3-TCR complex association domain derived from TCRα comprises, or consists of, an amino acid sequence having at least 70% amino acid sequence identity to SEQ ID NO:52 or 1.

70. The polypeptide complex according to para 69, wherein the CD3-TCR complex association domain derived from TCRα comprises a cysteine residue at the position corresponding to position 47 numbered according to SEQ ID NO: 1.

71. The polypeptide complex according to any one of paras 67 to 70, wherein the CD3-TCR complex association domain derived from TCRβ comprises, or consists of, an amino acid sequence having at least 70% amino acid sequence identity to one of SEQ ID NOs: 53, 5, 54 or 9.

72. The polypeptide complex according to para 71, wherein the CD3-TCR complex association domain derived from TCRα comprises a cysteine residue at the position corresponding to position 56 numbered according to SEQ ID NO:5.

73. The polypeptide complex according to any one of paras 67 to 72, wherein:
(i) in the first recombinant CD3-TCR complex polypeptide, the first component of an antigen-binding moiety is connected at its C-terminus to the N-terminus of the CD3-TCR complex association domain of the first recombinant CD3-TCR complex polypeptide, optionally through a linker sequence; and/or
(ii) in the second recombinant CD3-TCR complex polypeptide, the second component of an antigen-binding moiety is connected at its C-terminus to the N-terminus of the CD3-TCR complex association domain of the second recombinant CD3-TCR complex polypeptide, optionally through a linker sequence.

74. The polypeptide complex according to any one of paras 67 to 73, wherein the antigen-binding moiety comprises a VL region incorporating the following CDRs:
LC-CDR1 having the amino acid sequence of SEQ ID NO:69;
LC-CDR2 having the amino acid sequence of SEQ ID NO:70; and
LC-CDR3 having the amino acid sequence of SEQ ID NO:71.

75. The polypeptide complex according to any one of paras 67 to 74, wherein the antigen-binding moiety comprises a VH incorporating:
(i) the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:58;
or
(ii) the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58.

76. The polypeptide complex according to any one of paras 67 to 75, wherein the antigen-binding moiety comprises:
(a) (i) a VH region incorporating the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:68;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71;
or
(b) (i) a VH region incorporating the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58;

and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71.

77. The polypeptide complex according to any one of paras 67 to 76, wherein the antigen-binding moiety comprises a VL having an amino acid sequence having at least 70% amino acid sequence identity to SEQ ID NO:68.

78. The polypeptide complex according to any one of paras 67 to 77, wherein the antigen-binding moiety comprises a VH having an amino acid sequence having at least 70% amino acid sequence identity to SEQ ID NO:65, 63 or 55.

79. The polypeptide complex according to any one of paras 67 to 78, wherein the antigen-binding moiety comprises:
(a) (i) a VH having an amino acid sequence having at least 70% amino acid sequence identity to SEQ ID NO:65; and
   (ii) a VL having an amino acid sequence having at least 70% amino acid sequence identity to SEQ ID NO:68;
or
(b) (i) a VH having an amino acid sequence having at least 70% amino acid sequence identity to SEQ ID NO:63; and
   (ii) a VL having an amino acid sequence having at least 70% amino acid sequence identity to SEQ ID NO:68;
or
(c) (i) a VH having an amino acid sequence having at least 70% amino acid sequence identity to SEQ ID NO:55; and
   (ii) a VL having an amino acid sequence having at least 70% amino acid sequence identity to SEQ ID NO:68.

80. A CD3-TCR polypeptide complex, wherein the CD3-TCR polypeptide complex comprises a recombinant CD3-TCR complex polypeptide according to any one of paras 1 to 41, or a polypeptide complex according to any one of paras 42 to 79.

81. A composite polypeptide comprising:
(a) an amino acid sequence encoding a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
   (ii) a CD3-TCR complex association domain having an amino acid sequence derived from a CD3-TCR complex polypeptide; and
(b) an amino acid sequence encoding a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form a CD3-TCR complex comprising the antigen-binding moiety;
and wherein the composite polypeptide further comprises a cleavage site between the amino acid sequences of (a) and (b).

82. The composite polypeptide according to para 81, wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.

83. The composite polypeptide according to para 81 or para 82, wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

84. The composite polypeptide according to para 83, wherein the CD3-TCR complex association domain derived from TCRα comprises, or consists of, an amino acid sequence having at least 70% amino acid sequence identity to SEQ ID NO:52 or 1.

85. The composite polypeptide according to para 84, wherein the CD3-TCR complex association domain derived from TCRα comprises a cysteine residue at the position corresponding to position 47 numbered according to SEQ ID NO: 1.

86. The composite polypeptide according to any one of paras 83 to 85, wherein the CD3-TCR complex association domain derived from TCRβ comprises, or consists of, an amino acid sequence having at least 70% amino acid sequence identity to one of SEQ ID NOs: 53, 5, 54 or 9.

87. The composite polypeptide according to para 86, wherein the CD3-TCR complex association domain derived from TCRβ comprises a cysteine residue at the position corresponding to position 56 numbered according to SEQ ID NO:5.

88. The composite polypeptide according to any one of paras 81 to 87, wherein:
(i) in the first recombinant CD3-TCR complex polypeptide, the first component of an antigen-binding moiety is connected at its C-terminus to the N-terminus of the CD3-TCR complex association domain of the first recombinant CD3-TCR complex polypeptide, optionally through a linker sequence; and/or
(ii) in the second recombinant CD3-TCR complex polypeptide, the second component of an antigen-binding moiety is connected at its C-terminus to the N-terminus of the CD3-TCR complex association domain of the second recombinant CD3-TCR complex polypeptide, optionally through a linker sequence.

89. The composite polypeptide according to any one of paras 81 to 88, wherein the variant Fc domain binds to an Fc receptor with lower affinity than the affinity with which the reference Fc domain binds to the Fc receptor, optionally wherein the Fc receptor is an Fcγ receptor or neonatal Fc receptor (FcRn).

90. The composite polypeptide according to any one of paras 81 to 89, wherein the variant Fc domain comprises a CH2-CH3 region comprising an amino acid difference at one or more of the following positions, relative to the amino acid sequence of a CH2-CH3 region of the reference Fc domain according to EU numbering: L234, L235, I253, N297, S298, H310, P329, E333, K334 or H435.

91. The composite polypeptide according to any one of paras 81 to 90, wherein the variant Fc domain comprises a CH2-CH3 region comprising G329 according to EU numbering.

92. The composite polypeptide according to any one of paras 81 to 91, wherein the antigen-binding moiety comprises a VL region incorporating the following CDRs:
LC-CDR1 having the amino acid sequence of SEQ ID NO:69;
LC-CDR2 having the amino acid sequence of SEQ ID NO:70; and
LC-CDR3 having the amino acid sequence of SEQ ID NO:71.

93. The composite polypeptide according to any one of paras 81 to 92, wherein the antigen-binding moiety comprises a VH incorporating:
(i) the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:58;
or
(ii) the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58.

94. The composite polypeptide according to any one of paras 81 to 93, wherein the antigen-binding moiety comprises:
(a) (i) a VH region incorporating the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:68;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71;
or
(b) (i) a VH region incorporating the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71.

95. The composite polypeptide according to any one of paras 81 to 94, wherein the antigen-binding moiety comprises a VL having an amino acid sequence having at least 70% amino acid sequence identity to SEQ ID NO:68.

96. The composite polypeptide according to any one of paras 81 to 95, wherein the antigen-binding moiety comprises a VH having an amino acid sequence having at least 70% amino acid sequence identity to SEQ ID NO:65, 63 or 55.

97. The composite polypeptide according to any one of paras 81 to 96, wherein the antigen-binding moiety comprises:
(a) (i) a VH having an amino acid sequence having at least 70% amino acid sequence identity to SEQ ID NO:65; and
   (ii) a VL having an amino acid sequence having at least 70% amino acid sequence identity to SEQ ID NO:68;
or
(b) (i) a VH having an amino acid sequence having at least 70% amino acid sequence identity to SEQ ID NO:63; and
   (ii) a VL having an amino acid sequence having at least 70% amino acid sequence identity to SEQ ID NO:68;
or
(c) (i) a VH having an amino acid sequence having at least 70% amino acid sequence identity to SEQ ID NO:55; and
   (ii) a VL having an amino acid sequence having at least 70% amino acid sequence identity to SEQ ID NO:68.

98. The composite polypeptide according to any one of paras 81 to 90, wherein the variant Fc domain comprises a CH2-CH3 region comprising A298, A333 and A334 according to EU numbering.

99. The composite polypeptide according to any one of paras 81 to 90 or para 98, wherein the antigen-binding moiety comprises a VH region incorporating the following CDRs:
HC-CDR1 having the amino acid sequence of SEQ ID NO:77;
HC-CDR2 having the amino acid sequence of SEQ ID NO:78; and
HC-CDR3 having the amino acid sequence of SEQ ID NO:79.

100. The composite polypeptide according to any one of 81 to 90, para 98 or para 99, wherein the antigen-binding moiety comprises a VL region incorporating the following CDRs:
LC-CDR1 having the amino acid sequence of SEQ ID NO:85;
LC-CDR2 having the amino acid sequence of SEQ ID NO:86; and
LC-CDR3 having the amino acid sequence of SEQ ID NO:87.

101. The composite polypeptide according to any one of paras 81 to 90 or 98 to 100, wherein the antigen-binding moiety comprises:
(i) a VH region incorporating the following CDRs:
   HC-CDR1 having the amino acid sequence of SEQ ID NO:77;
   HC-CDR2 having the amino acid sequence of SEQ ID NO:78; and
   HC-CDR3 having the amino acid sequence of SEQ ID NO:79;
and
(ii) a VL region incorporating the following CDRs:
   LC-CDR1 having the amino acid sequence of SEQ ID NO:85;
   LC-CDR2 having the amino acid sequence of SEQ ID NO:86; and
   LC-CDR3 having the amino acid sequence of SEQ ID NO:87.

102. The composite polypeptide according to any one of paras 81 to 90 or 98 to 101, wherein the antigen-binding moiety comprises a VH having an amino acid sequence having at least 70% amino acid sequence identity to SEQ ID NO:76.

103. The composite polypeptide according to any one of paras 81 to 90 or 98 to 102, wherein the antigen-binding moiety comprises a VL having an amino acid sequence having at least 70% amino acid sequence identity to SEQ ID NO:84.

104. The composite polypeptide according to any one of paras 81 to 90 or 98 to 103, wherein the antigen-binding moiety comprises:
(i) a VH having an amino acid sequence having at least 70% amino acid sequence identity to SEQ ID NO:76; and
(ii) a VL having an amino acid sequence having at least 70% amino acid sequence identity to SEQ ID NO: 84.

105. A nucleic acid, or a plurality of nucleic acids, encoding a recombinant CD3-TCR complex polypeptide according to any one of paras 1 to 41, a polypeptide complex according to any one of paras 42 to 79, or a composite polypeptide according to any one of paras 81 to 104.

106. A nucleic acid, or a plurality of nucleic acids, encoding:
(a) a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
   (ii) a CD3-TCR complex association domain having an amino acid sequence derived from a CD3-TCR complex polypeptide; and
(b) a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form a CD3-TCR complex comprising the antigen-binding moiety.

107. The nucleic acid or plurality of nucleic acids according to para 106, wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.

108. The nucleic acid or plurality of nucleic acids according to para 106 or para 107, wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

109. The nucleic acid or plurality of nucleic acids according to para 108, wherein the CD3-TCR complex association domain derived from TCRα comprises, or consists of, an amino acid sequence having at least 70% amino acid sequence identity to SEQ ID NO:52 or 1.

110. The nucleic acid or plurality of nucleic acids according to para 109, wherein the CD3-TCR complex association domain derived from TCRα comprises a cysteine residue at the position corresponding to position 47 numbered according to SEQ ID NO:1.

111. The nucleic acid or plurality of nucleic acids according to any one of paras 108 to 110, wherein the CD3-TCR complex association domain derived from TCRβ comprises, or consists of, an amino acid sequence having at least 70% amino acid sequence identity to one of SEQ ID NOs: 53, 5, 54 or 9.

112. The nucleic acid or plurality of nucleic acids according to para 111, wherein the CD3-TCR complex association domain derived from TCRβ comprises a cysteine residue at the position corresponding to position 56 numbered according to SEQ ID NO:5.

113. The nucleic acid or plurality of nucleic acids according to any one of paras 106 to 112, wherein:
(i) in the first recombinant CD3-TCR complex polypeptide, the first component of an antigen-binding moiety is connected at its C-terminus to the N-terminus of the CD3-TCR complex association domain of the first recombinant CD3-TCR complex polypeptide, optionally through a linker sequence; and/or
(ii) in the second recombinant CD3-TCR complex polypeptide, the second component of an antigen-binding moiety is connected at its C-terminus to the N-terminus of the CD3-TCR complex association domain of the second recombinant CD3-TCR complex polypeptide, optionally through a linker sequence.

114. The nucleic acid or plurality of nucleic acids according to any one of paras 106 to 113, wherein the variant Fc domain binds to an Fc receptor with lower affinity than the affinity with which the reference Fc domain binds to the Fc receptor, optionally wherein the Fc receptor is an Fcγ receptor or neonatal Fc receptor (FcRn).

115. The nucleic acid or plurality of nucleic acids according to any one of paras 106 to 114, wherein the variant Fc domain comprises a CH2-CH3 region comprising an amino acid difference at one or more of the following positions, relative to the amino acid sequence of a CH2-CH3 region of the reference Fc domain according to EU numbering: L234, L235, I253, N297, S298, H310, P329, E333, K334 or H435.

116. The nucleic acid or plurality of nucleic acids according to any one of paras 106 to 115, wherein the variant Fc domain comprises a CH2-CH3 region comprising G329 according to EU numbering.

117. The nucleic acid or plurality of nucleic acids according to any one of paras 106 to 116, wherein the antigen-binding moiety comprises a VL region incorporating the following CDRs:
LC-CDR1 having the amino acid sequence of SEQ ID NO:69;
LC-CDR2 having the amino acid sequence of SEQ ID NO:70; and
LC-CDR3 having the amino acid sequence of SEQ ID NO:71.

118. The nucleic acid or plurality of nucleic acids according to any one of paras 106 to 117, wherein the antigen-binding moiety comprises a VH incorporating:
(i) the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:58;
or
(ii) the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58.

119. The nucleic acid or plurality of nucleic acids according to any one of paras 106 to 118, wherein the antigen-binding moiety comprises:
(a) (i) a VH region incorporating the following CDRs:
   HC-CDR1 having the amino acid sequence of SEQ ID NO:56;
   HC-CDR2 having the amino acid sequence of SEQ ID NO:64; and
   HC-CDR3 having the amino acid sequence of SEQ ID NO:68;
and
(ii) a VL region incorporating the following CDRs:
   LC-CDR1 having the amino acid sequence of SEQ ID NO:69;
   LC-CDR2 having the amino acid sequence of SEQ ID NO: 70; and
   LC-CDR3 having the amino acid sequence of SEQ ID NO:71;
or
(b) (i) a VH region incorporating the following CDRs:
   HC-CDR1 having the amino acid sequence of SEQ ID NO:56;
   HC-CDR2 having the amino acid sequence of SEQ ID NO:57; and
   HC-CDR3 having the amino acid sequence of SEQ ID NO:58;
and
(ii) a VL region incorporating the following CDRs:
   LC-CDR1 having the amino acid sequence of SEQ ID NO:69;
   LC-CDR2 having the amino acid sequence of SEQ ID NO:70; and
   LC-CDR3 having the amino acid sequence of SEQ ID NO:71.

120. The nucleic acid or plurality of nucleic acids according to any one of paras 106 to 119, wherein the antigen-binding moiety comprises a VL having an amino acid sequence having at least 70% amino acid sequence identity to SEQ ID NO:68.

121. The nucleic acid or plurality of nucleic acids according to any one of paras 106 to 120, wherein the antigen-binding moiety comprises a VH having an amino acid sequence having at least 70% amino acid sequence identity to SEQ ID NO:65, 63 or 55.

122. The nucleic acid or plurality of nucleic acids according to any one of paras 106 to 121, wherein the antigen-binding moiety comprises:
(a) (i) a VH having an amino acid sequence having at least 70% amino acid sequence identity to SEQ ID NO:65; and
   (ii) a VL having an amino acid sequence having at least 70% amino acid sequence identity to SEQ ID NO:68;
or
(b) (i) a VH having an amino acid sequence having at least 70% amino acid sequence identity to SEQ ID NO:63; and
   (ii) a VL having an amino acid sequence having at least 70% amino acid sequence identity to SEQ ID NO:68;
or
(c) (i) a VH having an amino acid sequence having at least 70% amino acid sequence identity to SEQ ID NO:55; and
   (ii) a VL having an amino acid sequence having at least 70% amino acid sequence identity to SEQ ID NO:68.

123. The nucleic acid or plurality of nucleic acids according to any one of paras 106 to 115, wherein the variant Fc domain comprises a CH2-CH3 region comprising A298, A333 and A334 according to EU numbering. 124. The nucleic acid or plurality of nucleic acids according to any one of paras 106 to 115 or para 123, wherein the antigen-binding moiety comprises a VH region incorporating the following CDRs:
HC-CDR1 having the amino acid sequence of SEQ ID NO:77;
HC-CDR2 having the amino acid sequence of SEQ ID NO:78; and
HC-CDR3 having the amino acid sequence of SEQ ID NO:79.

125. The nucleic acid or plurality of nucleic acids according to any one of paras 106 to 115, para 123 or para 124, wherein the antigen-binding moiety comprises a VL region incorporating the following CDRs:
LC-CDR1 having the amino acid sequence of SEQ ID NO:85;
LC-CDR2 having the amino acid sequence of SEQ ID NO:86; and
LC-CDR3 having the amino acid sequence of SEQ ID NO:87.

126. The nucleic acid or plurality of nucleic acids according to any one of paras 106 to 115 or 123 to 125, wherein the antigen-binding moiety comprises:
(i) a VH region incorporating the following CDRs:
   HC-CDR1 having the amino acid sequence of SEQ ID NO:77;
   HC-CDR2 having the amino acid sequence of SEQ ID NO:78; and
   HC-CDR3 having the amino acid sequence of SEQ ID NO:79;
and
(ii) a VL region incorporating the following CDRs:
   LC-CDR1 having the amino acid sequence of SEQ ID NO:85;
   LC-CDR2 having the amino acid sequence of SEQ ID NO:86; and
   LC-CDR3 having the amino acid sequence of SEQ ID NO:87.

127. The nucleic acid or plurality of nucleic acids according to any one of paras 106 to 115 or 123 to 125, wherein the antigen-binding moiety comprises a VH having an amino acid sequence having at least 70% amino acid sequence identity to SEQ ID NO:76.

128. The nucleic acid or plurality of nucleic acids according to any one of paras 106 to 115 or 123 to 127, wherein the antigen-binding moiety comprises a VL having an amino acid sequence having at least 70% amino acid sequence identity to SEQ ID NO: 84.

129. The nucleic acid or plurality of nucleic acids according to any one of paras 106 to 115 or 123 to 128, wherein the antigen-binding moiety comprises:
(i) a VH having an amino acid sequence having at least 70% amino acid sequence identity to SEQ ID NO:76; and
(ii) a VL having an amino acid sequence having at least 70% amino acid sequence identity to SEQ ID NO:84.

130. An expression vector, or a plurality of expression vectors, comprising a nucleic acid or a plurality of nucleic acids according to any one of paras 105 to 129.

131. A cell comprising a recombinant CD3-TCR complex polypeptide according to any one of paras 1 to 41, a polypeptide complex according to any one of paras 42 to 79, a CD3-TCR polypeptide complex according to para 80, a composite polypeptide according to any one of paras 81 to 104, a nucleic acid or a plurality of nucleic acids according to any one of paras 105 to 129, or an expression vector or a plurality of expression vectors according to para 130.

132. A pharmaceutical composition comprising a cell according to para 131.

133. A cell according to para 131, or a pharmaceutical composition according to para 132, for use in a method of medical treatment or prophylaxis.

134. A cell according to para 131, or a pharmaceutical composition according to para 132, for use in a method of treating or preventing a disease in which cells comprising or expressing a target antigen are pathologically-implicated, wherein the method comprises administering the cell or pharmaceutical composition to a subject to which an antigen-binding molecule has been or is to be administered;
wherein the antigen-binding molecule comprises: (a) an antigen-binding domain that binds to the target antigen, and (b) a variant Fc domain having an amino acid sequence comprising at least one amino acid difference relative to a reference Fc domain;
and wherein the antigen-binding moiety of the recombinant CD3-TCR complex polypeptide, polypeptide complex, CD3-TCR polypeptide complex or composite polypeptide comprised in the cell of para 131 or the cell comprised in the pharmaceutical composition of para 132, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell of para 131, or comprised in the cell comprised in the pharmaceutical composition of para 132, binds to the variant Fc domain.

135. A method for depleting or killing cells comprising or expressing a target antigen, comprising contacting cells comprising/expressing a target antigen with:
(i) a cell according to para 131, or a pharmaceutical composition according to para 132; and
(ii) an antigen-binding molecule comprising: (a) an antigen-binding domain that binds to the target antigen, and (b) a variant Fc domain having an amino acid sequence comprising at least one amino acid difference relative to a reference Fc domain;
wherein the antigen-binding moiety of the recombinant CD3-TCR complex polypeptide, polypeptide complex, CD3-TCR polypeptide complex or composite polypeptide comprised in the cell of para 131 or the cell comprised in the pharmaceutical composition of para 132, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell of para 131, or comprised in the cell comprised in the pharmaceutical composition of para 132, binds to the variant Fc domain.

136. A kit, comprising:
(i) a cell according to para 131, or a pharmaceutical composition according to para 132; and
(ii) an antigen-binding molecule comprising: (a) an antigen-binding domain that binds to a target antigen, and (b) a variant Fc domain having an amino acid sequence comprising at least one amino acid difference relative to a reference Fc domain;
wherein the antigen-binding moiety of the recombinant CD3-TCR complex polypeptide, polypeptide complex, CD3-TCR polypeptide complex or composite polypeptide comprised in the cell of para 131 or the cell comprised in the pharmaceutical composition of para 132, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell of para 131, or comprised in the cell comprised in the pharmaceutical composition of para 132, binds to the variant Fc domain.

137. A kit, comprising:
(i) a nucleic acid or a plurality of nucleic acids according to any one of paras 105 to 129, or an expression vector or a plurality of expression vectors according to para 130; and
(ii) an antigen-binding molecule comprising: (a) an antigen-binding domain that binds to a target antigen, and (b) a variant Fc domain having an amino acid sequence comprising at least one amino acid difference relative to a reference Fc domain;
wherein the antigen-binding moiety of the CD3-TCR complex polypeptide, polypeptide complex, CD3-TCR polypeptide complex or composite polypeptide encoded by the nucleic acid or plurality thereof according to any one of paras 105 to 129, or encoded by the nucleic acid or plurality thereof comprised in the expression vector or plurality thereof according to para 130, binds to the variant Fc domain.

Further numbered embodiments:

1B. A recombinant CD3-TCR complex polypeptide, comprising:
(i) an antigen-binding moiety, or a component thereof, wherein the antigen-binding moiety binds to a variant Fc domain having an amino acid sequence comprising at least one amino acid difference relative to a reference Fc domain, to which the antigen-binding moiety does not bind; and
(ii) a CD3-TCR complex association domain having an amino acid sequence derived from a CD3-TCR complex polypeptide.

2B. The recombinant CD3-TCR complex polypeptide according to para 1B, wherein the recombinant CD3-TCR complex polypeptide is capable of associating through its CD3-TCR complex association domain with one or more CD3-TCR complex polypeptides to form a CD3-TCR complex.

3B. The recombinant CD3-TCR complex polypeptide according to para 1B or para 2B, wherein the amino acid sequence derived from a CD3-TCR complex polypeptide is derived from CD3ε, TCRα or TCRβ.

4B. The recombinant CD3-TCR complex polypeptide according to any one of paras 1B to 3B, wherein the amino acid sequence derived from a CD3-TCR complex polypeptide is derived from CD38.

5B. The recombinant CD3-TCR complex polypeptide according to any one of paras 1B to 4B, wherein the antigen-binding moiety that binds to a variant Fc domain comprises the heavy chain variable (VH) region and light chain variable (VL) region of an antibody that binds to the variant Fc domain.

6B. The recombinant CD3-TCR complex polypeptide according to any one of paras 1B to 5B, wherein the antigen-binding moiety is or comprises an Fv, scFv, Fab, Fab′, Fab′-SH, F(ab′)₂, crossFab, scFab or dAb moiety. 7B. The recombinant CD3-TCR complex polypeptide according to any one of paras 1B to 6B, wherein the antigen-binding moiety is or comprises an scFV.

8B. The recombinant CD3-TCR complex polypeptide according to any one of paras 1B to 7B, wherein the antigen-binding moiety or component thereof is connected at its C-terminus to the N-terminus of the CD3-TCR complex association domain, optionally through a linker sequence.

9B. The recombinant CD3-TCR complex polypeptide according to any one of paras 1B to 8B, wherein the variant Fc domain comprises a CH2-CH3 region comprising G329 according to EU numbering.

10B. A recombinant CD3-TCR complex polypeptide, comprising:
(i) an antigen-binding moiety, which is an scFv, wherein the antigen-binding moiety binds to a variant Fc domain having an amino acid sequence comprising at least one amino acid difference relative to a reference Fc domain, to which the antigen-binding moiety does not bind, wherein the variant Fc domain comprises a CH2-CH3 region comprising G329 according to EU numbering; and
(ii) a CD3-TCR complex association domain having an amino acid sequence derived from a CD3-TCR complex polypeptide, wherein the amino acid sequence derived from a CD3-TCR complex polypeptide is derived from CD3.

11B. A polypeptide complex, comprising:
(a) a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
   (ii) a CD3-TCR complex association domain having an amino acid sequence derived from a CD3-TCR complex polypeptide; and
(b) a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form the antigen-binding moiety.

12B. The polypeptide complex according to para 11B, wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.

13B. The polypeptide complex according to para 11B or para 12B, wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

14B. A polypeptide complex, comprising:
(a) a first recombinant CD3-TCR complex polypeptide comprising:
   (i) a first component of an antigen-binding moiety, wherein the antigen-binding moiety binds to a variant Fc domain having an amino acid sequence comprising at least one amino acid difference relative to a reference Fc domain to which the antigen-binding moiety does not bind, wherein the variant Fc domain comprises a CH2-CH3 region comprising G329 according to EU numbering; and
   (ii) a CD3-TCR complex association domain having an amino acid sequence derived from a CD3-TCR complex polypeptide; and
(b) a second recombinant CD3-TCR complex polypeptide comprising:
   (i) a second component of the antigen-binding moiety of (a) (i); and
   (ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form the antigen-binding moiety;
wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain;
and wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

15B. A CD3-TCR polypeptide complex, wherein the CD3-TCR polypeptide complex comprises a recombinant CD3-TCR complex polypeptide according to any one of paras 1B to 10B, or a polypeptide complex according to any one of paras 11B to 14B.

Sequences

| SEQ | | |
|---|---|---|
| ID | | |
| NO: | DESCRIPTION | SEQUENCE |
| 1 | Human TRAC (UniProt: | IQNPDPAVYQLRDSKSSDKSVCLFTDFDSQTNVSQSKDSDVYITDKTVLDMRSMDFKSNSAV |
| | P01848) | AWSNKSDFACANAFNNSIIPEDTFFPSPESSCDVKLVEKSFETDTNLNFQNLSVIGFRILLLKVA |
| | | GFNLLMTLRLWSS |
| 2 | Human TRAC ECD | IQNPDPAVYQLRDSKSSDKSVCLFTDFDSQTNVSQSKDSDVYITDKTVLDMRSMDFKSNSAV |
| | | AWSNKSDFACANAFNNSIIPEDTFFPSPESSCDVKLVEKSFETDTNLNFQNLS |
| 3 | Human TRAC TMD | VIGFRILLLKVAGFNLLMTLRLW |
| 4 | Human TRAC ICD | SS |
| 5 | Human TRBC1 | DLNKVFPPEVAVFEPSEAEISHTQKATLVCLATGFFPDHVELSWWWVNGKEVHSGVSTDPQPL |
| | (UniProt: P01850) | KEQPALNDSRYCLSSRLRVSATFWQNPRNHFRCQVQFYGLSENDEWTQDRAKPVTQIVSAE |
| | | AWGRADCGFTSVSYQQGVLSATILYEILLGKATLYAVLVSALVLMAMVKRKDF |
| 6 | Human TRBC1 ECD | DLNKVFPPEVAVFEPSEAEISHTQKATLVCLATGFFPDHVELSWWWVNGKEVHSGVSTDPQPL |
| | | KEQPALNDSRYCLSSRLRVSATFWQNPRNHFRCQVQFYGLSENDEWTQDRAKPVTQIVSAE |
| | | AWGRADCGFTSVSYQQGVLSATILYE |
| 7 | Human TRBC1 TMD | ILLGKATLYAVLVSALVLMAM |
| 8 | Human TRBC1 ICD | VKRKDF |
| 9 | Human TRBC2 | DLKNVFPPKVAVFEPSEAEISHTQKATLVCLATGFYPDHVELSWWVNGKEVHSGVSTDPQPL |
| | (UniProt: A0A5B9) | KEQPALNDSRYCLSSRLRVSATFWQNPRNHFRCQVQFYGLSENDEWTQDRAKPVTQIVSAE |
| | | AWGRADCGFTSESYQQGVLSATILYEILLGKATLYAVLVSALVLMAMVKRKDSRG |
| 10 | Human TRBC2 ECD | DLKNVFPPKVAVFEPSEAEISHTQKATLVCLATGFYPDHVELSWWWVNGKEVHSGVSTDPQPL |
| | | KEQPALNDSRYCLSSRLRVSATFWQNPRNHFRCQVQFYGLSENDEWTQDRAKPVTQIVSAE |
| | | AWGRADCGFTSESYQQGVLSA |
| 11 | Human TRBC2 TMD | TILYEILLGKATLYAVLVSALVL |
| 12 | Human TRBC2 ICD | MAMVKRKDSRG |
| 13 | Human TCRγ1 (UniProt: | DKQLDADVSPKPTIFLPSIAETKLQKAGTYLCLLEKFFPDVIKIHWQEKKSNTILGSQEGNTMKT |
| | POCF51) | NDTYMKFSWLTVPEKSLDKEHRCIVRHENNKNGVDQEIIFPPIKTDVITMDPKDNCSKDANDTL |
| | | LLQLTNTSAYYMYLLLLLKSVVYFAIITCCLLRRTAFCQNGEKS |
| 14 | Human TCRγ1 ECD | DKQLDADVSPKPTIFLPSIAETKLQKAGTYLCLLEKFFPDVIKIHWQEKKSNTILGSQEGNTMKT |
| | | NDTYMKFSWLTVPEKSLDKEHRCIVRHENNKNGVDQEIIFPPIKTDVITMDPKDNCSKDANDTL |
| | | LLQLTNTSA |
| 15 | Human TCRγ1 TMD | YYMYLLLLLKSVVYFAIITCCLL |
| 16 | Human TCRγ1 ICD | RRTAFCCNGEKS |
| 17 | Human TCRγ2 (UniProt: | DKQLDADVSPKPTIFLPSIAETKLQKAGTYLCLLEKFFPDIIKIHWQEKKSNTILGSQEGNTMKT |
| | P03986) | NDTYMKFSWLTVPEESLDKEHRCIVRHENNKNGIDQEIIFPPIKTDVTTVDPKYNYSKDANDVIT |
| | | MDPKDNWSKDANDTLLLQLTNTSAYYTYLLLLLKSVVYFAIITCCLLRRTAFCCNGEKS |
| 18 | Human TCRγ2 ECD | DKQLDADVSPKPTIFLPSIAETKLQKAGTYLCLLEKFFPDIIKIHWQEKKSNTILGSQEGNTMKT |
| | | NDTYMKFSWLTVPEESLDKEHRCIVRHENNKNGIDQEIIFPPIKTDVTTVDPKYNYSKDANDVIT |
| | | MDPKDNWSKDANDTLLLQLTNTSA |
| 19 | Human TCRγ2 TMD | YYTYLLLLLKSVVYFAIITCCLL |
| 20 | Human TCRγ2 ICD | RRTAFCCNGEKS |
| 21 | Human TRDC (UniProt: | SQPHTKPSVFVMKNGTNVACLVKEFYPKDIRINLVSSKKITEFDPAIVISPSGKYNAVKLGKYED |
| | B7Z8K6) | SNSVTCSVQHDNKTVHSTDFEVKTDSTDHVKPKETENTKQPSKSCHKPKAIVHTEKVNMMSL |
| | | TVLGLRMLFAKTVAVNFLLTAKLFFL |
| 22 | Human TRDC ECD | SQPHTKPSVFVMKNGTNVACLVKEFYPKDIRINLVSSKKITEFDPAIVISPSGKYNAVKLGKYED |
| | | SNSVTCSVQHDNKTVHSTDFEVKTDSTDHVKPKETENTKQPSKSCHKPKAIVHTEKVNMMSL |
| | | TV |
| 23 | Human TRDC TMD | LGLRMLFAKTVAVNFLLTAKLFF |
| 24 | Human TRDC ICD | L |
| 25 | Human CD3ϵ (UniProt: | MQSGTHWRVLGLCLLSVGVWGQDGNEEMGGITQTPYKVSISGTTVILTCPQYPGSEILWQHN |
| | P07766) | DKNIGGDEDDKNIGSDEDHLSLKEFSELEQSGYYVCYPRGSKPEDANFYLYLRARVCENCME |
| | | MDVMSVATIVIVDICITGGLLLLVYYWSKNRKAKAKPVTRGAGAGGRQRGQNKERPPPVPNP |
| | | DYEPIRKGQRDLYSGLNQRRI |
| 26 | Human CD3ϵ SP | MQSGTHWRVLGLCLLSVGVWGQ |
| 27 | Human CD3ϵ ECD | DGNEEMGGITQTPYKVSISGTTVILTCPQYPGSEILWQHNDKNIGGDEDDKNIGSDEDHLSLKE |
| | | FSELEQSGYYVCYPRGSKPEDANFYLYLRARVCENCMEMD |
| 28 | Human CD3ϵ TMD | VMSVATIVIVDICITGGLLLLVYYWS |
| 29 | Human CD3ϵ ICD | KNRKAKAKPVTRGAGAGGRQRGQNKERPPPVPNPDYEPIRKGQRDLYSGLNQRRI |
| 30 | Mature human CD3ϵ | DGNEEMGGITQTPYKVSISGTTVILTCPQYPGSEILWQHNDKNIGGDEDDKNIGSDEDHLSLKE |
| | | FSELEQSGYYVCYPRGSKPEDANFYLYLRARVCENCMEMDVMSVATIVIVDICITGGLLLLVYY |
| | | WSKNRKAKAKPVTRGAGAGGRQRGQNKERPPPVPNPDYEPIRKGQRDLYSGLNQRRI |
| 31 | Human CD3δ (UniProt: | MEHSTFLSGLVLATLLSQVSPFKIPIEELEDRVFVNCNTSITWVEGTVGTLLSDITRLDLGKRILD |
| | P04234) | PRGIYRCNGTDIYKDKESTVQVHYRMCQSCVELDPATVAGIIVTDVIATLLLALGVFCFAGHET |
| | | GRLSGAADTQALLRNDQVYQPLRDRDDAQYSHLGGNWARNK |
| 32 | Human CD3δ SP | MEHSTFLSGLVLATLLSQVSP |
| 33 | Human CD3δ ECD | FKIPIEELEDRVFVNCNTSITWVEGTVGTLLSDITRLDLGKRILDPRGIYRCNGTDIYKDKESTVQ |
| | | VHYRMCQSCVELDPATVA |
| 34 | Human CD3δ TMD | GIIVTDVIATLLLALGVFCFA |
| 35 | Human CD3δ ICD | GHETGRLSGAADTQALLRNDQVYQPLRDRDDAQYSHLGGNWARNK |
| 36 | Mature human CD30 | FKIPIEELEDRVFVNCNTSITWVEGTVGTLLSDITRLDLGKRILDPRGIYRCNGTDIYKDKESTVQ |
| | | VHYRMCQSCVELDPATVAGIIVTDVIATLLLALGVFCFAGHETGRLSGAADTQALLRNDQVYQ |
| | | PLRDRDDAQYSHLGGNWARNK |
| 37 | Human CD3γ (UniProt: | MEHSTFLSGLVLATLLSQVSPFKIPIEELEDRVFVNCNTSITWVEGTVGTLLSDITRLDLGKRILD |
| | P04234) | PRGIYRCNGTDIYKDKESTVQVHYRMCQSCVELDPATVAGIIVTDVIATLLLALGVFCFAGHET |
| | | GRLSGAADTQALLRNDQVYQPLRDRDDAQYSHLGGNWARNK |
| 38 | Human CD3γ SP | MEHSTFLSGLVLATLLSQVSP |
| 39 | Human CD3γ ECD | FKIPIEELEDRVFVNCNTSITWVEGTVGTLLSDITRLDLGKRILDPRGIYRCNGTDIYKDKESTVQ |
| | | VHYRMCQSCVELDPATVA |
| 40 | Human CD3γ TMD | GIIVTDVIATLLLALGVFCFA |
| 41 | Human CD3γ ICD | GHETGRLSGAADTQALLRNDQVYQPLRDRDDAQYSHLGGNWARNK |
| 42 | Mature human CD3γ | FKIPIEELEDRVFVNCNTSITWVEGTVGTLLSDITRLDLGKRILDPRGIYRCNGTDIYKDKESTVQ |
| | | VHYRMCQSCVELDPATVAGIIVTDVIATLLLALGVFCFAGHETGRLSGAADTQALLRNDQVYQ |
| | | PLRDRDDAQYSHLGGNWARNK |
| 43 | Human CD3ζ (UniProt: | MKWKALFTAAILQAQLPITEAQSFGLLDPKLCYLLDGILFIYGVILTALFLRVKFSRSADAPAYQQ |
| | P20963-1) | GQNQLYNELNLGRREEYDVLDKRRGRDPEMGGKPQRRKNPQEGLYNELQKDKMAEAYSEI |
| | | GMKGERRRGKGHDGLYQGLSTATKDTYDALHMQALPPR |
| 44 | Human CD3ζ, CD3η SP | MKWKALFTAAILQAQLPITEA |
| 45 | Human CD3ζ, CD3η | QSFGLLDPK |
| | ECD | |
| 46 | Human CD3ζ, CD3η | LCYLLDGILFIYGVILTALFL |
| | TMD | |
| 47 | Human CD3ζ ICD | RVKFSRSADAPAYQQGQNQLYNELNLGRREEYDVLDKRRGRDPEMGGKPQRRKNPQEGLY |
| | | NELQKDKMAEAYSEIGMKGERRRGKGHDGLYQGLSTATKDTYDALHMQALPPR |
| 48 | Mature human CD3ζ | QSFGLLDPKLCYLLDGILFIYGVILTALFLRVKFSRSADAPAYQQGQNQLYNELNLGRREEYDV |
| | | LDKRRGRDPEMGGKPQRRKNPQEGLYNELQKDKMAEAYSEIGMKGERRRGKGHDGLYQGL |
| | | STATKDTYDALHMQALPPR |
| 49 | Human CD3η (UniProt: | MKWKALFTAAILQAQLPITEAQSFGLLDPKLCYLLDGILFIYGVILTALFLRVKFSRSADAPAYQQ |
| | P20963-2) | GQNQLYNELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 50 | Human CD3η ICD | RVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 51 | Mature human CD3η | QSFGLLDPKLCYLLDGILFIYGVILTALFLRVKFSRSADAPAYQQGQNQLYNELNLGRREEYDV |
| | | LDKRRGRDPEMGGKPRRKNPQEGLYNELQKDKMAEAYSEIGMKGERRRGKGHDGLYQGLS |
| | | TATKDTYDALHMQALPPR |
| 52 | Human TRAC_T47C | IQNPDPAVYQLRDSKSSDKSVCLFTDFDSQTNVSQSKDSDVYITDKCVLDMRSMDFKSNSAV |
| | | AWSNKSDFACANAFNNSIIPEDTFFPSPESSCDVKLVEKSFETDTNLNFQNLSVIGFRILLLKVA |
| | | GFNLLMTLRLWSS |
| 53 | Human TRBC1_S56C | DLNKVFPPEVAVFEPSEAEISHTQKATLVCLATGFFPDHVELSWWWVNGKEVHSGVCTDPQPL |
| | | KEQPALNDSRYCLSSRLRVSATFWQNPRNHFRCQVQFYGLSENDEWTQDRAKPVTQIVSAE |
| | | AWGRADCGFTSVSYQQGVLSATILYEILLGKATLYAVLVSALVLMAMVKRKDF |
| 54 | Human TRBC2_S56C | DLKNVFPPKVAVFEPSEAEISHTQKATLVCLATGFYPDHVELSWWWVNGKEVHSGVCTDPQPL |
| | | KEQPALNDSRYCLSSRLRVSATFWQNPRNHFRCQVQFYGLSENDEWTQDRAKPVTQIVSAE |
| | | AWGRADCGFTSESYQQGVLSATILYEILLGKATLYAVLVSALVLMAMVKRKDSRG |
| 55 | αP329G_VH1 | EVQLVESGGGLVQPGGSLRLSCAASGFDFSRYWMNWVRQAPGKGLEWVGEITPDSSTINYT |
| | | PSLKGRFTISRDNAKNSLYLQMNSLRAEDTAVYYCVRPYDYGAWFASWGQGTLVTVSS |
| 56 | αP329G_VH1, VH2, VH3 | RYWMN |
| | HC-CDR1 | |
| 57 | αP329G_VH1 HC-CDR2 | EITPDSSTINYTPSLKG |
| 58 | αP329G_VH1, VH2, VH3 | PYDYGAWFAS |
| | HC-CDR3 | |
| 59 | αP329G_VH1, VH2 HC- | EVQLVESGGGLVQPGGSLRLSCAASGFDFS |
| | FR1 | |
| 60 | αP329G_ VH1, VH2, | WVRQAPGKGLEWVG |
| | VH3 HC-FR2 | |
| 61 | αP329G_VH1, VH2 HC- | RFTISRDNAKNSLYLQMNSLRAEDTAVYYCVR |
| | FR3 | |
| 62 | αP329G_VH1, VH2, VH3 | WGQGTLVTVSS |
| | HC-FR4 | |
| 63 | αP329G_VH2 | EVQLVESGGGLVQPGGSLRLSCAASGFDFSRYWMNWVRQAPGKGLEWVGEITPDSSTINYA |
| | | PSLKGRFTISRDNAKNSLYLQMNSLRAEDTAVYYCVRPYDYGAWFASWGQGTLVTVSS |
| 64 | αP329G_VH2, VH3 HC- | EITPDSSTINYAPSLKG |
| | CDR2 | |
| 65 | αP329G_VH3 | EVQLVESGGGLVQPGGSLRLSCAASGFTFSRYWMNWVRQAPGKGLEWVGEITPDSSTINYA |
| | | PSLKGRFTISRDNAKNSLYLQMNSLRAEDTAVYYCARPYDYGAWFASWGQGTLVTVSS |
| 66 | αP329G_VH3 HC-FR1 | EVQLVESGGGLVQPGGSLRLSCAASGFTFS |
| 67 | αP329G_VH3 HC-FR3 | RFTISRDNAKNSLYLQMNSLRAEDTAVYYCAR |
| 68 | αP329G_VL1 | QAVVTQEPSLTVSPGGTVTLTCRSSTGAVTTSNYANWVQEKPDHLFTGLIGGTNKRAPGTPA |
| | | RFSGSLLGGKAALTLSGAQPEDEAEYYCALWYSNHWVFGGGTKLTVL |
| 69 | αP329G_VL1 LC-CDR1 | RSSTGAVTTSNYAN |
| 70 | αP329G_VL1 LC-CDR2 | GTNKRAP |
| 71 | αP329G_VL1 LC-CDR3 | ALWYSNHWV |
| 72 | αP329G_VL1 LC-FR1 | QAVVTQEPSLTVSPGGTVTLTC |
| 73 | αP329G_VL1 LC-FR2 | WVQEKPDHLFTGLIG |
| 74 | αP329G_VL1 LC-FR3 | GTPARFSGSLLGGKAALTLSGAQPEDEAEYYC |
| 75 | αP329G_VL1 LC-FR4 | FGGGTKLTVL |
| 76 | αAAA_VH | EVQLVESGGDLVKPGGSLKLSCAASGFTFSSYGMSWVRQTPDKRLEWVATISSGGSYIYYPD |
| | | SVKGRFTISRDNAKNTLYLQMSSLKSEDTAMYYCARLGMITTGYAMDYWGQGTSVTVSS |
| 77 | αAAA_VH HC-CDR1 | SYGMS |
| 78 | αAAA_VH HC-CDR2 | SSGGSY |
| 79 | αAAA_VH HC-CDR3 | LGMITTGYAMDY |
| 80 | αAAA VH HC-FR1 | EVQLVESGGDLVKPGGSLKLSCAASGFTFS |
| 81 | αAAA VH HC-FR2 | WRQTPDKRLEWVATI |
| 82 | αAAA_VH HC-FR3 | IYYPDSVKGRFTISRDNAKNTLYLQMSSLKSEDTAMYYCAR |
| 83 | αAAA VH HC-FR4 | WGQGTSVTVSS |
| 84 | αAAA_VL | DVLMTQTPLSLPVSLGDQASISCRSSQTIVHSTGHTYLEWFLQKPGQSPKLLIYKVSNRFSGV |
| | | PDRFSGSGSGTDFTLKISRVEAEDLGVYYCFQGSHVPYTFGGGTKLEIK |
| 85 | αAAA VL LC-CDR1 | RSSQTIVHSTGHTYLE |
| 86 | αAAA_VL LC-CDR2 | KVSNRFS |
| 87 | αAAA VL LC-CDR3 | FQGSHVPYT |
| 88 | αAAA_VL LC-FR1 | DVLMTQTPLSLPVSLGDQASISC |
| 89 | αAAA VL LC-FR2 | WFLQKPGQSPKLLIY |
| 90 | αAAA VL LC-FR3 | GVPDRFSGSGSGTDFTLKISRVEAEDLGVYYC |
| 91 | αAAA VL LC-FR4 | FGGGTKLEIK |
| 92 | G₄S | GGGGS |
| 93 | (G₄S)3 linker | GGGGSGGGGSGGGGS |
| 94 | αP329G VH1VL1 scFv | EVQLVESGGGLVQPGGSLRLSCAASGFDFSRYWMNWVRQAPGKGLEWVGEITPDSSTINYT |
| | | PSLKGRFTISRDNAKNSLYLQMNSLRAEDTAVYYCVRPYDYGAWFASWGQGTLVTVSSGGG |
| | | GSGGGGSGGGGSQAVVTQEPSLTVSPGGTVTLTCRSSTGAVTTSNYANWVQEKPDHLFTGL |
| | | IGGTNKRAPGTPARFSGSLLGGKAALTLSGAQPEDEAEYYCALWYSNHWVFGGGTKLTVL |
| 95 | αP329G VH2VL1 scFv | EVQLVESGGGLVQPGGSLRLSCAASGFDFSRYWMNWVRQAPGKGLEWVGEITPDSSTINYA |
| | | PSLKGRFTISRDNAKNSLYLQMNSLRAEDTAVYYCVRPYDYGAWFASWGQGTLVTVSSGGG |
| | | GSGGGGSGGGGSQAVVTQEPSLTVSPGGTVTLTCRSSTGAVTTSNYANWVQEKPDHLFTGL |
| | | IGGTNKRAPGTPARFSGSLLGGKAALTLSGAQPEDEAEYYCALWYSNHWVFGGGTKLTVL |
| 96 | αP329G VH3VL1 scFv | EVQLVESGGGLVQPGGSLRLSCAASGFTFSRYWMNWVRQAPGKGLEWVGEITPDSSTINYA |
| | | PSLKGRFTISRDNAKNSLYLQMNSLRAEDTAVYYCARPYDYGAWFASWGQGTLVTVSSGGG |
| | | GSGGGGSGGGGSQAVVTQEPSLTVSPGGTVTLTCRSSTGAVTTSNYANWVQEKPDHLFTGL |
| | | IGGTNKRAPGTPARFSGSLLGGKAALTLSGAQPEDEAEYYCALWYSNHWVFGGGTKLTVL |
| 97 | αAAA scFv | EVQLVESGGDLVKPGGSLKLSCAASGFTFSSYGMSWVRQTPDKRLEWVATISSGGSYIYYPD |
| | | SVKGRFTISRDNAKNTLYLQMSSLKSEDTAMYYCARLGMITTGYAMDYWGQGTSVTVSSGG |
| | | GGSGGGGSGGGGSDVLMTQTPLSLPVSLGDQASISCRSSQTIVHSTGHTYLEWFLQKPGQS |
| | | PKLLIYKVSNRFSGVPDRFSGSGSGTDFTLKISRVEAEDLGVYYCFQGSHVPYTFGGGTKLEIK |
| 98 | Signal Peptide | MGWSCIILFLVATATGVHS |
| 99 | Human CD8α stalk | AKPTTTPAPRPPTPAPTIASQPLSLRPEACRPAAGGAVHTRGLDFACD |
| 100 | Human CD8α TMD | IYIWAPLAGTCGVLLLSLVIT |
| 101 | Human CD137 ICD | KRGRKKLLYIFKQPFMRPVQTTQEEDGCSCRFPEEEEGGCEL |
| 102 | Human CD33 ICD | RVKFSRSADAPAYQQGQNQLYNELNLGRREEYDVLDKRRGRDPEMGGKPQRRKNPQEGLY |
| | | NELQKDKMAEAYSEIGMKGERRRGKGHDGLYQGLSTATKDTYDALHMQALPPR |
| 103 | eGFP | VSKGEELFTGVVPILVELDGDVNGHKFSVSGEGEGDATYGKLTLKFICTTGKLPVPWPTLVTTL |
| | | TYGVQCFSRYPDHMKQHDFFKSAMPEGYVQERTIFFKDDGNYKTRAEVKFEGDTLVNRIELK |
| | | GIDFKEDGNILGHKLEYNYNSHNVYIMADKQKNGIKVNFKIRHNIEDGSVQLADHYQQNTPIGD |
| | | GPVLLPDNHYLSTQSALSKDPNEKRDHMVLLEFVTAAGITLGMDELYK |
| 104 | T2A cleavage site | GSGEGRGSLLTCGDVEENPGP |
| 105 | P329G-CAR (with SP, | MGWSCIILFLVATATGVHSEVQLVESGGGLVQPGGSLRLSCAASGFTFSRYWMNWVRQAPG |
| | T2A, eGFP) | KGLEWVGEITPDSSTINYAPSLKGRFTISRDNAKNSLYLQMNSLRAEDTAVYYCARPYDYGAW |
| | [SP]-[VH3VL1 scFv]- | FASWGQGTLVTVSSGGGGSGGGGSGGGGSQAVVTQEPSLTVSPGGTVTLTCRSSTGAVTT |
| | [CD8a stalk]-[CD8a | SNYANWVQEKPDHLFTGLIGGTNKRAPGTPARFSGSLLGGKAALTLSGAQPEDEAEYYCALW |
| | TMD]-[CD137 ICD]- | YSNHWVFGGGTKLTVLGGGGSAKPTTTPAPRPPTPAPTIASQPLSLRPEACRPAAGGAVHTR |
| | [CD3ζ ICD]-[T2A]-[eGFP] | GLDFACDIYIWAPLAGTCGVLLLSLVITKRGRKKLLYIFKQPFMRPVQTTQEEDGCSCRFPEEE |
| | | EGGCELRVKFSRSADAPAYQQGQNQLYNELNLGRREEYDVLDKRRGRDPEMGGKPQRRKN |
| | | PQEGLYNELQKDKMAEAYSEIGMKGERRRGKGHDGLYQGLSTATKDTYDALHMQALPPRGS |
| | | GEGRGSLLTCGDVEENPGPVSKGEELFTGVVPILVELDGDVNGHKFSVSGEGEGDATYGKLT |
| | | LKFICTTGKLPVPWPTLVTTLTYGVQCFSRYPDHMKQHDFFKSAMPEGYVQERTIFFKDDGNY |
| | | KTRAEVKFEGDTLVNRIELKGIDFKEDGNILGHKLEYNYNSHNVYIMADKQKNGIKVNFKIRHNI |
| | | EDGSVQLADHYQQNTPIGDGPVLLPDNHYLSTQSALSKDPNEKRDHMVLLEFVTAAGITLGM |
| | | DELYK |
| 106 | P329G-CAR (with T2A, | EVQLVESGGGLVQPGGSLRLSCAASGFTFSRYWMNWVRQAPGKGLEWVGEITPDSSTINYA |
| | eGFP) | PSLKGRFTISRDNAKNSLYLQMNSLRAEDTAVYYCARPYDYGAWFASWGQGTLVTVSSGGG |
| | [VH3VL1 scFv]-[CD8a | GSGGGGGGGGSQAVVTQEPSLTVSPGGTVTLTCRSSTGAVTTSNYANWVQEKPDHLFTGL |
| | stalk]-[CD8a TMD]- | IGGTNKRAPGTPARFSGSLLGGKAALTLSGAQPEDEAEYYCALWYSNHWVFGGGTKLTVLG |
| | [CD137 ICD]-[CD3ζ ICD]- | GGGSAKPTTTPAPRPPTPAPTIASQPLSLRPEACRPAAGGAVHTRGLDFACDIYIWAPLAGTC |
| | [T2A]-[eGFP] | GVLLLSLVITKRGRKKLLYIFKQPFMRPVQTTQEEDGCSCRFPEEEEGGCELRVKFSRSADAP |
| | | AYQQGQNQLYNELNLGRREEYDVLDKRRGRDPEMGGKPQRRKNPQEGLYNELQKDKMAEA |
| | | YSEIGMKGERRRGKGHDGLYQGLSTATKDTYDALHMQALPPRGSGEGRGSLLTCGDVEENP |
| | | GPVSKGEELFTGVVPILVELDGDVNGHKFSVSGEGEGDATYGKLTLKFICTTGKLPVPWPTLV |
| | | TTLTYGVQCFSRYPDHMKQHDFFKSAMPEGYVQERTIFFKDDGNYKTRAEVKFEGDTLVNRI |
| | | ELKGIDFKEDGNILGHKLEYNYNSHNVYIMADKQKNGIKVNFKIRHNIEDGSVQLADHYQQNTP |
| | | IGDGPVLLPDNHYLSTQSALSKDPNEKRDHMVLLEFVTAAGITLGMDELYK |
| 107 | P329G-CAR (with SP) | MGWSCIILFLVATATGVHSEVQLVESGGGLVQPGGSLRLSCAASGFTFSRYWMNWVRQAPG |
| | [SP]-[VH3VL1 scFv]- | KGLEWVGEITPDSSTINYAPSLKGRFTISRDNAKNSLYLQMNSLRAEDTAVYYCARPYDYGAW |
| | [CD8a stalk]-[CD8a | FASWGQGTLVTVSSGGGGSGGGGSGGGGSQAVVTQEPSLTVSPGGTVTLTCRSSTGAVTT |
| | TMD]-[CD137 ICD]- | SNYANWVQEKPDHLFTGLIGGTNKRAPGTPARFSGSLLGGKAALTLSGAQPEDEAEYYCALW |
| | [CD3ζ ICD] | YSNHWVFGGGTKLTVLGGGGSAKPTTTPAPRPPTPAPTIASQPLSLRPEACRPAAGGAVHTR |
| | | GLDFACDIYIWAPLAGTCGVLLLSLVITKRGRKKLLYIFKQPFMRPVQTTQEEDGCSCRFPEEE |
| | | EGGCELRVKFSRSADAPAYQQGQNQLYNELNLGRREEYDVLDKRRGRDPEMGGKPQRRKN |
| | | PQEGLYNELQKDKMAEAYSEIGMKGERRRGKGHDGLYQGLSTATKDTYDALHMQALPPR |
| 108 | P329G-CAR (mature) | EVQLVESGGGLVQPGGSLRLSCAASGFTFSRYWMNWVRQAPGKGLEWVGEITPDSSTINYA |
| | [VH3VL1 scFv]-[CD8a | PSLKGRFTISRDNAKNSLYLQMNSLRAEDTAVYYCARPYDYGAWFASWGQGTLVTVSSGGG |
| | stalk]-[CD8a TMD]- | GSGGGGSGGGGSQAVVTQEPSLTVSPGGTVTLTCRSSTGAVTTSNYANWVQEKPDHLFTGL |
| | [CD137 ICD]-[CD3ζ ICD] | IGGTNKRAPGTPARFSGSLLGGKAALTLSGAQPEDEAEYYCALWYSNHWVFGGGTKLTVLG |
| | | GGGSAKPTTTPAPRPPTPAPTIASQPLSLRPEACRPAAGGAVHTRGLDFACDIYIWAPLAGTC |
| | | GVLLLSLVITKRGRKKLLYIFKQPFMRPVQTTQEEDGCSCRFPEEEEGGCELRVKFSRSADAP |
| | | AYQQGQNQLYNELNLGRREEYDVLDKRRGRDPEMGGKPQRRKNPQEGLYNELQKDKMAEA |
| | | YSEIGMKGERRRGKGHDGLYQGLSTATKDTYDALHMQALPPR |
| 109 | E2A linker | GSGQCTNYALLKLAGDVESNPGP |
| 110 | P329G VH1VL1 | MGWSCIILFLVATATGVHSEVQLVESGGGLVQPGGSLRLSCAASGFDFSRYWMNWVRQAPG |
| | CD3ϵ(with SP, T2A, | KGLEWVGEITPDSSTINYTPSLKGRFTISRDNAKNSLYLQMNSLRAEDTAVYYCVRPYDYGAW |
| | eGFP) | FASWGQGTLVTVSSGGGGSGGGGSGGGGSQAVVTQEPSLTVSPGGTVTLTCRSSTGAVTT |
| | [SP]-[VH1VL1 scFv]- | SNYANWVQEKPDHLFTGLIGGTNKRAPGTPARFSGSLLGGKAALTLSGAQPEDEAEYYCALW |
| | [CD3ϵ ECD]-[CD3ϵ | YSNHWVFGGGTKLTVLGGGGSGGGGSGGGGSDGNEEMGGITQTPYKVSISGTTVILTCPQY |
| | TMD]-[CD3ϵ ICD]-[T2A]- | PGSEILWQHNDKNIGGDEDDKNIGSDEDHLSLKEFSELEQSGYYVCYPRGSKPEDANFYLYL |
| | [eGFP] | RARVCENCMEMDVMSVATIVIVDICITGGLLLLVYYWSKNRKAKAKPVTRGAGAGGRQRGQN |
| | | KERPPPVPNPDYEPIRKGQRDLYSGLNQRRIGSGEGRGSLLTCGDVEENPGPVSKGEELFTG |
| | | VVPILVELDGDVNGHKFSVSGEGEGDATYGKLTLKFICTTGKLPVPWPTLVTTLTYGVQCFSR |
| | | YPDHMKQHDFFKSAMPEGYVQERTIFFKDDGNYKTRAEVKFEGDTLVNRIELKGIDFKEDGNI |
| | | LGHKLEYNYNSHNVYIMADKQKNGIKVNFKIRHNIEDGSVQLADHYQQNTPIGDGPVLLPDNH |
| | | YLSTQSALSKDPNEKRDHMVLLEFVTAAGITLGMDELYK |
| 111 | P329G VH1VL1 | EVQLVESGGGLVQPGGSLRLSCAASGFDFSRYWMNWVRQAPGKGLEWVGEITPDSSTINYT |
| | CD3ϵ(with T2A, eGFP) | PSLKGRFTISRDNAKNSLYLQMNSLRAEDTAVYYCVRPYDYGAWFASWGQGTLVTVSSGGG |
| | [VH1VL1 scFv]-[CD3ϵ | GSGGGGSGGGGSQAVVTQEPSLTVSPGGTVTLTCRSSTGAVTTSNYANWVQEKPDHLFTGL |
| | ECD]-[CD3ϵ TMD]-[CD3ϵ | IGGTNKRAPGTPARFSGSLLGGKAALTLSGAQPEDEAEYYCALWYSNHWVFGGGTKLTVLG |
| | ICD]-[T2A]-[eGFP] | GGGSGGGGSGGGGSDGNEEMGGITQTPYKVSISGTTVILTCPQYPGSEILWQHNDKNIGGD |
| | | EDDKNIGSDEDHLSLKEFSELEQSGYYVCYPRGSKPEDANFYLYLRARVCENCMEMDVMSV |
| | | ATIVIVDICITGGLLLLVYYWSKNRKAKAKPVTRGAGAGGRQRGQNKERPPPVPNPDYEPIRK |
| | | GQRDLYSGLNQRRIGSGEGRGSLLTCGDVEENPGPVSKGEELFTGWPILVELDGDVNGHKF |
| | | SVSGEGEGDATYGKLTLKFICTTGKLPVPWPTLVTTLTYGVQCFSRYPDHMKQHDFFKSAMP |
| | | EGYVQERTIFFKDDGNYKTRAEVKFEGDTLVNRIELKGIDFKEDGNILGHKLEYNYNSHNVYIM |
| | | ADKQKNGIKVNFKIRHNIEDGSVQLADHYQQNTPIGDGPVLLPDNHYLSTQSALSKDPNEKRD |
| | | HMVLLEFVTAAGITLGMDELYK |
| 112 | P329G VH1VL1 | MGWSCIILFLVATATGVHSEVQLVESGGGLVQPGGSLRLSCAASGFDFSRYWMNWVRQAPG |
| | CD3ϵ(with SP) | KGLEWVGEITPDSSTINYTPSLKGRFTISRDNAKNSLYLQMNSLRAEDTAVYYCVRPYDYGAW |
| | [SP]-[VH1VL1 scFv]- | FASWGQGTLVTVSSGGGGSGGGGSGGGGSQAVVTQEPSLTVSPGGTVTLTCRSSTGAVTT |
| | [CD3ϵ ECD]-[CD3ϵ | SNYANWVQEKPDHLFTGLIGGTNKRAPGTPARFSGSLLGGKAALTLSGAQPEDEAEYYCALW |
| | TMD]-[CD3ϵ ICD] | YSNHWVFGGGTKLTVLGGGGSGGGGSGGGGSDGNEEMGGITQTPYKVSISGTTVILTCPQY |
| | | PGSEILWQHNDKNIGGDEDDKNIGSDEDHLSLKEFSELEQSGYYVCYPRGSKPEDANFYLYL |
| | | RARVCENCMEMDVMSVATIVIVDICITGGLLLLVYYWSKNRKAKAKPVTRGAGAGGRQRGQN |
| | | KERPPPVPNPDYEPIRKGQRDLYSGLNQRRI |
| 113 | P329G VH1VL1 CD38 | EVQLVESGGGLVQPGGSLRLSCAASGFDFSRYWMNWVRQAPGKGLEWVGEITPDSSTINYT |
| | (mature) | PSLKGRFTISRDNAKNSLYLQMNSLRAEDTAVYYCVRPYDYGAWFASWGQGTLVTVSSGGG |
| | [VH1VL1 scFv]-[CD3ϵ | GSGGGGSGGGGSQAVVTQEPSLTVSPGGTVTLTCRSSTGAVTTSNYANWVQEKPDHLFTGL |
| | ECD]-[CD3ϵ TMD]-[CD3ϵ | IGGTNKRAPGTPARFSGSLLGGKAALTLSGAQPEDEAEYYCALWYSNHWVFGGGTKLTVLG |
| | ICD] | GGGSGGGGSGGGGSDGNEEMGGITQTPYKVSISGTTVILTCPQYPGSEILWQHNDKNIGGD |
| | | EDDKNIGSDEDHLSLKEFSELEQSGYYVCYPRGSKPEDANFYLYLRARVCENCMEMDVMSV |
| | | ATIVIVDICITGGLLLLVYYWSKNRKAKAKPVTRGAGAGGRQRGQNKERPPPVPNPDYEPIRK |
| | | GQRDLYSGLNQRRI |
| 114 | P329G VH2VL1 | MGWSCIILFLVATATGVHSEVQLVESGGGLVQPGGSLRLSCAASGFDFSRYWMNWVRQAPG |
| | CD3ϵ(with SP, T2A, | KGLEWVGEITPDSSTINYAPSLKGRFTISRDNAKNSLYLQMNSLRAEDTAVYYCVRPYDYGAW |
| | eGFP) | FASWGQGTLVTVSSGGGGSGGGGSGGGGSQAVVTQEPSLTVSPGGTVTLTCRSSTGAVTT |
| | [SP]-[VH2VL1 scFv]- | SNYANWVQEKPDHLFTGLIGGTNKRAPGTPARFSGSLLGGKAALTLSGAQPEDEAEYYCALW |
| | [CD3ϵ ECD]-[CD3ϵ | YSNHWVFGGGTKLTVLGGGGSGGGGSGGGGSDGNEEMGGITQTPYKVSISGTTVILTCPQY |
| | TMD]-[CD3ϵ ICD]-[T2A]- | PGSEILWQHNDKNIGGDEDDKNIGSDEDHLSLKEFSELEQSGYYVCYPRGSKPEDANFYLYL |
| | [eGFP] | RARVCENCMEMDVMSVATIVIVDICITGGLLLLVYYWSKNRKAKAKPVTRGAGAGGRQRGQN |
| | | KERPPPVPNPDYEPIRKGQRDLYSGLNQRRIGSGEGRGSLLTCGDVEENPGPVSKGEELFTG |
| | | VVPILVELDGDVNGHKFSVSGEGEGDATYGKLTLKFICTTGKLPVPWPTLVTTLTYGVQCFSR |
| | | YPDHMKQHDFFKSAMPEGYVQERTIFFKDDGNYKTRAEVKFEGDTLVNRIELKGIDFKEDGNI |
| | | LGHKLEYNYNSHNVYIMADKQKNGIKVNFKIRHNIEDGSVQLADHYQQNTPIGDGPVLLPDNH |
| | | YLSTQSALSKDPNEKRDHMVLLEFVTAAGITLGMDELYK |
| 115 | P329G VH2VL1 | EVQLVESGGGLVQPGGSLRLSCAASGFDFSRYWMNWVRQAPGKGLEWVGEITPDSSTINYA |
| | CD3ϵ(with T2A, eGFP) | PSLKGRFTISRDNAKNSLYLQMNSLRAEDTAVYYCVRPYDYGAWFASWGQGTLVTVSSGGG |
| | [VH2VL1 scFv]-[CD3ϵ | GSGGGGSGGGGSQAVVTQEPSLTVSPGGTVTLTCRSSTGAVTTSNYANWVQEKPDHLFTGL |
| | ECD]-[CD3ϵ TMD]-[CD3ϵ | IGGTNKRAPGTPARFSGSLLGGKAALTLSGAQPEDEAEYYCALWYSNHWVFGGGTKLTVLG |
| | ICD]-[T2A]-[eGFP] | GGGSGGGGSGGGGSDGNEEMGGITQTPYKVSISGTTVILTCPQYPGSEILWQHNDKNIGGD |
| | | EDDKNIGSDEDHLSLKEFSELEQSGYYVCYPRGSKPEDANFYLYLRARVCENCMEMDVMSV |
| | | ATIVIVDICITGGLLLLVYYWSKNRKAKAKPVTRGAGAGGRQRGQNKERPPPVPNPDYEPIRK |
| | | GQRDLYSGLNQRRIGSGEGRGSLLTCGDVEENPGPVSKGEELFTGVVPILVELDGDVNGHKF |
| | | SVSGEGEGDATYGKLTLKFICTTGKLPVPWPTLVTTLTYGVQCFSRYPDHMKQHDFFKSAMP |
| | | EGYVQERTIFFKDDGNYKTRAEVKFEGDTLVNRIELKGIDFKEDGNILGHKLEYNYNSHNVYIM |
| | | ADKQKNGIKVNFKIRHNIEDGSVQLADHYQQNTPIGDGPVLLPDNHYLSTQSALSKDPNEKRD |
| | | HMVLLEFVTAAGITLGMDELYK |
| 116 | P329G VH2VL1 | MGWSCIILFLVATATGVHSEVQLVESGGGLVQPGGSLRLSCAASGFDFSRYWMNWVRQAPG |
| | CD3ϵ(with SP) | KGLEWVGEITPDSSTINYAPSLKGRFTISRDNAKNSLYLQMNSLRAEDTAVYYCVRPYDYGAW |
| | [SP]-[VH2VL1 scFv]- | FASWGQGTLVTVSSGGGGSGGGGSGGGGSQAVVTQEPSLTVSPGGTVTLTCRSSTGAVTT |
| | [CD3ϵ ECD]-[CD3ϵ | SNYANWVQEKPDHLFTGLIGGTNKRAPGTPARFSGSLLGGKAALTLSGAQPEDEAEYYCALW |
| | TMD]-[CD3ϵ ICD] | YSNHWVFGGGTKLTVLGGGGSGGGGSGGGGSDGNEEMGGITQTPYKVSISGTTVILTCPQY |
| | | PGSEILWQHNDKNIGGDEDDKNIGSDEDHLSLKEFSELEQSGYYVCYPRGSKPEDANFYLYL |
| | | RARVCENCMEMDVMSVATIVIVDICITGGLLLLVYYWSKNRKAKAKPVTRGAGAGGRQRGQN |
| | | KERPPPVPNPDYEPIRKGQRDLYSGLNQRRI |
| 117 | P329G VH2VL1 CD38 | EVQLVESGGGLVQPGGSLRLSCAASGFDFSRYWMNWVRQAPGKGLEWVGEITPDSSTINYA |
| | (mature) | PSLKGRFTISRDNAKNSLYLQMNSLRAEDTAVYYCVRPYDYGAWFASWGQGTLVTVSSGGG |
| | [VH2VL1 scFv]-[CD3ϵ | GSGGGGSGGGGSQAVVTQEPSLTVSPGGTVTLTCRSSTGAVTTSNYANWVQEKPDHLFTGL |
| | ECD]-[CD3ϵ TMD]-[CD3ϵ | IGGTNKRAPGTPARFSGSLLGGKAALTLSGAQPEDEAEYYCALWYSNHWVFGGGTKLTVLG |
| | ICD] | GGGSGGGGSGGGGSDGNEEMGGITQTPYKVSISGTTVILTCPQYPGSEILWQHNDKNIGGD |
| | | EDDKNIGSDEDHLSLKEFSELEQSGYYVCYPRGSKPEDANFYLYLRARVCENCMEMDVMSV |
| | | ATIVIVDICITGGLLLLVYYWSKNRKAKAKPVTRGAGAGGRQRGQNKERPPPVPNPDYEPIRK |
| | | GQRDLYSGLNQRRI |
| 118 | P329G VH3VL1 | MGWSCIILFLVATATGVHSEVQLVESGGGLVQPGGSLRLSCAASGFTFSRYWMNWVRQAPG |
| | CD3ϵ(with SP, T2A, | KGLEWVGEITPDSSTINYAPSLKGRFTISRDNAKNSLYLQMNSLRAEDTAVYYCARPYDYGAW |
| | eGFP) | FASWGQGTLVTVSSGGGGSGGGGSGGGGSQAVVTQEPSLTVSPGGTVTLTCRSSTGAVTT |
| | [SP]-[VH3VL1 scFv]- | SNYANWVQEKPDHLFTGLIGGTNKRAPGTPARFSGSLLGGKAALTLSGAQPEDEAEYYCALW |
| | [CD3ϵ ECD]-[CD3ϵ | YSNHWVFGGGTKLTVLGGGGSGGGGSGGGGSDGNEEMGGITQTPYKVSISGTTVILTCPQY |
| | TMD]-[CD3ϵ ICD]-[T2A]- | PGSEILWQHNDKNIGGDEDDKNIGSDEDHLSLKEFSELEQSGYYVCYPRGSKPEDANFYLYL |
| | [eGFP] | RARVCENCMEMDVMSVATIVIVDICITGGLLLLVYYWSKNRKAKAKPVTRGAGAGGRQRGQN |
| | | KERPPPVPNPDYEPIRKGQRDLYSGLNQRRIGSGEGRGSLLTCGDVEENPGPVSKGEELFTG |
| | | VVPILVELDGDVNGHKFSVSGEGEGDATYGKLTLKFICTTGKLPVPWPTLVTTLTYGVQCFSR |
| | | YPDHMKQHDFFKSAMPEGYVQERTIFFKDDGNYKTRAEVKFEGDTLVNRIELKGIDFKEDGNI |
| | | LGHKLEYNYNSHNVYIMADKQKNGIKVNFKIRHNIEDGSVQLADHYQQNTPIGDGPVLLPDNH |
| | | YLSTQSALSKDPNEKRDHMVLLEFVTAAGITLGMDELYK |
| 119 | P329G VH3VL1 | EVQLVESGGGLVQPGGSLRLSCAASGFTFSRYWMNWVRQAPGKGLEWVGEITPDSSTINYA |
| | CD3€(with T2A, eGFP) | PSLKGRFTISRDNAKNSLYLQMNSLRAEDTAVYYCARPYDYGAWFASWGQGTLVTVSSGGG |
| | [VH3VL1 scFv]-[CD3ϵ | GSGGGGSGGGGSQAVVTQEPSLTVSPGGTVTLTCRSSTGAVTTSNYANWVQEKPDHLFTGL |
| | ECD]-[CD3ϵ TMD]-[CD3ϵ | IGGTNKRAPGTPARFSGSLLGGKAALTLSGAQPEDEAEYYCALWYSNHWVFGGGTKLTVLG |
| | ICD]-[T2A]-[eGFP] | GGGSGGGGSGGGGSDGNEEMGGITQTPYKVSISGTTVILTCPQYPGSEILWQHNDKNIGGD |
| | | EDDKNIGSDEDHLSLKEFSELEQSGYYVCYPRGSKPEDANFYLYLRARVCENCMEMDVMSV |
| | | ATIVIVDICITGGLLLLVYYWSKNRKAKAKPVTRGAGAGGRQRGQNKERPPPVPNPDYEPIRK |
| | | GQRDLYSGLNQRRIGSGEGRGSLLTCGDVEENPGPVSKGEELFTGVVPILVELDGDVNGHKF |
| | | SVSGEGEGDATYGKLTLKFICTTGKLPVPWPTLVTTLTYGVQCFSRYPDHMKQHDFFKSAMP |
| | | EGYVQERTIFFKDDGNYKTRAEVKFEGDTLVNRIELKGIDFKEDGNILGHKLEYNYNSHNVYIM |
| | | ADKQKNGIKVNFKIRHNIEDGSVQLADHYQQNTPIGDGPVLLPDNHYLSTQSALSKDPNEKRD |
| | | HMVLLEFVTAAGITLGMDELYK |
| 120 | P329G VH3VL1 | MGWSCIILFLVATATGVHSEVQLVESGGGLVQPGGSLRLSCAASGFTFSRYWMNWVRQAPG |
| | CD3ϵ(with SP) | KGLEWVGEITPDSSTINYAPSLKGRFTISRDNAKNSLYLQMNSLRAEDTAVYYCARPYDYGAW |
| | [SP]-[VH3VL1 scFv]- | FASWGQGTLVTVSSGGGGSGGGGSGGGGSQAVVTQEPSLTVSPGGTVTLTCRSSTGAVTT |
| | [CD3ϵ ECD]-[CD3ϵ | SNYANWVQEKPDHLFTGLIGGTNKRAPGTPARFSGSLLGGKAALTLSGAQPEDEAEYYCALW |
| | TMD]-[CD3ϵ ICD] | YSNHWVFGGGTKLTVLGGGGSGGGGSGGGGSDGNEEMGGITQTPYKVSISGTTVILTCPQY |
| | | PGSEILWQHNDKNIGGDEDDKNIGSDEDHLSLKEFSELEQSGYYVCYPRGSKPEDANFYLYL |
| | | RARVCENCMEMDVMSVATIVIVDICITGGLLLLVYYWSKNRKAKAKPVTRGAGAGGRQRGQN |
| | | KERPPPVPNPDYEPIRKGQRDLYSGLNQRRI |
| 121 | P329G VH3VL1 CD38 | EVQLVESGGGLVQPGGSLRLSCAASGFTFSRYWMNWVRQAPGKGLEWVGEITPDSSTINYA |
| | (mature) | PSLKGRFTISRDNAKNSLYLQMNSLRAEDTAVYYCARPYDYGAWFASWGQGTLVTVSSGGG |
| | [VH3VL1 scFv]-[CD3ϵ | GSGGGGSGGGGSQAVVTQEPSLTVSPGGTVTLTCRSSTGAVTTSNYANWVQEKPDHLFTGL |
| | ECD]-[CD3ϵ TMD]-[CD3ϵ | IGGTNKRAPGTPARFSGSLLGGKAALTLSGAQPEDEAEYYCALWYSNHWVFGGGTKLTVLG |
| | ICD] | GGGSGGGGSGGGGSDGNEEMGGITQTPYKVSISGTTVILTCPQYPGSEILWQHNDKNIGGD |
| | | EDDKNIGSDEDHLSLKEFSELEQSGYYVCYPRGSKPEDANFYLYLRARVCENCMEMDVMSV |
| | | ATIVIVDICITGGLLLLVYYWSKNRKAKAKPVTRGAGAGGRQRGQNKERPPPVPNPDYEPIRK |
| | | GQRDLYSGLNQRRI |
| 122 | AAA CD38 (with SP, | MGWSCIILFLVATATGVHSEVQLVESGGDLVKPGGSLKLSCAASGFTFSSYGMSWVRQTPDK |
| | T2A, eGFP) | RLEWVATISSGGSYIYYPDSVKGRFTISRDNAKNTLYLQMSSLKSEDTAMYYCARLGMITTGY |
| | [SP]-[αAAA scFv]-[CD3ϵ | AMDYWGQGTSVTVSSGGGGSGGGGSGGGGSDVLMTQTPLSLPVSLGDQASISCRSSQTIV |
| | ECD]-[CD3ϵ TMD]-[CD3ϵ | HSTGHTYLEWFLQKPGQSPKLLIYKVSNRFSGVPDRFSGSGSGTDFTLKISRVEAEDLGVYYC |
| | ICD]-[T2A]-[eGFP] | FQGSHVPYTFGGGTKLEIKGGGGSGGGGSGGGGSDGNEEMGGITQTPYKVSISGTTVILTCP |
| | | QYPGSEILWQHNDKNIGGDEDDKNIGSDEDHLSLKEFSELEQSGYYVCYPRGSKPEDANFYL |
| | | YLRARVCENCMEMDVMSVATIVIVDICITGGLLLLVYYWSKNRKAKAKPVTRGAGAGGRQRG |
| | | QNKERPPPVPNPDYEPIRKGQRDLYSGLNQRRIGSGEGRGSLLTCGDVEENPGPVSKGEELF |
| | | TGVVPILVELDGDVNGHKFSVSGEGEGDATYGKLTLKFICTTGKLPVPWPTLVTTLTYGVQCF |
| | | SRYPDHMKQHDFFKSAMPEGYVQERTIFFKDDGNYKTRAEVKFEGDTLVNRIELKGIDFKEDG |
| | | NILGHKLEYNYNSHNVYIMADKQKNGIKVNFKIRHNIEDGSVQLADHYQQNTPIGDGPVLLPDN |
| | | HYLSTQSALSKDPNEKRDHMVLLEFVTAAGITLGMDELYK |
| 123 | AAA CD38 (with T2A, | EVQLVESGGDLVKPGGSLKLSCAASGFTFSSYGMSWVRQTPDKRLEWVATISSGGSYIYYPD |
| | eGFP) | SVKGRFTISRDNAKNTLYLQMSSLKSEDTAMYYCARLGMITTGYAMDYWGQGTSVTVSSGG |
| | [αAAA scFv]-[CD3ϵ | GGSGGGGSGGGGSDVLMTQTPLSLPVSLGDQASISCRSSQTIVHSTGHTYLEWFLQKPGQS |
| | ECD]-[CD3ϵ TMD]-[CD3ϵ | PKLLIYKVSNRFSGVPDRFSGSGSGTDFTLKISRVEAEDLGVYYCFQGSHVPYTFGGGTKLEIK |
| | ICD]-[T2A]-[eGFP] | GGGGSGGGGSGGGGSDGNEEMGGITQTPYKVSISGTTVILTCPQYPGSEILWQHNDKNIGG |
| | | DEDDKNIGSDEDHLSLKEFSELEQSGYYVCYPRGSKPEDANFYLYLRARVCENCMEMDVMS |
| | | VATIVIVDICITGGLLLLVYYWSKNRKAKAKPVTRGAGAGGRQRGQNKERPPPVPNPDYEPIRK |
| | | GQRDLYSGLNQRRIGSGEGRGSLLTCGDVEENPGPVSKGEELFTGVVPILVELDGDVNGHKF |
| | | SVSGEGEGDATYGKLTLKFICTTGKLPVPWPTLVTTLTYGVQCFSRYPDHMKQHDFFKSAMP |
| | | EGYVQERTIFFKDDGNYKTRAEVKFEGDTLVNRIELKGIDFKEDGNILGHKLEYNYNSHNVYIM |
| | | ADKQKNGIKVNFKIRHNIEDGSVQLADHYQQNTPIGDGPVLLPDNHYLSTQSALSKDPNEKRD |
| | | HMVLLEFVTAAGITLGMDELYK |
| 124 | AAA CD3ϵ (with SP) | MGWSCIILFLVATATGVHSEVQLVESGGDLVKPGGSLKLSCAASGFTFSSYGMSWVRQTPDK |
| | [SP]-[αAAA scFv]-[CD3ϵ | RLEWVATISSGGSYIYYPDSVKGRFTISRDNAKNTLYLQMSSLKSEDTAMYYCARLGMITTGY |
| | ECD]-[CD3ϵ TMD]-[CD3ϵ | AMDYWGQGTSVTVSSGGGGSGGGGSGGGGSDVLMTQTPLSLPVSLGDQASISCRSSQTIV |
| | ICD] | HSTGHTYLEWFLQKPGQSPKLLIYKVSNRFSGVPDRFSGSGSGTDFTLKISRVEAEDLGVYYC |
| | | FQGSHVPYTFGGGTKLEIKGGGGSGGGGSGGGGSDGNEEMGGITQTPYKVSISGTTVILTCP |
| | | QYPGSEILWQHNDKNIGGDEDDKNIGSDEDHLSLKEFSELEQSGYYVCYPRGSKPEDANFYL |
| | | YLRARVCENCMEMDVMSVATIVIVDICITGGLLLLVYYWSKNRKAKAKPVTRGAGAGGRQRG |
| | | QNKERPPPVPNPDYEPIRKGQRDLYSGLNQRRI |
| 125 | AAA CD3ϵ (mature) | EVQLVESGGDLVKPGGSLKLSCAASGFTFSSYGMSWVRQTPDKRLEWVATISSGGSYIYYPD |
| | [αAAA scFv]-[CD3ϵ | SVKGRFTISRDNAKNTLYLQMSSLKSEDTAMYYCARLGMITTGYAMDYWGQGTSVTVSSGG |
| | ECD]-[CD3ϵ TMD]-[CD3ϵ | GGSGGGGSGGGGSDVLMTQTPLSLPVSLGDQASISCRSSQTIVHSTGHTYLEWFLQKPGQS |
| | ICD] | PKLLIYKVSNRFSGVPDRFSGSGSGTDFTLKISRVEAEDLGVYYCFQGSHVPYTFGGGTKLEIK |
| | | GGGGSGGGGSGGGGSDGNEEMGGITQTPYKVSISGTTVILTCPQYPGSEILWQHNDKNIGG |
| | | DEDDKNIGSDEDHLSLKEFSELEQSGYYVCYPRGSKPEDANFYLYLRARVCENCMEMDVMS |
| | | VATIVIVDICITGGLLLLVYYWSKNRKAKAKPVTRGAGAGGRQRGQNKERPPPVPNPDYEPIRK |
| | | GQRDLYSGLNQRRI |
| 126 | P329G TCRαβ- | MGWSCIILFLVATATGVHSEVQLVESGGGLVQPGGSLRLSCAASGFDFSRYWMNWVRQAPG |
| | VH1TRAC (with SP, T2A, | KGLEWVGEITPDSSTINYTPSLKGRFTISRDNAKNSLYLQMNSLRAEDTAVYYCVRPYDYGAW |
| | eGFP) | FASWGQGTLVTVSSIQNPDPAVYQLRDSKSSDKSVCLFTDFDSQTNVSQSKDSDVYITDKCV |
| | [SP]-[VH1]- | LDMRSMDFKSNSAVAWSNKSDFACANAFNNSIIPEDTFFPSPESSCDVKLVEKSFETDTNLNF |
| | [TRAC_T47C]-[T2A]- | QNLSVIGFRILLLKVAGFNLLMTLRLWSSGSGEGRGSLLTCGDVEENPGPVSKGEELFTGVVPI |
| | [eGFP] | LVELDGDVNGHKFSVSGEGEGDATYGKLTLKFICTTGKLPVPWPTLVTTLTYGVQCFSRYPDH |
| | | MKQHDFFKSAMPEGYVQERTIFFKDDGNYKTRAEVKFEGDTLVNRIELKGIDFKEDGNILGHK |
| | | LEYNYNSHNVYIMADKQKNGIKVNFKIRHNIEDGSVQLADHYQQNTPIGDGPVLLPDNHYLST |
| | | QSALSKDPNEKRDHMVLLEFVTAAGITLGMDELYK |
| 127 | P329G TCRαβ- | EVQLVESGGGLVQPGGSLRLSCAASGFDFSRYWMNWVRQAPGKGLEWVGEITPDSSTINYT |
| | VH1TRAC (with T2A, | PSLKGRFTISRDNAKNSLYLQMNSLRAEDTAVYYCVRPYDYGAWFASWGQGTLVTVSSIQNP |
| | eGFP) | DPAVYQLRDSKSSDKSVCLFTDFDSQTNVSQSKDSDVYITDKCVLDMRSMDFKSNSAVAWS |
| | [VH1]-[TRAC_T47C]- | NKSDFACANAFNNSIIPEDTFFPSPESSCDVKLVEKSFETDTNLNFQNLSVIGFRILLLKVAGFN |
| | [T2A]-[eGFP] | LLMTLRLWSSGSGEGRGSLLTCGDVEENPGPVSKGEELFTGVVPILVELDGDVNGHKFSVSG |
| | | EGEGDATYGKLTLKFICTTGKLPVPWPTLVTTLTYGVQCFSRYPDHMKQHDFFKSAMPEGYV |
| | | QERTIFFKDDGNYKTRAEVKFEGDTLVNRIELKGIDFKEDGNILGHKLEYNYNSHNVYIMADKQ |
| | | KNGIKVNFKIRHNIEDGSVQLADHYQQNTPIGDGPVLLPDNHYLSTQSALSKDPNEKRDHMVL |
| | | LEFVTAAGITLGMDELYK |
| 128 | P329G TCRαβ- | MGWSCIILFLVATATGVHSEVQLVESGGGLVQPGGSLRLSCAASGFDFSRYWMNWVRQAPG |
| | VH1TRAC (with SP) | KGLEWVGEITPDSSTINYTPSLKGRFTISRDNAKNSLYLQMNSLRAEDTAVYYCVRPYDYGAW |
| | [SP]-[VH1]-[TRAC_T47C] | FASWGQGTLVTVSSIQNPDPAVYQLRDSKSSDKSVCLFTDFDSQTNVSQSKDSDVYITDKCV |
| | | LDMRSMDFKSNSAVAWSNKSDFACANAFNNSIIPEDTFFPSPESSCDVKLVEKSFETDTNLNF |
| | | QNLSVIGFRILLLKVAGFNLLMTLRLWSS |
| 129 | P329G TCRαβ- | EVQLVESGGGLVQPGGSLRLSCAASGFDFSRYWMNWVRQAPGKGLEWVGEITPDSSTINYT |
| | VH1TRAC (mature) | PSLKGRFTISRDNAKNSLYLQMNSLRAEDTAVYYCVRPYDYGAWFASWGQGTLVTVSSIQNP |
| | [VH1]-[TRAC_T47C] | DPAVYQLRDSKSSDKSVCLFTDFDSQTNVSQSKDSDVYITDKCVLDMRSMDFKSNSAVAWS |
| | | NKSDFACANAFNNSIIPEDTFFPSPESSCDVKLVEKSFETDTNLNFQNLSVIGFRILLLKVAGEN |
| | | LLMTLRLWSS |
| 130 | P329G TCRαβ- | MGWSCIILFLVATATGVHSEVQLVESGGGLVQPGGSLRLSCAASGFDFSRYWMNWVRQAPG |
| | VH2TRAC (with SP, T2A, | KGLEWVGEITPDSSTINYAPSLKGRFTISRDNAKNSLYLQMNSLRAEDTAVYYCVRPYDYGAW |
| | eGFP) | FASWGQGTLVTVSSIQNPDPAVYQLRDSKSSDKSVCLFTDFDSQTNVSQSKDSDVYITDKCV |
| | [SP]-[VH2]- | LDMRSMDFKSNSAVAWSNKSDFACANAFNNSIIPEDTFFPSPESSCDVKLVEKSFETDTNLNF |
| | [TRAC_T47C]-[T2A]- | QNLSVIGFRILLLKVAGFNLLMTLRLWSSGSGEGRGSLLTCGDVEENPGPVSKGEELFTGVVPI |
| | [eGFP] | LVELDGDVNGHKFSVSGEGEGDATYGKLTLKFICTTGKLPVPWPTLVTTLTYGVQCFSRYPDH |
| | | MKQHDFFKSAMPEGYVQERTIFFKDDGNYKTRAEVKFEGDTLVNRIELKGIDFKEDGNILGHK |
| | | LEYNYNSHNVYIMADKQKNGIKVNFKIRHNIEDGSVQLADHYQQNTPIGDGPVLLPDNHYLST |
| | | QSALSKDPNEKRDHMVLLEFVTAAGITLGMDELYK |
| 131 | P329G TCRαβ- | EVQLVESGGGLVQPGGSLRLSCAASGFDFSRYWMNWVRQAPGKGLEWVGEITPDSSTINYA |
| | VH2TRAC (with T2A, | PSLKGRFTISRDNAKNSLYLQMNSLRAEDTAVYYCVRPYDYGAWFASWGQGTLVTVSSIQNP |
| | eGFP) | DPAVYQLRDSKSSDKSVCLFTDFDSQTNVSQSKDSDVYITDKCVLDMRSMDFKSNSAVAWS |
| | [VH2]-[TRAC_T47C]- | NKSDFACANAFNNSIIPEDTFFPSPESSCDVKLVEKSFETDTNLNFQNLSVIGFRILLLKVAGEN |
| | [T2A]-[eGFP] | LLMTLRLWSSGSGEGRGSLLTCGDVEENPGPVSKGEELFTGVVPILVELDGDVNGHKFSVSG |
| | | EGEGDATYGKLTLKFICTTGKLPVPWPTLVTTLTYGVQCFSRYPDHMKQHDFFKSAMPEGYV |
| | | QERTIFFKDDGNYKTRAEVKFEGDTLVNRIELKGIDFKEDGNILGHKLEYNYNSHNVYIMADKQ |
| | | KNGIKVNFKIRHNIEDGSVQLADHYQQNTPIGDGPVLLPDNHYLSTQSALSKDPNEKRDHMVL |
| | | LEFVTAAGITLGMDELYK |
| 132 | P329G TCRαβ- | MGWSCIILFLVATATGVHSEVQLVESGGGLVQPGGSLRLSCAASGFDFSRYWMNWVRQAPG |
| | VH2TRAC (with SP) | KGLEWVGEITPDSSTINYAPSLKGRFTISRDNAKNSLYLQMNSLRAEDTAVYYCVRPYDYGAW |
| | [SP]-[VH2]-[TRAC_T47C] | FASWGQGTLVTVSSIQNPDPAVYQLRDSKSSDKSVCLFTDFDSQTNVSQSKDSDVYITDKCV |
| | | LDMRSMDFKSNSAVAWSNKSDFACANAFNNSIIPEDTFFPSPESSCDVKLVEKSFETDTNLNF |
| | | QNLSVIGFRILLLKVAGFNLLMTLRLWSS |
| 133 | P329G TCRαβ- | EVQLVESGGGLVQPGGSLRLSCAASGFDFSRYWMNWVRQAPGKGLEWVGEITPDSSTINYA |
| | VH2TRAC (mature) | PSLKGRFTISRDNAKNSLYLQMNSLRAEDTAVYYCVRPYDYGAWFASWGQGTLVTVSSIQNP |
| | [VH2]-[TRAC_T47C] | DPAVYQLRDSKSSDKSVCLFTDFDSQTNVSQSKDSDVYITDKCVLDMRSMDFKSNSAVAWS |
| | | NKSDFACANAFNNSIIPEDTFFPSPESSCDVKLVEKSFETDTNLNFQNLSVIGFRILLLKVAGEN |
| | | LLMTLRLWSS |
| 134 | P329G TCRαβ- | MGWSCIILFLVATATGVHSEVQLVESGGGLVQPGGSLRLSCAASGFTFSRYWMNWVRQAPG |
| | VH3TRAC (with SP, T2A, | KGLEWVGEITPDSSTINYAPSLKGRFTISRDNAKNSLYLQMNSLRAEDTAVYYCARPYDYGAW |
| | eGFP) | FASWGQGTLVTVSSIQNPDPAVYQLRDSKSSDKSVCLFTDFDSQTNVSQSKDSDVYITDKCV |
| | [SP]-[VH3]- | LDMRSMDFKSNSAVAWSNKSDFACANAFNNSIIPEDTFFPSPESSCDVKLVEKSFETDTNLNF |
| | [TRAC_T47C]-[T2A]- | QNLSVIGFRILLLKVAGFNLLMTLRLWSSGSGEGRGSLLTCGDVEENPGPVSKGEELFTGVVPI |
| | [eGFP] | LVELDGDVNGHKFSVSGEGEGDATYGKLTLKFICTTGKLPVPWPTLVTTLTYGVQCFSRYPDH |
| | | MKQHDFFKSAMPEGYVQERTIFFKDDGNYKTRAEVKFEGDTLVNRIELKGIDFKEDGNILGHK |
| | | LEYNYNSHNVYIMADKQKNGIKVNFKIRHNIEDGSVQLADHYQQNTPIGDGPVLLPDNHYLST |
| | | QSALSKDPNEKRDHMVLLEFVTAAGITLGMDELYK |
| 135 | P329G TCRαβ- | EVQLVESGGGLVQPGGSLRLSCAASGFTFSRYWMNWVRQAPGKGLEWVGEITPDSSTINYA |
| | VH3TRAC (with T2A, | PSLKGRFTISRDNAKNSLYLQMNSLRAEDTAVYYCARPYDYGAWFASWGQGTLVTVSSIQNP |
| | eGFP) | DPAVYQLRDSKSSDKSVCLFTDFDSQTNVSQSKDSDVYITDKCVLDMRSMDFKSNSAVAWS |
| | [VH3]-[TRAC_T47C]- | NKSDFACANAFNNSIIPEDTFFPSPESSCDVKLVEKSFETDTNLNFQNLSVIGFRILLLKVAGEN |
| | [T2A]-[eGFP] | LLMTLRLWSSGSGEGRGSLLTCGDVEENPGPVSKGEELFTGVVPILVELDGDVNGHKFSVSG |
| | | EGEGDATYGKLTLKFICTTGKLPVPWPTLVTTLTYGVQCFSRYPDHMKQHDFFKSAMPEGYV |
| | | QERTIFFKDDGNYKTRAEVKFEGDTLVNRIELKGIDFKEDGNILGHKLEYNYNSHNVYIMADKQ |
| | | KNGIKVNFKIRHNIEDGSVQLADHYQQNTPIGDGPVLLPDNHYLSTQSALSKDPNEKRDHMVL |
| | | LEFVTAAGITLGMDELYK |
| 136 | P329G TCRαβ- | MGWSCIILFLVATATGVHSEVQLVESGGGLVQPGGSLRLSCAASGFTFSRYWMNWVRQAPG |
| | VH3TRAC (with SP) | KGLEWVGEITPDSSTINYAPSLKGRFTISRDNAKNSLYLQMNSLRAEDTAVYYCARPYDYGAW |
| | [SP]-[VH3]-[TRAC_T47C] | FASWGQGTLVTVSSIQNPDPAVYQLRDSKSSDKSVCLFTDFDSQTNVSQSKDSDVYITDKCV |
| | | LDMRSMDFKSNSAVAWSNKSDFACANAFNNSIIPEDTFFPSPESSCDVKLVEKSFETDTNLNF |
| | | QNLSVIGFRILLLKVAGFNLLMTLRLWSS |
| 137 | P329G TCRαβ- | EVQLVESGGGLVQPGGSLRLSCAASGFTFSRYWMNWVRQAPGKGLEWVGEITPDSSTINYA |
| | VH3TRAC (mature) | PSLKGRFTISRDNAKNSLYLQMNSLRAEDTAVYYCARPYDYGAWFASWGQGTLVTVSSIQNP |
| | [VH3]-[TRAC_T47C] | DPAVYQLRDSKSSDKSVCLFTDFDSQTNVSQSKDSDVYITDKCVLDMRSMDFKSNSAVAWS |
| | | NKSDFACANAFNNSIIPEDTFFPSPESSCDVKLVEKSFETDTNLNFQNLSVIGFRILLLKVAGEN |
| | | LLMTLRLWSS |
| 138 | P329G TCRαβ- | MGWSCIILFLVATATGVHSQAVVTQEPSLTVSPGGTVTLTCRSSTGAVTTSNYANWVQEKPD |
| | VL1TRAC (with SP, T2A, | HLFTGLIGGTNKRAPGTPARFSGSLLGGKAALTLSGAQPEDEAEYYCALWYSNHWVFGGGTK |
| | eGFP) | LTVLIQNPDPAVYQLRDSKSSDKSVCLFTDFDSQTNVSQSKDSDVYITDKCVLDMRSMDFKSN |
| | [SP]-[VL1]- | SAVAWSNKSDFACANAFNNSIIPEDTFFPSPESSCDVKLVEKSFETDTNLNFQNLSVIGFRILLL |
| | [TRAC_T47C]-[T2A]- | KVAGFNLLMTLRLWSSGSGEGRGSLLTCGDVEENPGPVSKGEELFTGVVPILVELDGDVNGH |
| | [eGFP] | KFSVSGEGEGDATYGKLTLKFICTTGKLPVPWPTLVTTLTYGVQCFSRYPDHMKQHDFFKSA |
| | | MPEGYVQERTIFFKDDGNYKTRAEVKFEGDTLVNRIELKGIDFKEDGNILGHKLEYNYNSHNV |
| | | YIMADKQKNGIKVNFKIRHNIEDGSVQLADHYQQNTPIGDGPVLLPDNHYLSTQSALSKDPNEK |
| | | RDHMVLLEFVTAAGITLGMDELYK |
| 139 | P329G TCRαβ- | QAVVTQEPSLTVSPGGTVTLTCRSSTGAVTTSNYANWVQEKPDHLFTGLIGGTNKRAPGTPA |
| | VL1TRAC (with T2A, | RFSGSLLGGKAALTLSGAQPEDEAEYYCALWYSNHWVFGGGTKLTVLIQNPDPAVYQLRDSK |
| | eGFP) | SSDKSVCLFTDFDSQTNVSQSKDSDVYITDKCVLDMRSMDFKSNSAVAWSNKSDFACANAF |
| | [VL1]-[TRAC_T47C]- | NNSIIPEDTFFPSPESSCDVKLVEKSFETDTNLNFQNLSVIGFRILLLKVAGFNLLMTLRLWSSG |
| | [T2A]-[eGFP] | SGEGRGSLLTCGDVEENPGPVSKGEELFTGVVPILVELDGDVNGHKFSVSGEGEGDATYGKL |
| | | TLKFICTTGKLPVPWPTLVTTLTYGVQCFSRYPDHMKQHDFFKSAMPEGYVQERTIFFKDDGN |
| | | YKTRAEVKFEGDTLVNRIELKGIDFKEDGNILGHKLEYNYNSHNVYIMADKQKNGIKVNFKIRH |
| | | NIEDGSVQLADHYQQNTPIGDGPVLLPDNHYLSTQSALSKDPNEKRDHMVLLEFVTAAGITLG |
| | | MDELYK |
| 140 | P329G TCRαβ- | MGWSCIILFLVATATGVHSQAVVTQEPSLTVSPGGTVTLTCRSSTGAVTTSNYANWVQEKPD |
| | VL1TRAC (with SP) | HLFTGLIGGTNKRAPGTPARFSGSLLGGKAALTLSGAQPEDEAEYYCALWYSNHWVFGGGTK |
| | [SP]-[VL1]-[TRAC_T47C] | LTVLIQNPDPAVYQLRDSKSSDKSVCLFTDFDSQTNVSQSKDSDVYITDKCVLDMRSMDFKSN |
| | | SAVAWSNKSDFACANAFNNSIIPEDTFFPSPESSCDVKLVEKSFETDTNLNFQNLSVIGFRILLL |
| | | KVAGFNLLMTLRLWSS |
| 141 | P329G TCRαβ- | QAVVTQEPSLTVSPGGTVTLTCRSSTGAVTTSNYANWVQEKPDHLFTGLIGGTNKRAPGTPA |
| | VL1TRAC (mature) | RFSGSLLGGKAALTLSGAQPEDEAEYYCALWYSNHWVFGGGTKLTVLIQNPDPAVYQLRDSK |
| | [VL1]-[TRAC_T47C] | SSDKSVCLFTDFDSQTNVSQSKDSDVYITDKCVLDMRSMDFKSNSAVAWSNKSDFACANAF |
| | | NNSIIPEDTFFPSPESSCDVKLVEKSFETDTNLNFQNLSVIGFRILLLKVAGFNLLMTLRLWSS |
| 142 | AAA TCRαβ-VHTRAC | MGWSCIILFLVATATGVHSEVQLVESGGDLVKPGGSLKLSCAASGFTFSSYGMSWVRQTPDK |
| | (with SP, T2A, eGFP) | RLEWVATISSGGSYIYYPDSVKGRFTISRDNAKNTLYLQMSSLKSEDTAMYYCARLGMITTGY |
| | [SP]-[VH]-[TRAC_T47C]- | AMDYWGQGTSVTVSSIQNPDPAVYQLRDSKSSDKSVCLFTDFDSQTNVSQSKDSDVYITDKC |
| | [T2A]-[eGFP] | VLDMRSMDFKSNSAVAWSNKSDFACANAFNNSIIPEDTFFPSPESSCDVKLVEKSFETDTNLN |
| | | FQNLSVIGFRILLLKVAGFNLLMTLRLWSSGSGEGRGSLLTCGDVEENPGPVSKGEELFTGVV |
| | | PILVELDGDVNGHKFSVSGEGEGDATYGKLTLKFICTTGKLPVPWPTLVTTLTYGVQCFSRYP |
| | | DHMKQHDFFKSAMPEGYVQERTIFFKDDGNYKTRAEVKFEGDTLVNRIELKGIDFKEDGNILG |
| | | HKLEYNYNSHNVYIMADKQKNGIKVNFKIRHNIEDGSVQLADHYQQNTPIGDGPVLLPDNHYL |
| | | STQSALSKDPNEKRDHMVLLEFVTAAGITLGMDELYK |
| 143 | AAA TCRαβ-VHTRAC | EVQLVESGGDLVKPGGSLKLSCAASGFTFSSYGMSWVRQTPDKRLEWVATISSGGSYIYYPD |
| | (with T2A, eGFP) | SVKGRFTISRDNAKNTLYLQMSSLKSEDTAMYYCARLGMITTGYAMDYWGQGTSVTVSSIQN |
| | [VH]-[TRAC_T47C]- | PDPAVYQLRDSKSSDKSVCLFTDFDSQTNVSQSKDSDVYITDKCVLDMRSMDFKSNSAVAW |
| | [T2A]-[eGFP] | SNKSDFACANAFNNSIIPEDTFFPSPESSCDVKLVEKSFETDTNLNFQNLSVIGFRILLLKVAGF |
| | | NLLMTLRLWSSGSGEGRGSLLTCGDVEENPGPVSKGEELFTGVVPILVELDGDVNGHKFSVS |
| | | GEGEGDATYGKLTLKFICTTGKLPVPWPTLVTTLTYGVQCFSRYPDHMKQHDFFKSAMPEGY |
| | | VQERTIFFKDDGNYKTRAEVKFEGDTLVNRIELKGIDFKEDGNILGHKLEYNYNSHNVYIMADK |
| | | QKNGIKVNFKIRHNIEDGSVQLADHYQQNTPIGDGPVLLPDNHYLSTQSALSKDPNEKRDHMV |
| | | LLEFVTAAGITLGMDELYK |
| 144 | AAA TCRαβ-VHTRAC | MGWSCIILFLVATATGVHSEVQLVESGGDLVKPGGSLKLSCAASGFTFSSYGMSWVRQTPDK |
| | (with SP) | RLEWVATISSGGSYIYYPDSVKGRFTISRDNAKNTLYLQMSSLKSEDTAMYYCARLGMITTGY |
| | [SP]-[VH]-[TRAC_T47C] | AMDYWGQGTSVTVSSIQNPDPAVYQLRDSKSSDKSVCLFTDFDSQTNVSQSKDSDVYITDKC |
| | | VLDMRSMDFKSNSAVAWSNKSDFACANAFNNSIIPEDTFFPSPESSCDVKLVEKSFETDTNLN |
| | | FQNLSVIGFRILLLKVAGFNLLMTLRLWSS |
| 145 | AAA TCRαβ-VHTRAC | EVQLVESGGDLVKPGGSLKLSCAASGFTFSSYGMSWVRQTPDKRLEWVATISSGGSYIYYPD |
| | (mature) | SVKGRFTISRDNAKNTLYLQMSSLKSEDTAMYYCARLGMITTGYAMDYWGQGTSVTVSSIQN |
| | [VH]-[TRAC_T47C] | PDPAVYQLRDSKSSDKSVCLFTDFDSQTNVSQSKDSDVYITDKCVLDMRSMDFKSNSAVAW |
| | | SNKSDFACANAFNNSIIPEDTFFPSPESSCDVKLVEKSFETDTNLNFQNLSVIGFRILLLKVAGF |
| | | NLLMTLRLWSS |
| 146 | AAA TCRαβ-VLTRAC | MGWSCIILFLVATATGVHSDVLMTQTPLSLPVSLGDQASISCRSSQTIVHSTGHTYLEWFLQKP |
| | (with SP, T2A, eGFP) | GQSPKLLIYKVSNRFSGVPDRFSGSGSGTDFTLKISRVEAEDLGVYYCFQGSHVPYTFGGGT |
| | [SP]-[VL]-[TRAC_T47C]- | KLEIKIQNPDPAVYQLRDSKSSDKSVCLFTDFDSQTNVSQSKDSDVYITDKCVLDMRSMDFKS |
| | [T2A]-[eGFP] | NSAVAWSNKSDFACANAFNNSIIPEDTFFPSPESSCDVKLVEKSFETDTNLNFQNLSVIGFRILL |
| | | LKVAGFNLLMTLRLWSSGSGEGRGSLLTCGDVEENPGPVSKGEELFTGVVPILVELDGDVNG |
| | | HKFSVSGEGEGDATYGKLTLKFICTTGKLPVPWPTLVTTLTYGVQCFSRYPDHMKQHDFFKS |
| | | AMPEGYVQERTIFFKDDGNYKTRAEVKFEGDTLVNRIELKGIDFKEDGNILGHKLEYNYNSHN |
| | | VYIMADKQKNGIKVNFKIRHNIEDGSVQLADHYQQNTPIGDGPVLLPDNHYLSTQSALSKDPNE |
| | | KRDHMVLLEFVTAAGITLGMDELYK |
| 147 | AAA TCRαβ-VLTRAC | DVLMTQTPLSLPVSLGDQASISCRSSQTIVHSTGHTYLEWFLQKPGQSPKLLIYKVSNRFSGV |
| | (with T2A, eGFP) | PDRFSGSGSGTDFTLKISRVEAEDLGVYYCFQGSHVPYTFGGGTKLEIKIQNPDPAVYQLRDS |
| | [VL]-[TRAC_T47C]- | KSSDKSVCLFTDFDSQTNVSQSKDSDVYITDKCVLDMRSMDFKSNSAVAWSNKSDFACANAF |
| | [T2A]-[eGFP] | NNSIIPEDTFFPSPESSCDVKLVEKSFETDTNLNFQNLSVIGFRILLLKVAGFNLLMTLRLWSSG |
| | | SGEGRGSLLTCGDVEENPGPVSKGEELFTGVVPILVELDGDVNGHKFSVSGEGEGDATYGKL |
| | | TLKFICTTGKLPVPWPTLVTTLTYGVQCFSRYPDHMKQHDFFKSAMPEGYVQERTIFFKDDGN |
| | | YKTRAEVKFEGDTLVNRIELKGIDFKEDGNILGHKLEYNYNSHNVYIMADKQKNGIKVNFKIRH |
| | | NIEDGSVQLADHYQQNTPIGDGPVLLPDNHYLSTQSALSKDPNEKRDHMVLLEFVTAAGITLG |
| | | MDELYK |
| 148 | AAA TCRαβ-VLTRAC | MGWSCIILFLVATATGVHSDVLMTQTPLSLPVSLGDQASISCRSSQTIVHSTGHTYLEWFLQKP |
| | (with SP) | GQSPKLLIYKVSNRFSGVPDRFSGSGSGTDFTLKISRVEAEDLGVYYCFQGSHVPYTFGGGT |
| | [SP]-[VL]-[TRAC_T47C] | KLEIKIQNPDPAVYQLRDSKSSDKSVCLFTDFDSQTNVSQSKDSDVYITDKCVLDMRSMDFKS |
| | | NSAVAWSNKSDFACANAFNNSIIPEDTFFPSPESSCDVKLVEKSFETDTNLNFQNLSVIGFRILL |
| | | LKVAGFNLLMTLRLWSS |
| 149 | AAA TCRαβ-VLTRAC | DVLMTQTPLSLPVSLGDQASISCRSSQTIVHSTGHTYLEWFLQKPGQSPKLLIYKVSNRFSGV |
| | (mature) | PDRFSGSGSGTDFTLKISRVEAEDLGVYYCFQGSHVPYTFGGGTKLEIKIQNPDPAVYQLRDS |
| | [VL]-[TRAC_T47C] | KSSDKSVCLFTDFDSQTNVSQSKDSDVYITDKCVLDMRSMDFKSNSAVAWSNKSDFACANAF |
| | | NNSIIPEDTFFPSPESSCDVKLVEKSFETDTNLNFQNLSVIGFRILLLKVAGFNLLMTLRLWSS |
| 150 | P329G TCRαβ- | MGWSCIILFLVATATGVHSEVQLVESGGGLVQPGGSLRLSCAASGFDFSRYWMNWVRQAPG |
| | VH1TRBC1 (with SP, | KGLEWVGEITPDSSTINYTPSLKGRFTISRDNAKNSLYLQMNSLRAEDTAVYYCVRPYDYGAW |
| | T2A, eGFP) | FASWGQGTLVTVSSDLNKVFPPEVAVFEPSEAEISHTQKATLVCLATGFFPDHVELSWWWVNG |
| | [SP]-[VH1]- | KEVHSGVCTDPQPLKEQPALNDSRYCLSSRLRVSATFWQNPRNHFRCQVQFYGLSENDEWT |
| | [TRBC1_S56C]-[T2A]- | QDRAKPVTQIVSAEAWGRADCGFTSVSYQQGVLSATILYEILLGKATLYAVLVSALVLMAMVK |
| | [eGFP] | RKDFGSGEGRGSLLTCGDVEENPGPVSKGEELFTGVVPILVELDGDVNGHKFSVSGEGEGD |
| | | ATYGKLTLKFICTTGKLPVPWPTLVTTLTYGVQCFSRYPDHMKQHDFFKSAMPEGYVQERTIF |
| | | FKDDGNYKTRAEVKFEGDTLVNRIELKGIDFKEDGNILGHKLEYNYNSHNVYIMADKQKNGIKV |
| | | NFKIRHNIEDGSVQLADHYQQNTPIGDGPVLLPDNHYLSTQSALSKDPNEKRDHMVLLEFVTA |
| | | AGITLGMDELYK |
| 151 | P329G TCRαβ- | EVQLVESGGGLVQPGGSLRLSCAASGFDFSRYWMNWVRQAPGKGLEWVGEITPDSSTINYT |
| | VH1TRBC1 (with T2A, | PSLKGRFTISRDNAKNSLYLQMNSLRAEDTAVYYCVRPYDYGAWFASWGQGTLVTVSSDLNK |
| | eGFP) | VFPPEVAVFEPSEAEISHTQKATLVCLATGFFPDHVELSWWWNGKEVHSGVCTDPQPLKEQP |
| | [VH1]-[TRBC1_S56C]- | ALNDSRYCLSSRLRVSATFWQNPRNHFRCQVQFYGLSENDEWTQDRAKPVTQIVSAEAWGR |
| | [T2A]-[eGFP] | ADCGFTSVSYQQGVLSATILYEILLGKATLYAVLVSALVLMAMVKRKDFGSGEGRGSLLTCGD |
| | | VEENPGPVSKGEELFTGVVPILVELDGDVNGHKFSVSGEGEGDATYGKLTLKFICTTGKLPVP |
| | | WPTLVTTLTYGVQCFSRYPDHMKQHDFFKSAMPEGYVQERTIFFKDDGNYKTRAEVKFEGDT |
| | | LVNRIELKGIDFKEDGNILGHKLEYNYNSHNVYIMADKQKNGIKVNFKIRHNIEDGSVQLADHYQ |
| | | QNTPIGDGPVLLPDNHYLSTQSALSKDPNEKRDHMVLLEFVTAAGITLGMDELYK |
| 152 | P329G TCRαβ- | MGWSCIILFLVATATGVHSEVQLVESGGGLVQPGGSLRLSCAASGFDFSRYWMNWVRQAPG |
| | VH1TRBC1 (with SP) | KGLEWVGEITPDSSTINYTPSLKGRFTISRDNAKNSLYLQMNSLRAEDTAVYYCVRPYDYGAW |
| | [SP]-[VH1]- | FASWGQGTLVTVSSDLNKVFPPEVAVFEPSEAEISHTQKATLVCLATGFFPDHVELSWWWNG |
| | [TRBC1_S56C] | KEVHSGVCTDPQPLKEQPALNDSRYCLSSRLRVSATFWQNPRNHFRCQVQFYGLSENDEWT |
| | | QDRAKPVTQIVSAEAWGRADCGFTSVSYQQGVLSATILYEILLGKATLYAVLVSALVLMAMVK |
| | | RKDF |
| 153 | P329G TCRαβ- | EVQLVESGGGLVQPGGSLRLSCAASGFDFSRYWMNWVRQAPGKGLEWVGEITPDSSTINYT |
| | VH1TRBC1_(mature) | PSLKGRFTISRDNAKNSLYLQMNSLRAEDTAVYYCVRPYDYGAWFASWGQGTLVTVSSDLNK |
| | [VH1]-[TRBC1_S56C] | VFPPEVAVFEPSEAEISHTQKATLVCLATGFFPDHVELSWWWWVNGKEVHSGVCTDPQPLKEQP |
| | | ALNDSRYCLSSRLRVSATFWQNPRNHFRCQVQFYGLSENDEWTQDRAKPVTQIVSAEAWGR |
| | | ADCGFTSVSYQQGVLSATILYEILLGKATLYAVLVSALVLMAMVKRKDF |
| 154 | P329G TCRαβ- | MGWSCIILFLVATATGVHSEVQLVESGGGLVQPGGSLRLSCAASGFDFSRYWMNWVRQAPG |
| | VH2TRBC1 (with SP, | KGLEWVGEITPDSSTINYAPSLKGRFTISRDNAKNSLYLQMNSLRAEDTAVYYCVRPYDYGAW |
| | T2A, eGFP) | FASWGQGTLVTVSSDLNKVFPPEVAVFEPSEAEISHTQKATLVCLATGFFPDHVELSWWWNG |
| | [SP]-[VH2]- | KEVHSGVCTDPQPLKEQPALNDSRYCLSSRLRVSATFWQNPRNHFRCQVQFYGLSENDEWT |
| | [TRBC1_S56C]-[T2A]- | QDRAKPVTQIVSAEAWGRADCGFTSVSYQQGVLSATILYEILLGKATLYAVLVSALVLMAMVK |
| | [eGFP] | RKDFGSGEGRGSLLTCGDVEENPGPVSKGEELFTGVVPILVELDGDVNGHKFSVSGEGEGD |
| | | ATYGKLTLKFICTTGKLPVPWPTLVTTLTYGVQCFSRYPDHMKQHDFFKSAMPEGYVQERTIF |
| | | FKDDGNYKTRAEVKFEGDTLVNRIELKGIDFKEDGNILGHKLEYNYNSHNVYIMADKQKNGIKV |
| | | NFKIRHNIEDGSVQLADHYQQNTPIGDGPVLLPDNHYLSTQSALSKDPNEKRDHMVLLEFVTA |
| | | AGITLGMDELYK |
| 155 | P329G TCRαβ- | EVQLVESGGGLVQPGGSLRLSCAASGFDFSRYWMNWVRQAPGKGLEWVGEITPDSSTINYA |
| | VH2TRBC1 (with T2A, | PSLKGRFTISRDNAKNSLYLQMNSLRAEDTAVYYCVRPYDYGAWFASWGQGTLVTVSSDLNK |
| | eGFP) | VFPPEVAVFEPSEAEISHTQKATLVCLATGFFPDHVELSWWWNGKEVHSGVCTDPQPLKEQP |
| | [VH2]-[TRBC1_S56C]- | ALNDSRYCLSSRLRVSATFWQNPRNHFRCQVQFYGLSENDEWTQDRAKPVTQIVSAEAWGR |
| | [T2A]-[eGFP] | ADCGFTSVSYQQGVLSATILYEILLGKATLYAVLVSALVLMAMVKRKDFGSGEGRGSLLTCGD |
| | | VEENPGPVSKGEELFTGVVPILVELDGDVNGHKFSVSGEGEGDATYGKLTLKFICTTGKLPVP |
| | | WPTLVTTLTYGVQCFSRYPDHMKQHDFFKSAMPEGYVQERTIFFKDDGNYKTRAEVKFEGDT |
| | | LVNRIELKGIDFKEDGNILGHKLEYNYNSHNVYIMADKQKNGIKVNFKIRHNIEDGSVQLADHYQ |
| | | QNTPIGDGPVLLPDNHYLSTQSALSKDPNEKRDHMVLLEFVTAAGITLGMDELYK |
| 156 | P329G TCRαβ- | MGWSCIILFLVATATGVHSEVQLVESGGGLVQPGGSLRLSCAASGFDFSRYWMNWVRQAPG |
| | VH2TRBC1 (with SP) | KGLEWVGEITPDSSTINYAPSLKGRFTISRDNAKNSLYLQMNSLRAEDTAVYYCVRPYDYGAW |
| | [SP]-[VH2]- | FASWGQGTLVTVSSDLNKVFPPEVAVFEPSEAEISHTQKATLVCLATGFFPDHVELSWWWVNG |
| | [TRBC1_S56C] | KEVHSGVCTDPQPLKEQPALNDSRYCLSSRLRVSATFWQNPRNHFRCQVQFYGLSENDEWT |
| | | QDRAKPVTQIVSAEAWGRADCGFTSVSYQQGVLSATILYEILLGKATLYAVLVSALVLMAMVK |
| | | RKDF |
| 157 | P329G TCRαβ- | EVQLVESGGGLVQPGGSLRLSCAASGFDFSRYWMNWVRQAPGKGLEWVGEITPDSSTINYA |
| | VH2TRBC1_(mature) | PSLKGRFTISRDNAKNSLYLQMNSLRAEDTAVYYCVRPYDYGAWFASWGQGTLVTVSSDLNK |
| | [VH2]-[TRBC1_S56C] | VFPPEVAVFEPSEAEISHTQKATLVCLATGFFPDHVELSWWWVNGKEVHSGVCTDPQPLKEQP |
| | | ALNDSRYCLSSRLRVSATFWQNPRNHFRCQVQFYGLSENDEWTQDRAKPVTQIVSAEAWGR |
| | | ADCGFTSVSYQQGVLSATILYEILLGKATLYAVLVSALVLMAMVKRKDF |
| 158 | P329G TCRαβ- | MGWSCIILFLVATATGVHSEVQLVESGGGLVQPGGSLRLSCAASGFTFSRYWMNWVRQAPG |
| | VH3TRBC1 (with SP, | KGLEWVGEITPDSSTINYAPSLKGRFTISRDNAKNSLYLQMNSLRAEDTAVYYCARPYDYGAW |
| | T2A, eGFP) | FASWGQGTLVTVSSDLNKVFPPEVAVFEPSEAEISHTQKATLVCLATGFFPDHVELSWWWVNG |
| | [SP]-[VH3]- | KEVHSGVCTDPQPLKEQPALNDSRYCLSSRLRVSATFWQNPRNHFRCQVQFYGLSENDEWT |
| | [TRBC1_S56C]-[T2A]- | QDRAKPVTQIVSAEAWGRADCGFTSVSYQQGVLSATILYEILLGKATLYAVLVSALVLMAMVK |
| | [eGFP] | RKDFGSGEGRGSLLTCGDVEENPGPVSKGEELFTGVVPILVELDGDVNGHKFSVSGEGEGD |
| | | ATYGKLTLKFICTTGKLPVPWPTLVTTLTYGVQCFSRYPDHMKQHDFFKSAMPEGYVQERTIF |
| | | FKDDGNYKTRAEVKFEGDTLVNRIELKGIDFKEDGNILGHKLEYNYNSHNVYIMADKQKNGIKV |
| | | NFKIRHNIEDGSVQLADHYQQNTPIGDGPVLLPDNHYLSTQSALSKDPNEKRDHMVLLEFVTA |
| | | AGITLGMDELYK |
| 159 | P329G TCRαβ- | EVQLVESGGGLVQPGGSLRLSCAASGFTFSRYWMNWVRQAPGKGLEWVGEITPDSSTINYA |
| | VH3TRBC1 (with T2A, | PSLKGRFTISRDNAKNSLYLQMNSLRAEDTAVYYCARPYDYGAWFASWGQGTLVTVSSDLNK |
| | eGFP) | VFPPEVAVFEPSEAEISHTQKATLVCLATGFFPDHVELSWWWVNGKEVHSGVCTDPQPLKEQP |
| | [VH3]-[TRBC1_S56C]- | ALNDSRYCLSSRLRVSATFWQNPRNHFRCQVQFYGLSENDEWTQDRAKPVTQIVSAEAWGR |
| | [T2A]-[eGFP] | ADCGFTSVSYQQGVLSATILYEILLGKATLYAVLVSALVLMAMVKRKDFGSGEGRGSLLTCGD |
| | | VEENPGPVSKGEELFTGVVPILVELDGDVNGHKFSVSGEGEGDATYGKLTLKFICTTGKLPVP |
| | | WPTLVTTLTYGVQCFSRYPDHMKQHDFFKSAMPEGYVQERTIFFKDDGNYKTRAEVKFEGDT |
| | | LVNRIELKGIDFKEDGNILGHKLEYNYNSHNVYIMADKQKNGIKVNFKIRHNIEDGSVQLADHYQ |
| | | QNTPIGDGPVLLPDNHYLSTQSALSKDPNEKRDHMVLLEFVTAAGITLGMDELYK |
| 160 | P329G TCRαβ- | MGWSCIILFLVATATGVHSEVQLVESGGGLVQPGGSLRLSCAASGFTFSRYWMNWVRQAPG |
| | VH3TRBC1 (with SP) | KGLEWVGEITPDSSTINYAPSLKGRFTISRDNAKNSLYLQMNSLRAEDTAVYYCARPYDYGAW |
| | [SP]-[VH3]- | FASWGQGTLVTVSSDLNKVFPPEVAVFEPSEAEISHTQKATLVCLATGFFPDHVELSWWWNG |
| | [TRBC1_S56C] | KEVHSGVCTDPQPLKEQPALNDSRYCLSSRLRVSATFWQNPRNHFRCQVQFYGLSENDEWT |
| | | QDRAKPVTQIVSAEAWGRADCGFTSVSYQQGVLSATILYEILLGKATLYAVLVSALVLMAMVK |
| | | RKDF |
| 161 | P329G TCRαβ- | EVQLVESGGGLVQPGGSLRLSCAASGFTFSRYWMNWVRQAPGKGLEWVGEITPDSSTINYA |
| | VH3TRBC1_(mature) | PSLKGRFTISRDNAKNSLYLQMNSLRAEDTAVYYCARPYDYGAWFASWGQGTLVTVSSDLNK |
| | [VH3]-[TRBC1_S56C] | VFPPEVAVFEPSEAEISHTQKATLVCLATGFFPDHVELSWWWVNGKEVHSGVCTDPQPLKEQP |
| | | ALNDSRYCLSSRLRVSATFWQNPRNHFRCQVQFYGLSENDEWTQDRAKPVTQIVSAEAWGR |
| | | ADCGFTSVSYQQGVLSATILYEILLGKATLYAVLVSALVLMAMVKRKDF |
| 162 | P329G TCRαβ- | MGWSCIILFLVATATGVHSQAVVTQEPSLTVSPGGTVTLTCRSSTGAVTTSNYANWVQEKPD |
| | VL1TRBC1 (with SP, | HLFTGLIGGTNKRAPGTPARFSGSLLGGKAALTLSGAQPEDEAEYYCALWYSNHWVFGGGTK |
| | T2A, eGFP) | LTVLDLNKVFPPEVAVFEPSEAEISHTQKATLVCLATGFFPDHVELSWWVNGKEVHSGVCTDP |
| | [SP]-[VL1]- | QPLKEQPALNDSRYCLSSRLRVSATFWQNPRNHFRCQVQFYGLSENDEWTQDRAKPVTQIV |
| | [TRBC1_S56C]-[T2A]- | SAEAWGRADCGFTSVSYQQGVLSATILYEILLGKATLYAVLVSALVLMAMVKRKDFGSGEGR |
| | [eGFP] | GSLLTCGDVEENPGPVSKGEELFTGVVPILVELDGDVNGHKFSVSGEGEGDATYGKLTLKFIC |
| | | TTGKLPVPWPTLVTTLTYGVQCFSRYPDHMKQHDFFKSAMPEGYVQERTIFFKDDGNYKTRA |
| | | EVKFEGDTLVNRIELKGIDFKEDGNILGHKLEYNYNSHNVYIMADKQKNGIKVNFKIRHNIEDGS |
| | | VQLADHYQQNTPIGDGPVLLPDNHYLSTQSALSKDPNEKRDHMVLLEFVTAAGITLGMDELYK |
| 163 | P329G TCRαβ- | QAVVTQEPSLTVSPGGTVTLTCRSSTGAVTTSNYANWVQEKPDHLFTGLIGGTNKRAPGTPA |
| | VL1TRBC1 (with T2A, | RFSGSLLGGKAALTLSGAQPEDEAEYYCALWYSNHWVFGGGTKLTVLDLNKVFPPEVAVFEP |
| | eGFP) | SEAEISHTQKATLVCLATGFFPDHVELSWWWVNGKEVHSGVCTDPQPLKEQPALNDSRYCLSS |
| | [VL1]-[TRBC1_S56C]- | RLRVSATFWQNPRNHFRCQVQFYGLSENDEWTQDRAKPVTQIVSAEAWGRADCGFTSVSY |
| | [T2A]-[eGFP] | QQGVLSATILYEILLGKATLYAVLVSALVLMAMVKRKDFGSGEGRGSLLTCGDVEENPGPVSK |
| | | GEELFTGVVPILVELDGDVNGHKFSVSGEGEGDATYGKLTLKFICTTGKLPVPWPTLVTTLTYG |
| | | VQCFSRYPDHMKQHDFFKSAMPEGYVQERTIFFKDDGNYKTRAEVKFEGDTLVNRIELKGIDF |
| | | KEDGNILGHKLEYNYNSHNVYIMADKQKNGIKVNFKIRHNIEDGSVQLADHYQQNTPIGDGPVL |
| | | LPDNHYLSTQSALSKDPNEKRDHMVLLEFVTAAGITLGMDELYK |
| 164 | P329G TCRαβ- | MGWSCIILFLVATATGVHSQAVVTQEPSLTVSPGGTVTLTCRSSTGAVTTSNYANWVQEKPD |
| | VL1TRBC1 (with SP) | HLFTGLIGGTNKRAPGTPARFSGSLLGGKAALTLSGAQPEDEAEYYCALWYSNHWVFGGGTK |
| | [SP]-[VL1]- | LTVLDLNKVFPPEVAVFEPSEAEISHTQKATLVCLATGFFPDHVELSWWVNGKEVHSGVCTDP |
| | [TRBC1_S56C] | QPLKEQPALNDSRYCLSSRLRVSATFWQNPRNHFRCQVQFYGLSENDEWTQDRAKPVTQIV |
| | | SAEAWGRADCGFTSVSYQQGVLSATILYEILLGKATLYAVLVSALVLMAMVKRKDF |
| 165 | P329G TCRαβ- | QAVVTQEPSLTVSPGGTVTLTCRSSTGAVTTSNYANWVQEKPDHLFTGLIGGTNKRAPGTPA |
| | VL1TRBC1_(mature) | RFSGSLLGGKAALTLSGAQPEDEAEYYCALWYSNHWVFGGGTKLTVLDLNKVFPPEVAVFEP |
| | [VL1]-[TRBC1_S56C] | SEAEISHTQKATLVCLATGFFPDHVELSWWVNGKEVHSGVCTDPQPLKEQPALNDSRYCLSS |
| | | RLRVSATFWQNPRNHFRCQVQFYGLSENDEWTQDRAKPVTQIVSAEAWGRADCGFTSVSY |
| | | QQGVLSATILYEILLGKATLYAVLVSALVLMAMVKRKDF |
| 166 | AAA TCRαβ-VHTRBC1 | MGWSCIILFLVATATGVHSEVQLVESGGDLVKPGGSLKLSCAASGFTFSSYGMSWVRQTPDK |
| | (with SP, T2A, eGFP) | RLEWVATISSGGSYIYYPDSVKGRFTISRDNAKNTLYLQMSSLKSEDTAMYYCARLGMITTGY |
| | [SP]-[VH]- | AMDYWGQGTSVTVSSDLNKVFPPEVAVFEPSEAEISHTQKATLVCLATGFFPDHVELSWWWVN |
| | [TRBC1_S56C]-[T2A]- | GKEVHSGVCTDPQPLKEQPALNDSRYCLSSRLRVSATFWQNPRNHFRCQVQFYGLSENDE |
| | [eGFP] | WTQDRAKPVTQIVSAEAWGRADCGFTSVSYQQGVLSATILYEILLGKATLYAVLVSALVLMAM |
| | | VKRKDFGSGEGRGSLLTCGDVEENPGPVSKGEELFTGVVPILVELDGDVNGHKFSVSGEGE |
| | | GDATYGKLTLKFICTTGKLPVPWPTLVTTLTYGVQCFSRYPDHMKQHDFFKSAMPEGYVQER |
| | | TIFFKDDGNYKTRAEVKFEGDTLVNRIELKGIDFKEDGNILGHKLEYNYNSHNVYIMADKQKNGI |
| | | KVNFKIRHNIEDGSVQLADHYQQNTPIGDGPVLLPDNHYLSTQSALSKDPNEKRDHMVLLEFV |
| | | TAAGITLGMDELYK |
| 167 | AAA TCRαβ-VHTRBC1 | EVQLVESGGDLVKPGGSLKLSCAASGFTFSSYGMSWVRQTPDKRLEWVATISSGGSYIYYPD |
| | (with T2A, eGFP) | SVKGRFTISRDNAKNTLYLQMSSLKSEDTAMYYCARLGMITTGYAMDYWGQGTSVTVSSDLN |
| | [VH]-[TRBC1_S56C]- | KVFPPEVAVFEPSEAEISHTQKATLVCLATGFFPDHVELSWWWVNGKEVHSGVCTDPQPLKE |
| | [T2A]-[eGFP] | QPALNDSRYCLSSRLRVSATFWQNPRNHFRCQVQFYGLSENDEWTQDRAKPVTQIVSAEAWG |
| | | RADCGFTSVSYQQGVLSATILYEILLGKATLYAVLVSALVLMAMVKRKDFGSGEGRGSLLTCG |
| | | DVEENPGPVSKGEELFTGVVPILVELDGDVNGHKFSVSGEGEGDATYGKLTLKFICTTGKLPV |
| | | PWPTLVTTLTYGVQCFSRYPDHMKQHDFFKSAMPEGYVQERTIFFKDDGNYKTRAEVKFEGDT |
| | | LVNRIELKGIDFKEDGNILGHKLEYNYNSHNVYIMADKQKNGIKVNFKIRHNIEDGSVQLADH |
| | | YQQNTPIGDGPVLLPDNHYLSTQSALSKDPNEKRDHMVLLEFVTAAGITLGMDELYK |
| 168 | AAA TCRαβ-VHTRBC1 | MGWSCIILFLVATATGVHSEVQLVESGGDLVKPGGSLKLSCAASGFTFSSYGMSWVRQTPDK |
| | (with SP) | RLEWVATISSGGSYIYYPDSVKGRFTISRDNAKNTLYLQMSSLKSEDTAMYYCARLGMITTGY |
| | [SP]-[VH]-[TRBC1_S56C] | AMDYWGQGTSVTVSSDLNKVFPPEVAVFEPSEAEISHTQKATLVCLATGFFPDHVELSWWWWV |
| | | NGKEVHSGVCTDPQPLKEQPALNDSRYCLSSRLRVSATFWQNPRNHFRCQVQFYGLSENDE |
| | | WTQDRAKPVTQIVSAEAWGRADCGFTSVSYQQGVLSATILYEILLGKATLYAVLVSALVLMAM |
| | | VKRKDF |
| 169 | AAA TCRαβ-VHTRBC1 | EVQLVESGGDLVKPGGSLKLSCAASGFTFSSYGMSWVRQTPDKRLEWVATISSGGSYIYYPD |
| | (mature) | SVKGRFTISRDNAKNTLYLQMSSLKSEDTAMYYCARLGMITTGYAMDYWGQGTSVTVSSDLN |
| | [VH]-[TRBC1_S56C] | KVFPPEVAVFEPSEAEISHTQKATLVCLATGFFPDHVELSWWWVNGKEVHSGVCTDPQPLKEQ |
| | | PALNDSRYCLSSRLRVSATFWQNPRNHFRCQVQFYGLSENDEWTQDRAKPVTQIVSAEAWG |
| | | RADCGFTSVSYQQGVLSATILYEILLGKATLYAVLVSALVLMAMVKRKDF |
| 170 | AAA TCRαβ-VLTRBC1 | MGWSCIILFLVATATGVHSDVLMTQTPLSLPVSLGDQASISCRSSQTIVHSTGHTYLEWFLQK |
| | (with SP, T2A, eGFP) | PGQSPKLLIYKVSNRFSGVPDRFSGSGSGTDFTLKISRVEAEDLGVYYCFQGSHVPYTFGGGT |
| | [SP]-[VL]- | KLEIKDLNKVFPPEVAVFEPSEAEISHTQKATLVCLATGFFPDHVELSWWWVNGKEVHSGVCT |
| | [TRBC1_S56C]-[T2A]- | DPQPLKEQPALNDSRYCLSSRLRVSATFWQNPRNHFRCQVQFYGLSENDEWTQDRAKPVTQI |
| | [eGFP] | VSAEAWGRADCGFTSVSYQQGVLSATILYEILLGKATLYAVLVSALVLMAMVKRKDFGSGEGR |
| | | GSLLTCGDVEENPGPVSKGEELFTGVVPILVELDGDVNGHKFSVSGEGEGDATYGKLTLKFIC |
| | | TTGKLPVPWPTLVTTLTYGVQCFSRYPDHMKQHDFFKSAMPEGYVQERTIFFKDDGNYKTRAE |
| | | VKFEGDTLVNRIELKGIDFKEDGNILGHKLEYNYNSHNVYIMADKQKNGIKVNFKIRHNIEDGS |
| | | VQLADHYQQNTPIGDGPVLLPDNHYLSTQSALSKDPNEKRDHMVLLEFVTAAGITLGMDELYK |
| 171 | AAA TCRαβ-VLTRBC1 | DVLMTQTPLSLPVSLGDQASISCRSSQTIVHSTGHTYLEWFLQKPGQSPKLLIYKVSNRFSGV |
| | (with T2A, eGFP) | PDRFSGSGSGTDFTLKISRVEAEDLGVYYCFQGSHVPYTFGGGTKLEIKDLNKVFPPEVAVFE |
| | [VL]-[TRBC1_S56C]- | PSEAEISHTQKATLVCLATGFFPDHVELSWWWVNGKEVHSGVCTDPQPLKEQPALNDSRYCLS |
| | [T2A]-[eGFP] | SRLRVSATFWQNPRNHFRCQVQFYGLSENDEWTQDRAKPVTQIVSAEAWGRADCGFTSVSY |
| | | QQGVLSATILYEILLGKATLYAVLVSALVLMAMVKRKDFGSGEGRGSLLTCGDVEENPGPVSK |
| | | GEELFTGVVPILVELDGDVNGHKFSVSGEGEGDATYGKLTLKFICTTGKLPVPWPTLVTTLTY |
| | | GVQCFSRYPDHMKQHDFFKSAMPEGYVQERTIFFKDDGNYKTRAEVKFEGDTLVNRIELKGI |
| | | DFKEDGNILGHKLEYNYNSHNVYIMADKQKNGIKVNFKIRHNIEDGSVQLADHYQQNTPIGDG |
| | | PVLLPDNHYLSTQSALSKDPNEKRDHMVLLEFVTAAGITLGMDELYK |
| 172 | AAA TCRαβ-VLTRBC1 | MGWSCIILFLVATATGVHSDVLMTQTPLSLPVSLGDQASISCRSSQTIVHSTGHTYLEWFLQK |
| | (with SP) | PGQSPKLLIYKVSNRFSGVPDRFSGSGSGTDFTLKISRVEAEDLGVYYCFQGSHVPYTFGGGT |
| | [SP]-[VL]-[TRBC1_S56C] | KLEIKDLNKVFPPEVAVFEPSEAEISHTQKATLVCLATGFFPDHVELSWWWVNGKEVHSGVCT |
| | | DPQPLKEQPALNDSRYCLSSRLRVSATFWQNPRNHFRCQVQFYGLSENDEWTQDRAKPVTQI |
| | | VSAEAWGRADCGFTSVSYQQGVLSATILYEILLGKATLYAVLVSALVLMAMVKRKDF |
| 173 | AAA TCRαβ-VLTRBC1 | DVLMTQTPLSLPVSLGDQASISCRSSQTIVHSTGHTYLEWFLQKPGQSPKLLIYKVSNRFSGV |
| | (mature) | PDRFSGSGSGTDFTLKISRVEAEDLGVYYCFQGSHVPYTFGGGTKLEIKDLNKVFPPEVAVFE |
| | [VL]-[TRBC1_S56C] | PSEAEISHTQKATLVCLATGFFPDHVELSWWWVNGKEVHSGVCTDPQPLKEQPALNDSRYCLS |
| | | SRLRVSATFWQNPRNHFRCQVQFYGLSENDEWTQDRAKPVTQIVSAEAWGRADCGFTSVS |
| | | YQQGVLSATILYEILLGKATLYAVLVSALVLMAMVKRKDF |
| 174 | P329G VH1VL1 TCRαβ | MGWSCIILFLVATATGVHSEVQLVESGGGLVQPGGSLRLSCAASGFDFSRYWMNWVRQAPG |
| | (with SP, T2A, eGFP) | KGLEWVGEITPDSSTINYTPSLKGRFTISRDNAKNSLYLQMNSLRAEDTAVYYCVRPYDYGAW |
| | [SP]-[VH1]- | FASWGQGTLVTVSSIQNPDPAVYQLRDSKSSDKSVCLFTDFDSQTNVSQSKDSDVYITDKCV |
| | [TRAC_T47C]-[T2A]- | LDMRSMDFKSNSAVAWSNKSDFACANAFNNSIIPEDTFFPSPESSCDVKLVEKSFETDTNLNF |
| | [SP]-[VL1]- | QNLSVIGFRILLLKVAGFNLLMTLRLWSSGSGEGRGSLLTCGDVEENPGPMGWSCIILFLVATA |
| | [TRBC1_S56C]-[T2A]- | TGVHSQAVVTQEPSLTVSPGGTVTLTCRSSTGAVTTSNYANWVQEKPDHLFTGLIGGTNKRA |
| | [eGFP] | PGTPARFSGSLLGGKAALTLSGAQPEDEAEYYCALWYSNHWVFGGGTKLTVLDLNKVFPPEV |
| | | AVFEPSEAEISHTQKATLVCLATGFFPDHVELSWWVNGKEVHSGVCTDPQPLKEQPALNDSR |
| | | YCLSSRLRVSATFWQNPRNHFRCQVQFYGLSENDEWTQDRAKPVTQIVSAEAWGRADCGFT |
| | | SVSYQQGVLSATILYEILLGKATLYAVLVSALVLMAMVKRKDFGSGEGRGSLLTCGDVEENPG |
| | | PVSKGEELFTGVVPILVELDGDVNGHKFSVSGEGEGDATYGKLTLKFICTTGKLPVPWPTLVT |
| | | TLTYGVQCFSRYPDHMKQHDFFKSAMPEGYVQERTIFFKDDGNYKTRAEVKFEGDTLVNRIE |
| | | LKGIDFKEDGNILGHKLEYNYNSHNVYIMADKQKNGIKVNFKIRHNIEDGSVQLADHYQQNTPI |
| | | GDGPVLLPDNHYLSTQSALSKDPNEKRDHMVLLEFVTAAGITLGMDELYK |
| 175 | P329G VH1VL1 TCRαβ | MGWSCIILFLVATATGVHSEVQLVESGGGLVQPGGSLRLSCAASGFDFSRYWMNWVRQAPG |
| | (with SP) | KGLEWVGEITPDSSTINYTPSLKGRFTISRDNAKNSLYLQMNSLRAEDTAVYYCVRPYDYGAW |
| | [SP]-[VH1]- | FASWGQGTLVTVSSIQNPDPAVYQLRDSKSSDKSVCLFTDFDSQTNVSQSKDSDVYITDKCV |
| | [TRAC_T47C]-[T2A]- | LDMRSMDFKSNSAVAWSNKSDFACANAFNNSIIPEDTFFPSPESSCDVKLVEKSFETDTNLNF |
| | [SP]-[VL1]- | QNLSVIGFRILLLKVAGFNLLMTLRLWSSGSGEGRGSLLTCGDVEENPGPMGWSCIILFLVATA |
| | [TRBC1_S56C] | TGVHSQAVVTQEPSLTVSPGGTVTLTCRSSTGAVTTSNYANWVQEKPDHLFTGLIGGTNKRA |
| | | PGTPARFSGSLLGGKAALTLSGAQPEDEAEYYCALWYSNHWVFGGGTKLTVLDLNKVFPPEV |
| | | AVFEPSEAEISHTQKATLVCLATGFFPDHVELSWWVNGKEVHSGVCTDPQPLKEQPALNDSR |
| | | YCLSSRLRVSATFWQNPRNHFRCQVQFYGLSENDEWTQDRAKPVTQIVSAEAWGRADCGFT |
| | | SVSYQQGVLSATILYEILLGKATLYAVLVSALVLMAMVKRKDF |
| 176 | P329G VH2VL1 TCRαβ | MGWSCIILFLVATATGVHSEVQLVESGGGLVQPGGSLRLSCAASGFDFSRYWMNWVRQAPG |
| | (with SP, T2A, eGFP) | KGLEWVGEITPDSSTINYAPSLKGRFTISRDNAKNSLYLQMNSLRAEDTAVYYCVRPYDYGAW |
| | [SP]-[VH2]- | FASWGQGTLVTVSSIQNPDPAVYQLRDSKSSDKSVCLFTDFDSQTNVSQSKDSDVYITDKCV |
| | [TRAC_T47C]-[T2A]- | LDMRSMDFKSNSAVAWSNKSDFACANAFNNSIIPEDTFFPSPESSCDVKLVEKSFETDTNLNF |
| | [SP]-[VL1]- | QNLSVIGFRILLLKVAGFNLLMTLRLWSSGSGEGRGSLLTCGDVEENPGPMGWSCIILFLVATA |
| | [TRBC1_S56C]-[T2A]- | TGVHSQAVVTQEPSLTVSPGGTVTLTCRSSTGAVTTSNYANWVQEKPDHLFTGLIGGTNKRA |
| | [eGFP] | PGTPARFSGSLLGGKAALTLSGAQPEDEAEYYCALWYSNHWVFGGGTKLTVLDLNKVFPPEV |
| | | AVFEPSEAEISHTQKATLVCLATGFFPDHVELSWWWVNGKEVHSGVCTDPQPLKEQPALNDSR |
| | | YCLSSRLRVSATFWQNPRNHFRCQVQFYGLSENDEWTQDRAKPVTQIVSAEAWGRADCGFT |
| | | SVSYQQGVLSATILYEILLGKATLYAVLVSALVLMAMVKRKDFGSGEGRGSLLTCGDVEENPG |
| | | PVSKGEELFTGVVPILVELDGDVNGHKFSVSGEGEGDATYGKLTLKFICTTGKLPVPWPTLVT |
| | | TLTYGVQCFSRYPDHMKQHDFFKSAMPEGYVQERTIFFKDDGNYKTRAEVKFEGDTLVNRIE |
| | | LKGIDFKEDGNILGHKLEYNYNSHNVYIMADKQKNGIKVNFKIRHNIEDGSVQLADHYQQNTPI |
| | | GDGPVLLPDNHYLSTQSALSKDPNEKRDHMVLLEFVTAAGITLGMDELYK |
| 177 | P329G VH2VL1 TCRαβ | MGWSCIILFLVATATGVHSEVQLVESGGGLVQPGGSLRLSCAASGFDFSRYWMNWVRQAPG |
| | (with SP) | KGLEWVGEITPDSSTINYAPSLKGRFTISRDNAKNSLYLQMNSLRAEDTAVYYCVRPYDYGAW |
| | [SP]-[VH2]- | FASWGQGTLVTVSSIQNPDPAVYQLRDSKSSDKSVCLFTDFDSQTNVSQSKDSDVYITDKCV |
| | [TRAC_T47C]-[T2A]- | LDMRSMDFKSNSAVAWSNKSDFACANAFNNSIIPEDTFFPSPESSCDVKLVEKSFETDTNLNF |
| | [SP]-[VL1]- | QNLSVIGFRILLLKVAGFNLLMTLRLWSSGSGEGRGSLLTCGDVEENPGPMGWSCIILFLVATA |
| | [TRBC1_S56C] | TGVHSQAVVTQEPSLTVSPGGTVTLTCRSSTGAVTTSNYANWVQEKPDHLFTGLIGGTNKRA |
| | | PGTPARFSGSLLGGKAALTLSGAQPEDEAEYYCALWYSNHWVFGGGTKLTVLDLNKVFPPEV |
| | | AVFEPSEAEISHTQKATLVCLATGFFPDHVELSWWVNGKEVHSGVCTDPQPLKEQPALNDSR |
| | | YCLSSRLRVSATFWQNPRNHFRCQVQFYGLSENDEWTQDRAKPVTQIVSAEAWGRADCGFT |
| | | SVSYQQGVLSATILYEILLGKATLYAVLVSALVLMAMVKRKDF |
| 178 | P329G VH3VL1 TCRαβ | MGWSCIILFLVATATGVHSEVQLVESGGGLVQPGGSLRLSCAASGFTFSRYWMNWVRQAPG |
| | (with SP, T2A, eGFP) | KGLEWVGEITPDSSTINYAPSLKGRFTISRDNAKNSLYLQMNSLRAEDTAVYYCARPYDYGAW |
| | [SP]-[VH3]- | FASWGQGTLVTVSSIQNPDPAVYQLRDSKSSDKSVCLFTDFDSQTNVSQSKDSDVYITDKCV |
| | [TRAC_T47C]-[T2A]- | LDMRSMDFKSNSAVAWSNKSDFACANAFNNSIIPEDTFFPSPESSCDVKLVEKSFETDTNLNF |
| | [SP]-[VL1]- | QNLSVIGFRILLLKVAGFNLLMTLRLWSSGSGEGRGSLLTCGDVEENPGPMGWSCIILFLVATA |
| | [TRBC1_S56C]-[T2A]- | TGVHSQAVVTQEPSLTVSPGGTVTLTCRSSTGAVTTSNYANWVQEKPDHLFTGLIGGTNKRA |
| | [eGFP] | PGTPARFSGSLLGGKAALTLSGAQPEDEAEYYCALWYSNHWVFGGGTKLTVLDLNKVFPPEV |
| | | AVFEPSEAEISHTQKATLVCLATGFFPDHVELSWWVNGKEVHSGVCTDPQPLKEQPALNDSR |
| | | YCLSSRLRVSATFWQNPRNHFRCQVQFYGLSENDEWTQDRAKPVTQIVSAEAWGRADCGFT |
| | | SVSYQQGVLSATILYEILLGKATLYAVLVSALVLMAMVKRKDFGSGEGRGSLLTCGDVEENPG |
| | | PVSKGEELFTGVVPILVELDGDVNGHKFSVSGEGEGDATYGKLTLKFICTTGKLPVPWPTLVT |
| | | TLTYGVQCFSRYPDHMKQHDFFKSAMPEGYVQERTIFFKDDGNYKTRAEVKFEGDTLVNRIE |
| | | LKGIDFKEDGNILGHKLEYNYNSHNVYIMADKQKNGIKVNFKIRHNIEDGSVQLADHYQQNTPI |
| | | GDGPVLLPDNHYLSTQSALSKDPNEKRDHMVLLEFVTAAGITLGMDELYK |
| 179 | P329G VH3VL1 TCRαβ | MGWSCIILFLVATATGVHSEVQLVESGGGLVQPGGSLRLSCAASGFTFSRYWMNWVRQAPG |
| | (with SP) | KGLEWVGEITPDSSTINYAPSLKGRFTISRDNAKNSLYLQMNSLRAEDTAVYYCARPYDYGAW |
| | [SP]-[VH3]- | FASWGQGTLVTVSSIQNPDPAVYQLRDSKSSDKSVCLFTDFDSQTNVSQSKDSDVYITDKCV |
| | [TRAC_T47C]-[T2A]- | LDMRSMDFKSNSAVAWSNKSDFACANAFNNSIIPEDTFFPSPESSCDVKLVEKSFETDTNLNF |
| | [SP]-[VL1]- | QNLSVIGFRILLLKVAGFNLLMTLRLWSSGSGEGRGSLLTCGDVEENPGPMGWSCIILFLVATA |
| | [TRBC1_S56C] | TGVHSQAVVTQEPSLTVSPGGTVTLTCRSSTGAVTTSNYANWVQEKPDHLFTGLIGGTNKRA |
| | | PGTPARFSGSLLGGKAALTLSGAQPEDEAEYYCALWYSNHWVFGGGTKLTVLDLNKVFPPEV |
| | | AVFEPSEAEISHTQKATLVCLATGFFPDHVELSWWWVNGKEVHSGVCTDPQPLKEQPALNDSR |
| | | YCLSSRLRVSATFWQNPRNHFRCQVQFYGLSENDEWTQDRAKPVTQIVSAEAWGRADCGFT |
| | | SVSYQQGVLSATILYEILLGKATLYAVLVSALVLMAMVKRKDF |
| 180 | AAA TCRαβ (with SP, | MGWSCIILFLVATATGVHSEVQLVESGGDLVKPGGSLKLSCAASGFTFSSYGMSWVRQTPDK |
| | T2A, eGFP) | RLEWVATISSGGSYIYYPDSVKGRFTISRDNAKNTLYLQMSSLKSEDTAMYYCARLGMITTGY |
| | [SP]-[VH]-[TRAC_T47C]- | AMDYWGQGTSVTVSSIQNPDPAVYQLRDSKSSDKSVCLFTDFDSQTNVSQSKDSDVYITDKC |
| | [T2A]-[SP]-[VL]- | VLDMRSMDFKSNSAVAWSNKSDFACANAFNNSIIPEDTFFPSPESSCDVKLVEKSFETDTNLN |
| | [TRBC1_S56C]-[T2A]- | FQNLSVIGFRILLLKVAGFNLLMTLRLWSSGSGEGRGSLLTCGDVEENPGPMGWSCIILFLVAT |
| | [eGFP] | ATGVHSDVLMTQTPLSLPVSLGDQASISCRSSQTIVHSTGHTYLEWFLQKPGQSPKLLIYKVS |
| | | NRFSGVPDRFSGSGSGTDFTLKISRVEAEDLGVYYCFQGSHVPYTFGGGTKLEIKDLNKVFPP |
| | | EVAVFEPSEAEISHTQKATLVCLATGFFPDHVELSWWWVNGKEVHSGVCTDPQPLKEQPALND |
| | | SRYCLSSRLRVSATFWQNPRNHFRCQVQFYGLSENDEWTQDRAKPVTQIVSAEAWGRADC |
| | | GFTSVSYQQGVLSATILYEILLGKATLYAVLVSALVLMAMVKRKDFGSGEGRGSLLTCGDVEE |
| | | NPGPVSKGEELFTGVVPILVELDGDVNGHKFSVSGEGEGDATYGKLTLKFICTTGKLPVPWPT |
| | | LVTTLTYGVQCFSRYPDHMKQHDFFKSAMPEGYVQERTIFFKDDGNYKTRAEVKFEGDTLVN |
| | | RIELKGIDFKEDGNILGHKLEYNYNSHNVYIMADKQKNGIKVNFKIRHNIEDGSVQLADHYQQN |
| | | TPIGDGPVLLPDNHYLSTQSALSKDPNEKRDHMVLLEFVTAAGITLGMDELYK |
| 181 | AAA TCRαβ (with SP) | MGWSCIILFLVATATGVHSEVQLVESGGDLVKPGGSLKLSCAASGFTFSSYGMSWVRQTPDK |
| | [SP]-[VH]-[TRAC_T47C]- | RLEWVATISSGGSYIYYPDSVKGRFTISRDNAKNTLYLQMSSLKSEDTAMYYCARLGMITTGY |
| | [T2A]-[SP]-[VL]- | AMDYWGQGTSVTVSSIQNPDPAVYQLRDSKSSDKSVCLFTDFDSQTNVSQSKDSDVYITDKC |
| | [TRBC1_S56C] | VLDMRSMDFKSNSAVAWSNKSDFACANAFNNSIIPEDTFFPSPESSCDVKLVEKSFETDTNLN |
| | | FQNLSVIGFRILLLKVAGFNLLMTLRLWSSGSGEGRGSLLTCGDVEENPGPMGWSCIILFLVAT |
| | | ATGVHSDVLMTQTPLSLPVSLGDQASISCRSSQTIVHSTGHTYLEWFLQKPGQSPKLLIYKVS |
| | | NRFSGVPDRFSGSGSGTDFTLKISRVEAEDLGVYYCFQGSHVPYTFGGGTKLEIKDLNKVFPP |
| | | EVAVFEPSEAEISHTQKATLVCLATGFFPDHVELSWWWVNGKEVHSGVCTDPQPLKEQPALND |
| | | SRYCLSSRLRVSATFWQNPRNHFRCQVQFYGLSENDEWTQDRAKPVTQIVSAEAWGRADC |
| | | GFTSVSYQQGVLSATILYEILLGKATLYAVLVSALVLMAMVKRKDF |
| 182 | P329G VH1VL1 TCRαβ | MGWSCIILFLVATATGVHSEVQLVESGGGLVQPGGSLRLSCAASGFDFSRYWMNWVRQAPG |
| | (with SP, T2A, eGFP) | KGLEWVGEITPDSSTINYTPSLKGRFTISRDNAKNSLYLQMNSLRAEDTAVYYCVRPYDYGAW |
| | [SP]-[VH1]- | FASWGQGTLVTVSSDLNKVFPPEVAVFEPSEAEISHTQKATLVCLATGFFPDHVELSWWWVNG |
| | [TRBC1_S56C]-[T2A]- | KEVHSGVCTDPQPLKEQPALNDSRYCLSSRLRVSATFWQNPRNHFRCQVQFYGLSENDEWT |
| | [SP]-[VL1]- | QDRAKPVTQIVSAEAWGRADCGFTSVSYQQGVLSATILYEILLGKATLYAVLVSALVLMAMVK |
| | [TRAC_T47C]-[T2A]- | RKDFGSGEGRGSLLTCGDVEENPGPMGWSCIILFLVATATGVHSQAVVTQEPSLTVSPGGTV |
| | [eGFP] | TLTCRSSTGAVTTSNYANWVQEKPDHLFTGLIGGTNKRAPGTPARFSGSLLGGKAALTLSGA |
| | | QPEDEAEYYCALWYSNHWVFGGGTKLTVLIQNPDPAVYQLRDSKSSDKSVCLFTDFDSQTNV |
| | | SQSKDSDVYITDKCVLDMRSMDFKSNSAVAWSNKSDFACANAFNNSIIPEDTFFPSPESSCDV |
| | | KLVEKSFETDTNLNFQNLSVIGFRILLLKVAGFNLLMTLRLWSSGSGEGRGSLLTCGDVEENP |
| | | GPVSKGEELFTGVVPILVELDGDVNGHKFSVSGEGEGDATYGKLTLKFICTTGKLPVPWPTLV |
| | | TTLTYGVQCFSRYPDHMKQHDFFKSAMPEGYVQERTIFFKDDGNYKTRAEVKFEGDTLVNRI |
| | | ELKGIDFKEDGNILGHKLEYNYNSHNVYIMADKQKNGIKVNFKIRHNIEDGSVQLADHYQQNTP |
| | | IGDGPVLLPDNHYLSTQSALSKDPNEKRDHMVLLEFVTAAGITLGMDELYK |
| 183 | P329G VH1VL1 TCRαβ | MGWSCIILFLVATATGVHSEVQLVESGGGLVQPGGSLRLSCAASGFDFSRYWMNWVRQAPG |
| | (with SP) | KGLEWVGEITPDSSTINYTPSLKGRFTISRDNAKNSLYLQMNSLRAEDTAVYYCVRPYDYGAW |
| | [SP]-[VH1]- | FASWGQGTLVTVSSDLNKVFPPEVAVFEPSEAEISHTQKATLVCLATGFFPDHVELSWWWNG |
| | [TRBC1_S56C]-[T2A]- | KEVHSGVCTDPQPLKEQPALNDSRYCLSSRLRVSATFWQNPRNHFRCQVQFYGLSENDEWT |
| | [SP]-[VL1]-[TRAC_T47C] | QDRAKPVTQIVSAEAWGRADCGFTSVSYQQGVLSATILYEILLGKATLYAVLVSALVLMAMVK |
| | | RKDFGSGEGRGSLLTCGDVEENPGPMGWSCIILFLVATATGVHSQAVVTQEPSLTVSPGGTV |
| | | TLTCRSSTGAVTTSNYANWVQEKPDHLFTGLIGGTNKRAPGTPARFSGSLLGGKAALTLSGA |
| | | QPEDEAEYYCALWYSNHWVFGGGTKLTVLIQNPDPAVYQLRDSKSSDKSVCLFTDFDSQTNV |
| | | SQSKDSDVYITDKCVLDMRSMDFKSNSAVAWSNKSDFACANAFNNSIIPEDTFFPSPESSCDV |
| | | KLVEKSFETDTNLNFQNLSVIGFRILLLKVAGFNLLMTLRLWSS |
| 184 | P329G VH2VL1 TCRαβ | MGWSCIILFLVATATGVHSEVQLVESGGGLVQPGGSLRLSCAASGFDFSRYWMNWVRQAPG |
| | (with SP, T2A, eGFP) | KGLEWVGEITPDSSTINYAPSLKGRFTISRDNAKNSLYLQMNSLRAEDTAVYYCVRPYDYGAW |
| | [SP]-[VH2]- | FASWGQGTLVTVSSDLNKVFPPEVAVFEPSEAEISHTQKATLVCLATGFFPDHVELSWWWWVNG |
| | [TRBC1_S56C]-[T2A]- | KEVHSGVCTDPQPLKEQPALNDSRYCLSSRLRVSATFWQNPRNHFRCQVQFYGLSENDEWT |
| | [SP]-[VL1]- | QDRAKPVTQIVSAEAWGRADCGFTSVSYQQGVLSATILYEILLGKATLYAVLVSALVLMAMVK |
| | [TRAC_T47C]-[T2A]- | RKDFGSGEGRGSLLTCGDVEENPGPMGWSCIILFLVATATGVHSQAVVTQEPSLTVSPGGTV |
| | [eGFP] | TLTCRSSTGAVTTSNYANWVQEKPDHLFTGLIGGTNKRAPGTPARFSGSLLGGKAALTLSGA |
| | | QPEDEAEYYCALWYSNHWVFGGGTKLTVLIQNPDPAVYQLRDSKSSDKSVCLFTDFDSQTNV |
| | | SQSKDSDVYITDKCVLDMRSMDFKSNSAVAWSNKSDFACANAFNNSIIPEDTFFPSPESSCDV |
| | | KLVEKSFETDTNLNFQNLSVIGFRILLLKVAGFNLLMTLRLWSSGSGEGRGSLLTCGDVEENP |
| | | GPVSKGEELFTGVVPILVELDGDVNGHKFSVSGEGEGDATYGKLTLKFICTTGKLPVPWPTLV |
| | | TTLTYGVQCFSRYPDHMKQHDFFKSAMPEGYVQERTIFFKDDGNYKTRAEVKFEGDTLVNRI |
| | | ELKGIDFKEDGNILGHKLEYNYNSHNVYIMADKQKNGIKVNFKIRHNIEDGSVQLADHYQQNTP |
| | | IGDGPVLLPDNHYLSTQSALSKDPNEKRDHMVLLEFVTAAGITLGMDELYK |
| 185 | P329G VH2VL1 TCRαβ | MGWSCIILFLVATATGVHSEVQLVESGGGLVQPGGSLRLSCAASGFDFSRYWMNWVRQAPG |
| | (with SP) | KGLEWVGEITPDSSTINYAPSLKGRFTISRDNAKNSLYLQMNSLRAEDTAVYYCVRPYDYGAW |
| | [SP]-[VH2]- | FASWGQGTLVTVSSDLNKVFPPEVAVFEPSEAEISHTQKATLVCLATGFFPDHVELSWWWVNG |
| | [TRBC1_S56C]-[T2A]- | KEVHSGVCTDPQPLKEQPALNDSRYCLSSRLRVSATFWQNPRNHFRCQVQFYGLSENDEWT |
| | [SP]-[VL1]-[TRAC_T47C] | QDRAKPVTQIVSAEAWGRADCGFTSVSYQQGVLSATILYEILLGKATLYAVLVSALVLMAMVK |
| | | RKDFGSGEGRGSLLTCGDVEENPGPMGWSCIILFLVATATGVHSQAVVTQEPSLTVSPGGTV |
| | | TLTCRSSTGAVTTSNYANWVQEKPDHLFTGLIGGTNKRAPGTPARFSGSLLGGKAALTLSGA |
| | | QPEDEAEYYCALWYSNHWVFGGGTKLTVLIQNPDPAVYQLRDSKSSDKSVCLFTDFDSQTNV |
| | | SQSKDSDVYITDKCVLDMRSMDFKSNSAVAWSNKSDFACANAFNNSIIPEDTFFPSPESSCDV |
| | | KLVEKSFETDTNLNFQNLSVIGFRILLLKVAGFNLLMTLRLWSS |
| 186 | P329G VH3VL1 TCRαβ | MGWSCIILFLVATATGVHSEVQLVESGGGLVQPGGSLRLSCAASGFTFSRYWMNWVRQAPG |
| | (with SP, T2A, eGFP) | KGLEWVGEITPDSSTINYAPSLKGRFTISRDNAKNSLYLQMNSLRAEDTAVYYCARPYDYGAW |
| | [SP]-[VH3]- | FASWGQGTLVTVSSDLNKVFPPEVAVFEPSEAEISHTQKATLVCLATGFFPDHVELSWWWNG |
| | [TRBC1_S56C]-[T2A]- | KEVHSGVCTDPQPLKEQPALNDSRYCLSSRLRVSATFWQNPRNHFRCQVQFYGLSENDEWT |
| | [SP]-[VL1]- | QDRAKPVTQIVSAEAWGRADCGFTSVSYQQGVLSATILYEILLGKATLYAVLVSALVLMAMVK |
| | [TRAC_T47C]-[T2A]- | RKDFGSGEGRGSLLTCGDVEENPGPMGWSCIILFLVATATGVHSQAVVTQEPSLTVSPGGTV |
| | [eGFP] | TLTCRSSTGAVTTSNYANWVQEKPDHLFTGLIGGTNKRAPGTPARFSGSLLGGKAALTLSGA |
| | | QPEDEAEYYCALWYSNHWVFGGGTKLTVLIQNPDPAVYQLRDSKSSDKSVCLFTDFDSQTNV |
| | | SQSKDSDVYITDKCVLDMRSMDFKSNSAVAWSNKSDFACANAFNNSIIPEDTFFPSPESSCDV |
| | | KLVEKSFETDTNLNFQNLSVIGFRILLLKVAGFNLLMTLRLWSSGSGEGRGSLLTCGDVEENP |
| | | GPVSKGEELFTGVVPILVELDGDVNGHKFSVSGEGEGDATYGKLTLKFICTTGKLPVPWPTLV |
| | | TTLTYGVQCFSRYPDHMKQHDFFKSAMPEGYVQERTIFFKDDGNYKTRAEVKFEGDTLVNRI |
| | | ELKGIDFKEDGNILGHKLEYNYNSHNVYIMADKQKNGIKVNFKIRHNIEDGSVQLADHYQQNTP |
| | | IGDGPVLLPDNHYLSTQSALSKDPNEKRDHMVLLEFVTAAGITLGMDELYK |
| 187 | P329G VH3VL1 TCRαβ | MGWSCIILFLVATATGVHSEVQLVESGGGLVQPGGSLRLSCAASGFTFSRYWMNWVRQAPG |
| | (with SP) | KGLEWVGEITPDSSTINYAPSLKGRFTISRDNAKNSLYLQMNSLRAEDTAVYYCARPYDYGAW |
| | [SP]-[VH3]- | FASWGQGTLVTVSSDLNKVFPPEVAVFEPSEAEISHTQKATLVCLATGFFPDHVELSWWWVNG |
| | [TRBC1_S56C]-[T2A]- | KEVHSGVCTDPQPLKEQPALNDSRYCLSSRLRVSATFWQNPRNHFRCQVQFYGLSENDEWT |
| | [SP]-[VL1]-[TRAC_T47C] | QDRAKPVTQIVSAEAWGRADCGFTSVSYQQGVLSATILYEILLGKATLYAVLVSALVLMAMVK |
| | | RKDFGSGEGRGSLLTCGDVEENPGPMGWSCIILFLVATATGVHSQAVVTQEPSLTVSPGGTV |
| | | TLTCRSSTGAVTTSNYANWVQEKPDHLFTGLIGGTNKRAPGTPARFSGSLLGGKAALTLSGA |
| | | QPEDEAEYYCALWYSNHWVFGGGTKLTVLIQNPDPAVYQLRDSKSSDKSVCLFTDFDSQTNV |
| | | SQSKDSDVYITDKCVLDMRSMDFKSNSAVAWSNKSDFACANAFNNSIIPEDTFFPSPESSCDV |
| | | KLVEKSFETDTNLNFQNLSVIGFRILLLKVAGFNLLMTLRLWSS |
| 188 | AAA TCRαβ (with SP, | MGWSCIILFLVATATGVHSEVQLVESGGDLVKPGGSLKLSCAASGFTFSSYGMSWVRQTPDK |
| | T2A, eGFP) | RLEWVATISSGGSYIYYPDSVKGRFTISRDNAKNTLYLQMSSLKSEDTAMYYCARLGMITTGY |
| | [SP]-[VH]- | AMDYWGQGTSVTVSSDLNKVFPPEVAVFEPSEAEISHTQKATLVCLATGFFPDHVELSWWWN |
| | [TRBC1_S56C]-[T2A]- | GKEVHSGVCTDPQPLKEQPALNDSRYCLSSRLRVSATFWQNPRNHFRCQVQFYGLSENDE |
| | [SP]-[VL]-[TRAC_T47C]- | WTQDRAKPVTQIVSAEAWGRADCGFTSVSYQQGVLSATILYEILLGKATLYAVLVSALVLMAM |
| | [T2A]-[eGFP] | VKRKDFGSGEGRGSLLTCGDVEENPGPMGWSCIILFLVATATGVHSDVLMTQTPLSLPVSLG |
| | | DQASISCRSSQTIVHSTGHTYLEWFLQKPGQSPKLLIYKVSNRFSGVPDRFSGSGSGTDFTLKI |
| | | SRVEAEDLGVYYCFQGSHVPYTFGGGTKLEIKIQNPDPAVYQLRDSKSSDKSVCLFTDFDSQT |
| | | NVSQSKDSDVYITDKCVLDMRSMDFKSNSAVAWSNKSDFACANAFNNSIIPEDTFFPSPESSC |
| | | DVKLVEKSFETDTNLNFQNLSVIGFRILLLKVAGFNLLMTLRLWSSGSGEGRGSLLTCGDVEE |
| | | NPGPVSKGEELFTGVVPILVELDGDVNGHKFSVSGEGEGDATYGKLTLKFICTTGKLPVPWPT |
| | | LVTTLTYGVQCFSRYPDHMKQHDFFKSAMPEGYVQERTIFFKDDGNYKTRAEVKFEGDTLVN |
| | | RIELKGIDFKEDGNILGHKLEYNYNSHNVYIMADKQKNGIKVNFKIRHNIEDGSVQLADHYQQN |
| | | TPIGDGPVLLPDNHYLSTQSALSKDPNEKRDHMVLLEFVTAAGITLGMDELYK |
| 189 | AAA TCRαβ (with SP) | MGWSCIILFLVATATGVHSEVQLVESGGDLVKPGGSLKLSCAASGFTFSSYGMSWVRQTPDK |
| | [SP]-[VH]- | RLEWVATISSGGSYIYYPDSVKGRFTISRDNAKNTLYLQMSSLKSEDTAMYYCARLGMITTGY |
| | [TRBC1_S56C]-[T2A]- | AMDYWGQGTSVTVSSDLNKVFPPEVAVFEPSEAEISHTQKATLVCLATGFFPDHVELSWWWVN |
| | [SP]-[VL]-[TRAC_T47C] | GKEVHSGVCTDPQPLKEQPALNDSRYCLSSRLRVSATFWQNPRNHFRCQVQFYGLSENDE |
| | | WTQDRAKPVTQIVSAEAWGRADCGFTSVSYQQGVLSATILYEILLGKATLYAVLVSALVLMAM |
| | | VKRKDFGSGEGRGSLLTCGDVEENPGPMGWSCIILFLVATATGVHSDVLMTQTPLSLPVSLG |
| | | DQASISCRSSQTIVHSTGHTYLEWFLQKPGQSPKLLIYKVSNRFSGVPDRFSGSGSGTDFTLKI |
| | | SRVEAEDLGVYYCFQGSHVPYTFGGGTKLEIKIQNPDPAVYQLRDSKSSDKSVCLFTDFDSQT |
| | | NVSQSKDSDVYITDKCVLDMRSMDFKSNSAVAWSNKSDFACANAFNNSIIPEDTFFPSPESSC |
| | | DVKLVEKSFETDTNLNFQNLSVIGFRILLLKVAGFNLLMTLRLWSS |
| 190 | P329G VH1VL1 TCRαβ | MGWSCIILFLVATATGVHSQAVVTQEPSLTVSPGGTVTLTCRSSTGAVTTSNYANWVQEKPD |
| | (with SP, T2A, eGFP) | HLFTGLIGGTNKRAPGTPARFSGSLLGGKAALTLSGAQPEDEAEYYCALWYSNHWVFGGGTK |
| | [SP]-[VL1]- | LTVLIQNPDPAVYQLRDSKSSDKSVCLFTDFDSQTNVSQSKDSDVYITDKCVLDMRSMDFKSN |
| | [TRAC_T47C]-[T2A]- | SAVAWSNKSDFACANAFNNSIIPEDTFFPSPESSCDVKLVEKSFETDTNLNFQNLSVIGFRILLL |
| | [SP]-[VH1]- | KVAGFNLLMTLRLWSSGSGEGRGSLLTCGDVEENPGPMGWSCIILFLVATATGVHSDVLMTQ |
| | [TRBC1_S56C]-[T2A]- | TPLSLPVSLGDQASISCRSSQTIVHSEVQLVESGGGLVQPGGSLRLSCAASGFDFSRYWMNW |
| | [eGFP] | VRQAPGKGLEWVGEITPDSSTINYTPSLKGRFTISRDNAKNSLYLQMNSLRAEDTAVYYCVRP |
| | | YDYGAWFASWGQGTLVTVSSDLNKVFPPEVAVFEPSEAEISHTQKATLVCLATGFFPDHVEL |
| | | SWWWVNGKEVHSGVCTDPQPLKEQPALNDSRYCLSSRLRVSATFWQNPRNHFRCQVQFYGL |
| | | SENDEWTQDRAKPVTQIVSAEAWGRADCGFTSVSYQQGVLSATILYEILLGKATLYAVLVSAL |
| | | VLMAMVKRKDFGSGEGRGSLLTCGDVEENPGPVSKGEELFTGVVPILVELDGDVNGHKFSVS |
| | | GEGEGDATYGKLTLKFICTTGKLPVPWPTLVTTLTYGVQCFSRYPDHMKQHDFFKSAMPEGY |
| | | VQERTIFFKDDGNYKTRAEVKFEGDTLVNRIELKGIDFKEDGNILGHKLEYNYNSHNVYIMADK |
| | | QKNGIKVNFKIRHNIEDGSVQLADHYQQNTPIGDGPVLLPDNHYLSTQSALSKDPNEKRDHMV |
| | | LLEFVTAAGITLGMDELYK |
| 191 | P329G VH1VL1 TCRαβ | MGWSCIILFLVATATGVHSQAVVTQEPSLTVSPGGTVTLTCRSSTGAVTTSNYANWVQEKPD |
| | (with SP) | HLFTGLIGGTNKRAPGTPARFSGSLLGGKAALTLSGAQPEDEAEYYCALWYSNHWVFGGGTK |
| | [SP]-[VL1]- | LTVLIQNPDPAVYQLRDSKSSDKSVCLFTDFDSQTNVSQSKDSDVYITDKCVLDMRSMDFKSN |
| | [TRAC_T47C]-[T2A]- | SAVAWSNKSDFACANAFNNSIIPEDTFFPSPESSCDVKLVEKSFETDTNLNFQNLSVIGFRILLL |
| | [SP]-[VH1]- | KVAGFNLLMTLRLWSSGSGEGRGSLLTCGDVEENPGPMGWSCIILFLVATATGVHSDVLMTQ |
| | [TRBC1_S56C] | TPLSLPVSLGDQASISCRSSQTIVHSEVQLVESGGGLVQPGGSLRLSCAASGFDFSRYWMNW |
| | | VRQAPGKGLEWVGEITPDSSTINYTPSLKGRFTISRDNAKNSLYLQMNSLRAEDTAVYYCVRP |
| | | YDYGAWFASWGQGTLVTVSSDLNKVFPPEVAVFEPSEAEISHTQKATLVCLATGFFPDHVEL |
| | | SWWWVNGKEVHSGVCTDPQPLKEQPALNDSRYCLSSRLRVSATFWQNPRNHFRCQVQFYGL |
| | | SENDEWTQDRAKPVTQIVSAEAWGRADCGFTSVSYQQGVLSATILYEILLGKATLYAVLVSAL |
| | | VLMAMVKRKDF |
| 192 | P329G VH2VL1 TCRαβ | MGWSCIILFLVATATGVHSQAVVTQEPSLTVSPGGTVTLTCRSSTGAVTTSNYANWVQEKPD |
| | (with SP, T2A, eGFP) | HLFTGLIGGTNKRAPGTPARFSGSLLGGKAALTLSGAQPEDEAEYYCALWYSNHWVFGGGTK |
| | [SP]-[VL1]- | LTVLIQNPDPAVYQLRDSKSSDKSVCLFTDFDSQTNVSQSKDSDVYITDKCVLDMRSMDFKSN |
| | [TRAC_T47C]-[T2A]- | SAVAWSNKSDFACANAFNNSIIPEDTFFPSPESSCDVKLVEKSFETDTNLNFQNLSVIGFRILLL |
| | [SP]-[VH2]- | KVAGFNLLMTLRLWSSGSGEGRGSLLTCGDVEENPGPMGWSCIILFLVATATGVHSDVLMTQ |
| | [TRBC1_S56C]-[T2A]- | TPLSLPVSLGDQASISCRSSQTIVHSEVQLVESGGGLVQPGGSLRLSCAASGFDFSRYWMNW |
| | [eGFP] | VRQAPGKGLEWVGEITPDSSTINYAPSLKGRFTISRDNAKNSLYLQMNSLRAEDTAVYYCVRP |
| | | YDYGAWFASWGQGTLVTVSSDLNKVFPPEVAVFEPSEAEISHTQKATLVCLATGFFPDHVEL |
| | | SWWWVNGKEVHSGVCTDPQPLKEQPALNDSRYCLSSRLRVSATFWQNPRNHFRCQVQFYGL |
| | | SENDEWTQDRAKPVTQIVSAEAWGRADCGFTSVSYQQGVLSATILYEILLGKATLYAVLVSAL |
| | | VLMAMVKRKDFGSGEGRGSLLTCGDVEENPGPVSKGEELFTGVVPILVELDGDVNGHKFSVS |
| | | GEGEGDATYGKLTLKFICTTGKLPVPWPTLVTTLTYGVQCFSRYPDHMKQHDFFKSAMPEGY |
| | | VQERTIFFKDDGNYKTRAEVKFEGDTLVNRIELKGIDFKEDGNILGHKLEYNYNSHNVYIMADK |
| | | QKNGIKVNFKIRHNIEDGSVQLADHYQQNTPIGDGPVLLPDNHYLSTQSALSKDPNEKRDHMV |
| | | LLEFVTAAGITLGMDELYK |
| 193 | P329G VH2VL1 TCRαβ | MGWSCIILFLVATATGVHSQAVVTQEPSLTVSPGGTVTLTCRSSTGAVTTSNYANWVQEKPD |
| | (with SP) | HLFTGLIGGTNKRAPGTPARFSGSLLGGKAALTLSGAQPEDEAEYYCALWYSNHWVFGGGTK |
| | [SP]-[VL1]- | LTVLIQNPDPAVYQLRDSKSSDKSVCLFTDFDSQTNVSQSKDSDVYITDKCVLDMRSMDFKSN |
| | [TRAC_T47C]-[T2A]- | SAVAWSNKSDFACANAFNNSIIPEDTFFPSPESSCDVKLVEKSFETDTNLNFQNLSVIGFRILLL |
| | [SP]-[VH2]- | KVAGFNLLMTLRLWSSGSGEGRGSLLTCGDVEENPGPMGWSCIILFLVATATGVHSDVLMTQ |
| | [TRBC1_S56C] | TPLSLPVSLGDQASISCRSSQTIVHSEVQLVESGGGLVQPGGSLRLSCAASGFDFSRYWMNW |
| | | VRQAPGKGLEWVGEITPDSSTINYAPSLKGRFTISRDNAKNSLYLQMNSLRAEDTAVYYCVRP |
| | | YDYGAWFASWGQGTLVTVSSDLNKVFPPEVAVFEPSEAEISHTQKATLVCLATGFFPDHVEL |
| | | SWWWVNGKEVHSGVCTDPQPLKEQPALNDSRYCLSSRLRVSATFWQNPRNHFRCQVQFYGL |
| | | SENDEWTQDRAKPVTQIVSAEAWGRADCGFTSVSYQQGVLSATILYEILLGKATLYAVLVSAL |
| | | VLMAMVKRKDF |
| 194 | P329G VH3VL1 TCRαβ | MGWSCIILFLVATATGVHSQAVVTQEPSLTVSPGGTVTLTCRSSTGAVTTSNYANWVQEKPD |
| | (with SP, T2A, eGFP) | HLFTGLIGGTNKRAPGTPARFSGSLLGGKAALTLSGAQPEDEAEYYCALWYSNHWVFGGGTK |
| | [SP]-[VL1]- | LTVLIQNPDPAVYQLRDSKSSDKSVCLFTDFDSQTNVSQSKDSDVYITDKCVLDMRSMDFKSN |
| | [TRAC_T47C]-[T2A]- | SAVAWSNKSDFACANAFNNSIIPEDTFFPSPESSCDVKLVEKSFETDTNLNFQNLSVIGFRILLL |
| | [SP]-[VH3]- | KVAGFNLLMTLRLWSSGSGEGRGSLLTCGDVEENPGPMGWSCIILFLVATATGVHSDVLMTQ |
| | [TRBC1_S56C]-[T2A]- | TPLSLPVSLGDQASISCRSSQTIVHSEVQLVESGGGLVQPGGSLRLSCAASGFTFSRYWMNW |
| | [eGFP] | VRQAPGKGLEWVGEITPDSSTINYAPSLKGRFTISRDNAKNSLYLQMNSLRAEDTAVYYCARP |
| | | YDYGAWFASWGQGTLVTVSSDLNKVFPPEVAVFEPSEAEISHTQKATLVCLATGFFPDHVEL |
| | | SWWWVNGKEVHSGVCTDPQPLKEQPALNDSRYCLSSRLRVSATFWQNPRNHFRCQVQFYGL |
| | | SENDEWTQDRAKPVTQIVSAEAWGRADCGFTSVSYQQGVLSATILYEILLGKATLYAVLVSAL |
| | | VLMAMVKRKDFGSGEGRGSLLTCGDVEENPGPVSKGEELFTGVVPILVELDGDVNGHKFSVS |
| | | GEGEGDATYGKLTLKFICTTGKLPVPWPTLVTTLTYGVQCFSRYPDHMKQHDFFKSAMPEGY |
| | | VQERTIFFKDDGNYKTRAEVKFEGDTLVNRIELKGIDFKEDGNILGHKLEYNYNSHNVYIMADK |
| | | QKNGIKVNFKIRHNIEDGSVQLADHYQQNTPIGDGPVLLPDNHYLSTQSALSKDPNEKRDHMV |
| | | LLEFVTAAGITLGMDELYK |
| 195 | P329G VH3VL1 TCRαβ | MGWSCIILFLVATATGVHSQAVVTQEPSLTVSPGGTVTLTCRSSTGAVTTSNYANWVQEKPD |
| | (with SP) | HLFTGLIGGTNKRAPGTPARFSGSLLGGKAALTLSGAQPEDEAEYYCALWYSNHWVFGGGTK |
| | [SP]-[VL1]- | LTVLIQNPDPAVYQLRDSKSSDKSVCLFTDFDSQTNVSQSKDSDVYITDKCVLDMRSMDFKSN |
| | [TRAC_T47C]-[T2A]- | SAVAWSNKSDFACANAFNNSIIPEDTFFPSPESSCDVKLVEKSFETDTNLNFQNLSVIGFRILLL |
| | [SP]-[VH3]- | KVAGFNLLMTLRLWSSGSGEGRGSLLTCGDVEENPGPMGWSCIILFLVATATGVHSDVLMTQ |
| | [TRBC1_S56C] | TPLSLPVSLGDQASISCRSSQTIVHSEVQLVESGGGLVQPGGSLRLSCAASGFTFSRYWMNW |
| | | VRQAPGKGLEWVGEITPDSSTINYAPSLKGRFTISRDNAKNSLYLQMNSLRAEDTAVYYCARP |
| | | YDYGAWFASWGQGTLVTVSSDLNKVFPPEVAVFEPSEAEISHTQKATLVCLATGFFPDHVEL |
| | | SWWWVNGKEVHSGVCTDPQPLKEQPALNDSRYCLSSRLRVSATFWQNPRNHFRCQVQFYGL |
| | | SENDEWTQDRAKPVTQIVSAEAWGRADCGFTSVSYQQGVLSATILYEILLGKATLYAVLVSAL |
| | | VLMAMVKRKDF |
| 196 | AAA TCRαβ (with SP, | MGWSCIILFLVATATGVHSDVLMTQTPLSLPVSLGDQASISCRSSQTIVHSTGHTYLEWFLQKP |
| | T2A, eGFP) | GQSPKLLIYKVSNRFSGVPDRFSGSGSGTDFTLKISRVEAEDLGVYYCFQGSHVPYTFGGGT |
| | [SP]-[VL]-[TRAC_T47C]- | KLEIKIQNPDPAVYQLRDSKSSDKSVCLFTDFDSQTNVSQSKDSDVYITDKCVLDMRSMDFKS |
| | [T2A]-[SP]-[VH]- | NSAVAWSNKSDFACANAFNNSIIPEDTFFPSPESSCDVKLVEKSFETDTNLNFQNLSVIGFRILL |
| | [TRBC1_S56C]-[T2A]- | LKVAGFNLLMTLRLWSSGSGEGRGSLLTCGDVEENPGPMGWSCIILFLVATATGVHSDVLMT |
| | [eGFP] | QTPLSLPVSLGDQASISCRSSQTIVHSEVQLVESGGDLVKPGGSLKLSCAASGFTFSSYGMS |
| | | WVRQTPDKRLEWVATISSGGSYIYYPDSVKGRFTISRDNAKNTLYLQMSSLKSEDTAMYYCA |
| | | RLGMITTGYAMDYWGQGTSVTVSSDLNKVFPPEVAVFEPSEAEISHTQKATLVCLATGFFPDH |
| | | VELSWWWVNGKEVHSGVCTDPQPLKEQPALNDSRYCLSSRLRVSATFWQNPRNHFRCQVQF |
| | | YGLSENDEWTQDRAKPVTQIVSAEAWGRADCGFTSVSYQQGVLSATILYEILLGKATLYAVLV |
| | | SALVLMAMVKRKDFGSGEGRGSLLTCGDVEENPGPVSKGEELFTGVVPILVELDGDVNGHKF |
| | | SVSGEGEGDATYGKLTLKFICTTGKLPVPWPTLVTTLTYGVQCFSRYPDHMKQHDFFKSAMP |
| | | EGYVQERTIFFKDDGNYKTRAEVKFEGDTLVNRIELKGIDFKEDGNILGHKLEYNYNSHNVYIM |
| | | ADKQKNGIKVNFKIRHNIEDGSVQLADHYQQNTPIGDGPVLLPDNHYLSTQSALSKDPNEKRD |
| | | HMVLLEFVTAAGITLGMDELYK |
| 197 | AAA TCRαβ (with SP) | MGWSCIILFLVATATGVHSDVLMTQTPLSLPVSLGDQASISCRSSQTIVHSTGHTYLEWFLQKP |
| | [SP]-[VL]-[TRAC_T47C]- | GQSPKLLIYKVSNRFSGVPDRFSGSGSGTDFTLKISRVEAEDLGVYYCFQGSHVPYTFGGGT |
| | [T2A]-[SP]-[VH]- | KLEIKIQNPDPAVYQLRDSKSSDKSVCLFTDFDSQTNVSQSKDSDVYITDKCVLDMRSMDFKS |
| | [TRBC1_S56C] | NSAVAWSNKSDFACANAFNNSIIPEDTFFPSPESSCDVKLVEKSFETDTNLNFQNLSVIGFRILL |
| | | LKVAGFNLLMTLRLWSSGSGEGRGSLLTCGDVEENPGPMGWSCIILFLVATATGVHSDVLMT |
| | | QTPLSLPVSLGDQASISCRSSQTIVHSEVQLVESGGDLVKPGGSLKLSCAASGFTFSSYGMS |
| | | WVRQTPDKRLEWVATISSGGSYIYYPDSVKGRFTISRDNAKNTLYLQMSSLKSEDTAMYYCA |
| | | RLGMITTGYAMDYWGQGTSVTVSSDLNKVFPPEVAVFEPSEAEISHTQKATLVCLATGFFPDH |
| | | VELSWWWWVNGKEVHSGVCTDPQPLKEQPALNDSRYCLSSRLRVSATFWQNPRNHFRCQVQF |
| | | YGLSENDEWTQDRAKPVTQIVSAEAWGRADCGFTSVSYQQGVLSATILYEILLGKATLYAVLV |
| | | SALVLMAMVKRKDF |
| 198 | P329G VH1VL1 TCRαβ | MGWSCIILFLVATATGVHSQAVVTQEPSLTVSPGGTVTLTCRSSTGAVTTSNYANWVQEKPD |
| | (with SP, T2A, eGFP) | HLFTGLIGGTNKRAPGTPARFSGSLLGGKAALTLSGAQPEDEAEYYCALWYSNHWVFGGGTK |
| | [SP]-[VL1]- | LTVLDLNKVFPPEVAVFEPSEAEISHTQKATLVCLATGFFPDHVELSWWWVNGKEVHSGVCTDP |
| | [TRBC1_S56C]-[T2A]- | QPLKEQPALNDSRYCLSSRLRVSATFWQNPRNHFRCQVQFYGLSENDEWTQDRAKPVTQIV |
| | [SP]-[VH1]- | SAEAWGRADCGFTSVSYQQGVLSATILYEILLGKATLYAVLVSALVLMAMVKRKDFGSGEGR |
| | [TRAC_T47C]-[T2A]- | GSLLTCGDVEENPGPMGWSCIILFLVATATGVHSDVLMTQTPLSLPVSLGDQASISCRSSQTIV |
| | [eGFP] | HSEVQLVESGGGLVQPGGSLRLSCAASGFDFSRYWMNWVRQAPGKGLEWVGEITPDSSTIN |
| | | YTPSLKGRFTISRDNAKNSLYLQMNSLRAEDTAVYYCVRPYDYGAWFASWGQGTLVTVSSIQ |
| | | NPDPAVYQLRDSKSSDKSVCLFTDFDSQTNVSQSKDSDVYITDKCVLDMRSMDFKSNSAVA |
| | | WSNKSDFACANAFNNSIIPEDTFFPSPESSCDVKLVEKSFETDTNLNFQNLSVIGFRILLLKVAG |
| | | FNLLMTLRLWSSGSGEGRGSLLTCGDVEENPGPVSKGEELFTGVVPILVELDGDVNGHKFSV |
| | | SGEGEGDATYGKLTLKFICTTGKLPVPWPTLVTTLTYGVQCFSRYPDHMKQHDFFKSAMPEG |
| | | YVQERTIFFKDDGNYKTRAEVKFEGDTLVNRIELKGIDFKEDGNILGHKLEYNYNSHNVYIMAD |
| | | KQKNGIKVNFKIRHNIEDGSVQLADHYQQNTPIGDGPVLLPDNHYLSTQSALSKDPNEKRDHM |
| | | VLLEFVTAAGITLGMDELYK |
| 199 | P329G VH1VL1 TCRαβ | MGWSCIILFLVATATGVHSQAVVTQEPSLTVSPGGTVTLTCRSSTGAVTTSNYANWVQEKPD |
| | (with SP) | HLFTGLIGGTNKRAPGTPARFSGSLLGGKAALTLSGAQPEDEAEYYCALWYSNHWVFGGGTK |
| | [SP]-[VL1]- | LTVLDLNKVFPPEVAVFEPSEAEISHTQKATLVCLATGFFPDHVELSWWWVNGKEVHSGVCTDP |
| | [TRBC1_S56C]-[T2A]- | QPLKEQPALNDSRYCLSSRLRVSATFWQNPRNHFRCQVQFYGLSENDEWTQDRAKPVTQIV |
| | [SP]-[VH1]-[TRAC_T47C] | SAEAWGRADCGFTSVSYQQGVLSATILYEILLGKATLYAVLVSALVLMAMVKRKDFGSGEGR |
| | | GSLLTCGDVEENPGPMGWSCIILFLVATATGVHSDVLMTQTPLSLPVSLGDQASISCRSSQTIV |
| | | HSEVQLVESGGGLVQPGGSLRLSCAASGFDFSRYWMNWVRQAPGKGLEWVGEITPDSSTIN |
| | | YTPSLKGRFTISRDNAKNSLYLQMNSLRAEDTAVYYCVRPYDYGAWFASWGQGTLVTVSSIQ |
| | | NPDPAVYQLRDSKSSDKSVCLFTDFDSQTNVSQSKDSDVYITDKCVLDMRSMDFKSNSAVA |
| | | WSNKSDFACANAFNNSIIPEDTFFPSPESSCDVKLVEKSFETDTNLNFQNLSVIGFRILLLKVAG |
| | | FNLLMTLRLWSS |
| 200 | P329G VH2VL1 TCRαβ | MGWSCIILFLVATATGVHSQAVVTQEPSLTVSPGGTVTLTCRSSTGAVTTSNYANWVQEKPD |
| | (with SP, T2A, eGFP) | HLFTGLIGGTNKRAPGTPARFSGSLLGGKAALTLSGAQPEDEAEYYCALWYSNHWVFGGGTK |
| | [SP]-[VL1]- | LTVLDLNKVFPPEVAVFEPSEAEISHTQKATLVCLATGFFPDHVELSWWWVNGKEVHSGVCTDP |
| | [TRBC1_S56C]-[T2A]- | QPLKEQPALNDSRYCLSSRLRVSATFWQNPRNHFRCQVQFYGLSENDEWTQDRAKPVTQIV |
| | [SP]-[VH2]- | SAEAWGRADCGFTSVSYQQGVLSATILYEILLGKATLYAVLVSALVLMAMVKRKDFGSGEGR |
| | [TRAC_T47C]-[T2A]- | GSLLTCGDVEENPGPMGWSCIILFLVATATGVHSDVLMTQTPLSLPVSLGDQASISCRSSQTIV |
| | [eGFP] | HSEVQLVESGGGLVQPGGSLRLSCAASGFDFSRYWMNWVRQAPGKGLEWVGEITPDSSTIN |
| | | YAPSLKGRFTISRDNAKNSLYLQMNSLRAEDTAVYYCVRPYDYGAWFASWGQGTLVTVSSIQ |
| | | NPDPAVYQLRDSKSSDKSVCLFTDFDSQTNVSQSKDSDVYITDKCVLDMRSMDFKSNSAVA |
| | | WSNKSDFACANAFNNSIIPEDTFFPSPESSCDVKLVEKSFETDTNLNFQNLSVIGFRILLLKVAG |
| | | FNLLMTLRLWSSGSGEGRGSLLTCGDVEENPGPVSKGEELFTGVVPILVELDGDVNGHKFSV |
| | | SGEGEGDATYGKLTLKFICTTGKLPVPWPTLVTTLTYGVQCFSRYPDHMKQHDFFKSAMPEG |
| | | YVQERTIFFKDDGNYKTRAEVKFEGDTLVNRIELKGIDFKEDGNILGHKLEYNYNSHNVYIMAD |
| | | KQKNGIKVNFKIRHNIEDGSVQLADHYQQNTPIGDGPVLLPDNHYLSTQSALSKDPNEKRDHM |
| | | VLLEFVTAAGITLGMDELYK |
| 201 | P329G VH2VL1 TCRαβ | MGWSCIILFLVATATGVHSQAVVTQEPSLTVSPGGTVTLTCRSSTGAVTTSNYANWVQEKPD |
| | (with SP) | HLFTGLIGGTNKRAPGTPARFSGSLLGGKAALTLSGAQPEDEAEYYCALWYSNHWVFGGGTK |
| | [SP]-[VL1]- | LTVLDLNKVFPPEVAVFEPSEAEISHTQKATLVCLATGFFPDHVELSWWWVNGKEVHSGVCTDP |
| | [TRBC1_S56C]-[T2A]- | QPLKEQPALNDSRYCLSSRLRVSATFWQNPRNHFRCQVQFYGLSENDEWTQDRAKPVTQIV |
| | [SP]-[VH2]-[TRAC_T47C] | SAEAWGRADCGFTSVSYQQGVLSATILYEILLGKATLYAVLVSALVLMAMVKRKDFGSGEGR |
| | | GSLLTCGDVEENPGPMGWSCIILFLVATATGVHSDVLMTQTPLSLPVSLGDQASISCRSSQTIV |
| | | HSEVQLVESGGGLVQPGGSLRLSCAASGFDFSRYWMNWVRQAPGKGLEWVGEITPDSSTIN |
| | | YAPSLKGRFTISRDNAKNSLYLQMNSLRAEDTAVYYCVRPYDYGAWFASWGQGTLVTVSSIQ |
| | | NPDPAVYQLRDSKSSDKSVCLFTDFDSQTNVSQSKDSDVYITDKCVLDMRSMDFKSNSAVA |
| | | WSNKSDFACANAFNNSIIPEDTFFPSPESSCDVKLVEKSFETDTNLNFQNLSVIGFRILLLKVAG |
| | | FNLLMTLRLWSS |
| 202 | P329G VH3VL1 TCRαβ | MGWSCIILFLVATATGVHSQAVVTQEPSLTVSPGGTVTLTCRSSTGAVTTSNYANWVQEKPD |
| | (with SP, T2A, eGFP) | HLFTGLIGGTNKRAPGTPARFSGSLLGGKAALTLSGAQPEDEAEYYCALWYSNHWVFGGGTK |
| | [SP]-[VL1]- | LTVLDLNKVFPPEVAVFEPSEAEISHTQKATLVCLATGFFPDHVELSWWWVNGKEVHSGVCTDP |
| | [TRBC1_S56C]-[T2A]- | QPLKEQPALNDSRYCLSSRLRVSATFWQNPRNHFRCQVQFYGLSENDEWTQDRAKPVTQIV |
| | [SP]-[VH3]- | SAEAWGRADCGFTSVSYQQGVLSATILYEILLGKATLYAVLVSALVLMAMVKRKDFGSGEGR |
| | [TRAC_T47C]-[T2A]- | GSLLTCGDVEENPGPMGWSCIILFLVATATGVHSDVLMTQTPLSLPVSLGDQASISCRSSQTIV |
| | [eGFP] | HSEVQLVESGGGLVQPGGSLRLSCAASGFTFSRYWMNWVRQAPGKGLEWVGEITPDSSTIN |
| | | YAPSLKGRFTISRDNAKNSLYLQMNSLRAEDTAVYYCARPYDYGAWFASWGQGTLVTVSSIQ |
| | | NPDPAVYQLRDSKSSDKSVCLFTDFDSQTNVSQSKDSDVYITDKCVLDMRSMDFKSNSAVA |
| | | WSNKSDFACANAFNNSIIPEDTFFPSPESSCDVKLVEKSFETDTNLNFQNLSVIGFRILLLKVAG |
| | | FNLLMTLRLWSSGSGEGRGSLLTCGDVEENPGPVSKGEELFTGVVPILVELDGDVNGHKFSV |
| | | SGEGEGDATYGKLTLKFICTTGKLPVPWPTLVTTLTYGVQCFSRYPDHMKQHDFFKSAMPEG |
| | | YVQERTIFFKDDGNYKTRAEVKFEGDTLVNRIELKGIDFKEDGNILGHKLEYNYNSHNVYIMAD |
| | | KQKNGIKVNFKIRHNIEDGSVQLADHYQQNTPIGDGPVLLPDNHYLSTQSALSKDPNEKRDHM |
| | | VLLEFVTAAGITLGMDELYK |
| 203 | P329G VH3VL1 TCRαβ | MGWSCIILFLVATATGVHSQAVVTQEPSLTVSPGGTVTLTCRSSTGAVTTSNYANWVQEKPD |
| | (with SP) | HLFTGLIGGTNKRAPGTPARFSGSLLGGKAALTLSGAQPEDEAEYYCALWYSNHWVFGGGTK |
| | [SP]-[VL1]- | LTVLDLNKVFPPEVAVFEPSEAEISHTQKATLVCLATGFFPDHVELSWWVNGKEVHSGVCTDP |
| | [TRBC1_S56C]-[T2A]- | QPLKEQPALNDSRYCLSSRLRVSATFWQNPRNHFRCQVQFYGLSENDEWTQDRAKPVTQIV |
| | [SP]-[VH3]-[TRAC_T47C] | SAEAWGRADCGFTSVSYQQGVLSATILYEILLGKATLYAVLVSALVLMAMVKRKDFGSGEGR |
| | | GSLLTCGDVEENPGPMGWSCIILFLVATATGVHSDVLMTQTPLSLPVSLGDQASISCRSSQTIV |
| | | HSEVQLVESGGGLVQPGGSLRLSCAASGFTFSRYWMNWVRQAPGKGLEWVGEITPDSSTIN |
| | | YAPSLKGRFTISRDNAKNSLYLQMNSLRAEDTAVYYCARPYDYGAWFASWGQGTLVTVSSIQ |
| | | NPDPAVYQLRDSKSSDKSVCLFTDFDSQTNVSQSKDSDVYITDKCVLDMRSMDFKSNSAVA |
| | | WSNKSDFACANAFNNSIIPEDTFFPSPESSCDVKLVEKSFETDTNLNFQNLSVIGFRILLLKVAG |
| | | FNLLMTLRLWSS |
| 204 | AAA TCRαβ (with SP, | MGWSCIILFLVATATGVHSDVLMTQTPLSLPVSLGDQASISCRSSQTIVHSTGHTYLEWFLQKP |
| | T2A, eGFP) | GQSPKLLIYKVSNRFSGVPDRFSGSGSGTDFTLKISRVEAEDLGVYYCFQGSHVPYTFGGGT |
| | [SP]-[VL]- | KLEIKDLNKVFPPEVAVFEPSEAEISHTQKATLVCLATGFFPDHVELSWWWWVNGKEVHSGVCTD |
| | [TRBC1_S56C]-[T2A]- | PQPLKEQPALNDSRYCLSSRLRVSATFWQNPRNHFRCQVQFYGLSENDEWTQDRAKPVTQI |
| | [SP]-[VH]-[TRAC_T47C]- | VSAEAWGRADCGFTSVSYQQGVLSATILYEILLGKATLYAVLVSALVLMAMVKRKDFGSGEGR |
| | [T2A]-[eGFP] | GSLLTCGDVEENPGPMGWSCIILFLVATATGVHSDVLMTQTPLSLPVSLGDQASISCRSSQTIV |
| | | HSEVQLVESGGDLVKPGGSLKLSCAASGFTFSSYGMSWVRQTPDKRLEWVATISSGGSYIYY |
| | | PDSVKGRFTISRDNAKNTLYLQMSSLKSEDTAMYYCARLGMITTGYAMDYWGQGTSVTVSSI |
| | | QNPDPAVYQLRDSKSSDKSVCLFTDFDSQTNVSQSKDSDVYITDKCVLDMRSMDFKSNSAVA |
| | | WSNKSDFACANAFNNSIIPEDTFFPSPESSCDVKLVEKSFETDTNLNFQNLSVIGFRILLLKVAG |
| | | FNLLMTLRLWSSGSGEGRGSLLTCGDVEENPGPVSKGEELFTGVVPILVELDGDVNGHKFSV |
| | | SGEGEGDATYGKLTLKFICTTGKLPVPWPTLVTTLTYGVQCFSRYPDHMKQHDFFKSAMPEG |
| | | YVQERTIFFKDDGNYKTRAEVKFEGDTLVNRIELKGIDFKEDGNILGHKLEYNYNSHNVYIMAD |
| | | KQKNGIKVNFKIRHNIEDGSVQLADHYQQNTPIGDGPVLLPDNHYLSTQSALSKDPNEKRDHM |
| | | VLLEFVTAAGITLGMDELYK |
| 205 | AAA TCRαβ (with SP) | MGWSCIILFLVATATGVHSDVLMTQTPLSLPVSLGDQASISCRSSQTIVHSTGHTYLEWFLQKP |
| | [SP]-[VL]- | GQSPKLLIYKVSNRFSGVPDRFSGSGSGTDFTLKISRVEAEDLGVYYCFQGSHVPYTFGGGT |
| | [TRBC1_S56C]-[T2A]- | KLEIKDLNKVFPPEVAVFEPSEAEISHTQKATLVCLATGFFPDHVELSWWVNGKEVHSGVCTD |
| | [SP]-[VH]-[TRAC_T47C] | PQPLKEQPALNDSRYCLSSRLRVSATFWQNPRNHFRCQVQFYGLSENDEWTQDRAKPVTQI |
| | | VSAEAWGRADCGFTSVSYQQGVLSATILYEILLGKATLYAVLVSALVLMAMVKRKDFGSGEGR |
| | | GSLLTCGDVEENPGPMGWSCIILFLVATATGVHSDVLMTQTPLSLPVSLGDQASISCRSSQTIV |
| | | HSEVQLVESGGDLVKPGGSLKLSCAASGFTFSSYGMSWVRQTPDKRLEWVATISSGGSYIYY |
| | | PDSVKGRFTISRDNAKNTLYLQMSSLKSEDTAMYYCARLGMITTGYAMDYWGQGTSVTVSSI |
| | | QNPDPAVYQLRDSKSSDKSVCLFTDFDSQTNVSQSKDSDVYITDKCVLDMRSMDFKSNSAVA |
| | | WSNKSDFACANAFNNSIIPEDTFFPSPESSCDVKLVEKSFETDTNLNFQNLSVIGFRILLLKVAG |
| | | FNLLMTLRLWSS |
| 206 | Human IgG1 CH2-CH3 | APELLGGPSVFLFPPKPKDTLMISRTPEVTCVVVDVSHEDPEVKFNWYVDGVEVHNAKTKPR |
| | region (G1m1) | EEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 207 | Human IgG1 CH2-CH3 | APELLGGPSVFLFPPKPKDTLMISRTPEVTCVVVDVSHEDPEVKFNWYVDGVEVHNAKTKPR |
| | region (G1m3) | EEQYNSTYRVVSVLTVLHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 208 | Human IgG2 CH2-CH3 | APPVAGPSVFLFPPKPKDTLMISRTPEVTCVWVDVSHEDPEVQFNWYVDGVEVHNAKTKPRE |
| | region | EQFNSTFRVVSVLTVVHQDWLNGKEYKCKVSNKGLPAPIEKTISKTKGQPREPQVYTLPPSRE |
| | | EMTKNQVSLTCLVKGFYPSDISVEWESNGQPENNYKTTPPMLDSDGSFFLYSKLTVDKSRWQ |
| | | QGNVFSCSVMHEALHNHYTQKSLSLSPGK |
| 209 | Human IgG3 CH2-CH3 | APELLGGPSVFLFPPKPKDTLMISRTPEVTCVVVDVSHEDPEVQFKWYVDGVEVHNAKTKPR |
| | region | EEQYNSTFRVVSVLTVLHQDWLNGKEYKCKVSNKALPAPIEKTISKTKGQPREPQVYTLPPSR |
| | | EEMTKNQVSLTCLVKGFYPSDIAVEWESSGQPENNYNTTPPMLDSDGSFFLYSKLTVDKSRW |
| | | QQGNIFSCSVMHEALHNRFTQKSLSLSPGK |
| 210 | Human IgG4 CH2-CH3 | APEFLGGPSVFLFPPKPKDTLMISRTPEVTCVVVDVSQEDPEVQFNWYVDGVEVHNAKTKPR |
| | region | EEQFNSTYRVVSVLTVLHQDWLNGKEYKCKVSNKGLPSSIEKTISKAKGQPREPQVYTLPPSQ |
| | | EEMTKNQVSLTCLVKGFYPSDIAVEWESNGQPENNYKTTPPVLDSDGSFFLYSRLTVDKSRW |
| | | QEGNVFSCSVMHEALHNHYTQKSLSLSLGK |
| 211 | Human IgG1 CH2-CH3 | APELLGGPSVFLFPPKPKDTLMISRTPEVTCVVVDVSHEDPEVKFNWYVDGVEVHNAKTKPR |
| | (G1m1) region P329G | EEQYNSTYRVVSVLTVLHQDWLNGKEYKCKVSNKALGAPIEKTISKAKGQPREPQVYTLPPSR |
| | | DELTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 212 | Human IgG1 CH2-CH3 | APELLGGPSVFLFPPKPKDTLMISRTPEVTCVVVDVSHEDPEVKFNWYVDGVEVHNAKTKPR |
| | (G1m1) region S298A, | EEQYNATYRVVSVLTVLHQDWLNGKEYKCKVSNKALPAPIAATISKAKGQPREPQVYTLPPSR |
| | E333A, K334A | DELTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 213 | Human IgG1 CH2-CH3 | APEAAGGPSVFLFPPKPKDTLMISRTPEVTCVVVDVSHEDPEVKFNWYVDGVEVHNAKTKPR |
| | (G1m1) region L234A, | EEQYNSTYRVVSVLTVLHQDWLNGKEYKCKVSNKALGAPIEKTISKAKGQPREPQVYTLPPSR |
| | L235A, P329G | DELTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 214 | Human IgG1 CH2-CH3 | APELLGGPSVFLFPPKPKDTLMISRTPEVTCVVVDVSHEDPEVKFNWYVDGVEVHNAKTKPR |
| | (G1m3) region P329G | EEQYNSTYRVVSVLTVLHQDWLNGKEYKCKVSNKALGAPIEKTISKAKGQPREPQVYTLPPSR |
| | | EEMTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 215 | Human IgG1 CH2-CH3 | APELLGGPSVFLFPPKPKDTLMISRTPEVTCVVVDVSHEDPEVKFNWYVDGVEVHNAKTKPR |
| | (G1m3) region S298A, | EEQYNATYRVVSVLTVLHQDWLNGKEYKCKVSNKALPAPIAATISKAKGQPREPQVYTLPPSR |
| | E333A, K334A | EEMTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 216 | Human IgG1 CH2-CH3 | APEAAGGPSVFLFPPKPKDTLMISRTPEVTCVVVDVSHEDPEVKFNWYVDGVEVHNAKTKPR |
| | (G1m3) region L234A, | EEQYNSTYRVVSVLTVLHQDWLNGKEYKCKVSNKALGAPIEKTISKAKGQPREPQVYTLPPSR |
| | L235A, P329G | EEMTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| 217 | sgRNA targeting Exon 7 | GGUGGCCACAAUUGUCAUAGGUUUUAGAGCUAGAAAUAGCAAGUUAAAAUAAGGCUAG |
| | of CD3E | UCCGUUAUCAACUUGAAAAAGUGGCACCGAGUCGGUGCUUUU |
| 218 | sgRNA targeting TRBC1 | GGAGAAUGACGAGUGGACCCGUUUUAGAGCUAGAAAUAGCAAGUUAAAAUAA |
| | | GGCUAGUCCGUUAUCAACUUGAAAAAGUGGCACCGAGUCGGUGCUUUU |
| 219 | sgRNA targeting TRAC | AGAGUCUCUCAGCUGGUACAGUUUUAGAGCUAGAAAUAGCAAGUUAAAAUAAG |
| | | GCUAGUCCGUUAUCAACUUGAAAAAGUGGCACCGAGUCGGUGCUUUU |
| 220 | P2A cleavage site | GSGATNFSLLKQAGDVEENPGP |
| 221 | F2A cleavage site | GSGVKQTLNFDLLKLAGDVESNPGP |
| 222 | ITAM consensus | YXXL/I |
| | | wherein X = any amino acid |
| 223 | Larger ITAM consensus | YXXL/I(X)₆₋₈YXXL/I |
| | | wherein X = any amino acid |
| 224 | Nucleotide encoding | GAAGTGCAGCTGGTTGAGTCTGGCGGAGGACTGGTTCAACCTGGCGGAAGCCTGAGACT |
| | P329G VH3VL1 CD38 | GTCTTGTGCCGCCAGCGGCTTCACCTTCAGCAGATACTGGATGAACTGGGTGAGACAGG |
| | (mature) | CCCCTGGCAAAGGACTGGAGTGGGTGGGAGAGATCACCCCTGACAGCAGCACCATCAAT |
| | [VH3VL1 scFv]-[CD3ϵ | TACGCCCCTAGCCTGAAGGGCAGATTCACCATCTCCAGAGACAACGCCAAGAACAGCCT |
| | ECD]-[CD3ϵ TMD]-[CD3ϵ | GTACCTGCAGATGAACTCCCTGAGAGCCGAGGACACCGCCGTGTACTACTGTGCCAGAC |
| | ICD] | CTTACGATTACGGCGCCTGGTTTGCTTCTTGGGGCCAGGGAACACTGGTGACAGTTAGCT |
| | | CTGGAGGAGGAGGATCAGGCGGAGGCGGAAGTGGTGGTGGAGGATCTCAGGCTGTGGT |
| | | GACACAAGAGCCAAGCCTGACAGTGTCTCCAGGCGGCACAGTGACCCTGACCTGTAGAT |
| | | CTTCTACCGGAGCCGTGACCACCAGCAACTACGCCAATTGGGTGCAAGAGAAGCCCGAC |
| | | CACCTGTTTACAGGCCTGATCGGCGGCACCAATAAGAGAGCACCTGGCACACCAGCCAG |
| | | ATTCTCTGGATCTCTGCTCGGAGGAAAGGCCGCTCTGACACTGTCTGGAGCCCAGCCAG |
| | | AGGATGAGGCCGAGTATTATTGTGCCCTGTGGTACAGCAACCACTGGGTGTTTGGCGGC |
| | | GGAACAAAGCTGACAGTTCTTGGAGGTGGTGGAAGCGGTGGAGGTGGATCTGGAGGTGG |
| | | CGGATCCGATGGAAATGAGGAGATGGGCGGCATCACCCAGACACCTTACAAGGTGAGCA |
| | | TCAGCGGCACCACCGTGATCCTGACATGTCCTCAGTACCCTGGCAGCGAGATCCTGTGG |
| | | CAGCACAACGATAAGAACATCGGCGGCGACGAGGACGACAAGAATATCGGCTCTGACGA |
| | | GGATCACCTGTCTCTGAAGGAGTTCAGCGAGCTGGAGCAGAGCGGCTACTACGTGTGTT |
| | | ACCCTAGAGGCAGCAAGCCCGAGGACGCCAACTTCTACCTGTATCTGAGAGCCAGAGTG |
| | | TGCGAGAACTGCATGGAGATGGATGTGATGAGTGTCGCTACCATCGTGATCGTCGACATC |
| | | TGTATCACAGGCGGCCTGCTGCTGCTGGTGTACTATTGGAGCAAGAACCGGAAGGCCAA |
| | | AGCCAAGCCAGTGACAAGAGGAGCAGGAGCCGGAGGTAGACAGAGAGGCCAGAACAAA |
| | | GAAAGGCCTCCACCGGTGCCTAATCCTGACTACGAGCCCATCAGAAAGGGCCAGAGAGA |
| | | TCTGTACAGCGGCCTGAACCAGAGAAGAATC |
| 225 | Nucleotide encoding | GAGGTGCAGCTGGTGGAGAGCGGCGGCGGCCTGGTGCAGCCCGGCGGCAGCCTGAGG |
| | P329G TCRαβ- | CTGAGCTGCGCCGCCAGCGGCTTCACCTTCAGCAGGTACTGGATGAACTGGGTGAGGCA |
| | VH3TRAC (mature) | GGCCCCCGGCAAGGGCCTGGAGTGGGTGGGCGAGATCACCCCCGACAGCAGCACCATC |
| | [VH3]-[TRAC_T47C] | AACTACGCCCCCAGCCTGAAGGGCAGGTTCACCATCAGCAGGGACAACGCCAAGAACAG |
| | | CCTGTACCTGCAGATGAACAGCCTGAGGGCCGAGGACACCGCCGTGTACTACTGCGCCA |
| | | GGCCCTACGACTACGGCGCCTGGTTCGCCAGCTGGGGCCAGGGCACCCTGGTGACCGT |
| | | GAGCAGCATCCAGAACCCCGACCCCGCAGTCTATCAATTGCGTGATAGTAAGAGTAGCGA |
| | | CAAGTCTGTGTGCCTGTTCACCGATTTCGACAGTCAGACAAATGTTTCTCAGAGTAAGGAC |
| | | AGTGATGTGTACATCACCGACAAATGCGTCCTGGACATGCGCTCCATGGACTTCAAGAGT |
| | | AATAGCGCCGTAGCCTGGAGCAACAAAAGCGACTTCGCATGTGCCAACGCGTTCAATAAC |
| | | TCCATTATCCCTGAAGATACTTTTTTCCCCTCCCCCGAAAGTAGCTGCGATGTTAAACTCG |
| | | TTGAGAAGTCCTTCGAAACTGACACCAACCTGAACTTCCAGAACCTGAGTGTGATTGGCTT |
| | | CCGCATCCTGCTCCTGAAAGTGGCCGGCTTTAACCTGCTCATGACACTGCGTCTGTGGTC |
| | | AAGC |
| 226 | Nucleotide encoding | CAGGCCGTGGTGACCCAGGAGCCCAGCCTGACCGTGAGCCCCGGCGGCACCGTGACCC |
| | P329G TCRαβ- | TGACCTGCAGGAGCAGCACCGGCGCCGTGACCACCAGCAACTACGCCAACTGGGTGCA |
| | VL1TRBC1_(mature) | GGAGAAGCCCGACCACCTGTTCACCGGCCTGATCGGCGGCACCAACAAGAGGGCCCCC |
| | [VL1]-[TRBC1_S56C] | GGCACCCCCGCCAGGTTCAGCGGCAGCCTGCTGGGCGGCAAGGCCGCCCTGACCCTGA |
| | | GCGGCGCCCAGCCCGAGGACGAGGCCGAGTACTACTGCGCCCTGTGGTACAGCAACCA |
| | | CTGGGTGTTCGGCGGCGGCACCAAGCTGACCGTCCTAGACCTGAATAAAGTGTTTCCCC |
| | | CAGAGGTCGCCGTGTTTGAACCGAGCGAGGCTGAGATCTCCCACACCCAGAAGGCTACA |
| | | CTGGTCTGTCTTGCCACCGGTTTCTTTCCTGACCACGTGGAACTGTCTTGGTGGGTTAAC |
| | | GGTAAGGAGGTGCACTCCGGTGTGTGCACTGATCCCCAGCCGTTGAAAGAACAGCCGGC |
| | | TTTGAACGACTCCCGGTACTGCCTTAGCTCCCGCCTGCGCGTGTCTGCTACATTCTGGCA |
| | | GAACCCCCGCAACCACTTTCGGTGCCAGGTGCAGTTTTACGGCCTGTCCGAGAACGATG |
| | | AATGGACACAAGACCGGGCTAAGCCCGTGACTCAGATCGTGAGTGCTGAGGCTTGGGGG |
| | | CGCGCCGATTGCGGCTTCACCTCAGTCAGCTACCAACAGGGCGTTCTGAGTGCCACAAT |
| | | CCTGTATGAGATCCTCCTGGGAAAGGCAACTCTGTACGCTGTTCTCGTCTCCGCGCTTGT |
| | | TCTGATGGCTATGGTGAAGAGAAAGGATTTC |
| 227 | Nucleotide encoding | CAGGCCGTGGTGACCCAGGAGCCCAGCCTGACCGTGAGCCCCGGCGGCACCGTGACCC |
| | P329G TCRαβ- | TGACCTGCAGGAGCAGCACCGGCGCCGTGACCACCAGCAACTACGCCAACTGGGTGCA |
| | VL1TRAC (mature) | GGAGAAGCCCGACCACCTGTTCACCGGCCTGATCGGCGGCACCAACAAGAGGGCCCCC |
| | [VL1]-[TRAC_T47C] | GGCACCCCCGCCAGGTTCAGCGGCAGCCTGCTGGGCGGCAAGGCCGCCCTGACCCTGA |
| | | GCGGCGCCCAGCCCGAGGACGAGGCCGAGTACTACTGCGCCCTGTGGTACAGCAACCA |
| | | CTGGGTGTTCGGCGGCGGCACCAAGCTGACCGTCCTAATCCAGAACCCCGACCCCGCAG |
| | | TCTATCAATTGCGTGATAGTAAGAGTAGCGACAAGTCTGTGTGCCTGTTCACCGATTTCGA |
| | | CAGTCAGACAAATGTTTCTCAGAGTAAGGACAGTGATGTGTACATCACCGACAAATGCGT |
| | | CCTGGACATGCGCTCCATGGACTTCAAGAGTAATAGCGCCGTAGCCTGGAGCAACAAAAG |
| | | CGACTTCGCATGTGCCAACGCGTTCAATAACTCCATTATCCCTGAAGATACTTTTTTCCCC |
| | | TCCCCCGAAAGTAGCTGCGATGTTAAACTCGTTGAGAAGTCCTTCGAAACTGACACCAAC |
| | | CTGAACTTCCAGAACCTGAGTGTGATTGGCTTCCGCATCCTGCTCCTGAAAGTGGCCGGC |
| | | TTTAACCTGCTCATGACACTGCGTCTGTGGTCAAGC |
| 228 | Nucleotide encoding | GAGGTGCAGCTGGTGGAGAGCGGCGGCGGCCTGGTGCAGCCCGGCGGCAGCCTGAGG |
| | P329G TCRαβ- | CTGAGCTGCGCCGCCAGCGGCTTCACCTTCAGCAGGTACTGGATGAACTGGGTGAGGCA |
| | VH3TRBC1_(mature) | GGCCCCCGGCAAGGGCCTGGAGTGGGTGGGCGAGATCACCCCCGACAGCAGCACCATC |
| | [VH3]-[TRBC1_S56C] | AACTACGCCCCCAGCCTGAAGGGCAGGTTCACCATCAGCAGGGACAACGCCAAGAACAG |
| | | CCTGTACCTGCAGATGAACAGCCTGAGGGCCGAGGACACCGCCGTGTACTACTGCGCCA |
| | | GGCCCTACGACTACGGCGCCTGGTTCGCCAGCTGGGGCCAGGGCACCCTGGTGACCGT |
| | | GAGCAGCGACCTGAATAAAGTGTTTCCCCCAGAGGTCGCCGTGTTTGAACCGAGCGAGG |
| | | CTGAGATCTCCCACACCCAGAAGGCTACACTGGTCTGTCTTGCCACCGGTTTCTTTCCTG |
| | | ACCACGTGGAACTGTCTTGGTGGGTTAACGGTAAGGAGGTGCACTCCGGTGTGTGCACT |
| | | GATCCCCAGCCGTTGAAAGAACAGCCGGCTTTGAACGACTCCCGGTACTGCCTTAGCTCC |
| | | CGCCTGCGCGTGTCTGCTACATTCTGGCAGAACCCCCGCAACCACTTTCGGTGCCAGGT |
| | | GCAGTTTTACGGCCTGTCCGAGAACGATGAATGGACACAAGACCGGGCTAAGCCCGTGA |
| | | CTCAGATCGTGAGTGCTGAGGCTTGGGGGCGCGCCGATTGCGGCTTCACCTCAGTCAGC |
| | | TACCAACAGGGCGTTCTGAGTGCCACAATCCTGTATGAGATCCTCCTGGGAAAGGCAACT |
| | | CTGTACGCTGTTCTCGTCTCCGCGCTTGTTCTGATGGCTATGGTGAAGAGAAAGGATTTC |

TABLE A

| | Column A | | | Column B | | |
|---|---|---|---|---|---|---|
| | VH | | | VL | | |
| Antibody | HC-CDR1 | HC-CDR2 | HC-CDR3 | LC-CDR1 | LC-CDR2 | LC-CDR3 |
| αP329G_VH1/VL1 | SEQ ID NO: 56 | SEQ ID NO: 57 | SEQ ID NO: 58 | SEQ ID NO: 69 | SEQ ID NO: 70 | SEQ ID NO: 71 |
| αP329G_VH2/VL1 | SEQ ID NO: 56 | SEQ ID NO: 64 | SEQ ID NO: 58 | SEQ ID NO: 69 | SEQ ID NO: 70 | SEQ ID NO: 71 |
| αP329G_VH3/VL1 | SEQ ID NO: 56 | SEQ ID NO: 64 | SEQ ID NO: 58 | SEQ ID NO: 69 | SEQ ID NO: 70 | SEQ ID NO: 71 |
| αAAA | SEQ ID NO: 77 | SEQ ID NO: 78 | SEQ ID NO: 79 | SEQ ID NO: 85 | SEQ ID NO: 86 | SEQ ID NO: 87 |

TABLE B

| | Column A | | | | Column B | | | |
|---|---|---|---|---|---|---|---|---|
| | VH | | | | VL | | | |
| Antibody | HC-FR1 | HC-FR2 | HC-FR3 | HC-FR4 | LC-FR1 | LC-FR2 | LC-FR3 | LC-FR4 |
| αP329G_VH1/VL1 | SEQ ID | SEQ ID | SEQ ID | SEQ ID | SEQ ID | SEQ ID | SEQ ID | SEQ ID |
| | NO: 59 | NO: 60 | NO: 61 | NO: 62 | NO: 72 | NO: 73 | NO: 74 | NO: 75 |
| αP329G_VH2/VL1 | SEQ ID | SEQ ID | SEQ ID | SEQ ID | SEQ ID | SEQ ID | SEQ ID | SEQ ID |
| | NO: 59 | NO: 60 | NO: 61 | NO: 62 | NO: 72 | NO: 73 | NO: 74 | NO: 75 |
| αP329G_VH3/VL1 | SEQ ID | SEQ ID | SEQ ID | SEQ ID | SEQ ID | SEQ ID | SEQ ID | SEQ ID |
| | NO: 66 | NO: 60 | NO: 67 | NO: 62 | NO: 72 | NO: 73 | NO: 74 | NO: 75 |
| αAAA | SEQ ID | SEQ ID | SEQ ID | SEQ ID | SEQ ID | SEQ ID | SEQ ID | SEQ ID |
| | NO: 80 | NO: 81 | NO: 82 | NO: 83 | NO: 88 | NO: 89 | NO: 90 | NO: 91 |

TABLE C

| | Column A | Column B |
|---|---|---|
| Antibody | VH | VL |
| αP329G_VH1/VL1 | SEQ ID NO: 55 | SEQ ID NO: 68 |
| αP329G_VH2/VL1 | SEQ ID NO: 63 | SEQ ID NO: 68 |
| αP329G_VH3/VL1 | SEQ ID NO: 65 | SEQ ID NO: 68 |
| αAAA | SEQ ID NO: 76 | SEQ ID NO: 84 |

The present disclosure includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

As used herein, a ‘peptide’ refers to a chain of two or more amino acid monomers linked by peptide bonds. A peptide typically has a length in the region of about 2 to 50 amino acids. A ‘polypeptide’ is a polymer chain of two or more peptides. Polypeptides typically have a length greater than about 50 amino acids.

As used herein, an amino acid sequence, or a region of a polypeptide which ‘corresponds’ to a specified reference amino acid sequence or region of a polypeptide has at least 60%, e.g. one of at least ≥65%, ≥70%, ≥75%, ≥80%, ≥85%, ≥90%, ≥91%, ≥92%, ≥93%, ≥94%, ≥95%, ≥96%, ≥97%, ≥98%, ≥99% or 100% sequence identity to the amino acid sequence of the amino acid sequence/polypeptide/region. An amino acid sequence/region/position of a polypeptide/amino acid sequence which ‘corresponds’ to a specified reference amino acid sequence/region/position of a polypeptide/amino acid sequence can be identified by sequence alignment of the subject sequence to the reference sequence, e.g. using sequence alignment software such as ClustalOmega (Söding, J. 2005, Bioinformatics 21, 951-960).

It must be noted that, as used in the specification and the appended claims, the singular forms ‘a,’‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIGS. 1A to 1C: Schematic representation of P329G-CAR and P329G-CD3ε/Cαβ constructs. FIG. 1A depicts a second generation chimeric antigen receptor (CAR) with the anti-P329G binding moiety in the scFv format. FIGS. 1B and 1C show the P329G-CD3ε/P329G-Cαβ constructs in the context of the endogenous TCR complex. The anti-P329G scFv is either fused to the CD3ε chain (1B, P329G-CD3& TOR complex) or the VH was fused to the Cα TOR domain and the VL fused to the Cβ TCR domain (1C, P329G-Cαβ TCR complex). The P329G-Cαβ construct can be further stabilized by introducing an interchain disulfide bond between the Cα and Cβ extracellular domains.

FIGS. 2A to 2C: Schematic representation of the gene constructs corresponding to the P329G-CAR (2A), P329G-CD3ε (2B) or P329G-Cαβ (2C)

FIGS. 3A and 3B: Staining of Jurkat NFAT (TCR/CD3 Effector Cells (NFAT), Promega, #J1601) wildtype (wt) or Jurkat NFAT after CRISPR-Cas9 knock-out of endogenous CD3ε with anti-CD3ε-FITC (1:50, Biolegend, #300406) antibody. FIG. 3A depicts the staining after the knock-out, with Jurkat NFAT wildtype cells as control. FIG. 3B shows the population before and after sorting for CD3s negative cells, leading to a 99.7% CD3ε negative population.

FIGS. 4A and 4B: eGFP expression in Jurkat NFAT CD3ε KO cells after lentiviral transduction of P329G-CD3ε (4A) or P329G-CAR (4B) and pool sorting for living, eGFP positive cells. As negative control served mock transduced cells (cells transduced with empty virus-like particles (VLPs)).

FIGS. 5A-5C: Surface expression of P329G-CAR or P329G-CD3ε TCR in Jurkat NFAT CD3ε KO cells (sorted pools) was confirmed by staining with AF647 labeled Fc-P329G LALA as illustrated in 5A (1) with the corresponding staining histograms depicted in 5B (1). The integration into the TOR complex and its expression on the cell surface was assessed by staining with anti-TCRαβ-BV421 (1:50, Biolegend, #306722) and anti-CD3ε-PE (1:50, Biolegend, #300408) antibodies (5A (2, 3). The corresponding stainings are shown in FIG. 5B (2, 3) and FIG. 5C (2, 3). As negative control for the stainings served mock transduced cells (light gray).

FIGS. 6A and 6B: Activation of Jurkat NFAT CD3ε KO cells transduced with P329G-CAR (sorted pool) or P329G-CD3ε (sorted pool) in the presence of FolR1-target cells with high (HeLa) or low (HT-29) target expression levels upon stimulation with anti-FolR1 (clone 16D5) IgG containing the P329G LALA mutations. Activation was assessed by quantification of the intensity of TOR/CD3 downstream signaling reported by NFAT promoter-controlled luciferase expression. Schematic representation of the assay (6A). Dose-dependent activation of transduced Jurkat cells in the presence of HT29 or HeLa (6B) as target cells. Depicted are technical average values from triplicates, error bars indicate SD.

FIGS. 7A and 7B: Activation of Jurkat NFAT CD3ε KO cells transduced with P329G-CAR (sorted pool) of P329G-CD3ε (sorted pool) in the presence of CD19-target cells with high (Nalm-6) or low (Z138) target expression levels upon stimulation with anti-CD19 (affinity maturated 2B11) IgG containing the P329G LALA mutations. Activation was assessed by quantification of the intensity of TOR/CD3 downstream signaling reported by NFAT promoter-controlled luciferase expression. Schematic representation of the assay (7A). Dose-dependent activation of transduced Jurkat cells in the presence of Z138 or Nalm-6 (7B) as target cells. Depicted are technical average values from triplicates, error bars indicate SD.

FIGS. 8A and 8B: eGFP expression in Jurkat TCRαβ KO-CD4+ cells (T Cell Activation Bioassay (TCRαβ-KO), Promega, #GA1172) after lentiviral transduction of P329G-Cαβ (8A) or P329G-CAR (8B) and pool sorting for living, eGFP positive cells. As negative control served mock transduced cells.

FIGS. 9A-9C: Surface expression of the P329G-Cαβ TOR or P329G-CAR on Jurkat TCRαβ KO-CD4+ cells (sorted pool) was checked by staining with an IgG containing the P329G LALA mutation (anti-FolR1 IgG P329G LALA) and detection of binding by secondary PE-F(ab)2 fragment anti-huIgG (F(ab)2 fragment specific) (Jackson ImmunoResearch, #109-116-097) (A (1)). The incorporation of the VH-TCRα and VL-TCRβ chains was confirmed by staining with anti-TCRαβ-BV421 (1:50, Biolegend, #306722) and anti-CD3s-APC (1:50, Biolegend, #300412) antibodies (9A (2,3). The corresponding staining results are shown in FIG. 9B (1, 2, 3) and FIG. 9C (1, 2, 3). For all stainings (1, 2, 3) the staining of mock transduced cells served as negative control (light gray). As additional negative control for the P329G staining (1) the transduced cells were also stained with secondary antibody only (staining overlaying with mock transduced control (light gray)).

FIGS. 10A and 10B: Activation of Jurkat TCRαβ KO-CD4+ cells transduced with P329G-Cαβ or P329G-CAR (sorted pool) in the presence of FolR1-target cells with high (HeLa) or low (HT-29) target expression levels upon stimulation with anti-FolR1 (clone 16D5) IgG containing the P329G LALA mutation. Activation was assessed by quantification of the intensity of TOR/CD3 downstream signaling reported by IL2 promoter-controlled luciferase expression. Schematic representation of the assay (10A). Dose-dependent activation of transduced Jurkat cells in the presence of HT29 or HeLa (10B) as target cells. Depicted are technical average values from triplicates, error bars indicate SD.

FIGS. 11A and 11B: Activation of Jurkat TCRαβ KO-CD4+ cells transduced with P329G-Cαβ or P329G-CAR (sorted pool) in the presence of CD19 target cells with high (Nalm-6) or low (Z138) target expression levels upon stimulation with anti-CD19 (affinity maturated 2B11) IgG containing the P329G LALA mutation. Activation was assessed by quantification of the intensity of TCR/CD3 downstream signaling reported by IL2 promoter-controlled luciferase expression. Schematic representation of the assay (11A). Dose-dependent activation of transduced Jurkat cells in the presence of Z138 or Nalm-6 (11B) as target cells. Depicted are technical average values from triplicates, error bars indicate SD.

FIG. 12A-12C: Human Pan T cells of two donors were transduced with P329G-CAR, P329G-Cap or P329G-CD3ε respectively. In the case of the P329G-Cαβ construct the endogenous TCRα and TCRβ chains and in the case of the P329G-CD3ε construct the endogenous CD3s were knocked-out using CRISPR-Cas9. (12A) shows the eGFP expression after transduction and knock-out of the respective endogenous TOR chains in both donors. The surface expression of the different constructs was determined by staining with AF647 labeled Fo-P329G LALA (12B). Staining with anti-CD3ε-PE (1:50, Biolegend, #300408) and Fc-P329G LALA-AF647 to check the percentage of correctly assembled P329G-CD3E TCR or P329G-Cαβ TCR complexes is shown in (12C).

FIG. 13A-13L: P329G-CAR T cells (13A-13D)/P329G-Cαβ TCR T cells (13E-13H) or P329G-CD3ε TCR T cells (13I-13L) were incubated with Hela NLR (NucLight Red) cells in an effector (eGFP+ T cell) to target ratio of 1:1 and the adaptor molecule (anti-FolR1 IgG P329G LALA) was titrated from 10 nM-0.01 PM (13A, 13B, 13E, 13F, 13I, 13J). The red cell count was tracked with the Incucyte® system over 4 days. As control served the non-targeted DP47 IgG P329G, no adaptor molecule and non-transduced wildtype T cells with 10 nM of adaptor molecule or 10 nM anti-FolR1 TCB (13C, 13D, 13G, 13H, 13K, 13L). Dose-dependent red cell count reduction (cancer cell killing) or cancer cell growth was observed. Depicted are technical average values from duplicates, error bars indicate SD. Controls (lower graphs) were analyzed in the same experiments but are depicted in separate graphs for clarity.

FIG. 14A-14L: The generated P329G-CAR T cells (13A-13D)/P329G-Cαβ TOR T cells (13E-13H) or P329G-CD3c TCR T cells (13I-13L) were incubated with MKN45 NLR (NucLight Red) cells in an effector (eGFP+ cell) to target ratio of 1:1 and the adaptor molecule (anti-CEACAM5 IgG P329G) was titrated from 10 nM-0.01 pM (13A, 13B, 13E, 13F, 13I, 13J). The red cell count was tracked with the Incucyte@ system over 4 days. As control served the non-targeted DP47 IgG P329G, no adaptor molecule and non-transduced wildtype T cells with 10 nM of adaptor molecule or 10 nM anti-CEACAM5 TCB (13C, 13D, 13G, 13H, 13K, 13L). Dose-dependent red cell court reduction (cancer cell killing) or cancer cell growth was observed. Depicted are technical average values from duplicates, error bars indicate SD. Controls (lower graphs) were analyzed in the same experiments but are depicted in separate graphs for clarity.

### EXAMPLES

In the following Examples, the inventors describe the production and characterisation of T cells engineered to express CD3-TCR complexes comprising recombinant CD3 complex polypeptides. In particular, T cells expressing anti-P329G CD3ε uniTCR or anti-P329G TCRαβ uniTCR are evaluated, and are unexpectedly found to be activated to a similar or greater extent by cells expressing CD19 or FOL1R in the presence of anti-CD19 or anti-FOL1R antibodies comprising an Fc region having P329G, as compared to T cells expressing anti-P329G uniCAR.

### Example 1: Materials and Methods

### 1.1 Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturers' instructions. General information regarding the nucleotide sequences of human immunoglobulins light and heavy chains is given in: Kabat, E. A. et al., (1991) Sequences of Proteins of Immunological Interest, 5th ed., NIH Publication No. 91-3242.

### 1.2 DNA Sequencing

DNA sequences were determined by double-strand Sanger sequencing.

### 1.3 Gene Synthesis

Desired gene segments where required were either generated by PCR using appropriate templates or were synthesized by Genscript Biotech (New Jersey, US) or GeneArt (Thermo Fisher Scientific, Regensburg, Germany) from synthetic oligonucleotides and PCR products by automated gene synthesis. The gene segments flanked by single restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### 1.4 Production of IgG-Like Proteins in Expi293F Cells

Antibodies and bispecific antibodies were generated by transient transfection of Expi293F cells. Cells were seeded in Expi293 media (Gibco, #1435101) at a density of 2.5×10⁵/ml. Expression vectors and ExpiFectamine (Gibco, ExpiFectamine transfection kit, #13385544) were separately mixed in OptiMEM (Gibco, #11520386). After 5 min, both solutions were combined, mixed by pipetting and incubated for 25 minutes at room temperature. Cells were added to the vector/ExpiFectamine solution and incubated for 24 hours at 37° C. in a shaking incubator with a 5% CO₂ atmosphere. One day post transfection, supplements (Enhancer 1+2, ExpiFectamine transfection kit) were added. Cell supernatants were harvested after 4-5 days by centrifugation and subsequent filtration (0.2 μm filter), and proteins were purified from the harvested supernatant by standard methods as indicated below.

### 1.5 Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate PH neutralization of the sample. The protein was concentrated by centrifugation (Millipore Amicon® ULTRA-15, #UFC903096), and aggregated protein was separated from monomeric protein by size exclusion chromatography in 20 mM histidine, 140 mM sodium chloride, pH 6.0.

### 1.6 Production of IgG-Like Proteins in CHO K1 Cells

Alternatively, the antibodies and bispecific antibodies described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and afterwards purified from the harvested supernatant by standard methods.

### 1.7 Analytics of IgG-Like Proteins

The concentrations of purified proteins were determined by measuring the absorption at 280 nm using the mass extinction coefficient calculated on the basis of the amino acid sequence according to Pace et al., Protein Science (1995) 4:2411-1423. Purity and molecular weight of the proteins were analyzed by CE-SDS in the presence and absence of a reducing agent using a LabChipGXII or LabChip GX Touch (Perkin Elmer) Determination of the aggregate content was performed by HPLC chromatography at 25° C. using analytical size-exclusion column (TSKgel G3000 SW XL or UP-SW3000) equilibrated in running buffer (200 mM KH₂PO₄, 250 mM KCl pH 6.2, 0.02% NaN₃).

### 1.8 Preparation of Virus Like Particles (VLPs)

Lipofectamine LTX™-based transfection was performed using ˜70% confluent Lenti-X™ 293T cells (Takara, #632180) and the construct encoding transfer vectors as well as packaging vectors pCAG-VSVG and psPAX2 at a 2:1:2 molar ratio (Giry-Laterriere M et al., Methods Mol Biol. 2011; 737:183-209, Myburgh R et al., Mol Ther Nucleic Acids. 2014). As control for every experiment, mock virus-like particles (VLPs) using only the packaging vectors, but no transfer vector, were produced. After 48 hours, the supernatant was collected and centrifuged for 5 min at 350×g to remove remaining cells and purify the virus particles. VLPs were used directly or concentrated 10-fold (Lenti-x-Concentrator, Takara, #631231). For storage the VLPs were aliquoted in Eppendorf tubes and snap frozen in liquid nitrogen, before being stored at −80° C.

### 1.9 CRISPR/Cas9 Mediated Knockout in Jurkat Cells or Primary T Cells

For the CRISPR-Cas9 KO ribonucleoprotein complexes (RNPs) were prepared by carefully mixing 6 μl Cas9 (TrueCut Cas9, Invitrogen, #A36499) with 9 μl single sgRNA (100 uM, Integrated DNA Technologies (IDT)) The mixture was incubated for 10 min at room temperature. One million Jurkat NFAT cells (TOR/CD3 Effector Cells (NFAT), #J1601, Promega) were spun down (350×g, 3 min) and washed once with Dulbecco's PBS (DPBS) (Gibco, #14190-136). The SE 4D-Nucleofector™ X solution (Lonza, #V4SC-1096) was adapted to room temperature and the cell pellet was resuspended in 100 μl of the buffer. The RNPs were added to the cell suspension, mixed and transferred it to an electroporation cuvette without creating bubbles. Electroporation with the 4D-Nucleofector unit (Lonza) was performed using pulse code CL-120

### 1.10 Transduction of Jurkat NFAT or TCRαβ-KO CD4+ Cells

One million Jurkat cells/well were seeded in a 24-well plate. VLPs were used fresh or thawed at 37° C. and 300 ul were added together with 8 μg/ml Polybrene (Sigma Aldrich) and Lentiboost P (1:100) (Sinon Biotech, #SB-P-LV-101-12) to a 24 well plate for spinfection of Jurkat NFAT (TCR/CD3 Effector Cells (NFAT), Promega, #J1601) with CD36 CRISPR-Cas9 knock-out or Jurkat TCRαβ-KO CD4+ (T Cell Activation Bioassay (TCRαβ-KO), Promega, #GA1172) cells at 1100×g for 99 min and 31° C. The cells were incubated for at least 72 hours at 37° C., 5% CO₂ before the transduction was checked by flow cytometry

1.11 Sorting of Jurkat NFAT or TCRαβ-KO CD4+ Cells after Knockout or Transduction

In order to have clean cell populations, the cells were pool sorted for CD3ε negative (CD3ε knock-out) or in case of transduction, eGFP/anti-P329G positive cells. Between 3-10 million cells were collected and spun down (400×g, 4 min). The media was removed and the cells were resuspended in MACS buffer (Miltenyi Biotec, #130-091-222) supplemented with 5% BSA (Miltenyi Biotec, #130-091-376). The transduced cells were stained with live/dead dye (LIVE/DEAD™ Fixable Near-IR Dead Cell Stain Kit, Invitrogen, #L.34976) and 100 nM of Fc-P329G LALA-AF647 and pool sorted for eGFP and/or AF647 positive cells on the BD FACSArialII. For sorting of the CD3s knock-out cells they were also stained with live/dead dye and anti-CD3-FITC (1:50, Biolegend, #300406) and sorted for living, FITC negative cells. The sorting was performed using the 100 micron nozzle and the 4-way purity precision mode was applied.

### 1.12 Jurkat NFAT or TCRαβ-KO CD4+ Activation Assay

The Jurkat activation assay measures TCR/CD3 signaling of a human acute lymphatic leukemia reporter cell line (TCR/CD3 Effector Cells (NFAT), Promega, #J1601 or T Cell Activation Bioassay TCRαβ-KO CD4+, Promega, #GA1172). Those immortalized T cell lines are genetically engineered to stably express a luciferase reporter driven by TOR/CD3 signaling. After transduction, the cell line expresses a chimeric antigen receptor (CAR) construct possessing a CD37, signaling domain (P329G-CAR) or a chimeric T cell receptor (TOR) construct (P329G-Cαβ or P329G-CD3ε) which is integrated in the TOR complex containing the endogenous CD37, Binding of the CAR or chimeric TOR to an immobilized adapter molecule (e.g. a tumor antigen bound adapter molecule) leads to CAR/TCR cross linking resulting in T cell activation and in the expression of luciferase. After addition of a substrate, the activity can be measured as relative luminescence units. The assay was performed in a 384-well plate (Falcon, #353988). Effector cells (P329G-CAR, P329G-Cαβ or P329G-CD3ε positive Jurkat cells) and target cells were seeded in a 2.5:1 ratio (20 000 effector cells and 8000 target cells) in 20 ul or 10 μl respectively, in RPMI-1640 (Gibco, #42401-018)+10% FOS (Sigma, #F4135-600ML)+1% Glutamax (Gibco, #35050-038) (growth medium) in triplicates. Further, a serial dilution of the antibody of interest was prepared in growth medium and 10 μl were added to the wells to obtain final concentrations ranging from 100 nM to 0 nM in the assay plate with a final volume of 40 μl per well in total. For the Jurkat NFAT assay the readout was performed using GloSensor CAMP Reagent (Promega, #E1291) and 2% of the end volume (here: 40 ul, resulting in 0.8 ul/well) were added to the wells during the assay setup. The 384-well plate was centrifuged for 1 min at 300×g at RT and incubated for 4-7 hours at 37° C. and 5% CO₂ in a humidified atmosphere. After the incubation, the plates were adjusted to room temperature for 10 minutes before measurement. For the activation assay with the Jurkat TCRαβ-KO CD4+ cells 100% of the final volume (40 ul) of Bio-Glo-NL reagent (Promega, #J3082) was added after the 4-7 h incubation period, plates were centrifuged for 1 min at 350×g and incubated for 5-10 minutes at room temperature. Afterwards, the relative luminescence units (RLU) per s/well were measured immediately using a Tecan microplate reader. Concentration-response curves were fitted and EC₅₀ values were calculated using GraphPadPrism version 8.

1.13 Isolation of Primary T Cells from Buffy Coats

Buffy coats were ordered from Blutspende Zürich (Rütistrasse 19, 8952 Schlieren) A Leucosep tube with 15 mL, of room temperature Histopaque density gradient medium (Sigma-Aldrich, #10771) was prepared and centrifuged at 400×g for 5 minutes, until the Histopaque passed the filter. The blood was transferred to a T75 flask and an equal volume of DPBS was added. 30 ml of the blood/buffer mixture was added to the Leucosep Tubes and they were centrifuged 1200×g for 20 min with the breaks off. The band containing the peripheral blood mononuclear cells (PBMCs) was carefully pipetted into a fresh 50 ml falcon tube and topped up to 50 ml with DPBS. The tubes were centrifuged at 300×g for 10 minutes, then the supernatant was discarded. This step was repeated two more times, before the cells were resuspended in DPBS and counted. Pan T cell isolation was performed by negative selection according to the manufacturer's instructions using the Pan T cell isolation kit (Miltenyi, #130-096-535). The cells were either frozen or used directly after isolation. Cells were cultured in advanced RPMI (Gibco, #11530446), 10% FBS (Sigma, #F4135-500ML), 1% Glutamax (Gibco, #35050-038), 50 IU/Proleukin (Novartis), 25 ng/ml IL-7 (Miltenyi, #130-095-364) and 50 ng/ml IL-15 (Miltenyi, #130-095-766) (T cell medium).

### 1.14 Transduction of Primary T Cells

T cells were activated for 16-24 hours using ImmunoCult™ Human CD3/CD28/CD2 T Cell Activator (StemCell, #10990) according to the manufacturer's manual. Then the activated cells were resuspended and counted and 1.5 million cells/well were seeded in a 24-well plate. VLPs were used fresh or thawed at 37° C. and 300 ul were added together with 8 ug/ml Polybrene (Sigma Aldrich) and Lentiboost P (1:100) (Sirion Biotech, #SB-P-LV-101-12) to the cells in a 24 well plate for spinfection at 1100×g for 99 min and 31° C. The cells were incubated for at least 72 hours at 37° C., 5% CO₂, before the transduction was checked by flow cytometry.

### 1.15 CRISPR/Cas9 Mediated Knockout in Transduced Primary T Cells

For single CRISPR KOs, ribonucleoprotein complexes (RNPs) were prepared by carefully mixing 2 μl Cas9 (TrueCut Cas9, Invitrogen, #A36499) with 3 μl single sgRNA (100 uM, Integrated DNA Technologies (IDT). For double knockouts 1 ul Cas9 was mixed with 1.5 ul of single sgRNA 1 and 1 ul Cas9 was mixed with 1.5 ul of single sgRNA 2. The mixtures were incubated for 10 minutes at room temperature and in case of a double KO the two separately formed RNPs were mixed together after the incubation. 24 hours after transduction, one million primary T cells were spun down (350×g, 3 min) and washed once with DPBS. The cell pellet was resuspended in 20 μl P3 Primary Cell Nucleofector™ Solution (Lonza, #V4XP-3024) which was prior adapted to room temperature. The RNPs were added to the cell suspension, mixed and transferred it to a well of an electroporation stripe. Electroporation with the 4D-Nucleofector unit (Lonza) was performed using pulse code EH-115. Then the cells were resuspended in prewarmed T cell medium and incubated at 37° C., 5% CO₂ until the next steps (at least 3 days).

### 1.16 Incucyte® Immune Cell Killing Assay

Cancer cell lines (Hela and MKN45) were in house transduced with the Incucyte@ NucLight Red Lentivirus (EF1α, Puro, #4476) and stable cell lines were created under puromycin selection (Hela NLR, MKN45 NLR). Human pan T cells were isolated, transduced with the desired constructs and in case of the P329G-CD3ε or P329G-Cαβ additionally the endogenous CD3ε or TCRα+β was knocked out. On day 5 after transduction the Incucyte® killing assay was set up. 10.000 HeLa NLR or MKN45 NLR cells were resuspended in RPMI-1640 (Gibco, #42401-018)+2% FCS (Sigma, #F4135-500ML)+1% Glutamax (Gibco, #35050-038) (killing medium) and seeded in 100 ul in each well of a flat bottom 96-well plate. The plate was incubated for 2-4 hours at 37° C., 5% CO₂, until the cells were slightly attached. The primary T cells were counted and adjusted to 10 000 eGFP+ cells/50 ul or 20.000 non transduced T cells/50 ul (as control) in the killing medium and added to the attached cancer cells. Adaptor and control antibodies were diluted in killing medium to the desired concentrations and 50 ul were added to the wells. Every condition was pipetted in duplicates. Bubbles were removed from the wells surface and the plates were placed in the Incucyte® S3 machine. Five images/well were captured every 4 hours over a course of 5 days. The reduction in cancer cell numbers was quantified using an analysis mask counting the amount of red cells.

### Example 2: Preparation of P329G-CAR, P329G-CD3E and P329G-Cαβ Constructs

DNA sequences encoding the heavy (VH) and light (VL) variable domains of the anti-P329G antibody (VH3VL1), which is specific to the human Fc portion featuring the P329G mutation, were used as single chain variable fragment (scFv) with a (G4S) 4 linker between the variable domains. The amino acid sequence of anti-P329G VH3VL1 scFv is shown in SEQ ID NO:96.

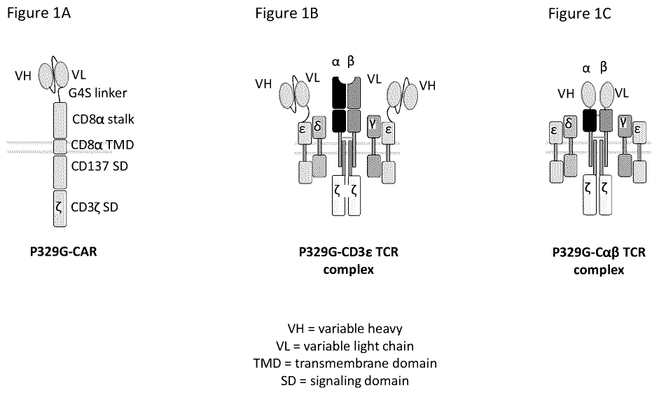

In the P329G chimeric antigen receptor (P329-CAR), the scFv was used in a 4-1BB-CD3Z CAR format. The scFv was fused to an extracellular stalk (Uniprot P01732 [135-182]) and transmembrane domain of CD8a (TMD) (Uniprot P01732 [183-203]) via a G4S linker, followed by the intracellular co-stimulatory signaling domain of 4-1BB (CD137) (Uniprot Q07011 [214-255]) and the intracellular signaling domain of CD3ζ (Uniprot P20963 [52-164]) (FIG. 1A). The mature amino acid sequence of the P329G-CAR (VH3VL1) is shown in SEQ ID NO:108.

For the P329G-CD3ε construct, the scFv was fused to the CD3ε chain (Uniprot P07766 [23-207]) of the TCR complex via a (G₄S)₃ linker (FIG. 1B). The mature amino acid sequence of P329G-(VH3VL1)-CD3ε is shown in SEQ ID NO: 121.

In the P329G-Cαβ construct, the VH and VL of the anti-P329G domains were directly fused to the constant regions of the TCRα and TCRβ chains (Uniprot P01848 [1-140] and Uniprot P01850 [1-176]), respectively. VH and VL thereby replace the Vα and Vβ domains of the natural TCRαβ chains as indicated in (FIG. 1C). In both chimeric TCR formats, the chains are thought to naturally integrate into the TCR complex (FIG. 1B-C). The mature amino acid sequences of the polypeptides forming the P329G-(VH3VL1)-Cαβ constructs are shown in SEQ ID NO: 137 and SEQ ID NO: 165.

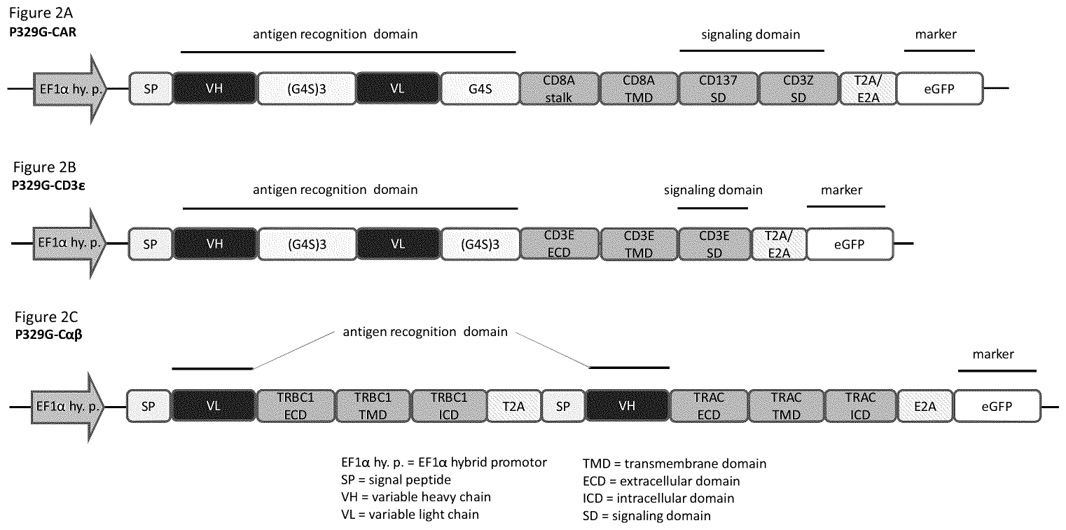

A graphical representation of an exemplary expression construct including the enhanced green fluorescent protein (eGFP) expression marker is shown in FIG. 2A for the P329G-CAR and in FIGS. 2B and 2C for the P329G-CD3ε and P329G-Cαβ respectively. The individual protein coding genes are separated by T2A or E2A self-cleaving peptide sequences.

### Example 3: Gene Knockout of Endogenous CD3E in Jurkat NFAT Cells

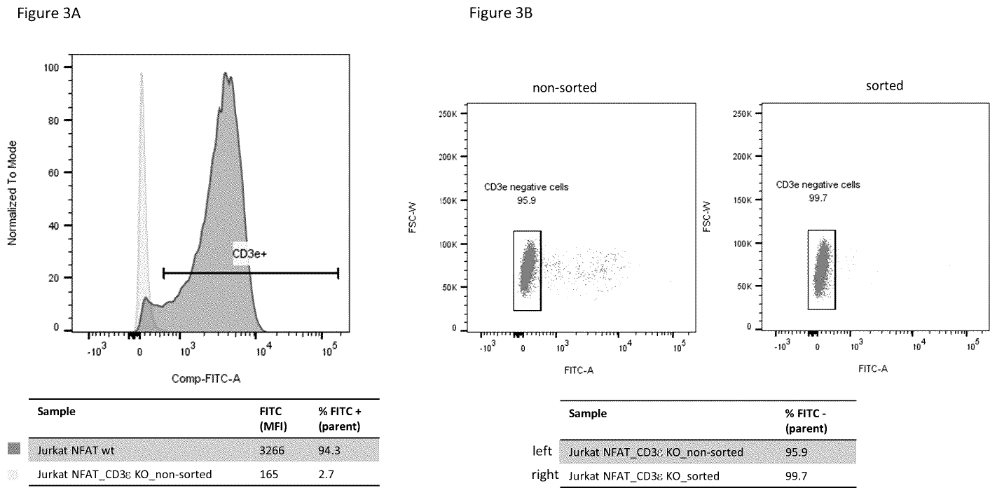

In order to characterize the P329G-CD3ε TCR complex, CD3ε-negative Jurkat NFAT reporter cells were generated by CRISPR/Cas9-mediated gene knockout of the endogenous CD3E gene. The knockout prevents the formation of mixed TCR complexes containing the wild type CD3ε as well as the modified P329G-CD3ε chains. RNPs using the sgRNA targeting Exon 7 of CD3E (SEQ ID NO:217) were generated and the knockout was performed as described above. The cells were afterwards resuspended in 1 ml of RPMI-1640, 10% FBS, 1% Glutamax (no antibiotics) and incubated for 3 days (37° C., 5% CO₂, humidified) before flow cytometry analysis to verify the CD3E gene knockout (FIG. 3A). Cells were purified by sorting using the FACSAria™ III gated on CD3ε negative, living cells (as described earlier) and then reanalyzed by flow cytometry (see FIG. 3B). The cells were 99.7% CD3ε-negative after this procedure and ready to be used for further experiments.

### Example 4: Expression of P329G-CAR or P329G CD3ε or P329G-Cαβ in Jurkat NFAT CD3ε KO or Jurkat TCRαβ KO CD4+ Cells

The P329G-CAR, P329G-CD3ε or P329G-Cαβ receptors were transduced with virus-like particles (VLPs) into Jurkat NFAT CD3ε KO or Jurkat TCRαβ KO CD4+ cells, as described above. Cells were pool sorted for eGFP expression or eGFP and anti-P329G co-expression. Expression of chimeric receptors was assessed and compared by flow cytometry. Transduced Jurkat cells were harvested, washed with DPBS and seeded at 100,000 cells per well in a 96 well U bottom plate. The cells were stained with LIVE/DEAD™ Fixable Near-IR Dead (Invitrogen, #L34976) dye (1:1000 in DPBS) for 20 minutes at 4° C., and washed twice with FACS-buffer (1×DPBS, 2% FBS, 5 mM EDTA pH 8.0, 0.05% NaN₃). Then the cells were resuspended in 50 ul FACS buffer with 100 nM fluorescently labeled (Alexa Fluor 647) Fc fragments featuring the previously described P329G LALA mutations (Fc-P329G LALA-AF647). To assess the integration in the endogenous TCR complex, the cells were also stained with anti-CD3s (1:50, -PE, Biolegend, #300408 or 1:50,-APC, Biolegend, #300412) and anti-TCRα⊖-BV421 (1:50, Biolegend, #306721) and incubated for 20 minutes at 4° C. After two washing steps the cells were fixed (BD CytoFix, #554655) and analyzed on the FACS.

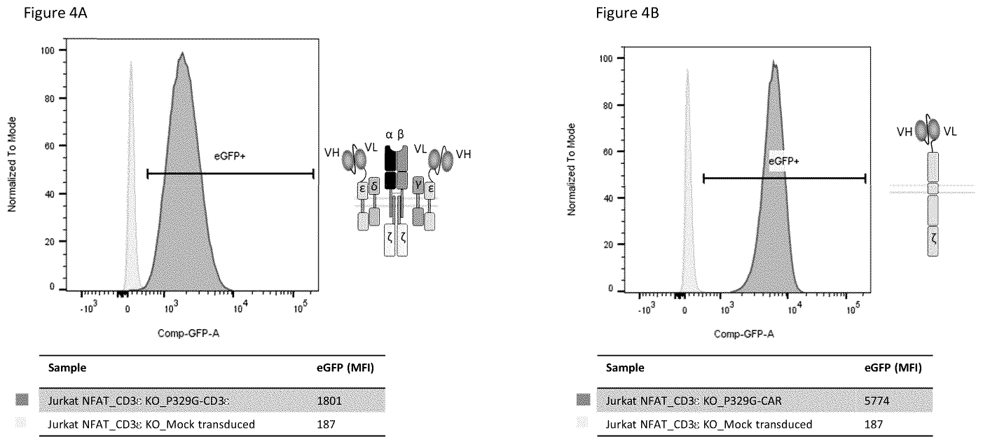

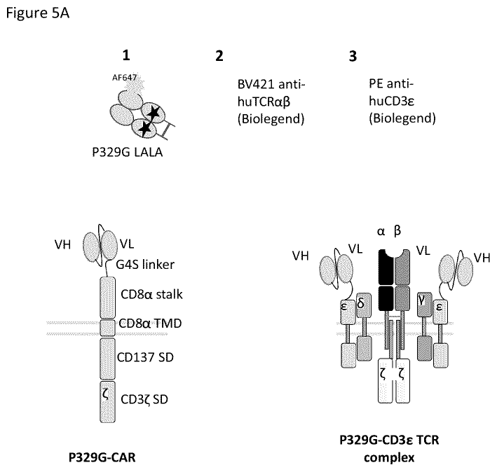

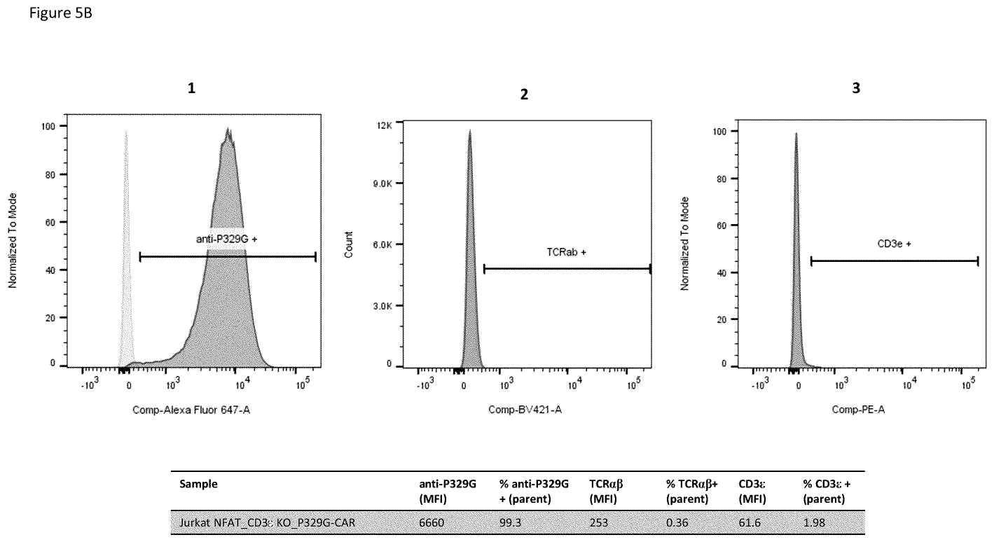

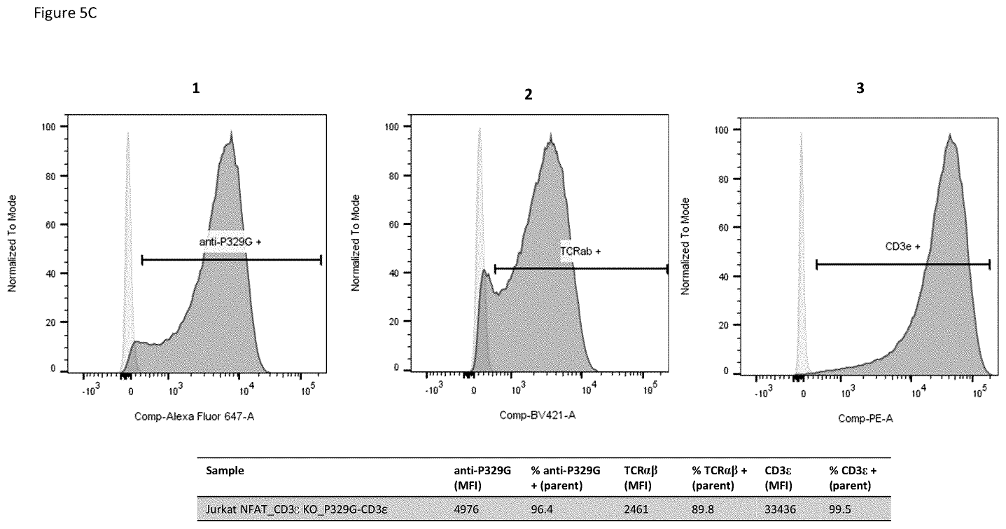

Intracellular eGFP expression of the pool-sorted transgenic Jurkat NFAT CD3ε KO cells expressing the P329G-CAR or P329-CD3ε (FIGS. 4A and 4B) shows that all the cells have integrated the construct of interest in the genome. FIG. 5B (1) and FIG. 5C (1) shows the surface expression of the receptors (96-99% positive), while FIG. 5B (2+3) and FIG. 5C (2+3) show the integration of the P329G-CD3ε construct into the endogenous TCR complex, as the TCRαβ chains are only detectable after transduction with the chimeric CD3ε construct.

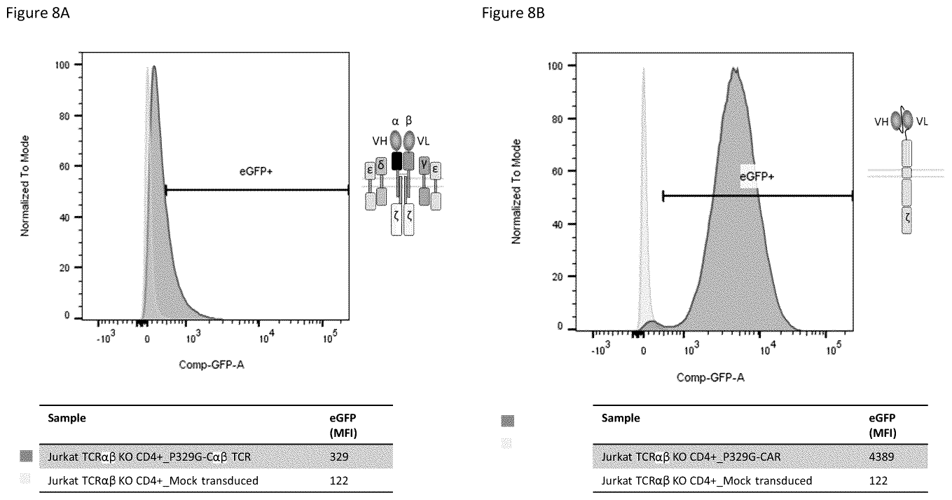

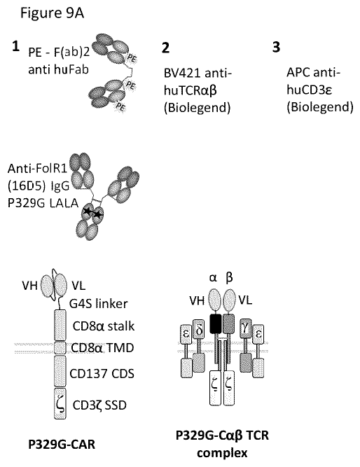

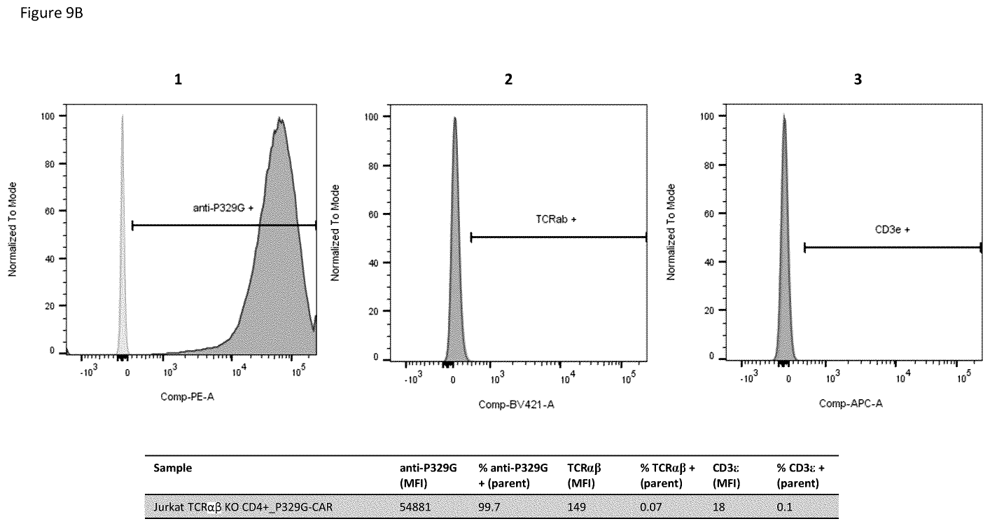

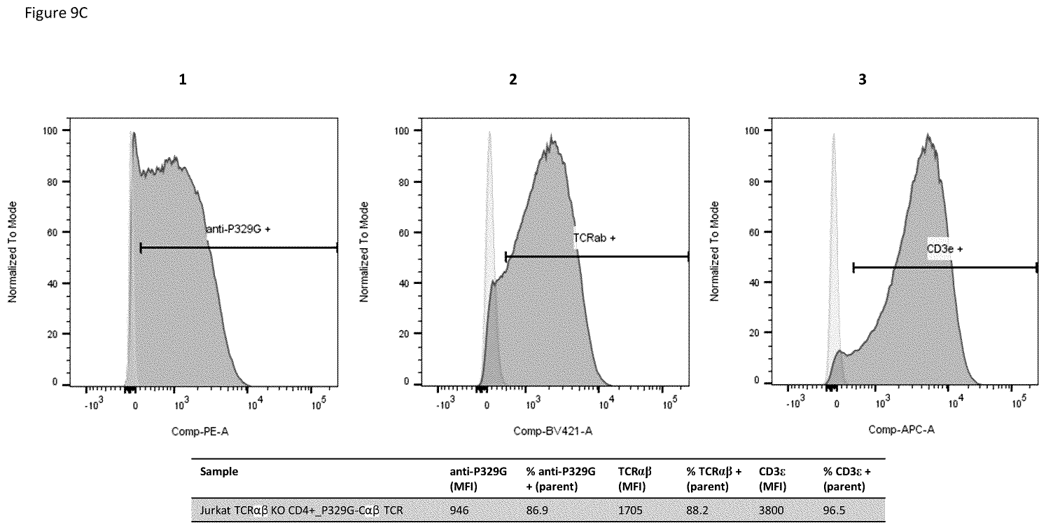

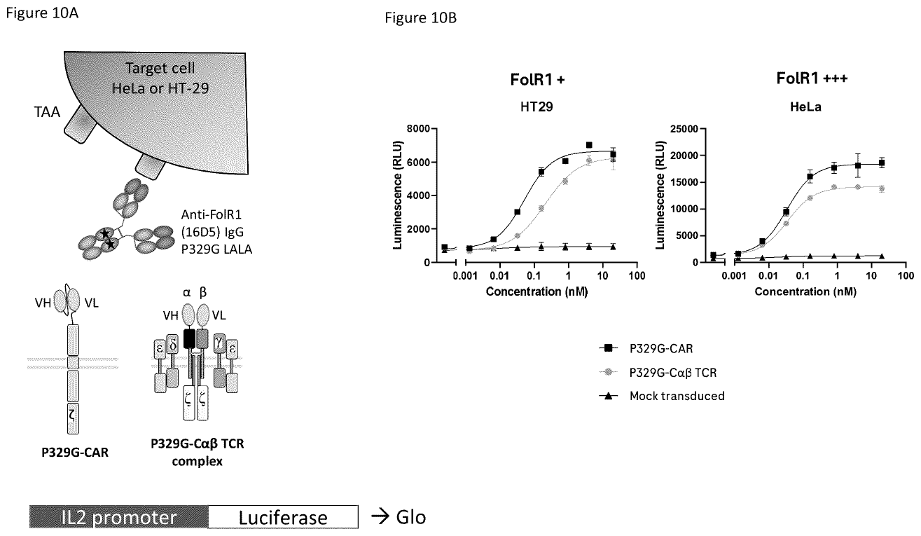

FIGS. 8A and 8B show the eGFP expression of the transgenic Jurkat TCRαβ KO CD4+ cells expressing the P329G-CAR or P329G-Cαβ constructs. Notably, the eGFP levels of the Jurkat cells transduced with the P329G-Cαβ construct were much lower compared to the eGFP expression of the P329-CAR Jurkat cells. This might be due to the larger size of the construct and also the position of the eGFP gene in the construct (third gene vs. second gene). FIG. 9B (1) and 9C (1) show the surface expression of the receptors (87-99% positive), while FIG. 9B (2+3) and 9C (2+3) show the integration of the P329G-Cαβ construct into the endogenous TCR complex, as the CD3ε chains are only detectable after transduction with the chimeric Cαβ construct.

In summary, the P329G-CAR, P329G-CD3ε and P329G-Cαβ constructs were shown to be expressed on the surface of the Jurkat cells and the Cαβ or CD3ε fusion constructs were shown to integrate into the natural TCR complex of the cells.

The functionality of the different anti-P329G receptors was assessed in subsequent Jurkat activation assays.

### Example 5: Specific T Cell Activation in the Presence of Adaptor Antibody Comprising the P329G Mutation

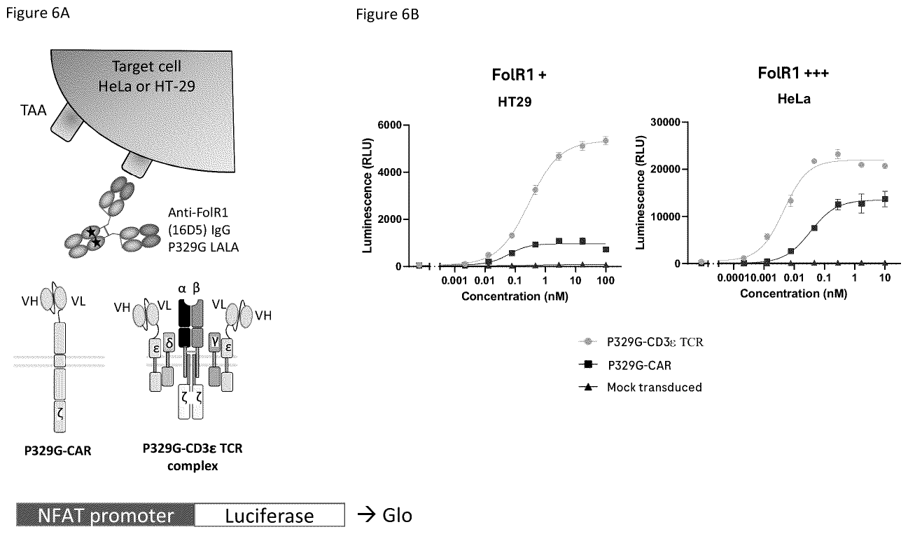

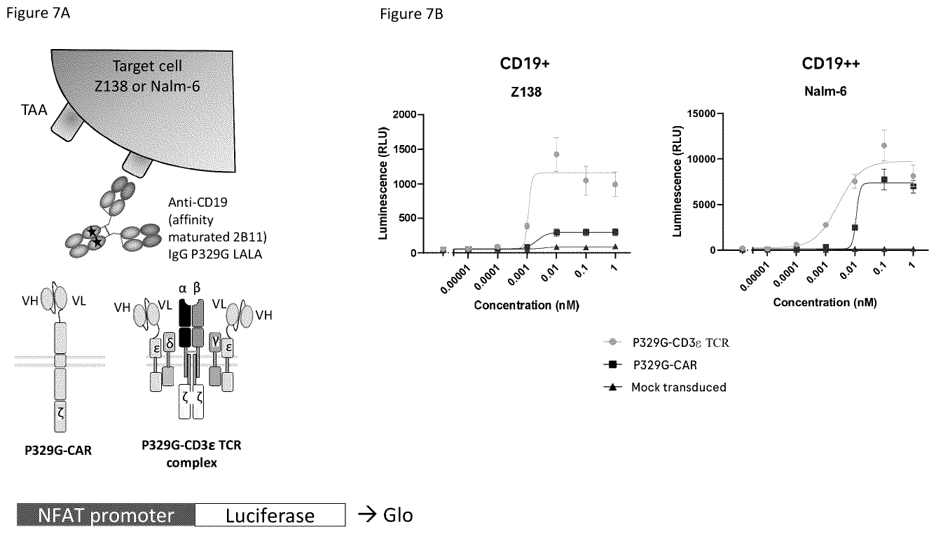

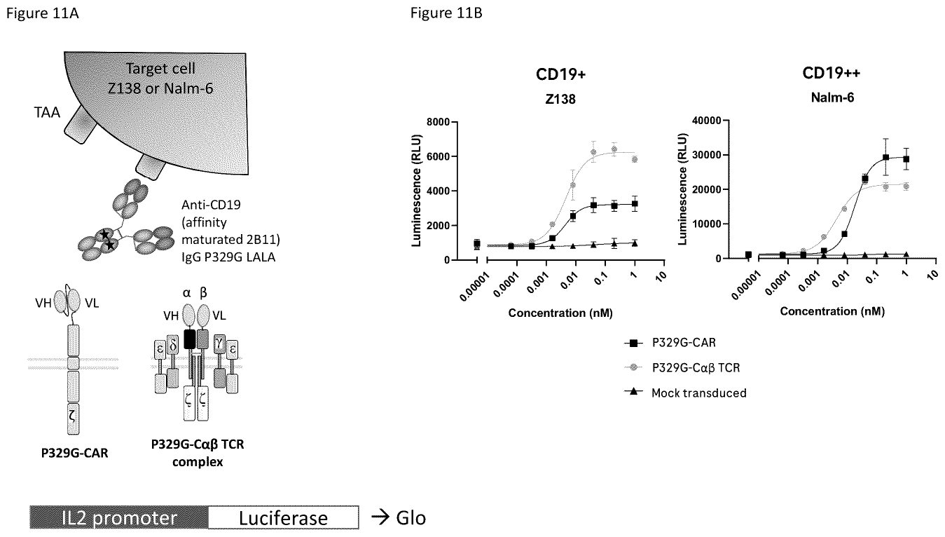

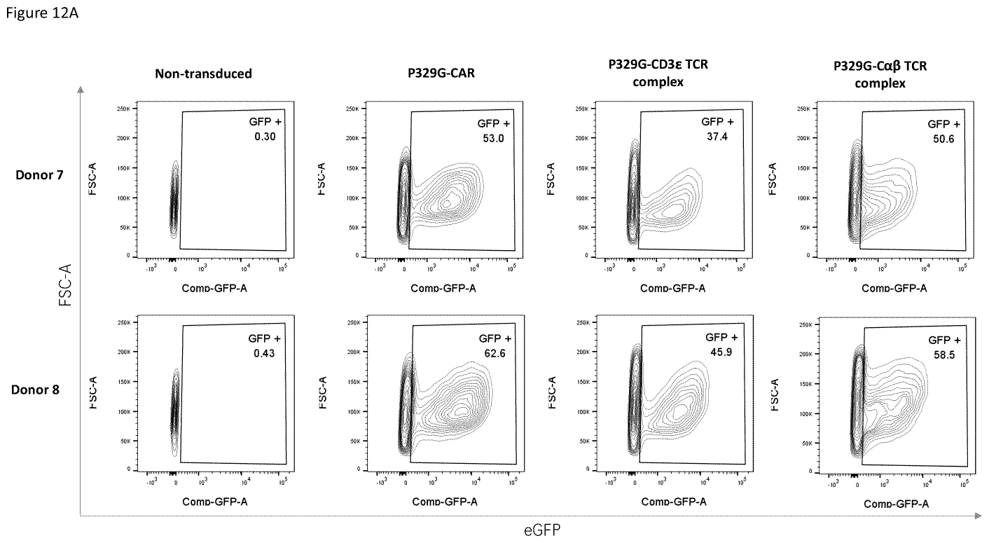

To assess and compare specific T cell activation for T cells expressing P329G receptors having the formats shown in FIG. 1, P329G-CAR, P329G CD3ε or P329-Cαβ transduced Jurkat cells were evaluated for their activation in the presence of FolR1-positive target cells, and anti-FolR1 IgG P329G LALA as targeting adaptor (FIGS. 6A and 10A). The same transgenic Jurkat cell pools were also analysed for their activation in the presence of CD19-positive target cells, and anti-CD19 IgG P329G LALA (FIGS. 7A and 11A). More specifically, transgenic P329G-receptor-positive Jurkat cells were tested with cell lines expressing FolR1 at high (HeLa) or low (HT29) levels. Similarly, cells expressing CD19 at high (Nalm-6) or low (Z138) levels were evaluated. Mock-transduced Jurkat cells (transduced with VLPs lacking a transgene vector) served as a negative control. The Jurkat activation assay was performed as described in detail above.

Dose-dependent and also antigen level-dependent activation of the transgenic Jurkat cells was observed with all tested P329G-specific constructs. In all target antigen-expressing cell lines investigated, the P329G-CD3ε Jurkat cells displayed higher activation compared to cells expressing P329G-CAR. The difference was especially pronounced with cell lines expressing the relevant target antigen at low levels. The mock-transduced control cells displayed were not activated in the presence of anti-FolR1 or anti-CD19 IgG P329G LALA adaptor molecules and any of the target antigen-expressing cell lines investigated (FIGS. 6B, 7B).

The level of activation of T cells expressing the P329G-Cap was similar to the level of activation observed for T cells expressing P329G-CAR (FIGS. 10A, 10B and 11B), although the level of surface expression of P329G-Cαβ was much lower than the level of surface expression of P329G-CAR (FIG. 9B (1) and 9C (1)). In the Z138 model (in which the cells express low levels of CD19), T cells expressing P329G-Cαβ showed higher activation than T cells expressing P329G-CAR (FIG. 11B).

In summary, P329G-CAR, P329-CD3ε and P329G-Cαβ constructs were shown to be functional and selective activation by adaptor IgGs comprising the P329G mutation was observed. The P329G-CD3ε construct showed superior activation compared to the P329G-CAR in all of the models tested, while the P329-CD3ε construct showed similar activation compared to the P329G-CAR, despite having lower overall expression at the cell surface. The next step was to test and compare the expression and activity of the construct in primary T cells.

### Example 6: Expression of P329G-CAR, P329-CD3ε or P329G-Cαβ In Primary T Cells

Constructs encoding the P329G-CAR, P329G-CD3ε or P329G-Cαβ receptors were transduced with virus-like particles (VLPs) into human Pan T cells of two donors as described above. For cells engineered to express P329G-CD3s or P329G-Cαβ, the endogenous nucleic acid encoding CD3ε or TCRαβ (respectively) were knocked-out using CRISPR-Cas9, 24 hours after transduction (see above). The sgRNAs were designed in such a way to cut the endogenous CD3E or TRBC1/TRAC loci, but not the chimeric constructs, by removing the Protospacer Adjacent Motif (PAM) and adding several mismatches to the binding site. The CD3ε KO was performed using a sgRNA targeting Exon 7 of huCD3E (SEQ ID NO: 217). The TCRαβ KOs were performed using sgRNA targeting human TRBC1 (SEQ ID NO:218), and sgRNA targeting human TRAC (SEQ ID NO:219).

The expression of the chimeric receptors and gene knockout was assessed and compared by flow cytometry on day 5 after transduction, as follows. Transduced T cells were harvested, washed with DPBS and seeded at 100,000 cells per well in a 96-well U bottom plate. The cells were stained with LIVE/DEAD™ Fixable Near-IR Dead (Invitrogen, #L34976) dye (1:1000 in DPBS) for 20 minutes at 4° C. and then washed twice with FACS-buffer (1×DPBS, 2% FBS, 5 mM EDTA pH 8.0, 0.05% NaN3) Then the cells were resuspended in 50 ul FACS buffer with 100 nM Fc-P329G LALA-AF647 and anti-CD3ε-PE (1:50, Biolegend, #300408) and incubated for 20 minutes at 4° C. After another two washing steps the cells were fixed (BD CytoFix, #554655) and analyzed on the FACS.

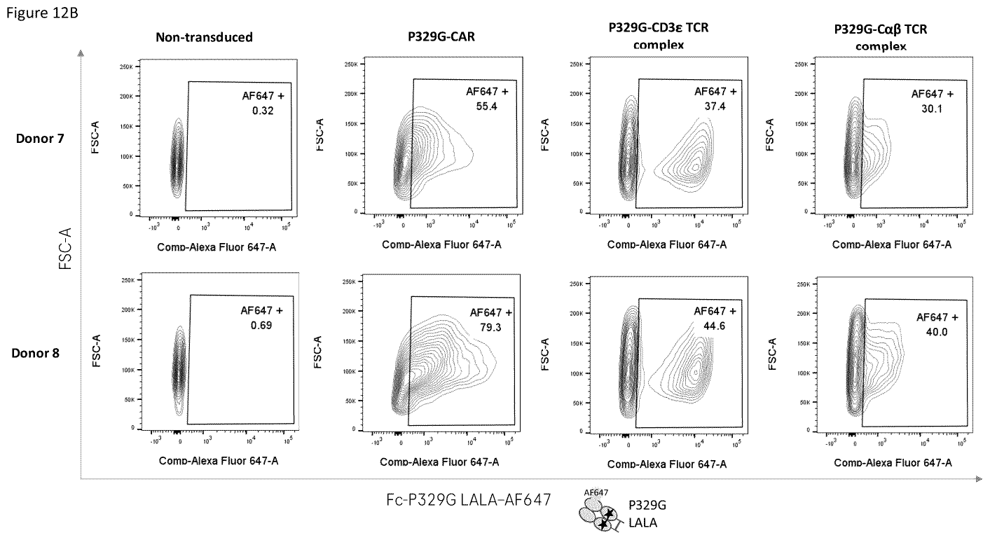

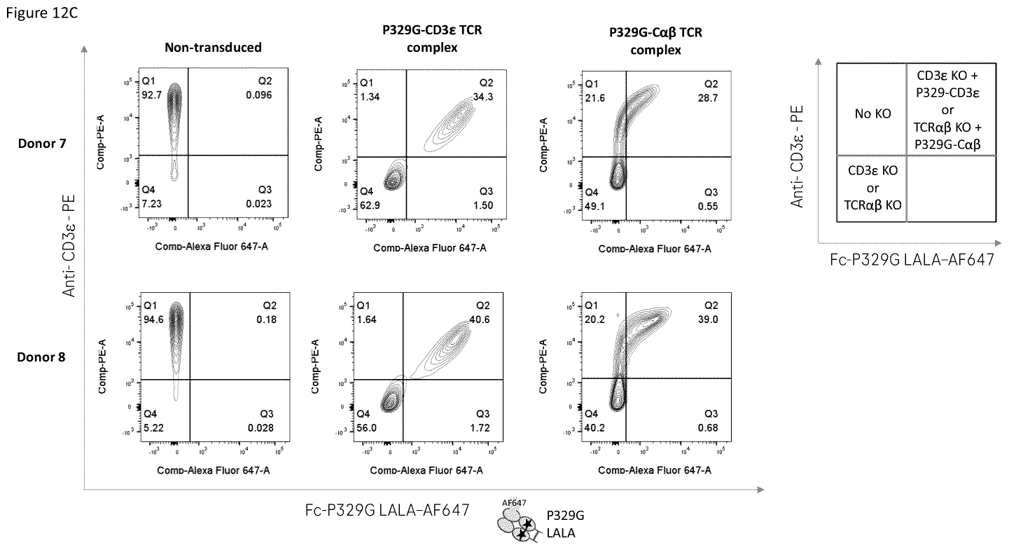

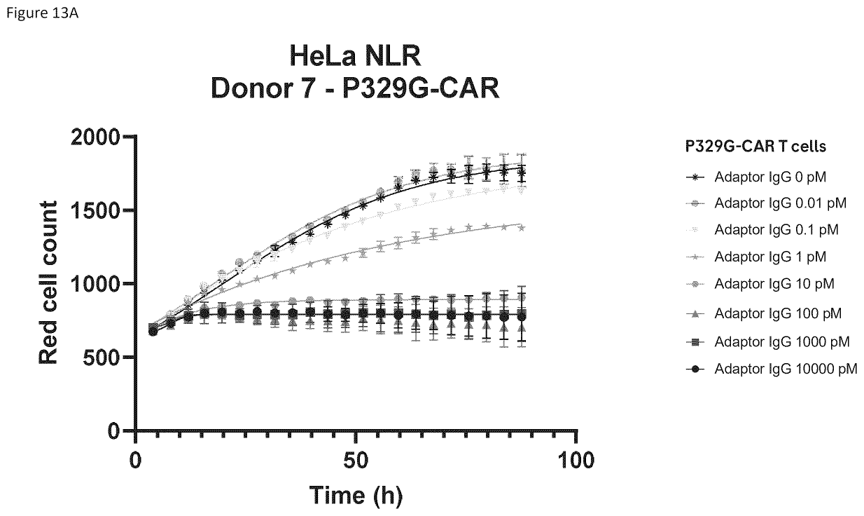

The intracellular eGFP expression is shown in FIG. 12A. For donor 7, 37-53% of the cells were eGFP positive, depending on the construct. Donor 8 showed slightly higher eGFP levels following transduction (46-63%). FIG. 12B shows the receptors' surface expression and their ability to bind to Fc-P329G LALA. Plotting the CD3 expression and Fc-P329G LALA-AF647 expression (FIG. 12C) revealed that for the P329G-CD3ε, the CD3: KO was almost complete (only 1.34 (Donor 7) or 1.64 (Donor 8) CD3ε+/Fc-P329G LALA-AF647-cells) and 34-40% of the T cells express the P329G-CD3E TCR complex. For the P329G-Cαβ+TCRαβ-/Fc-P329G LALA-AF647+ population of 29-39% was achieved.

The P329G-CAR and the P329G-CD3ε or P329G-Cαβ constructs were shown to be transduced and expressed on the surface of the primary T cells. To test the functionality of the constructs, they were assessed using an Incucyte® immune cell killing assay.

### Example 7: Incucyte® Immune Cell Killing Assay with P329G-CAR, P329G-CD3ε or P329G-Cαβ T Cells

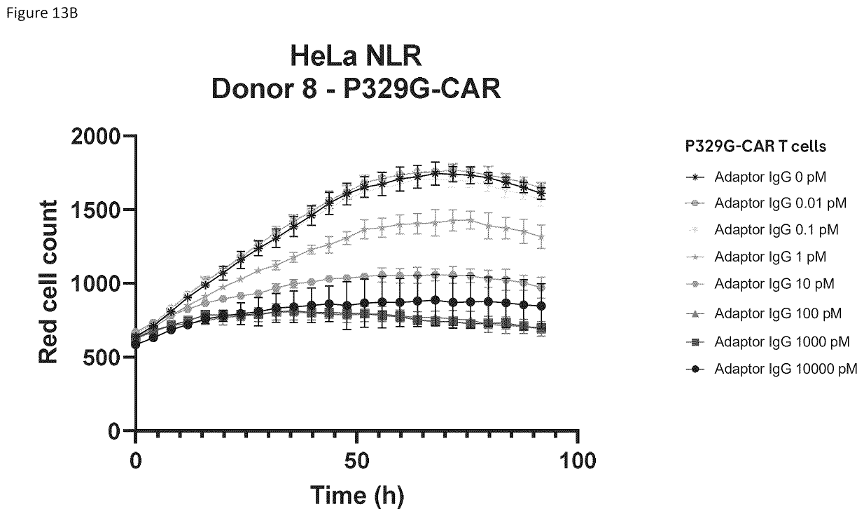

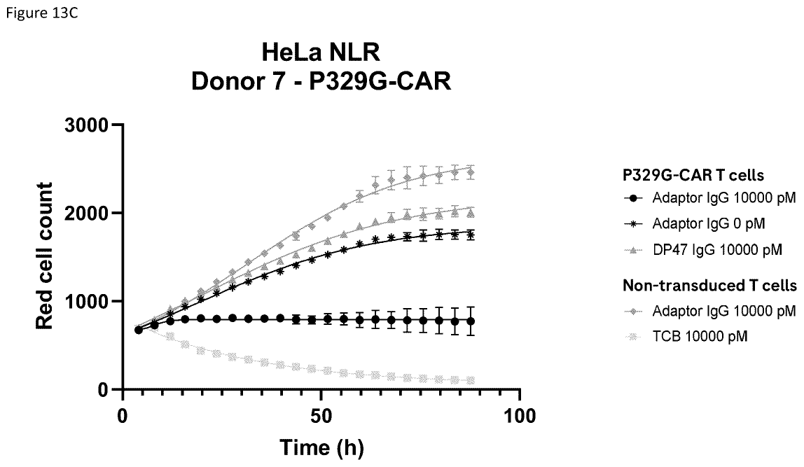

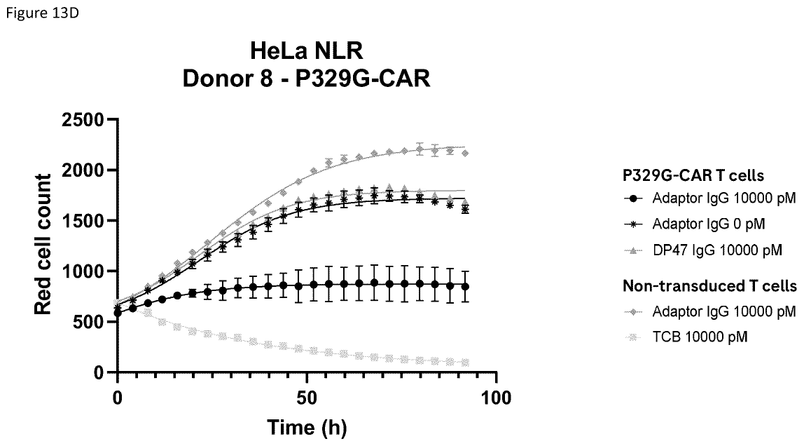

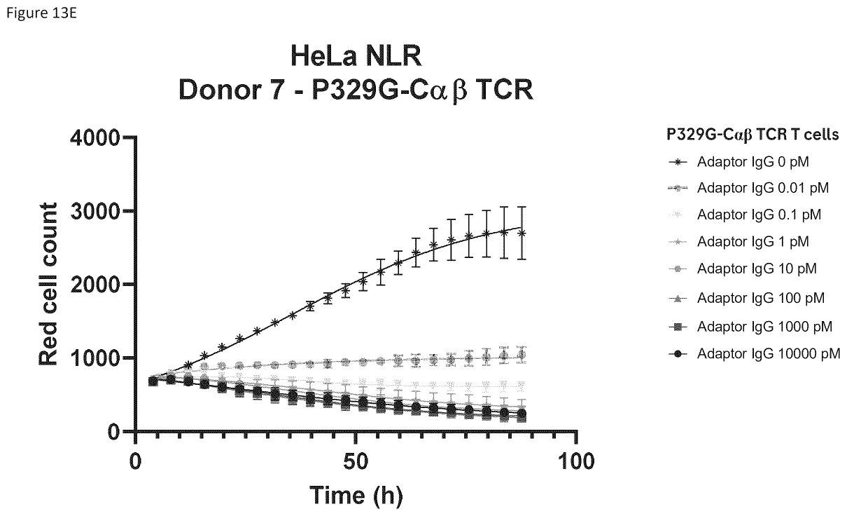

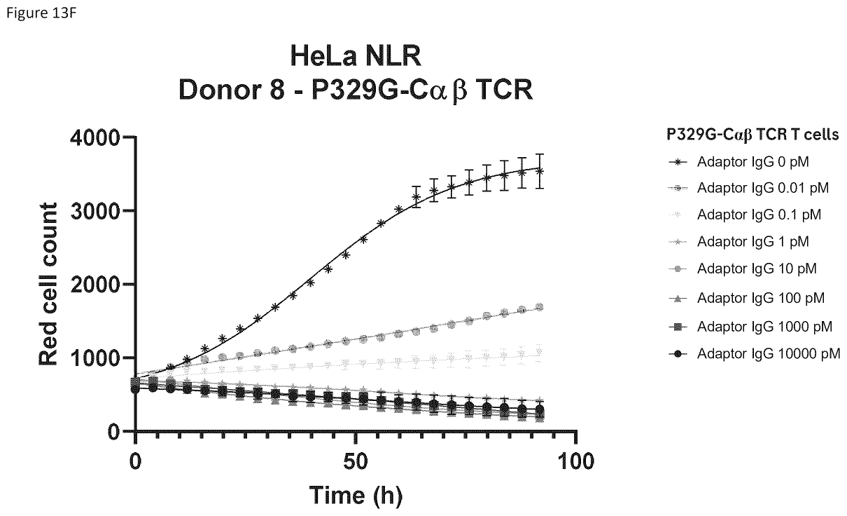

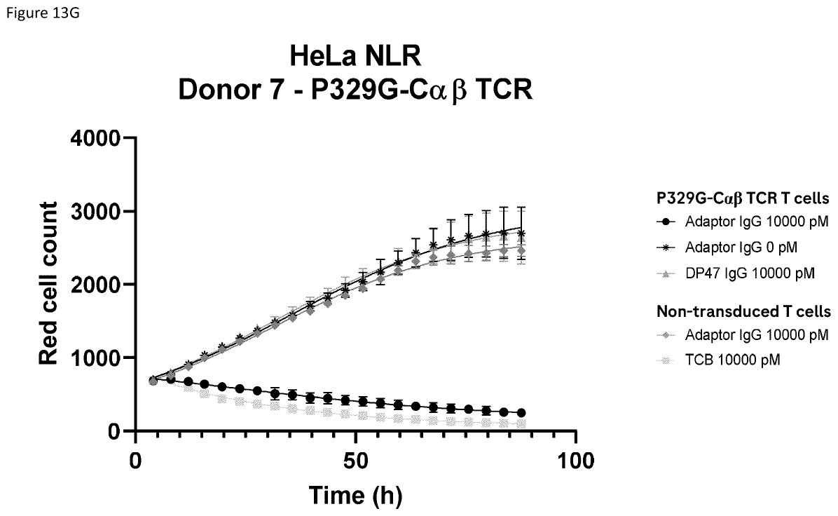

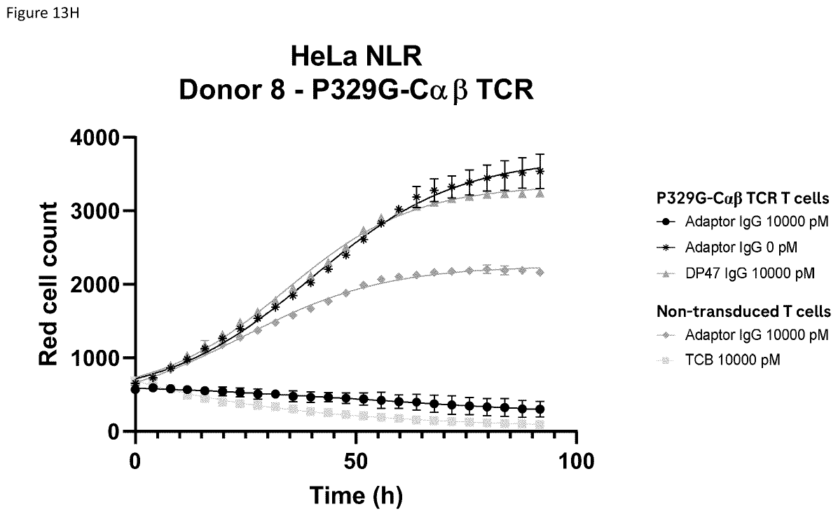

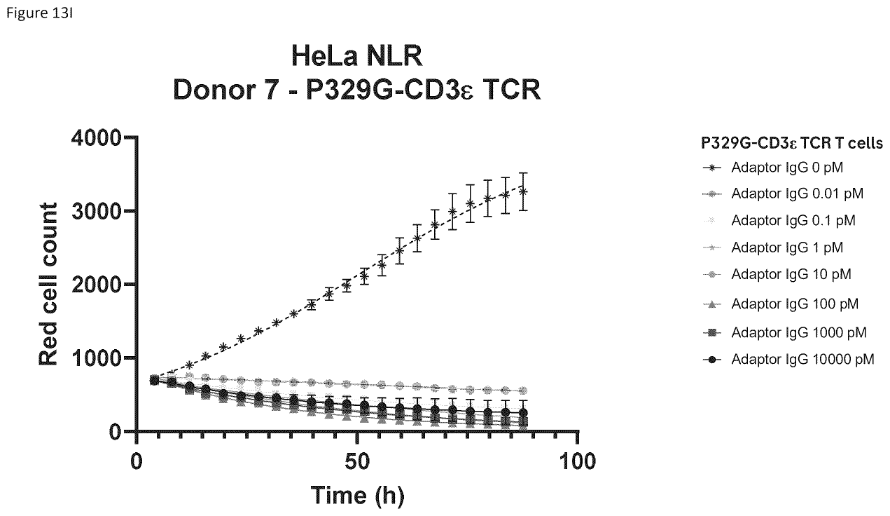

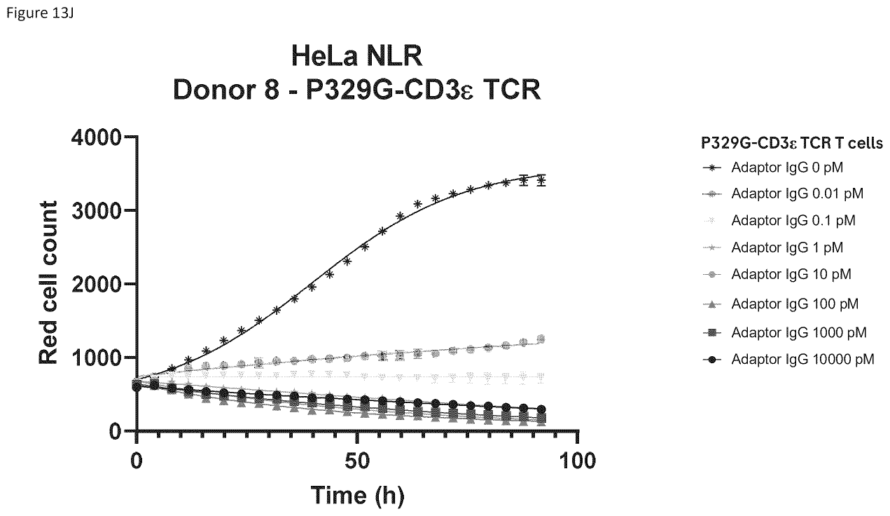

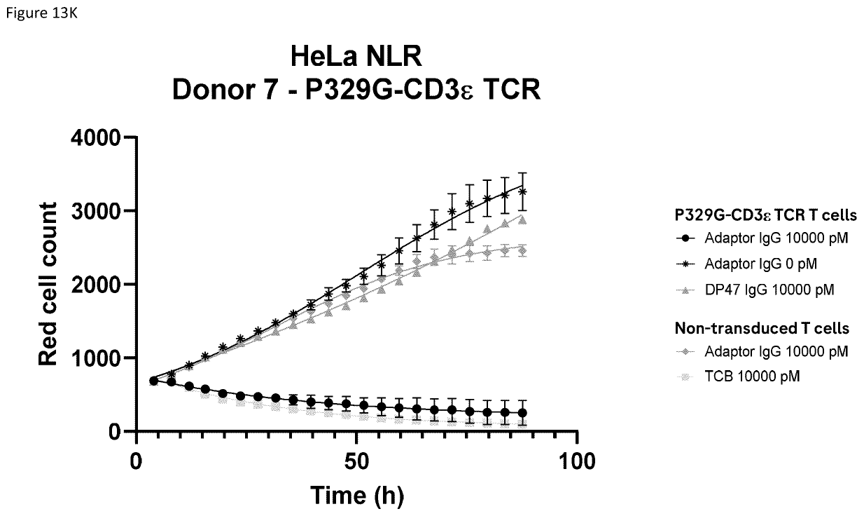

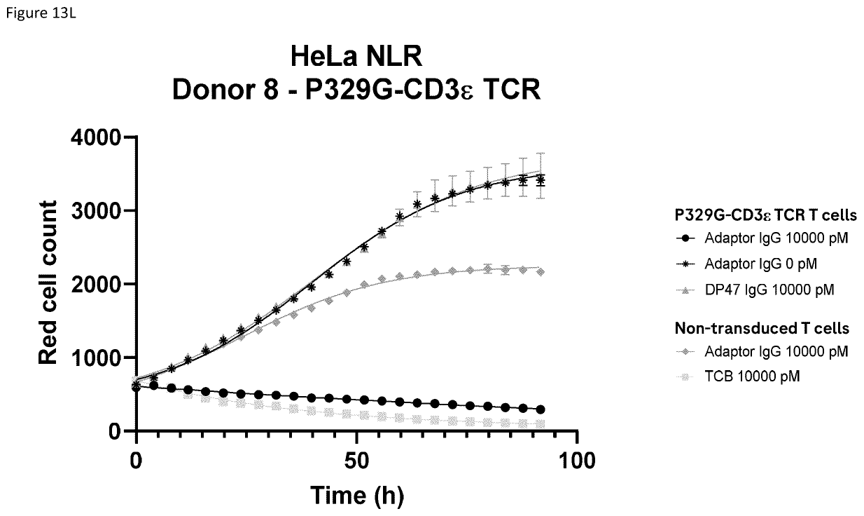

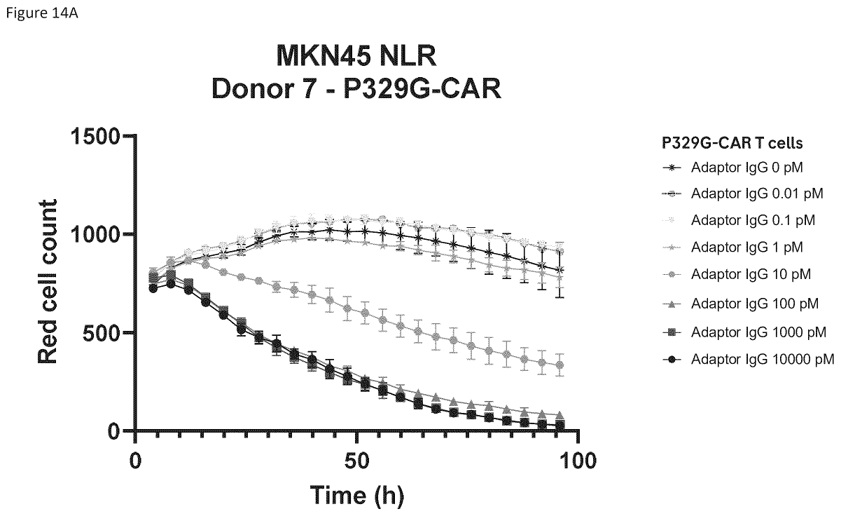

To assess the cytotoxicity of the P329G-CAR, P329G-CD3s and P329G-Cαβ receptors, a killing assay was performed. In order to compare the different constructs, the number of T cells per well was normalised to the same percentage of eGFP+ cells, resulting in 10,000 target cells and 10,000 eGFP+πT cells/well. The killing assay was performed as described above. As an adaptor IgG for FolR1-expressing HeLa-NLR cells, an anti-FolR1 IgG P329G LALA was titrated from 0 pM to 10 nM (1:10). Dose-dependent growth inhibition was observed with all chimeric constructs. In the assay with the P329G-CD3ε T cells, a concentration of 0.1 pM (Donor 7) or 1 pM (Donor 8) was enough to achieve full cancer cell killing (FIGS. 13I and 13J). Similar results were observed with the T cells transduced with the P329G-Cαβ receptor (1 pM for Donors 7 and 8) (FIGS. 13E and 13F). By contrast, P329G-CAR T cells could only inhibit HeLa-NLR cell growth at a concentration of 10 pM (Donor 7) or 100 pM (Donor 8), as evidence by the red cell count, which stayed similar to the starting cell count (0 h) over the course of 4 days (see FIGS. 13A and 13B). As control for non-targeted killing, the non-specific DP47 IgG P329G LALA was used (10 nM). With all of the chimeric receptors, the DP47 IgG P329G LALA did not hinder the cancer cell growth, which was comparable to the control wells containing no adaptor IgG. When the level of cell killing was compared to that observed with a 2+1 anti-FolR1 T cell bispecific (TCB) antibody combined with non-transduced T cells of the same donor, only the P329G-CD3ε and P329G-Cαβ receptors achieved similar results, while the P329G-CAR could not achieve the same level of tumor cell killing (see FIGS. 13C, 13D, 13G, 13H, 13K, 13L).

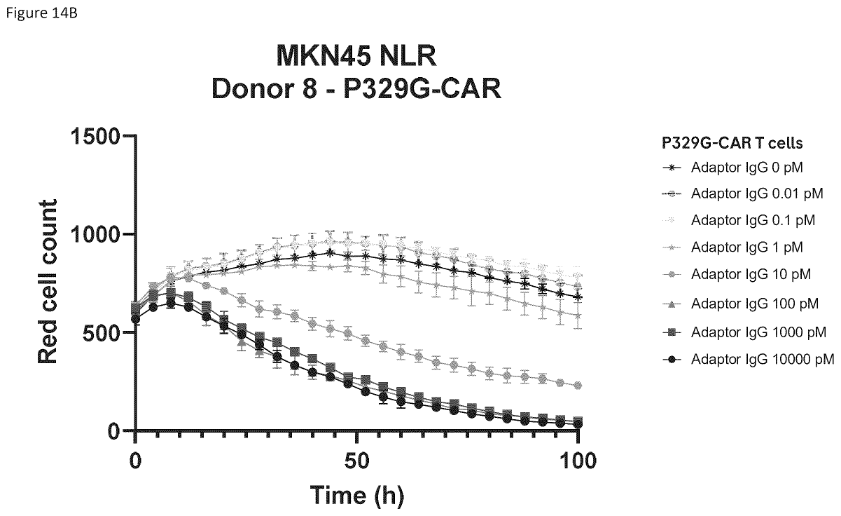

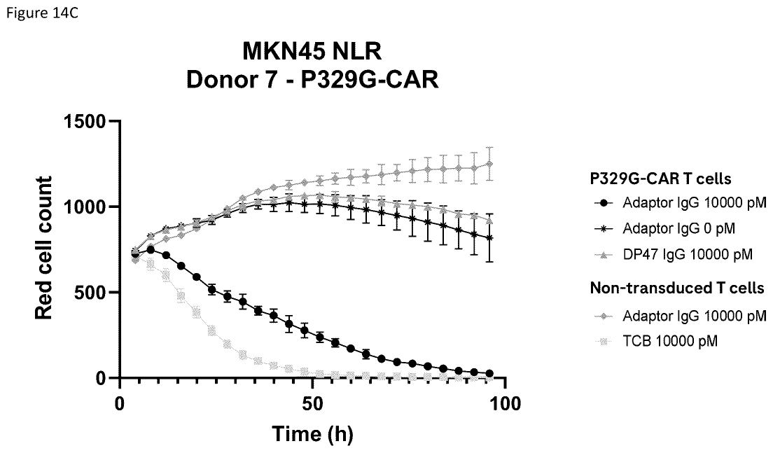

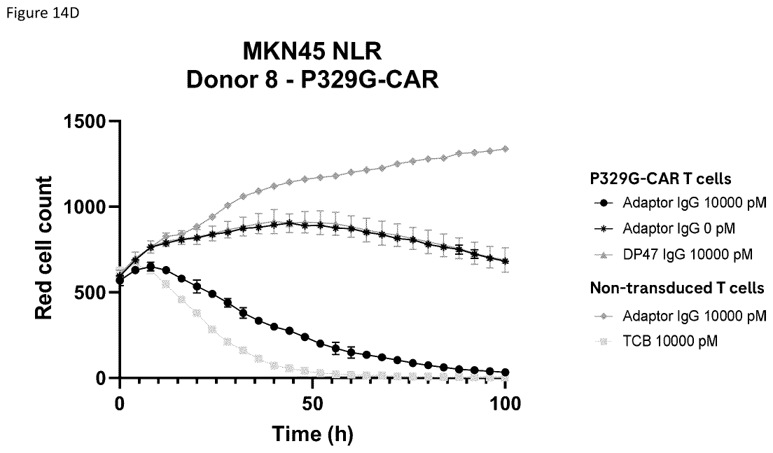

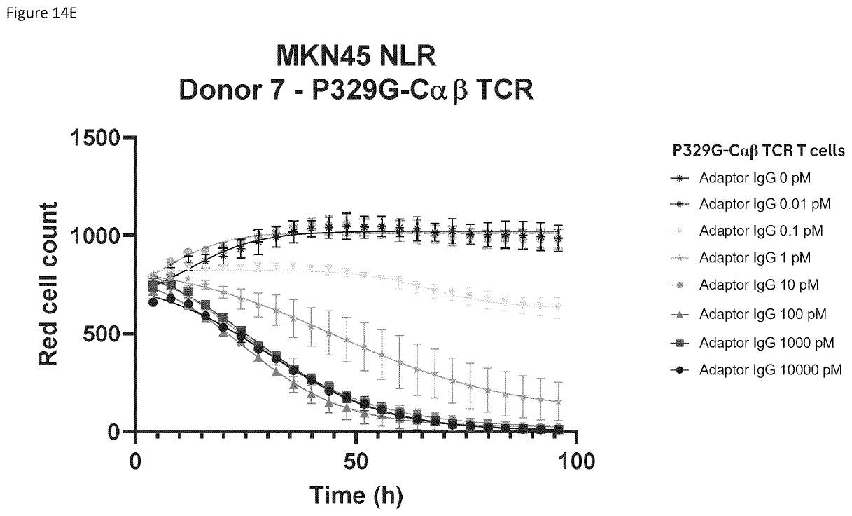

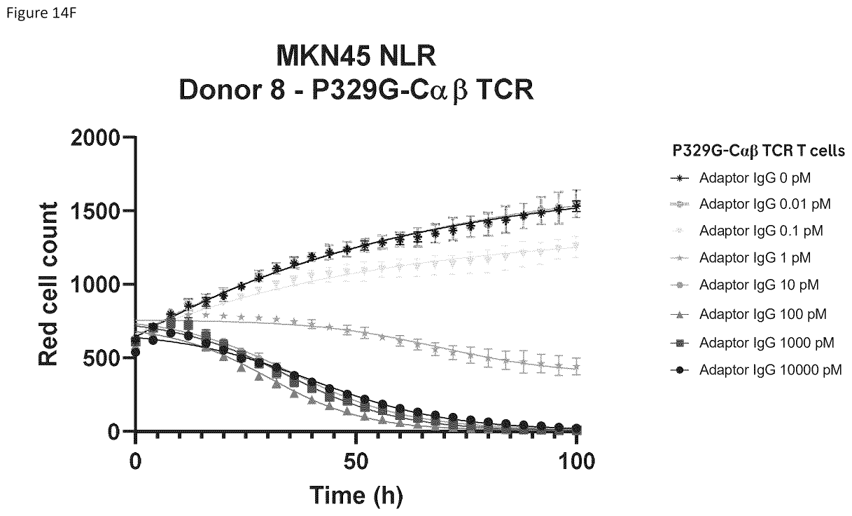

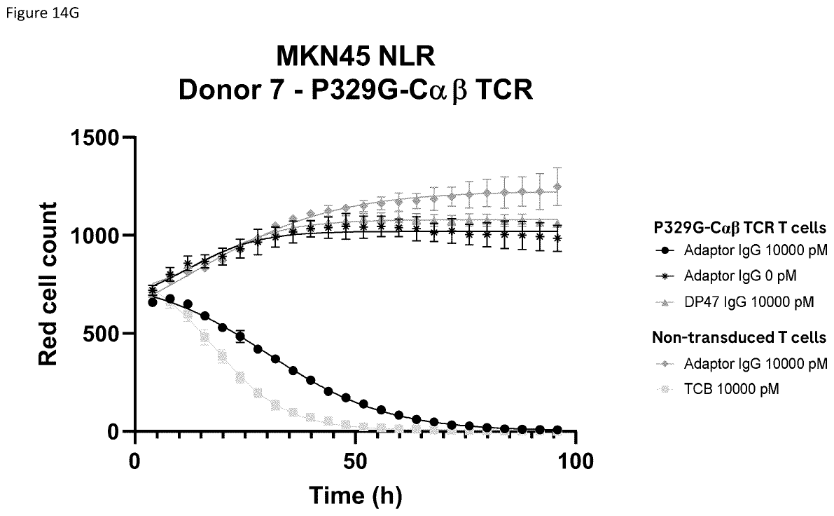

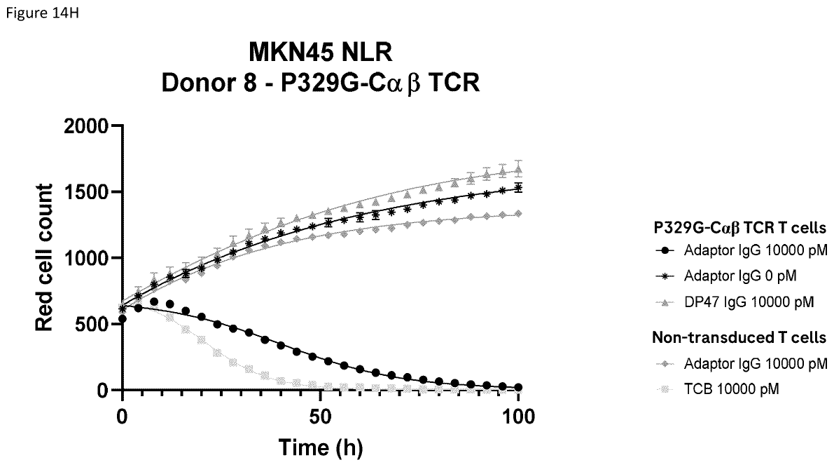

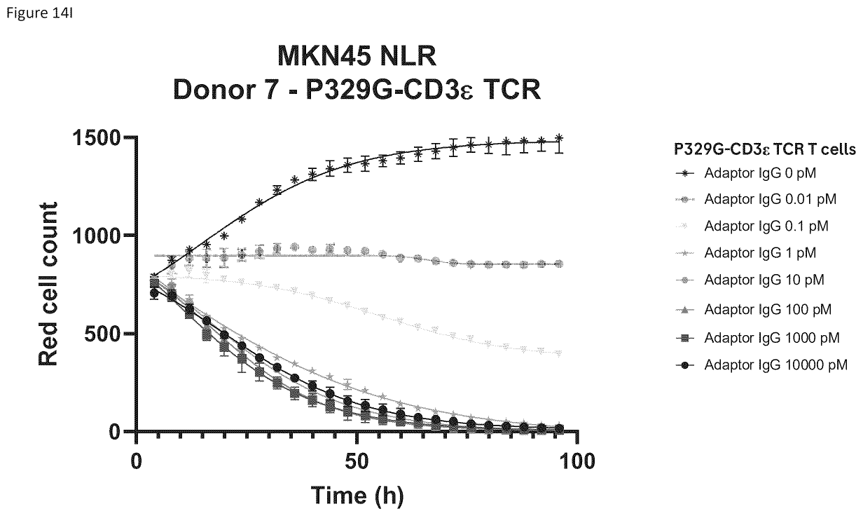

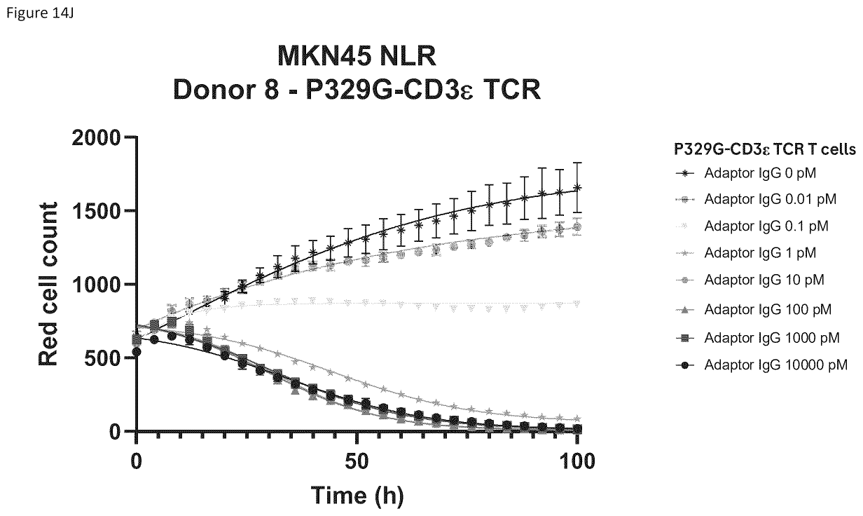

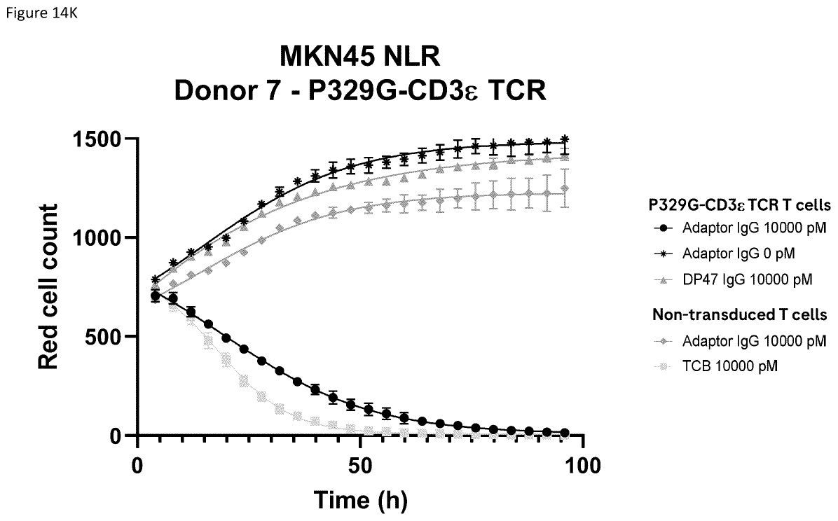

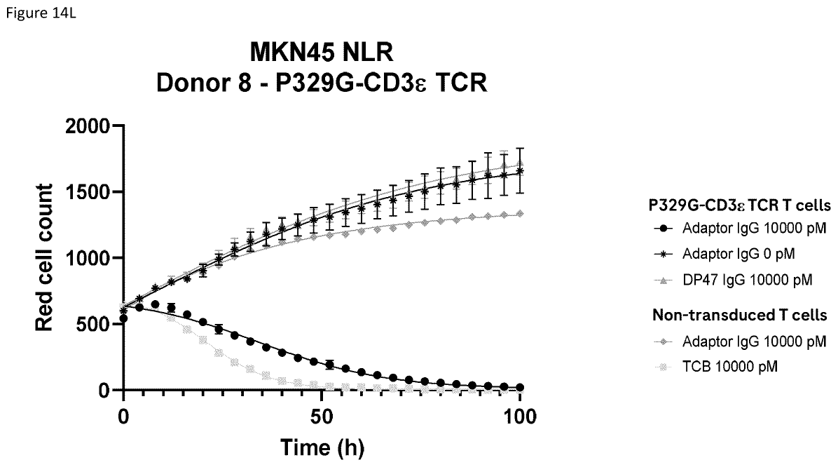

CEACAM5-expressing MKN45-NLR cells were targeted with an anti-CEACAM5 IgG P329G LALA (0 pM-10 nM, (1:10). Dose-dependent growth inhibition was similarly observed with all chimeric constructs. In the assay with the P329G-CD3ε T cells, a concentration of 1 pM (Donors 7 and 8) was sufficient to achieve complete cancer cell killing (FIGS. 14I and 14J). For the conditions with the T cells transduced with the P329G-Cαβ receptor, 10 pM anti-CEACAM5 IgG P329G LALA was required for full cancer cell killing (see FIGS. 14E and 14F) The P329G-CAR T cells achieved a complete cancer cell count reduction with 100 pM of adaptor IgG (FIGS. 14A and 148), although the growth of the MKN45-NLR cells also seemed to be inhibited in the absence of adaptor IgG or DP47 control IgG P329G LALA (FIGS. 14A-14D). This effect was not observed for the chimeric TOR receptors (FIGS. 14G, 14H, 14K, 14L). When the level of cell killing was compared to that observed with anti-CEACAM5 T cell bispecific (TCB) antibody combined with non-transduced T cells of the same donor, all chimeric receptors achieved similar results (see FIGS. 14C, 14D, 14G, 14H, 14K, 14L).

The results lead to the conclusion that the P329G-CD3ε and P329G-Cαβ TOR complex-expressing T cells display high sensitivity in both models, and are able achieve a level of cell killing of target antigen-expressing cells comparable to the level observed with T cell bispecific antibodies, while no cancer cell growth inhibition was observed without adaptor IgG or DP47 IgG P329G LALA. The P329G-CAR T cells were less efficacious in both model systems compared to cells expressing the TOR-based anti-P329G receptors.

1. A recombinant CD3-TCR complex polypeptide, comprising:
(i) an antigen-binding moiety, or a component thereof, wherein the antigen-binding moiety binds to a variant Fc domain having an amino acid sequence comprising at least one amino acid difference relative to a reference Fc domain, to which the antigen-binding moiety does not bind; and
(ii) a CD3-TCR complex association domain having an amino acid sequence derived from a CD3-TCR complex polypeptide.

2. The recombinant CD3-TCR complex polypeptide according to claim 1, wherein the recombinant CD3-TCR complex polypeptide is capable of associating through its CD3-TCR complex association domain with one or more CD3-TCR complex polypeptides to form a CD3-TCR complex.

3. The recombinant CD3-TCR complex polypeptide according to claim 1 or claim 2,
wherein the amino acid sequence derived from a CD3-TCR complex polypeptide is derived from CD3ε, TCRα or TCRβ.

4-9. (canceled)

10. The recombinant CD3-TCR complex polypeptide according to claim 3, wherein the antigen-binding moiety is or comprises an Fv, scFv, Fab, Fab′, Fab′-SH, F(ab′)₂, crossFab, scFab or dAb moiety.

11-12. (canceled)

13. The recombinant CD3-TCR complex polypeptide according to claim 3, wherein the antigen-binding moiety or component thereof is connected at its C-terminus to the N-terminus of the CD3-TCR complex association domain, optionally through a linker sequence.

14. The recombinant CD3-TCR complex polypeptide according to claim 3, wherein the variant Fc domain binds to an Fc receptor with lower affinity than the affinity with which the reference Fc domain binds to the Fc receptor, optionally wherein the Fc receptor is an Fcγ receptor or neonatal Fc receptor (FcRn).

15. The recombinant CD3-TCR complex polypeptide according to claim 3, wherein the variant Fc domain comprises a CH2-CH3 region comprising an amino acid difference at one or more of the following positions, relative to the amino acid sequence of a CH2-CH3 region of the reference Fc domain according to EU numbering: L234, L235, I253, N297, S298, H310, P329, E333, K334 or H435.

16. The recombinant CD3-TCR complex polypeptide according to claim 3, wherein the variant Fc domain comprises a CH2-CH3 region comprising G329 according to EU numbering.

17-22. (canceled)

23. The recombinant CD3-TCR complex polypeptide according to claim 3, wherein the variant Fc domain comprises a CH2-CH3 region comprising A298, A333 and A334 according to EU numbering.

24-29. (canceled)

30. A recombinant CD3-TCR complex polypeptide, comprising:
(i) an antigen-binding moiety, which is an scFv, wherein the antigen-binding moiety binds to a variant Fc domain having an amino acid sequence comprising at least one amino acid difference relative to a reference Fc domain, to which the antigen-binding moiety does not bind, wherein the variant Fc domain comprises a CH2-CH3 region comprising G329 according to EU numbering; and
(ii) a CD3-TCR complex association domain having an amino acid sequence derived from a CD3-TCR complex polypeptide, wherein the amino acid sequence derived from a CD3-TCR complex polypeptide is derived from CD38.

31. A polypeptide complex, comprising:
(a) a first recombinant CD3-TCR complex polypeptide comprising:
(i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
(ii) a CD3-TCR complex association domain having an amino acid sequence derived from a CD3-TCR complex polypeptide; and
(b) a second recombinant CD3-TCR complex polypeptide comprising:
(i) a second component of the antigen-binding moiety of (a) (i); and
(ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form the antigen-binding moiety.

32. The polypeptide complex according to claim 31, wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.

33. The polypeptide complex according to claim 31 or claim 32, wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

34-35. (canceled)

36. A polypeptide complex, comprising:
(a) a first recombinant CD3-TCR complex polypeptide comprising:
(i) a first component of an antigen-binding moiety, wherein the antigen-binding moiety binds to a variant Fc domain having an amino acid sequence comprising at least one amino acid difference relative to a reference Fc domain to which the antigen-binding moiety does not bind, wherein the variant Fc domain comprises a CH2-CH3 region comprising G329 according to EU numbering; and
(ii) a CD3-TCR complex association domain having an amino acid sequence derived from a CD3-TCR complex polypeptide; and
(b) a second recombinant CD3-TCR complex polypeptide comprising:
(i) a second component of the antigen-binding moiety of (a) (i); and
(ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form the antigen-binding moiety;
wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain;
and wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

37. A CD3-TCR polypeptide complex, wherein the CD3-TCR polypeptide complex comprises a recombinant CD3-TCR complex polypeptide according to claim 1, or a polypeptide complex according to claim 31.

38. A composite polypeptide comprising:
(a) an amino acid sequence encoding a first recombinant CD3-TCR complex polypeptide comprising:
(i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
(ii) a CD3-TCR complex association domain having an amino acid sequence derived from a CD3-TCR complex polypeptide; and
(b) an amino acid sequence encoding a second recombinant CD3-TCR complex polypeptide comprising:
(i) a second component of the antigen-binding moiety of (a) (i); and
(ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form a CD3-TCR complex comprising the antigen-binding moiety;
and wherein the composite polypeptide further comprises a cleavage site between the amino acid sequences of (a) and (b).

39. The composite polypeptide according to claim 38, wherein the first component of an antigen-binding moiety is or comprises the heavy chain variable (VH) region of an antibody that binds to the variant Fc domain, and wherein the second component of the antigen-binding moiety is or comprises the light chain variable (VL) region of the antibody that binds to the variant Fc domain.

40. The composite polypeptide according to claim 38 or claim 39, wherein:
(i) the CD3-TCR complex association domain of the first recombinant CD3-TCR complex polypeptide is derived from TCRα, and the CD3-TCR complex association domain of the second recombinant CD3-TCR complex polypeptide is derived from TCRβ, or
(ii) the CD3-TCR complex association domain of the first recombinant CD3-TCR complex polypeptide is derived from TCRβ, and the CD3-TCR complex association domain of the second recombinant CD3-TCR complex polypeptide is derived from TCRα.

41-43. (canceled)

44. A nucleic acid, or a plurality of nucleic acids, encoding:
(a) a first recombinant CD3-TCR complex polypeptide comprising:
(i) a first component of an antigen-binding moiety, wherein the antigen-binding moiety binds to a variant Fc domain having an amino acid sequence comprising at least one amino acid difference relative to a reference Fc domain to which the antigen-binding moiety does not bind; and
(ii) a CD3-TCR complex association domain having an amino acid sequence derived from a CD3-TCR complex polypeptide; and
(b) a second recombinant CD3-TCR complex polypeptide comprising:
(i) a second component of the antigen-binding moiety of (a) (i); and
(ii) a CD3-TCR complex association domain having an amino acid sequence derived from a CD3-TCR complex polypeptide;
wherein the first and second recombinant CD3-TCR complex polypeptides are capable of associating through their CD3-TCR complex association domains to form a CD3-TCR complex comprising the antigen-binding moiety.

45. An expression vector, or a plurality of expression vectors, comprising a nucleic acid or a plurality of nucleic acids according to claim 43 of claim 44.

46. A cell comprising a recombinant CD3-TCR complex polypeptide according to claim 1, a polypeptide complex according to claim 31, a CD3-TCR polypeptide complex according to claim 15, a composite polypeptide according to claim 38, a nucleic acid or a plurality of nucleic acids according to claim 44, or an expression vector or a plurality of expression vectors according to claim 45.

47. A pharmaceutical composition comprising a cell according to claim 46.

48. A cell according to claim 46, or a pharmaceutical composition according to claim 47, for use in a method of medical treatment or prophylaxis.

49. A cell according to claim 46, or a pharmaceutical composition according to claim 47,
for use in a method of treating or preventing a disease in which cells comprising or expressing a target antigen are pathologically-implicated, wherein the method comprises administering the cell or pharmaceutical composition to a subject to which an antigen-binding molecule has been or is to be administered;
wherein the antigen-binding molecule comprises: (a) an antigen-binding domain that binds to the target antigen, and (b) a variant Fc domain having an amino acid sequence comprising at least one amino acid difference relative to a reference Fc domain;
and wherein the antigen-binding moiety of the recombinant CD3-TCR complex polypeptide, polypeptide complex, CD3-TCR polypeptide complex or composite polypeptide comprised in the cell of claim 46 or the cell comprised in the pharmaceutical composition of claim 47, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell of claim 46, or comprised in the cell comprised in the pharmaceutical composition of claim 47, binds to the variant Fc domain.

50. A method for depleting or killing cells comprising or expressing a target antigen, comprising contacting cells comprising/expressing a target antigen with:
(i) a cell according to claim 46, or a pharmaceutical composition according to claim 47; and
(ii) an antigen-binding molecule comprising: (a) an antigen-binding domain that binds to the target antigen, and (b) a variant Fc domain having an amino acid sequence comprising at least one amino acid difference relative to a reference Fc domain;
wherein the antigen-binding moiety of the recombinant CD3-TCR complex polypeptide, polypeptide complex, CD3-TCR polypeptide complex or composite polypeptide comprised in the cell of claim 46 or the cell comprised in the pharmaceutical composition of claim 47, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell of claim 46, or comprised in the cell comprised in the pharmaceutical composition of claim 47, binds to the variant Fc domain.

51. A kit, comprising:
(i) a cell according to claim 46, or a pharmaceutical composition according to claim 47; and
(ii) an antigen-binding molecule comprising: (a) an antigen-binding domain that binds to a target antigen, and (b) a variant Fc domain having an amino acid sequence comprising at least one amino acid difference relative to a reference Fc domain;
wherein the antigen-binding moiety of the recombinant CD3-TCR complex polypeptide, polypeptide complex, CD3-TCR polypeptide complex or composite polypeptide comprised in the cell of claim 46 or the cell comprised in the pharmaceutical composition of claim 47, or the antigen-binding moiety of the CD3-TCR complex polypeptide, polypeptide complex, CD3-TCR polypeptide complex or composite polypeptide encoded by the nucleic acid or plurality thereof or the expression vector or plurality thereof comprised in the cell of claim 46, or comprised in the cell comprised in the pharmaceutical composition of claim 47, binds to the variant Fc domain.

52. A kit, comprising:
(i) a nucleic acid or a plurality of nucleic acids according to claim 43 of claim 44, or an expression vector or a plurality of expression vectors according to claim 45; and
(ii) an antigen-binding molecule comprising: (a) an antigen-binding domain that binds to a target antigen, and (b) a variant Fc domain having an amino acid sequence comprising at least one amino acid difference relative to a reference Fc domain;
wherein the antigen-binding moiety of the CD3-TCR complex polypeptide, polypeptide complex, CD3-TCR polypeptide complex or composite polypeptide encoded by the nucleic acid or plurality thereof according to claim 43 or claim 44, or encoded by the nucleic acid or plurality thereof comprised in the expression vector or plurality thereof according to claim 45, binds to the variant Fc domain.
